(19) United States
(12) Patent Application Publication
(10) Pub. No.: US 2022/0121972 A9
(43) Pub. Date: Apr. 21, 2022

(54) CONTROL SYSTEM

(71) Applicant: Margot STUBBS, Sydney (AU)

(72) Inventor: Margot STUBBS, Sydney (AU)

(21) Appl. No.: 17/025,207

(22) Filed: Sep. 18, 2020

Related U.S. Application Data
(63) Continuation-in-part of application No. 14/769,762, filed on Aug. 21, 2015, now abandoned.

(30) Foreign Application Priority Data
Feb. 25, 2013 (AU) 2013900631

(51) Int. Cl. G06N 5/04; G06Q 10/10; G06Q 30/00; G06Q 50/18; G16H 50/20; G06Q 40/08; G06F 16/22; G06F 3/0482; G06F 3/0484

(52) U.S. Cl. CPC G06N 5/045; G06Q 10/10; G06Q 30/018; G06Q 50/18; G16H 40/20; G06Q 40/08; G06F 16/2237; G06F 3/0482; G06F 3/04845; G16H 50/20

(57) ABSTRACT

Broadly there is provided a control system in the form of a decision support system for a matter; the matter of indeterminate duration; and during the course of the matter third-party inputs are received consecutively and periodically for the duration of the matter; the decision support system allowing replication of an expert's approach to a complex problem; said system comprising:
At least a primary clipboard into which data is entered by the user into one or more data windows;
a database of public information comprising data modules for selective input into a Data Matrix relevant to the matter;
preconfigured specialist algorithms assembled by an expert knowledgeable in decision-making in relation to the matter; the specialist algorithms contained in a Decision Support mode of the decision support system;
an input system by which the third-party inputs comprising each item of data presented to the User as a matter progresses are consecutively and periodically assembled by a user at first instance on to the primary clipboard;
an input system by which the user selects portions of the public information and portions of the preconfigured specialist algorithms and assembles them at first instance onto the primary clipboard;
and wherein the selections and assembly made in response to all items of data received during the matter are entered onto the primary clipboard;
thereby to assemble a primary clipboard of public information portions, specialist algorithm portions and third-party input portions in a continuous format so as to present a chronological record of all items of data and decisions made by the user as the matter progresses with the assistance of the specialist algorithms and data modules relevant to the matter and as selected by the user as the matter progresses.

### TECHNICAL FIELD

This invention concerns a control system that makes use of a computer-user interface to control a process having plural different stages of operation.

### BACKGROUND ART

Control systems are widely used to control processes, from washing machines to steel rolling mills. Some processes are fully automated using computers, whereas others require some human input. For instance, the control of each cycle of an internal combustion engine may be fully automated from fuel injection to the processing of the exhaust gases. However, more complex processes require human intervention, for instance large complex chemical processing plants or power stations have sophisticated control rooms where people oversee and regulate operations.

Any discussion of documents, acts, materials, devices, articles or the like which has been included in the present specification is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

A control system for control of a multi-stage process, the system comprising:

a computer memory that stores:
a decision tree having a network of plural interconnected nodes, each of which represents a stage of the multi-stage process being controlled.
historical patterns of entries made by users between first and second nodes in the network
plural different types of resource information for each stage of the multi stage process.

A computer input device to receive commands from a user:

To select a node in the decision tree.
To enter data into a selected node.

A computer processor operable to interpret computer program code, resource information and user input.

A computer screen that, in use, displays a user interface to a user including

At least part of the decision tree including the selected node and at least one generation of downstream nodes;
And in another mode at least part of the decision tree including the selected node

### Wherein in use:

Each node requires an input to satisfy a specified criterion.

In response to input into a selected node that satisfies a specified criterion, one of the downstream nodes is automatically selected and the computer screen is updated.

And input is made by a user either directly into the currently selected node, or automatically into a string of one or more successive nodes as determined by a selected historical pattern of entries.

Input into nodes includes input into linked resources such as a Data Window linked to the node

Such a control system permits a user to select any stage of the process, to check resource information relevant to that stage and to ensure correct data is entered for the corresponding node in the decision tree, so that the stage is properly before the next stage is begun.

The user interface may include a persistent menu identifying the plural different types of resource information.

The computer processor may operate to cause the resource information accessible via the persistent menu to automatically change, in a context aware manner, each time the user selects a different node in the decision tree.

The control system may include a navigation tool that allows backtracking to any earlier node of the decision tree.

The control system may include a search function to permit search of the nodes of the decision tree

The control system may further include a data entry port to receive updated resource information.

The control system may further include a data entry port to receive data relating to the stages of the process. Such data could be provided, for instance from sensors associated with corresponding stages of the process

The computer memory of the control system may store status information about one or more of the stages of the process; for instance ‘processing complete’ or ‘process incomplete’ status.

The computer memory of the control system may store user input provided at one or more nodes so that the same input is available for consideration or reuse at a later time.

Optional features of any aspect of the invention described here are also optional features of the other aspects of the invention described here.

According to one broad form of the invention there is provided a decision support system for a matter; the matter of indeterminate duration; and during the course of the matter third-party inputs are received consecutively and periodically for the duration of the matter; The decision support system allowing replication of an expert's approach to a complex problem; said system comprising:

a primary clipboard into which data is entered by the user into one or more data windows;

generated multiple sub-clipboards from the primary clipboard and wherein

all sub-clipboards contain plural data windows;

a database of public information comprising data modules for selective input into a Data Matrix relevant to the matter;

preconfigured specialist algorithms assembled by an expert knowledgeable in decision-making in relation to the matter; the specialist algorithms contained in a Decision Support mode of the decision support system;

an input system by which the third-party inputs comprising each item of data presented to the User as a matter progresses are consecutively and periodically assembled by a user at first instance on to the primary clipboard;

an input system by which the user selects portions of the public information and portions of the preconfigured specialist algorithms and assembles them at first instance onto the primary clipboard;

and wherein the selections and assembly made in response to all items of data received during the matter are entered onto the primary clipboard;

thereby to assemble a primary clipboard of public information portions, specialist algorithm portions and third-party input portions (define) in a continuous, timestamp format so as to present a chronological record of all items of data and decisions made by the user as the matter progresses with the assistance of the specialist algorithms and data modules relevant to the matter and as selected by the user as the matter progresses; and wherein

the items of the data on the primary clipboard are

tagged by the user with tags during the course of the matter or by the User when the user is preparing their decision or report;

and wherein the tag is a categorising device by which an item of data is characterised according to a criterion selected by the user from the Decision Support System;

and wherein Every item of data entered into the Primary Clipboard is assembled by the User as the matter progresses; every item of data able to be edited by way of both imported and user generated multiple tags to enable the content of the Primary Clipboard to be filtered into the Sub-Clipboards in accordance with the tags selected by the User; and

wherein the User generates a specialist display of the system in which the User may populate a Report (in the instant legal application, a Judgment) which display presents the selected decision support algorithm, as well as a sub-clipboard into which the User may select any sub clipboard containing the fact findings the User has made which are relevant to the selected decision support algorithm; and which display allows the user to assemble their Report (in the In-Use example a Judgment) by copying relevant data windows (or data from data window) from the decision support algorithm into the Report (or Judgment) Clipboard and then to populate that data window in the Report or Judgment Clipboard with data from the sub-clipboard in the adjacent display of the program containing the Users findings of fact, and which allows the User to so work sequentially though the topics from the expert decision algorithm on which the User must make an outcome decision; and thereby generating a Report (or Judgment) as the ultimate outcome of the process of applying the expert decision topics (in the in-use example the law) to the facts across the matter, being those facts identified as relevant to every decision in the decision algorithm through the assistance provided in other models of operation of the system.

Preferably, the decision support system as described above incorporating further Plural integrated tagging functionality including colour tagging data windows, linking data windows to documents and other media saved into the decision system and generation of a chronology of all items of significance in the matter.

Preferably, an item of data may be tagged with more than one tag thereby categorising the item of data according to more than one criterion.

Preferably, all items of data which are tagged by a tag according to a particular criterion may be assembled by the system, on command of the user, into a sub clipboard or independent data window in a compilation mode of the system's operation.

Preferably, system extracting from the field of data collected by the user onto the Primary Clipboard during the Matter into Sub-Clipboards containing all data windows related to the Users selected tagging criteria.

Preferably, the system allows a user to select one or more sub clipboards in a particular visual display and from those sources open and compare multiple data windows to view them at the same time then each independent data window displaying links to related documents which a user may also bring into that specialist display; items of data tagged according to a different criterion so that the user is enabled to select one or more sub clipboards in a particular way to view more than one independent data window from multiple Sub Clipboards at the same time; each independent data window displaying items of data tagged according to a different criterion in each independent data window.

Preferably, the matter relates to a legal investigation in the form of a court case.

Preferably, the matter relates to an investigation process relating to a medical investigation.

Preferably, the matter relates to a scientific investigation.

Preferably, the matter relates to an insurance investigation.

Preferably, a user may invoke steps in the specialist algorithm in order from less complex to more complex-only to the level needed for the particular matter.

Preferably, the criterion for a tag may be determined by the user or automatically imported, as part of a specialist decision algorithm which is imported into the decision system by the user.

Preferably, Tags may be imported by the user.

Preferably, the system comprises the primary clipboard which allows all data relevant to the matter from plural third-party sources from the system in data matrix and decision support mode to be input into the primary clipboard through a system of data windows which permits a user a powerful system to manage, link, and compare all of the data on the primary clipboard in any configuration selected by the user utilising the tagging function in each data window.

Preferably, the system allows an expert decision algorithm to contain tags which will be automatically incorporated into the tagging functionality of the system which relates to the plural factual matters the user has to decide to correctly apply the law and wherein the user will also be able to add their own plural tags as they may consider appropriate.

Preferably, the system has colour functionality for each data window.

Preferably, the user may attach a colour to each data window to operate as a reminder to the user when reviewing the items in the primary clipboard in accordance with a user selected criteria by reference to the colours.

Preferably, the system colours window according to a reminder idea—e.g. red means come back to this later; red indicates concern about legal principle; blue means you accept this witness evidence.

Preferably, the data window in the selection of the chronology button permits the user to incorporate the event in the data window into a timeline which will be generated during the conduct of the matter.

Preferably, the chronology will be available to the user in every mode of the system.

Preferably, there are at least 6 visual interfaces/modes of operation comprising Matters, Data Matrix, Decision Support, Compilation, Judgment and Chronology.

Preferably, the specialist algorithms are assembled by an expert knowledgeable in decision-making in relation to the matter contained in Decision Support mode of the Program.

Preferably, the system is modular and permits the user to generate and export from the system their own data matrix modules and their own decision support modules to be available for incorporation by other users of the system into their respective decision support algorithm lists or their data matrix, of public information.

### BRIEF DESCRIPTION OF THE DRAWINGS

Examples of the invention will now be described with reference to the accompanying drawings, in which:

FIG. 1 illustrates a computer system for control of a multi-stage process.

FIG. 2 illustrates a method for controlling a multi-stage process as performed by processor.

FIG. 3 illustrates a decision tree.

FIG. 4 illustrate a user interface.

FIG. 5 illustrates a user interface after selecting a Topic Node.

FIG. 6 illustrates a user interface resulting from selecting a primary node.

FIG. 7 illustrates a user interface after the decision maker then selects the sub node topic.

FIG. 8 illustrates a data matrix.

FIG. 9 illustrates a user interface where the Decision Maker selects a node in the decision tree.

FIG. 10 illustrates another user interface.

FIG. 11 illustrates yet another user interface including a generated Data Window.

FIG. 12 illustrates yet another user interface representing a next step.

FIG. 13 illustrates another user interface 1300 where the decision maker selects the primary node topic.

FIGS. 14-202 illustrate user interfaces illustrating the control systems for the multi-stage processes according to a first and further embodiment of the invention.

FIG. 14 illustrates the user interface.

FIG. 15 illustrates the decision support system.

FIG. 16 illustrates when the program is activated and opened, the decision maker is taken to the Portal.

FIG. 17 illustrates a visual display of the decision support system opened in Data Matrix mode.

FIG. 18 shows the Data Matrix mode of a Judicial Decision Support System.

FIG. 19A shows the decision maker having selecting a Tier 1 node.

FIG. 19B shows the sub-nodes are in the computer system 100 shown as Tier 2 nodes.

FIGS. 20 and 21 illustrates the use of the scroll bar by the decision maker to navigate through all of the nodes within the primary node Tier of decision system.

FIG. 22 illustrates that the visual display may be resized by the decision maker to any size to enable more accessible visual access to the interface to the program.

FIG. 23 illustrates a computer screen visual display after the decision maker selects a node from Tier 1.

FIG. 24 illustrates the visual display of the downstream (Tier 2) nodes displayed by computer system.

FIG. 25 illustrates that every node has at least one data window.

FIGS. 26 and 27 illustrate that the number of Tier 2 nodes are not restricted by the size of the visual display.

FIG. 28 illustrates a decision maker selecting a further sub or downstream node in relation to the “Bail” node.

FIG. 29 illustrates that when the decision maker selects the Tier 2 node.

FIG. 30 illustrates the decision maker selecting the node “Application of s. 32”.

FIG. 31 illustrates computer system 100 updating the screen display when activated by user.

FIG. 32 illustrates the user selecting the node in respect of which they require further assistance.

FIG. 33 illustrates computer system displays the legal topic nodes.

FIG. 34 illustrates the decision maker selecting the topic node Bail from Tier 1 nodes.

FIG. 35 illustrates that the selection of the Bail node within computer system brings into the visual display the sub-topic nodes relevant to the node Bail.

FIG. 36 illustrates the decision maker selecting the node.

FIG. 37 illustrates the decision maker selecting the first Tier 3 node.

FIGS. 38 and 39 illustrate the decision maker may use the scroll bar in the Data Windows column to bring into the visual display of the computer screen.

FIG. 40 illustrates how a Data Window may be opened by the decision maker by selecting it.

FIG. 41 illustrates the location of the scroll bar function.

FIGS. 42 and 43 illustrates the decision maker re-sizing the Data Window.

FIGS. 44 and 45 illustrates the decision maker using the scroll bar facility to scroll through the data contents of a resized Data Window.

FIG. 46 illustrates a decision maker selecting the Add to Clipboard button and copying the Data Window onto the Clipboard.

FIG. 47 illustrates a decision maker selecting the Data Window “Prosecution Commences the Application” by activating the Add to Clipboard Button.

FIG. 48 illustrates the Data Window now added to the Clipboard.

FIG. 49 illustrates the decision maker re-sizing a Data Window from the Data Window column.

FIG. 50 illustrates a decision maker using the cut and paste function to select part of the data in that Data Window.

FIG. 51 illustrates the decision maker using the Add to Clipboard function to copy an empty Data Window onto the Clipboard.

FIG. 52 illustrates the decision maker using the paste function to copy the selected part of the Data Window in the Data Windows Column to the empty Data Window in the Clipboard.

FIGS. 53-56 illustrate the decision maker is accessing the Data Matrix in relation to the Tier 1 node of General Orders, then the Tier 2 node Compensation and Restitution, and then the Tier 3 node Time for Making Order.

FIGS. 57 and 58 and 59 illustrates the decision maker opening the Data Window, “Sample Order”, resizing that window to read its content clearly, then uses the Add to Clipboard button to copy that Data Window onto the Clipboard.

FIG. 60 illustrates the decision maker scrolling through the nodes in Tier 1 of the decision system to the node “Commonly Occurring Penalties”.

FIG. 61 illustrates the node “Commonly Occurring Penalties” being selected by the decision maker.

FIG. 62 illustrates the visual display when the node “Commonly Occurring Penalties” in FIG. 61 is selected.

FIG. 63 illustrates the decision maker selecting the node “Prescribed Content of Alcohol Offences”.

FIG. 64 illustrates the downstream or sub-nodes relevant to the node “Prescribed Content of Alcohol Offences” shown as Tier 3 topic nodes.

FIGS. 65 and 66 shows the decision maker selecting the node “High Range PCA” and the Data Window relevant to that node appears in Data Windows.

FIGS. 67 and 68 illustrate the decision maker selecting the “Guideline Judgment” node from the Tier 3 nodes and using the Copy to Clipboard function, copy that Data Window to the Clipboard.

FIGS. 69-72, show a decision maker selecting Tier 1, Commonly Occurring Penalties, then Serious Driving Offences, then Negligent Driving and the specific offences and penalties relevant to Negligent Driving causing Grievous Bodily Harm.

FIGS. 73, 74 and 75 illustrate the decision maker using the search function in relation to the term “assault”, selecting the appropriate node, and computer system then updating the visual display to take user to the selected node.

FIGS. 76 and 77 illustrates the decision maker using the navigation bar to retrace the steps the decision maker has taken though computer system.

FIG. 78 illustrates the visual display when the decision maker has activated Decision Support mode.

FIG. 79 illustrates computer system 100 when Decision Assistance mode has been activated.

FIG. 80 illustrates the use of one data window in Decision Assistance mode but computer system 100 may use any number of Data Windows linked to one node.

FIG. 81 illustrates the Clipboard.

FIG. 82 illustrates the windows in the visual display of computer system.

FIG. 83A shows a decision maker selecting the node “Criminal Offences” in Window 1.

FIG. 83B illustrates the computer system then causes all sub-nodes or downstream nodes relevant to the node “Criminal Offences” to appear in Window 2.

FIG. 83C illustrates the node is selected and the decision steps applicable to that offence are then brought into the visual display by computer system 100 in Window 3.

FIG. 84 illustrates the decision maker selects the first step or node in the expert decision chain, “Physical Elements”.

FIG. 85 illustrates the selection of FIG. 84 causes the downstream nodes relevant to “Physical Elements” to appear in the visual display.

FIGS. 86A and 86B illustrate the computer systems.

FIG. 87 illustrates the decision maker opening Window 1 on Decision Assistance mode and selects Sentencing—NSW.

FIG. 88 illustrates the sub-nodes relevant to Sentencing.

FIG. 89A illustrates the decision maker selecting Sentencing Template.

FIG. 89B illustrates the steps involved in a judicial decision maker approaching sentencing an offender then appear in Window 3.

FIG. 90 illustrates the selection of the node Aggravating Circumstances will cause computer system 100 to update the visual display and bring into the visual display the relevant statutory principle.

FIG. 91 illustrates the decision maker selecting the node “Aggravating Factors”.

FIG. 92 illustrates the sub-node topics relevant to Aggravating Factors is brought into Window 6.

FIG. 93 illustrates the decision maker then selects the particular matter relevant to the circumstances before them for sentence.

FIG. 94 illustrates the Data Window relevant to the law applicable to setting the appropriate sentence when the victim of the offence is an emergency worker then appears in its related Data Window.

FIG. 95 shows the decision maker may move the entire Data Window to the clipboard by activating the “Add to Clipboard” button.

FIG. 96 shows the decision maker re-sizing that Data Window and editing its content.

FIG. 97 illustrates the decision maker may wish to obtain assistance in solving an Evidence problem that may arise in the course of a hearing.

FIG. 98 illustrates user opening computer system in Decision Assistance mode.

FIG. 99 illustrates user selecting Judgment Templates from Window 1.

FIG. 100 illustrates user selecting Judgment Template—Criminal from Window 2.

FIG. 101 illustrates uses commencing the hearing by selecting Case History from Window 3, the decision steps in the conduct of a criminal trial.

FIG. 102 Illustrates user selecting “Add to Clipboard” function in relation to the Case History Data Window.

FIG. 103 illustrates user opening Data Window “Case History” and adding the information.

FIG. 104 illustrates user selecting the next step in the decision chain in Window 3, Charges and Particulars.

FIG. 105 illustrates the user selecting the “Add to Clipboard” function and copying this Data Window to the Clipboard.

FIG. 106 illustrates user entering the charge and the particulars into the Data Window.

FIG. 107 illustrates user selecting Data Matrix mode.

FIG. 108 illustrates user selecting the node “Commonly Occurring Penalties” at Tier 1.

FIG. 109 illustrates user then selecting the Criminal Offences node at Tier 2.

FIG. 110A “Assaults” is brought into the visual display by computer system 100 and is selected by user.

FIG. 110B then illustrates the downstream nodes related to the node “Assault”.

FIGS. 111A and 111B illustrate user selecting the Data Window Assault Occasioning Actual Bodily Harm and copying it to the Clipboard.

FIG. 112 illustrates the judge then selecting decision Assistance mode.

FIG. 113 illustrates the how the Data Window selected in Data Matrix mode retains its position in the Data Windows in the Clipboard relative to the Data Windows selected in Decision Assistance mode.

FIG. 114 illustrates the judge then selecting the node “Appearances”.

FIG. 115 illustrates the judge selecting the Data Window for the node “Appearances” and copying it to the Clipboard.

FIG. 116 illustrates the judge opening the Data Window for “Appearances” in the Clipboard and entering the data relevant from the matter proceeding before the judge.

FIG. 117 illustrates that if the parties make opening addresses, the judge may select the “Opening Addresses” node from the decision chain in Window 3.

FIG. 118 illustrates the judge copying the Data Window relevant to the node “Opening Addresses” the Clipboard and the judge may then make their notes as appropriate.

FIG. 119 illustrates the judge selecting the “Witness Summary” node from the Decision chain in Window 3.

FIG. 120 illustrates the judge copying the Data Window relevant to the node “List of Witnesses” onto the Clipboard.

FIG. 121A illustrates the judge entering the name of the first witness. FIG. 121B illustrates how user may resize the Data Window to facilitate reading and editing that Data Window.

FIG. 122 illustrates the judge then selecting the node “Prosecution Witness” from the steps in the decision chain in Window 3.

FIG. 123 illustrates the judge then copying the Data Window relevant to the node “Prosecution Witness” in Window 3 to the Clipboard.

FIG. 124 then illustrates the judge opening the Data Window in the Clipboard, and entering that witnesses name and making notes in respect of that witnesses evidence.

FIG. 125 illustrates the operation of computer system 100 in respect of the tender of a document.

FIG. 126 illustrates the judge returns to Window 1 of the Decision Support mode and selects the node.

FIG. 127 shows the steps in the decision chain relevant to solving a problem in respect of the law of relevance then appear in Window 3.

FIG. 128 illustrates the decision maker selecting the node “Definition of Relevance” and the Data Window relevant to that node opens.

FIG. 129 illustrates the decision maker may copy that Data Window to the Clipboard by selecting the “Add to Clipboard” function and the Data Window then appears in the Clipboard.

FIG. 130 illustrates the decision maker opening and editing that Data Window on the Clipboard as is appropriate by reference to the specific problem in the matter before the judge.

FIG. 131 illustrates the judge returning to the node “Judgment Template—Criminal” in Window 1.

FIG. 132 illustrates the judge then selecting the node “Exhibit” from Window 3 and its Data Window opens.

FIG. 133 illustrates the judge copying the Data Window “Exhibit” onto the Clipboard.

FIG. 134 illustrates the judge then opening the Data Window in the Clipboard and entering the details of the Exhibit.

FIG. 135 illustrates if an interlocutory application is made during the proceedings, such as the defence making what in Australia is referred to as a “Prasad Application”, the decision maker may return to Window 1 and select the node “Criminal Procedure”.

FIG. 136 illustrates the sub node topics available within computer system in relation to the node “Criminal Procedure”.

FIG. 137 illustrates the decision maker scrolling though these sub-node topics relevant to the node “Criminal Procedure” until the decision maker locates “Prasad Direction”.

FIG. 138 illustrates the Decision Maker selecting the node “Prasad Application” and its related sub-node topics.

FIG. 139 illustrates the decision step nodes.

FIG. 140 illustrates the decision maker may proceed though each step in the decision chain and may select the node “Criteria for Prasad Direction”.

FIG. 141 illustrates the decision maker may select the first step in the criteria to be applied and the Data Window relevant to that node opens.

FIG. 142 illustrates the decision maker copying the Data Box relevant to the node “To Be Sparingly Applied” into the Clipboard.

FIGS. 143 and 144 illustrates the decision maker resizing the Data Window to edit its contents and add data relevant to the matter proceeding before the judge.

FIG. 145 illustrates the decision maker returns to the Judgment Templates in Window 1.

FIG. 146 shows the decision maker then selects Judgment Template—Criminal in Window 2.

FIG. 147 illustrates the decision maker selecting the node “Prosecution Witness”.

FIG. 148 shows the appearance of the Data Box.

FIG. 149 shows that Data Window copied to the Clipboard.

FIG. 150 shows the decision maker enters details of the witness and notes of the witnesses evidence.

FIG. 151 illustrates the decision maker selecting the node “Prosecution Submissions”.

FIG. 152 illustrates the Data Window relevant to that node being copied onto the Clipboard.

FIG. 153 illustrates the judge opening that Data Window and entering the judges notes in relation to the submissions.

FIGS. 154, 155 and 156 illustrates the decision maker taking the same steps in relation to the submissions made on behalf of the defendant.

FIG. 157 illustrates the decision maker returning to Window 1 and selecting the node “Criminal Offences”.

FIG. 158 shows the list of offences for which decision support is available in computer system.

FIG. 159 illustrates the decision maker/judge then selecting the offence.

FIG. 160 illustrates the judge selecting the node “Assault Occasioning Actual Bodily Harm”.

FIG. 161 illustrates the decision maker selecting the node of the first step in the decision chain, Physical Elements.

FIG. 162 illustrates computer system bringing the sub-node topics relevant to the node “Physical Elements” into the visual display.

FIG. 163 illustrates the decision maker selecting the node Assault.

FIGS. 164, 165, 166 and 167 illustrate the user selecting each of the above Window 4 topics and computer system 100 bringing into the visual display the Data Windows relevant to those nodes.

FIG. 168 shows the decision maker judge selects the Mental Element node in Window 3.

FIG. 169 illustrates computer system brings into the visual display the downstream nodes relevant to the node Mental Element in Window 4.

FIG. 170 illustrates the user selecting the Window 4 node.

FIG. 171 illustrates the decision maker selecting the next step in the decision chain in Window 3.

FIG. 172 illustrates the decision maker selecting the node “Without Lawful Excuse” from Window 1 of the visual display.

FIG. 173 illustrates the selection of the node “Without Lawful Excuse” causes computer system to perform 2 functions.

FIG. 174 illustrates the decision maker selecting the defence of self-defence, raised on the facts before the judge in the hearing.

FIG. 175 illustrates that the Data Window informs the judicial decision maker.

FIG. 176 illustrates the decision maker selecting the link in the Data Window Self-Defence in the decision chain relevant to Assault Occasioning Actual Bodily Harm.

FIG. 177 illustrates computer system taking the decision maker to the node Criminal Defences in Window 1.

FIG. 178 illustrates the steps an expert has prepared to guide a user.

FIG. 179 illustrates the decision maker judge selecting the node Statutory Provision from Window 3.

FIG. 180 illustrates computer system bringing into the visual display in Window 4 the related sub-node or downstream node.

FIG. 181 illustrates the decision maker selecting that node and illustrates computer system bringing that node into the Data Window.

FIG. 182 illustrates the decision maker progressing though the decision steps in Window 1.

FIG. 183 illustrates the selection of the node “When Defence is Available” opens the Data Window relevant to that node.

FIG. 184 illustrates the decision maker selecting the node “Test to be Applied”.

FIG. 185 provides a further example of the decision maker selecting the Window 4 node “Step 1—Subjective Assessment”.

FIG. 186 illustrates how the selection of this node causes its related Data Window to open in Column 3.

FIG. 187 illustrates that Data Window may be copied in whole or in part to the Clipboard by the user in the same steps described in respect.

FIG. 188 illustrates the decision maker having copied the Data Window “Step 2—Objective Assessment” onto the Clipboard.

FIG. 189 illustrates the decision maker then selecting the node “Assault Occasioning Actual Bodily Harm”.

FIG. 190 illustrates the user then returns to the decision chain in relation to the offence of assault occasioning actual bodily harm.

FIG. 191 illustrates the decision maker selecting the node “Trial Direction”.

FIG. 192 illustrates the User may open the Trial Decision on the Clipboard and resize it.

FIGS. 193-194 illustrate the operation of the search function in computer system.

FIG. 195 illustrates a user opening computer system in Report or Judgment mode.

FIG. 196 illustrates computer system 100 in Report or Judgment mode.

FIG. 197 illustrates User may re-order the location of the Data Windows on the Clipboard in Report mode.

FIG. 198 illustrates how User may re-size a Data Window.

FIG. 199 illustrates a Data Window in Clipboard made resized.

FIG. 200 illustrates the User may remove the Data Window.

FIG. 201 illustrate user may also open any Data Window on the Clipboard and select and copy the contents (or part thereof) into the report or Judgment using the select, copy and paste function.

FIG. 202 illustrates user 101 opening and resizing the Report or Judgment Window.

FIGS. 203-257 illustrate user interfaces of the system in accordance with a third embodiment.

FIG. 203 illustrates the Matters List of the Program.

FIG. 204 illustrates Summary Interface of Decision-Maker/Users Current Matters.

FIG. 205 illustrates the plural Summary of aspects of a Matter the User/Judge may wish to access at different points in time across the course of the Matter.

FIG. 206 illustrates the Summaries of Key items a User/Judge may have ready access in the Matters Interface of the Program.

FIG. 207 Illustrates User/Judge having immediate access to many documents to which constant referral is likely to occur across both the conduct of the matter as well as when the User/Judge is writing their Judgment.

FIG. 208 illustrates the Programs incorporation of all documents (including multi-media documents) relevant to the Matter into the Program through the Matters Interface and which will allow all documents to be linked to the evidence of any party in any data window or to any other item of Evidence in Tagging and Compilation mode.

FIG. 209 illustrates the Program may be customisable in the Matters Interface depending on the nature of the documents (including multimedia data) the specific User may require.

FIG. 210 illustrates User may use import function in Library Edit mode in Matter Interface to import Decision Support Algorithms prepared by other Users of the Program, as well as Data Matrix Modules specific to the decision requirements.

FIG. 211 illustrates how User may import a Decision Support Algorithm into the Program.

FIG. 212 illustrates how the Program allows the User to import a Decision Support Algorithm prepared by another User of the Program.

FIG. 213 illustration of how the modularity of the system allows a User to highly customise the decision support algorithms they may need to use in their specific application of the Program.

FIG. 214 illustrates the User/Judge selecting a best practice Algorithm to record entry of data from the Hearing or Trial the Judge is conducting and loading it into the visual display.

FIG. 215A illustrates how Data Matrix mode may be used by the Judge to rapidly access information necessary for a Judge to utilise in the course of a Hearing or Trial.

FIG. 215B illustrates the User/Judge then selecting May v O'Sullivan dismissal from the list of all available orders for decision and the Program then brining the applicable legal principles to apply to the Matter before the Judge into the visual display.

FIG. 215C illustrates User/Judge selecting the legal principle applicable to a May/O'Sullivan dismissal and copying it onto the Primary Clipboard.

FIG. 215D illustrates the User re-sizing the Data Window for ease of reading access when the User opens this level of the Data Windows editing function,

FIG. 215E illustrates User being able to import data from Data Matrix into the Primary Clipboard which remains constant in Data Matrix and Decision Support mode of Program.

FIG. 215F illustrates the new data windows and document management functionality of the Program.

FIG. 215G illustrates the first level of editing and review provided by the Program to the User/Judge.

FIG. 216 illustrates the User/Judge selecting the Enhanced Multi-Clipboard functionality of the Program selected by double-clicking the Data Window in the Primary Clipboard.

FIG. 217 illustrates the Tagging functionality.

FIG. 218 illustrates further aspects of the Tagging functionality of the Program.

FIG. 219 illustrates colour tagging functionality of the program.

FIG. 220 illustrates the colour tagging function of the program colouring the Data Window to which it applies.

FIG. 221 illustrates the Program may link a document from any source (external via a hyperlink) or to any document contained in the Documents in the Document Management function in the Matters mode.

FIG. 222 illustrates Chronology compilation function of the Program.

FIG. 223 illustrates the Programs application in organising, managing and assisting the User/Judge in their fact finding function in Compilation mode of the Program.

FIG. 224 illustrates the use of the Chronology or timeline function in Compilation mode.

FIG. 225 illustrates User's application of the Compilation function to extract from the entire field of data of the Matter (Hearing or Trial) all of evidence related the tag filters selected by the User/Judge.

FIG. 226 illustrates the Program facilitating the User/Judge being able to bring into the visual interface multiple Sub-Clipboards enabling the User/Judge to compare and contrast (for example evidence from different sources on the one topic).

FIG. 227 illustrates the Program opening multiple Sub—Clipboards greatly facilitating the User/Judge's ability to compare, contrast and evaluate the relevant evidence to assist them in making the most informed Judgment about what occurred in respect of that event.

FIG. 228 illustrates the User being able to select, open and compare the evidence in Data Windows contained in may Sub—Clipboards in the visual display.

FIG. 229 illustrates User/Judge being able to rapidly identify and contrast relevant plural items of evidence in the Compilation mode.

FIG. 230 illustrates the User/Judge may have open multiple Sib-Clipboards which will size to conform to the visual display.

FIG. 231 illustrates the Programs functionality in allowing the User/Judge to manage the data in the data field of the entire Matter (Hearing or Trial) to identify, extract, link, compare and evaluate those items of data necessary for the Judge to be able to make a decision which takes into account all evidence related to that part of their decision.

FIG. 232 illustrates User/Judge turning to Judgment mode after they have competed their fact finding duties in Compilation mode.

FIG. 233 illustrates how program facilitates the User/Judge applying their findings of fact utilising Compilation mode to a best-standard or expert application of the law applicable to the Matter (Hearing or Trial).

In Judgment mode, the User/Judge selects the list of expert algorithms the specific legal application required to be applied in the resolution of the Matter.

FIG. 234 illustrates the User/Judge using Judgment compilation mode to apply the facts they have found in relevant aspects of the matter to the best-practice (expert) application of the related legal principles.

FIG. 235 provides further example of types of specialist algorithms that may be selected and applied by the user in the preparation of their Judgment.

FIG. 235A illustrates Program facilitating User/Judge applying their findings of fact to the correct (expert) application of the law contained in the Decision Algorithm in the visual display.

FIG. 235B illustrates how the Program facilitates the application of the Facts found by the Judge in Compilation mode to the experts Decision Algorithm on the legal topics or areas of law the Judge has to apply to the facts so found to generate a decision outcome in the Matter, that is their Judgement in the Matter.

FIG. 236 illustrates how Decision Support Algorithms may be utilised by the User during the progress of a Matter (ie during the course of a Hearing or Trial).

FIG. 237 illustrates the User/Judge bringing into the visual display an expert decision support algorithm to assist them make a legal decision relating to an aspect of the progress of the matter before them.

FIG. 238 illustrates the User may copy the relevant law onto the Primary Clipboard and may also access the Sub-Clipboard List to retrieve any other data that may be relevant to the issue.

FIG. 239 provides an illustration of the User accessing a specialist Decision Algorithm during the conduct of a Matter (Hearing or Trial) whereby the User has to make important legal decisions about (inter alia) the admissibility of evidence.

FIG. 240 illustrates how the Program takes the user to an experts (accurate) specialist steps in applying the law correctly and enables it to be applied to the facts before the User/Judge.

FIG. 241 illustrates how User/Judge incorporates the ruling on the item of evidence in question onto the Primary Clipboard.

FIG. 242 illustrates how the different levels of editing and tagging may operate to assist the Decision-Maker in all aspects of the operation of the Program.

FIG. 243 illustrates how User/Judge may access the enhanced editing and data window management functions in respect of any mode of the operation of the Program.

FIG. 244 illustrates that the expert algorithm on this legal topic (the application of the law of Relevance) may take the User/Judge as far into the complexity of the application of the law as the User/Judges case may require.

FIG. 245 illustrates how the Program provides a structure which enables the expert algorithm to identify the relevant law and will also alert and guide the User/Judge to circumstances where the law applies in a more specialised manner and enables the algorithm to take the User/Judge through that more specialised application.

FIG. 246 illustrates how the Program enables the User to be taken though the best-practice approach to the application of the correct legal principles contained in the Decision Support expert Algorithm.

FIG. 247 illustrates how a User/Judge having utilised the program to make a Ruling (Decision) on a legal issue during the course of the Matter (Hearing or Trial) then returns to their List of Algorithms and selects the Criminal Hearing Algorithm and returns to continue with the Matter.

FIG. 248 illustrates the operation of the Sort function in relation to all Clipboards generated by the Program.

FIG. 249 illustrates how Compilation mode may be used to manage all procedural, evidential, and interlocutory rulings made during the course of the Matter (Hearing or Trial) so that they may readily and easily be accessed in their own Separate Sub-Clipboards.

FIG. 250 illustrates application of Judgment mode of the Program, permitting User/Judge in this example to apply the law relating assault prepared by an expert to the findings of fact the Judge has made in compilation mode.

FIG. 251 illustrates application of program in Judgment mode assisting the User/Judge to apply the law to the facts contained in the Sub-Clipboard generated in Compilation mode.

FIG. 252 illustrates how Conclusions of Fact on specific elements of an legal offence, having been evaluated by the User in Compilation ode, may be generated into their own Sub-Clipboards illustrated in this diagram and be loaded into the Sub-Clipboard window for incorporation into correct statement of legal principle the User has loaded into the Judgment Clipboard.

FIG. 253 illustrates how Program assists user in applying the facts found to the experts identification of the correct legal principles in the Expert Decision Algorithm

FIG. 254 illustrates Program facilitating User/Judge being able to resize any data window to be opened and edited at any stage during the operation of the Program.

FIG. 255 illustrates User/Judge may utilise Compilation mode and generate a Draft of their Judgment at any stage as it evolves to finality.

FIG. 256 illustrates the Program allowing the User/Judge to re-order or sort the Data Windows in the Draft Judgement Sub-Clipboard depending upon how that Particular User may wish to structure their Judgment.

FIG. 257 illustrates Sort function in Draft Judgment mode enabling the User/Judge to structure their Judgment in any way that they may wish.

### BEST MODES OF THE INVENTION

FIG. 1 illustrates a computer system 100 for control of a multi-stage process for a user 101. The computer system 100 comprises a display device 102 and a computer 104 including a processor 106, a data memory 108 and a program memory 110. The display device 102 is connected to the processor 106 of computer 104 via input port 112 and output port 114 of computer 104 and the processor 106 is connected to data memory 108 and program memory 110 via memory port 116. In use, the processor 106 creates a user interface and sends the user interface to display device 102 via output port 114.

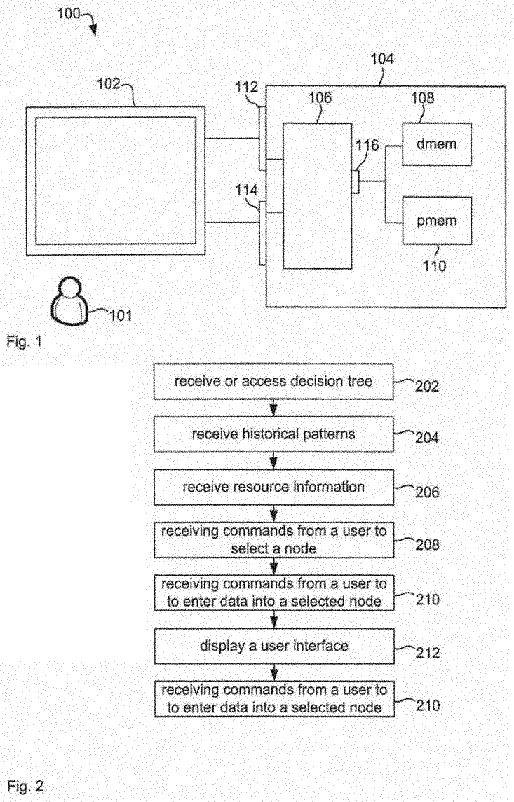

Therefore, processor 106 executes software stored on program memory 110 that causes the processor 106 to perform the method of FIG. 2. The sending of the user interface may be as pixel values, such as a png image attached to an email, as display commands, such as Direct X, as vector graphics, or as a website using Flash and/or HTML. Processor 106 may also send the user interface to the data memory 108 to be stored for later use.

In this example, the data memory 108 and program memory 110 are both non-volatile hard disk memory, but of course, other memories may be used, such as RAM, on chip cache, optical disks or flash memory. In some examples, the computer system is implemented in a cloud computing environment. In such examples, a number of processors may be deployed as virtual machines to create the calendar display and the data memory 108 as well as the program memory 110 are implemented as cloud storage. In such examples, the display device 102 may be on a mobile device that is connected to the cloud via the Internet.

Computer system 100 may also be a desktop, laptop, netbook or other stand-alone computer with or without internet connection and with or without using cloud services.

In this example, display 102 is a touch screen display, such as the display of a tablet computer. As a result, the display 102 is used by the user as an input device and the processor 106 receives the input, updated resource information or data relating to stages of the process via input port 112 from the input device 102.

The data memory 108 stores a decision tree, and the processor 106 queries the data memory 108 in order to create the user interface. As a result, the processor 106 receives data from the data memory 108 via memory port 116. The memory 108 stores status information about one or more of the stages of the process and user input provided at one or more nodes so that the same input is available for consideration or reuse at a later time.

Although input port 112, output port 114 and memory port 116 are shown as distinct entities, it is to be understood that any kind of data port may be used to receive and send data, such as a network connection, a memory interface, a pin of the chip package of processor 106, or logical ports, such as IP sockets or parameters of functions stored on program memory 110 and executed by processor 106. These parameters may be handled by-value or by-reference in the source code. The processor 106 may receive data through all these interfaces, which includes memory access of volatile memory, such as cache or RAM, or non-volatile memory, such as an optical disk drive, hard disk drive, storage server or cloud storage.

FIG. 2 illustrates a method 200 for controlling a multi-stage process as performed by processor 106. Processor 106 commences performing method 200 by receiving or accessing 202 a decision tree.

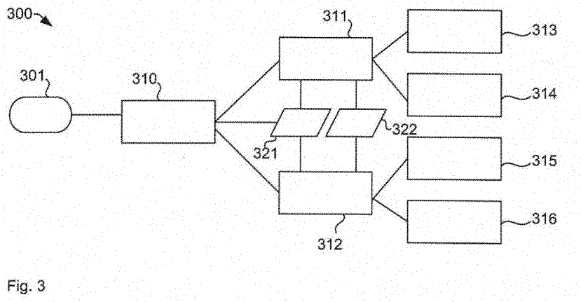

FIG. 3 illustrates a decision tree 300 having a start 301, a network of interconnected nodes 310 to 312 and terminals 313 to 316. Each node 310 to 312 represents a stage of the

multi-stage process. The decision tree 300 is physically stored on data memory 108 in a suitable data format, such as at a linked list or an MySQL database where each node of the tree 300 is one record in the database and each record also stores a predecessor of each node.

In one example, computer system 100 is used by a judicial officer as a tool to facilitate access to information required for decision making. The computer system 100 provides access to the data the judicial officer needs within the decision chain relevant to the resolution of that particular problem. The judicial officer does not have to extract the data from the law reports, on line services, legislation databases, textbooks or loose leaf services, nor will they have to organise and marshal that data as appropriate to the resolution of the issue or problem before them. That task will be performed by and within the system 100.

The computer system 100 also provides guidance in that it will make available to a Magistrate historical patterns, such as the decision chain of a high level expert in respect of the legal issue or problem. In this way, the high level expert, such as a professor of law, makes entries between two nodes in the network of the decision tree 300.

The computer system 100 will not bind or remove judicial discretion, or undercut the independence of the judicial function. What it will do is provide a structure which will make readily accessible all data relevant to the resolution of the legal problem, and provide the assistance (if the judicial officer wishes to avail themselves of it) of the decision chain a high-level expert in relation to that problem would apply to all its resolution.

The computer system 100 provides a structured but flexible decision matrix which provides the decision maker with access to relevant data around the decision chain required to resolve the problem.

The organisation and complexity of the decision criteria, and the access the decision maker has to relevant and current data at each stage of the decision chain, determines the quality of the decision generated.

The computer system 100 allows an (infinitely) multidimensional decision matrix which integrates the decision steps to be taken with current data.

The next step of method 200 is receiving 204 historical patterns of entries made by users between first and second nodes in the network. The historical patterns may be court decision that constitute applicable common law. The processor 106 may receive the historical patterns by accessing a data matrix that stores the historical patterns on data store 108.

Then, the processor 106 performing method 200 receives 206 plural different types of resource information for each stage of the multi stage process. In one example, the resource information includes court decisions, statutes and other legal texts. The resource information may also be a link to a manual or technical specification of a particular machine.

In one example, the computer system 100 may be referred to as a Neural Net Decision System (NNDS). A Data Matrix is at the core of the Neural Net Decision System.

The provided Figures provide worked examples the operation of the NNDS in Data Matrix Mode.

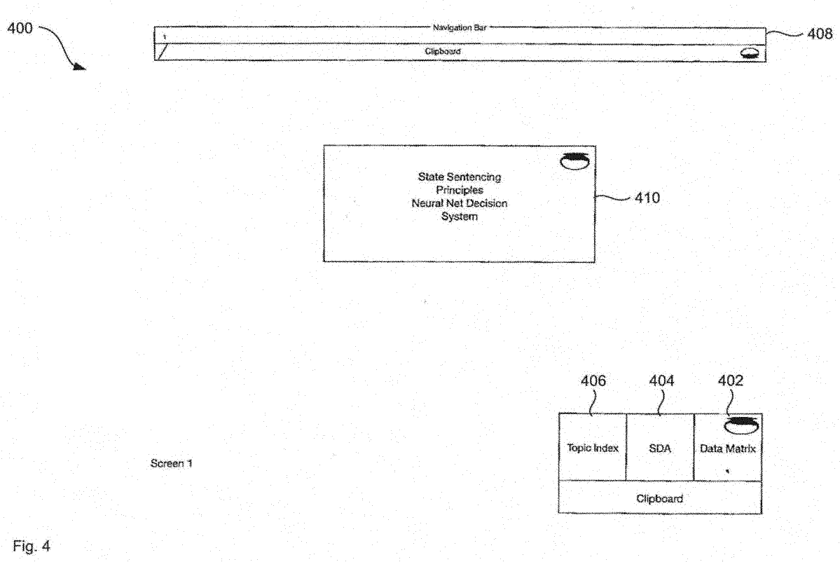

FIG. 4 illustrates a visual display 400 of the NNDS comprising a persistent menu identifying the plural different types of resource information, such as the Data Matrix 402, Structured Decision Assistance (SDA) 404, Topic Index Windows 406 and the Navigation Bar 408. The Navigation Bar 408 provides regions that the decision maker can activate in order to backtrack to earlier nodes of the decision tree. When the programme is activated and opened, the first dimension 400 of the display opens. The Decision Maker selects the Data Matrix mode 402.

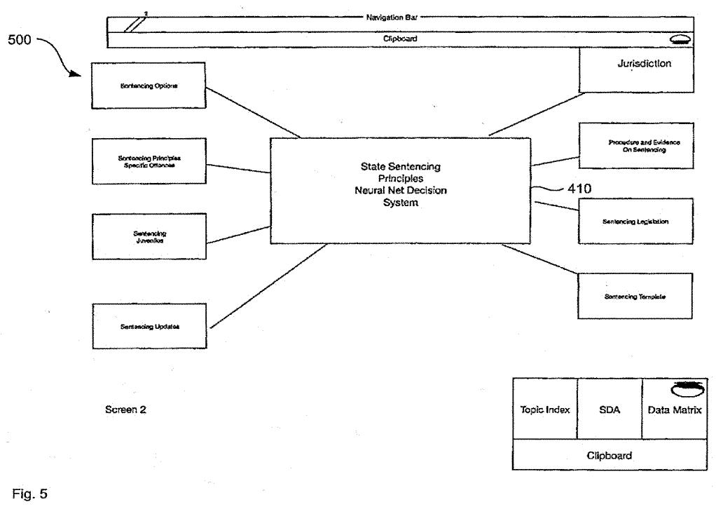

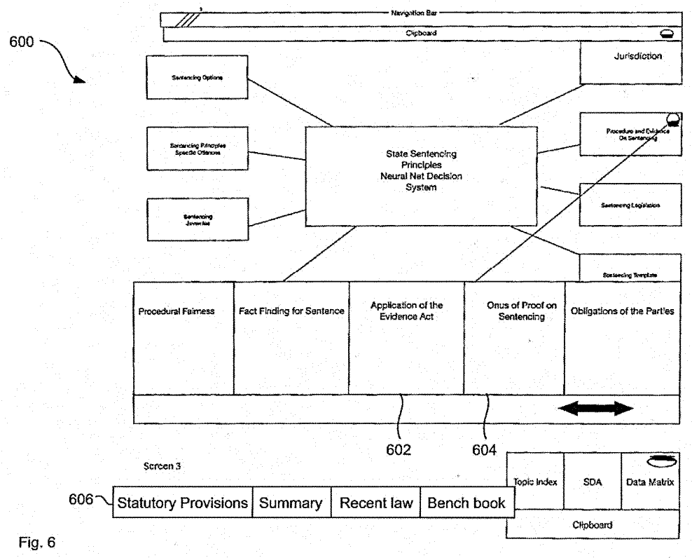

FIG. 5 illustrates a visual display 500 after selecting a Topic Node 410 for the Sentencing Neural Net. This selection takes the Decision Maker to the next dimension the key subject areas that arise in respect of the topic “Sentencing Law in New South Wales”. Each Node (whether Topic, Primary, Secondary, or Sub-Node) may have links to other Nodes in the NNDS.

By way of example, the decision maker may have an issue before them in relation to procedure and evidence on sentence. Selecting that primary node in the NNDS will open a sub-node which will have the sub topics relevant to that primary Node topic.

FIG. 6 illustrates the resulting user interface 600. The sub-node as a directional arrow allowing the decision maker to scroll through the topics in the sub-node. The topics in the sub-node are not restricted, accordingly, by the size or space of the visual display on the computer screen.

In one example, the user interface 600 comprises a further persistent menu 606 identifying the plural different types of resource information. When the decision maker selects node 602, the persistent menu 606 provides a set of options that is specific to node 602. The set of options are different types of resource information, such as “Statutory Provisions”, “Summary”, “Recent law” and “Bench book. This set of options may not be applicable when the decision maker selects a different node, such as node 604 and the set changes. This way the resource information accessible via the persistent menu changes automatically in a context aware manner each time the user selects a different node in the decision tree.

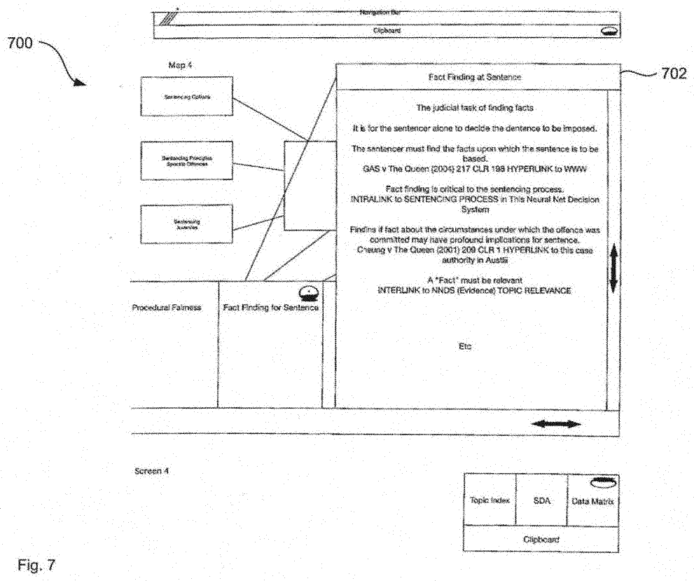

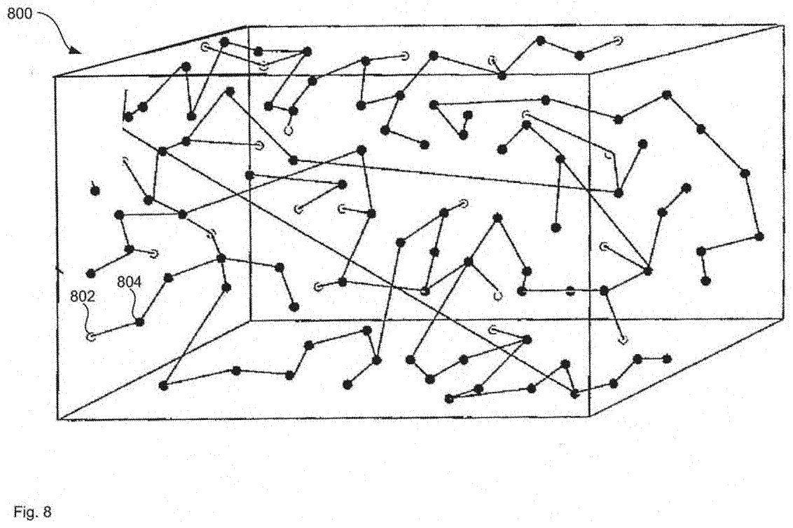

FIG. 7 illustrates a user interface 700 after the decision maker then selects the sub node topic relevant to the legal issue or problem. Selecting that sub node topic opens its associated data window. The sub-node topic selected by the decision maker in the illustrated example is “Fact Finding at Sentence”. The “Fact Finding at Sentence” data window opens.

The Data Window 700 has a Header 702 which has the same topic as it is related sub node topic. The Data Window may contain plural different types of resource information:

(1) Intralinks—between specific content in the Data Window to other pages and nodes within that neural net decision system (in this case, NNDS Sentencing);

(2) Interlinks—between specific content in the Data Window and specific nodes in linked Neural Net Decision Systems (ie NNDS (Evidence NSW)), to data files on the host computer or networked computer systems, and

(3) Hyperlinks—between specific content in the Data Window and to sites on the World Wide Web.

Selecting the Intralink associated with “Fact Finding is Central to the Sentencing Process” will take the Decision Maker to the topic “Sentencing Process” within the NNDS Sentencing; and Selecting the Interlink to “A Fact Must Be Relevant” in the

NNDS Sentencing exemplar will take the Decision Maker out of the NNDS (Sentencing) to a related Node in a linked NNDS—for example, an NNDS (Evidence)

Selecting the hyperlink to the case authority Gas v The Queen (2004) or the hyperlink to Cheung v The Queen (2001) will take the Decision Maker directly to a related site on the World Wide Web (ie for example, Austlii). After the decision maker has appraised themselves all the information they require from the “Fact Finding at Sentence” Data Window, the decision maker will close that window 700 and the visual display returns to the Sub—Node topics relevant to Procedure and Evidence on Sentence.

The number of topics on new screens is the measure of the “depth” of the system. It may be represented by a nodal index, N(x), where X is a measure of the number of topics on new pages within the NNDS. An NNDS has an infinite plasticity or depth, which will be determined by the volume of data contained within the NNDS system and the extent of the interrelationships between the nodes.

The following description explains the Structured Decision Assistance (SDA), which is a meta-function of the NNDS. SDA mode provides a replication of historical patterns, that is, the steps in the decision chain taken by a high-level expert in the resolution of a problem by way of a structured paradigms of questions linked to data embedded in the Data Matrix.

Structured Decision Assistance functions is an overlay to the Data Matrix. The system 100 takes the decision maker through the data embedded in the Data Matrix through a series of questions. Each question requires an answer as an input from the decision maker. The system 100 provides a very high level of support for a decision maker in relation to the criteria to be applied to those specific steps necessary to comprehensively work their way through a problem contained in or what the subject of a particular Neural Net Decision System. This means that the answers from the decision maker needs to satisfy the specified criterion.

The responses provided by the decision maker to the criteria and questions within the Structured Decision Assistance mode will take the decision maker to different Nodal routes though the Data Matrix. This means that the system 100 responds to the input into the selected node by selecting one of the downstream nodes and the computer screen 102 is updated.

The purpose of Structured Decision Assistance is to allow a decision maker to be guided through the Data Matrix by the responses to a series of questions designed by a high-level expert in respect of the topic of the specific NNDS.

By way of illustration, in relation to the NNDS Sentencing example, the SDA may be designed by a Supreme Court judge, who has 30 years experience and an established jurisprudential reputation. This way, the historical patterns stored in the SDA between two nodes in the network are made by the Supreme Court judge. The SDA may of course be collaboratively designed. There may well be a court specific Neural Net system on a particular topic, and specific decision criteria may also be generated by collaborative work with, for example, academics in this subject area.

The experience, knowledge and expertise that this senior or judicial officer offers in respect of the approach to sentencing may be made available in SDA mode in the NNDS Sentencing to any person, whether Judge, practising lawyer, Law student or lay person who has access to this NNDS system.

FIG. 8 illustrates a data matrix 800, where white dots, such as white dot 802, represent a link within the NNDS from within a Data Window. That link may be a link to another Node, another Data Window, or to an external side (a data file on a computer or computer network or a site on the WWW). Black dots, such as black dot 804, represent a Node in the Decision System. The Nodes embed data within the Data Matrix. Each Node (including Tooic Nodes, Primary Nodes, Secondary Nodes, and Sub-Nodes) with their related Data Windows may be linked to other Nodes (within this NNDS or a linked NNDS) or to external links (to data files on the Decision Makers computer or computer network) or to sites on the WWW.

Each Node represents a different topic. The Topic Index contains a link to each Node within the NNDS. By selecting a topic from the Tooic Index, the user may be taken directly to that Node in the Data Matrix and that page will open within the NNDS.

Referring back to FIG. 2, the next step of method 200 is to receive 208 commands from a user, such as the decision maker, to select a node in the decision tree.

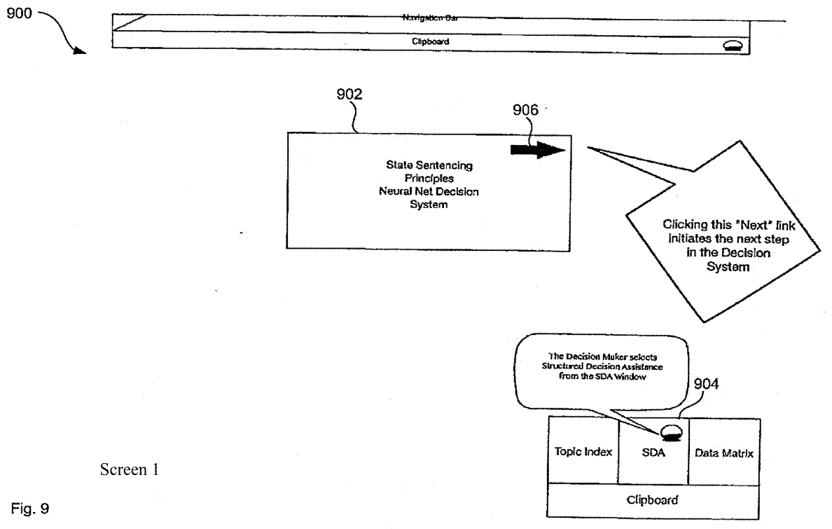

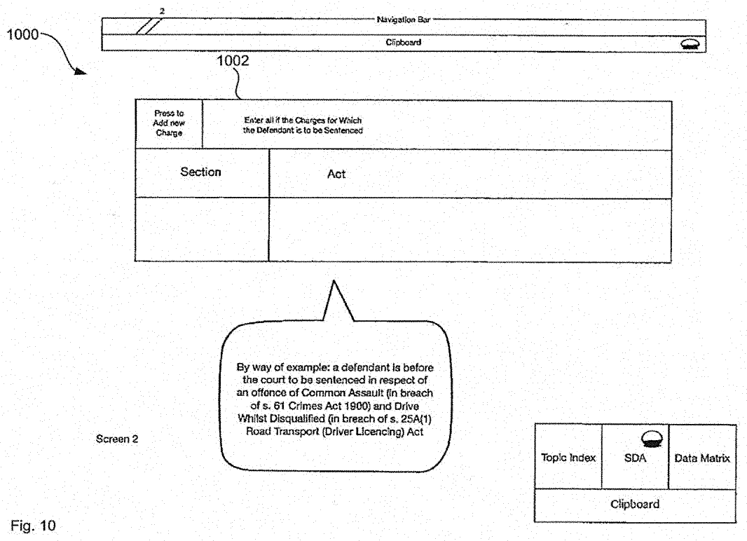

FIG. 9 illustrates a user interface where the Decision Maker selects a node in the decision tree by opening the NNDS Sentencing, and the first window opens showing the Topic Node, “state sentencing principles, neural net decision system” 902. The decision maker selects the SDA node 904 from the SDA Window.

Sentencing is typically commenced by reference to the identification of the charge and the maximum penalty which is open to the court to impose.

The first step in the NNDS Sentencing SDA, accordingly, is generated when the “Next” button 906 or link activates the next step in the sentencing decision chain, that being the identification of the specific maximum penalty applicable in relation to the offence for which the offender is before the court or sentence.

FIG. 10 illustrates another user interface 1000 as an NNDS SDA Screen that opens a window 1002 allowing for the entry of the specific section and the specific Act for which the offender is before the court for sentence.

Referring back to FIG. 2, the next step of method 200 is to receive 210 commands from a user, such as the decision maker, to enter data into a selected node.

The decision maker enters data into the selected node by entering the section and the Act. For example, an offender is before the court for sentence (inter alia) in relation to a charge of common assault in breach of s. 61 Crimes Act 1900 (NSW). That data is entered and a button indicated in the display is selected.

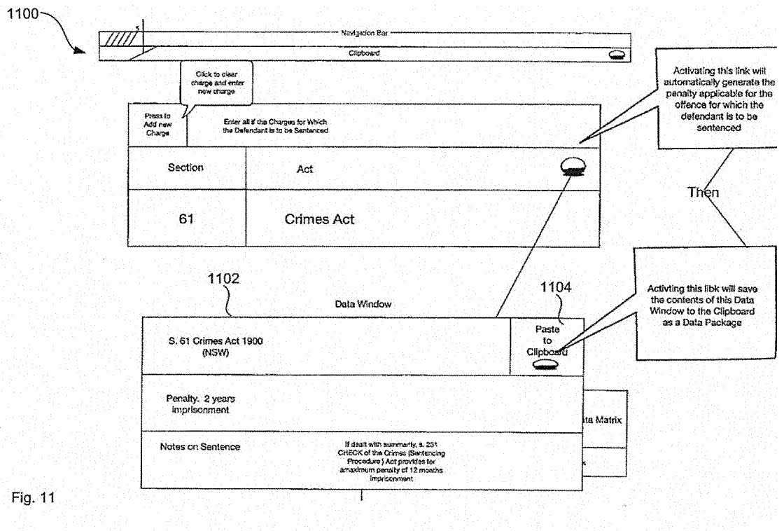

FIG. 11 illustrates another user interface 1100 including a generated Data Window 1102 which will identify the section and the Act, the applicable maximum penalty open to the court, and any specific notes applicable to that statutory offence that the decision made should be aware of all the required to take into account in relation to the penalty.

The button, “paste to clipboard” 1104, in NNDS SDA Screen 1100, will save that Data as a Data Package to the Clipboard.

The Decision Maker may repeat this process for every offence for which the offender is before the court for sentence. An offender is of course commonly before the court to be sentenced for more than one appendix.

Accordingly, before the decision maker progresses to the next step in SDA mode, the statutory provision the subject all the charge, the penalty applicable, and any specific notes on the applicable offence, are saved as Data Packages to the Clipboard.

This shows how in response to input into a selected node that satisfies a specified criterion, one of the downstream nodes is automatically selected and the computer screen is updated.

Apart from entering directly into the currently selected node, a user may also select a historical pattern of entries. As a result, the input is made automatically into a string of successive nodes as determined by the selected historical pattern of entries.

The Decision Maker then proceeds to the next step in the SDA by activating a “Next” link (not shown) in SDA mode. Activating the “Next” link will take the decision maker to the next step in the decision chain.

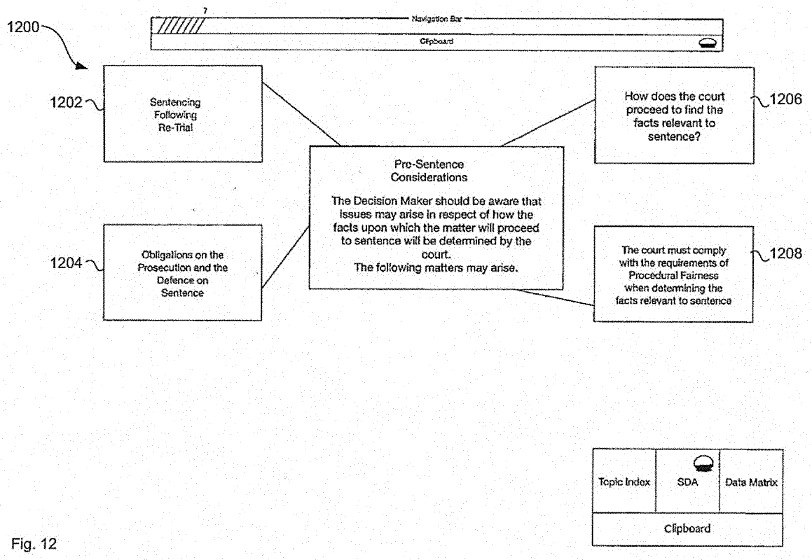

FIG. 12 illustrates another user interface 1200 representing that next step. That screen 1200 draws the decision-makers attention to the fact that in any sentencing exercise, there are 4 areas 1202, 1204, 1206 and 1208 that may require the decision maker to consider prior to embarking upon the sentencing exercise. The SDA draws the decision makers attention to each of these matters.

By way of illustration, a decision maker may not be aware how a court should appropriately proceed to find the facts relevant to sentence. The decision maker may not be aware of what legal principles and procedures should guide the judicial officer in the important function of deciding what facts are to constitute the basis upon which the court will proceeded to determine sentence, if there is a dispute between the prosecution and the defence in respect of those facts.

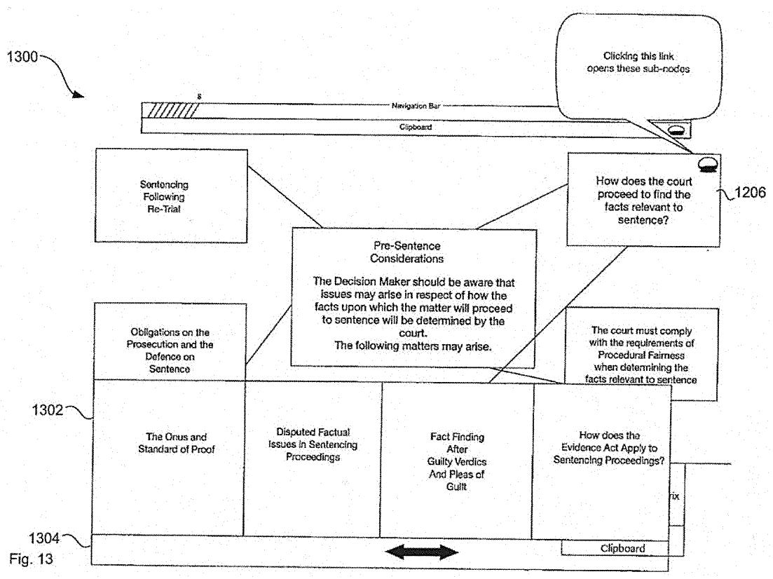

FIG. 13 illustrates another user interface 1300 where the decision maker selects the primary node topic, “how does the Court proceeds to find the facts relevant to sentence” 1206 at the NNDS SDA Screen. The topics that inform the decision maker how they should answer that question appear in the sub node. Note that the Sub-Node window 1302 has a scrollbar 1304 so that the topics which are able to be displayed are not limited by the size of the visual display.

Referring back to FIG. 2, the next step of method 200 is to display 212 a user interface including at least part of the decision tree including the selected node and at least one generation of downstream nodes. This can best be seen in FIGS. 6 and 7 where the node 602 “Application of the Evidence Act” is selected and one generation of downstream nodes, in this case node 604 “Onus of Proof on Sentencing”, is displayed.

The user interface further includes a persistent menu as explained with reference to FIG. 4.

The Decision Maker may sequentially work their way through each topic in the Sub-Node window 1302, or go straight to the topic in relation to which they have a specific query. In the illustrated example, the decision maker may wish to know whether the onus of proving a particular fact relevant to sentence lies on the prosecution or the defence (ie whether the defendant has some mental infirmity at the time of the commission of the offence).

The decision maker opens the Sub-Node topic, “The Onus and Standard of Proof and its related Data Window opens. The link between the SDA and the Data Matrix is maintained through the Header Topic in the Data Window.

If the information or summary of the applicable legal principle contained in the data window in SDA mode is not sufficiently precise all detailed for the decision makers purposes, the decision maker may activate the link in the data window in SDA mode and be taken directly to that same topic in the Data Matrix mode.

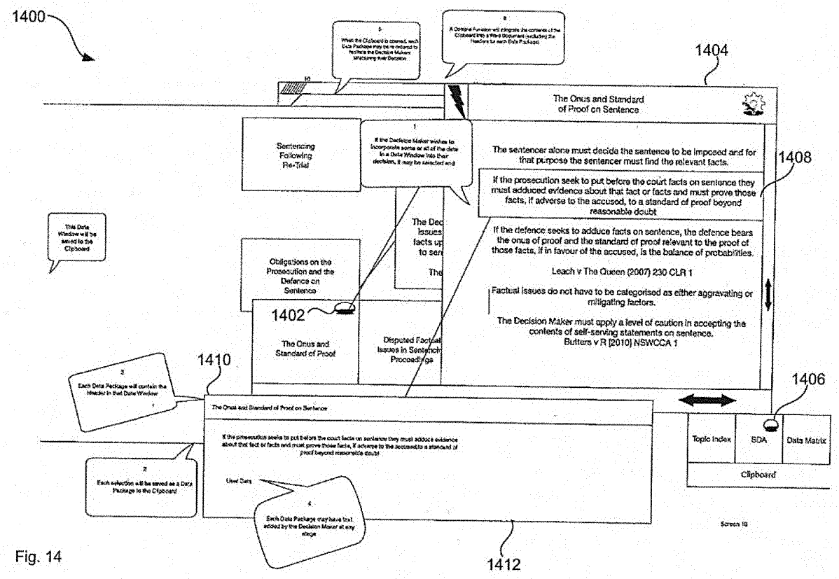

FIG. 14 illustrates another user interface 1400 that shows this a feature of the NNDS system and the mechanism through which the Data Matrix and Structured Decision Assistance modes integrate. If the link 1402 to the data matrix is activated, and the decision maker is taken to the Data Matrix mode, the decision maker may then explore all the available data in respect of that topic which is contained in the Data Matrix in window 1404.

If the decision maker has taken themselves into the terrain of the Data Matrix, and there obtains the data required (ie by cutting and pasting it then copying it to the Clipboard), the decision maker may then click on the SDA link 1406 in the permanent display and that will take the Decision Maker back to that step in the SDA mode.

For the purposes of illustrating how the Clipboard may facilitate a decision-making in SDA mode, it is noted that when a decision maker has identified the particular item of data that they require for their decision, they may utilise the Clipboard function to extract that data for inclusion in their decision or judgement.

The decision-makers selects that part of the data 1408 they wish to use and it is cut and pasted to the Clipboard. Each selected item of heart is saved into a separate Data Package on the Clipboard. Whilst in Clipboard mode, each Data Package retains the Header 1410 of the Data Window from which it was selected and cut. This will facilitate the decision maker organising the Data Packages when ultimately preparing to export the contents of the Clipboard into a word processing package utilising the Compilation function for the purposes of generating the decision-makers report or decision.

The decision maker may at any time open any Data Package, to edit its contents to add data in relation to the specific problem the decision maker is seeking to address to the data contained in that Data Package. For that purpose, the user interface 1400 provides an input window 1412.

To return to the illustrative example, once the decision maker has resolved that aspect of the problem relevant to the topic Node “Pre Sentence Considerations”, the decision maker activates a “Next” link to progress to the next stage in the decision system.

An input is made by a user either directly into the currently selected node, or automatically into a string of one or more successive nodes as determined by a selected historical pattern of entries.

The decision tree 300 also comprises resource information 321 and 322. The resource information 321 and 322 may be of different types, such as stored historical data or links to user manuals or other documentation.

The nodes 310 to 312 require inputs to satisfy a specified criterion. The input may be provided manually by a user via an input device. When the specified criterion is satisfied, one of the downstream nodes 311 and 312 for performing the next stage, is automatically selected. That way, the decision tree is traversed in a sequential manner from the start 301 to one of the terminals 313 to 316.

As described above, in one example, the input is provided directly by a user. In another example, the input is made automatically into a string of one or more successive nodes as determined by a selected historical pattern of entries. That is, a user enters training sequences that include the input. Each of these training sequences defines a path through the decision tree 300. As a result, each training sequence provides input to all the nodes in the path defined by that training sequence, that is into a string of nodes.

While in this example, the process control is based on a structure without reconverging paths, in other examples, a more general graph structure may be used, such that equivalent terminals and even equivalent sub-trees are merged.

The memory 402 stores the decision tree explained with reference to FIG. 3 and historical patterns of entries made by users between first and second nodes.

One example for which the control system may be used is for providing a decision aid in the legal context. Court cases are decided based on facts, statutes and common law references that are the inputs to different stages in the proceeding. Previously decided cases define paths through the stages and at each stage the input of the path and the next stage of this court case is stored. In order to be consistent with previous cases, a current case with similar facts needs to follow a similar path through the stages as the previous cases.

In one example, the input device 404 is a conventional computer keyboard and is connected to the processor 406 to enable a developer of the control process to input parameters, or the training sequence and refine the control. Using the input device, the developer may also select a node in the decision tree and enter data directly into the node. This may be necessary, for example, in cases where the learning based on the training sequences does not yield the required control outcome.

When in use, the screen 102 displays a user interface including a part of the decision tree or the entire decision tree. When the user selects a node, the user interface also includes the selected node and at least one generation of downstream nodes. In the example of FIG. 3, when the user selects the node 310, the user interface includes node 310 and downstream nodes 311 and 312. In examples where screen 102 is a touch screen the user

can select a node of the decision tree 300 and enter data into the selected node by tapping the surface of the touch screen.

Although some of the above examples relate to legal decision making, it is to be understood that the system is equally applicable in other areas such as medical decision making or education and learning, such as in collaborative research systems and organisation of research data. Other areas include complex safety decision making, such as aircraft safety diagnostic manuals, or creative endeavours.

It will be appreciated by persons skilled in the art that numerous variations and/or modifications may be made to the specific embodiments without departing from the scope as defined in the claims.

It should be understood that the techniques of the present disclosure might be implemented using a variety of technologies. For example, the methods described herein may be implemented by a series of computer executable instructions residing on a suitable computer readable medium. \hat{ }Suitable computer readable media may. include volatile (e.g. RAM) and/or non-volatile (e.g. ROM, disk) memory, carrier waves and transmission media. Exemplary carrier waves may take the form of electrical, electromagnetic or optical signals conveying digital data steams along a local network or a publically accessible network such as the internet.

It should also be understood that, unless specifically stated otherwise as apparent from the following discussion, it is appreciated that throughout the description, discussions utilizing terms such as “estimating” or “processing” or “computing” or “calculating”, “optimizing” or “determining” or “displaying” or “maximising” or the like, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

### Further Embodiment of the Invention

The Neural Net Decision Support System has 4 user interfaces.

A revised version of the invention has been prepared. The revised version of the invention has been illustrated in its operation using the tools available in Filemaker Pro. The first user interface is the portal providing access to the Decision System.

The portal provides access to the computer system 100. The computer system may operate on the computer being utilised by user 101 or may be access by user 101 though that computer on a cloud computing environment.

The portal provides secure log in access to the computer system and contains a list of all saved decisions of the user 101, whether those decisions are completed decisions or incomplete decisions and the decision maker may return to those previously saved decisions at any time.

In one example, the decisions include judgments which have been finalised or completed by a judge or which are partially completed by a judge.

In one example, they may include diagnostic reports or decisions a medical practitioner has made in relation to a patient and those saved files may be forwarded by email or other modes of electronic transfer to other medical practitioners (for example medical specialists to whom the patient may be on-referred)

The second user interface is the Data Matrix. It is selected by user 101 selecting the Data Matrix button on the permanent display.

The third user interface is Decision Assistance. It is selected by user 101 selecting the Decision Assistance button on the permanent display.

The fourth user interface is the Report (or Judgment) interface. It is selected by user 101 selecting the Decision Assistance button on the permanent display.

### Description of the Additional Drawings in Relation to the Further Embodiment of the Invention

14.

15. etc

Structure of the Decision System

There are 4 modes.

1. The Portal

2. Data Matrix

3. Decision Assistance

4. Report (Judgment)

The Portal

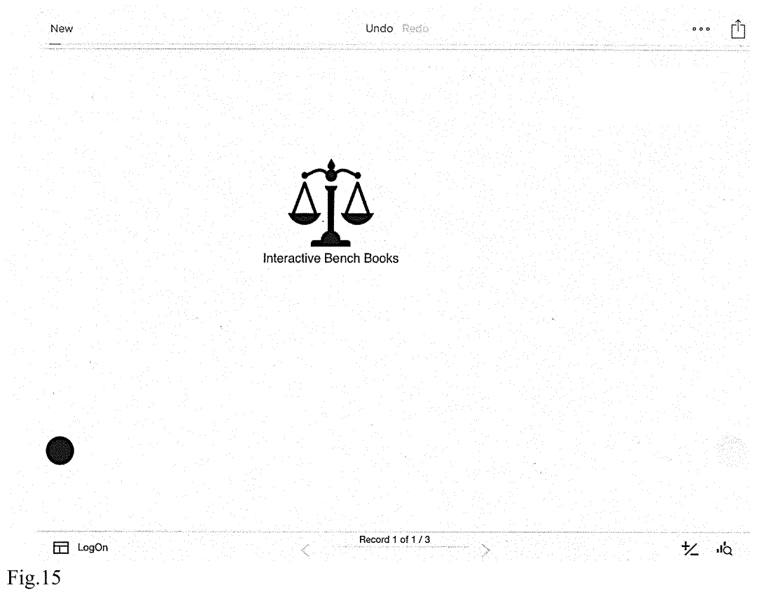

FIG. 15 illustrates the decision support system which will be used as a working example in this patent application. It is a prototype prepared using the tools in FilemakerPro. The example of a computer system 100 decision support system relates to the data a judge requires for the discharge of their judicial function of deciding cases, hearing matters and applying the law. The decision system for the purposes of illustrating the various functions of computer system 100 has been called “Interactive Bench Book”.

In this one example, the Decision Support System is for resource materials available to Judges (or any judicial officer such as a Magistrate) in the discharge of their judicial function.

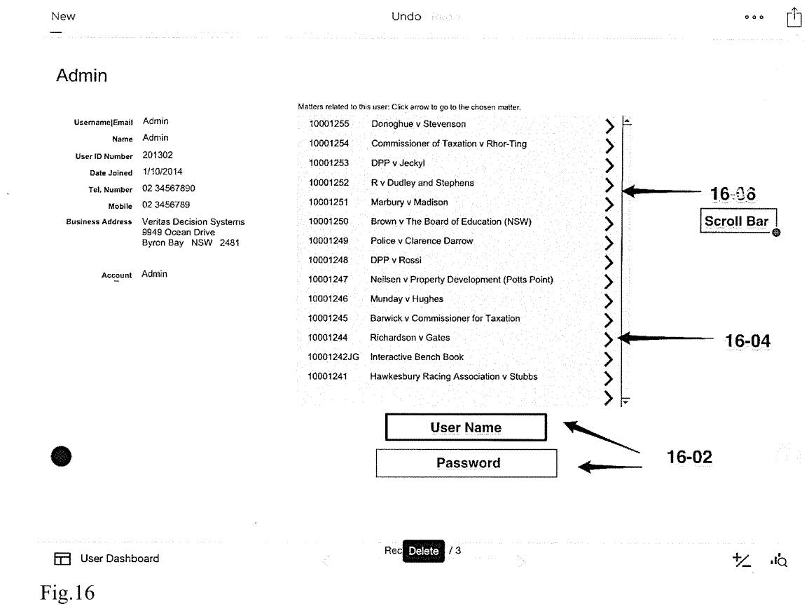

In FIG. 16, when the program is activated and opened, the decision maker is taken to the Portal. There may be a secure log in facility restricting access to the program and its data contents by means of password or other secure log in process—16-02.

The decision maker selects a new decision file, or opens an earlier (complete or partially complete) decision file —16-04.

A scroll bar may be utilised by user 101 to bring all decision files into the visual display—16-06.

User 101 selects an existing matter or in selecting Interactive Bench Book starts a new matter. The decision support system then opens in Data Matrix mode. The Data Matrix mode is the default display of computer system 100 on screen 102 when user 101 opens a file 16-04 from the Portal.

The Data Matrix

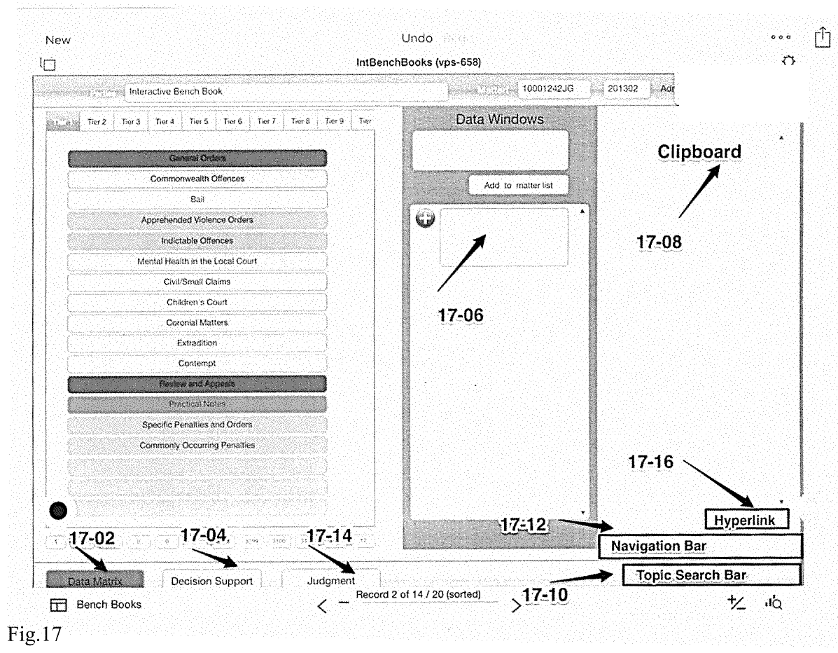

FIG. 17 illustrates a visual display of the decision support system opened in Data Matrix mode comprising a persistent menu identifying the plural different types of resource information, such as the Data Matrix button 17-02, Decision Assistance button 17-04, Data Windows 17-06, Clipboard 17-08, Topic Index Search 17-10, Navigation Bar 17-12 and Report (or in this example Judgment) button 17-14 and Hyperlink button 17-16.

The Navigation Bar 17-12 provides regions that the decision maker can activate in order to backtrack to an earlier node in the decision tree or to move from such a location further forward through the decision steps followed by user 101.

When the program is activated and opened, the program is activated in Data Matrix mode (which is computer system 100 default mode) and the decision maker is taken to Data Matrix mode by default. The decision maker may at any stage after the program is activated select Decision Support mode or Report (Judgment) mode by selecting the buttons for those modes, 17-04 or 17-14. User 101 may move freely and interchangeably between these modes at any stage when using computer system 100.

One example of the operation of computer system 100 will be a decision support system for use by a Judge or Magistrate in the discharge of their judicial function. This example will be used for the purposes of illustrating the operation of computer system 100 but there is no limit to the nature of applications to which computer system 100 may be applied, including medical diagnoses, engineering and architectural applications, preparation of teaching and learning materials, instruction manuals for any applications, presentation of teaching and learning materials, and product safety manuals (such as decision systems for diagnosis of faults in aircraft operation systems and steps for rectifying or responding to such faults).

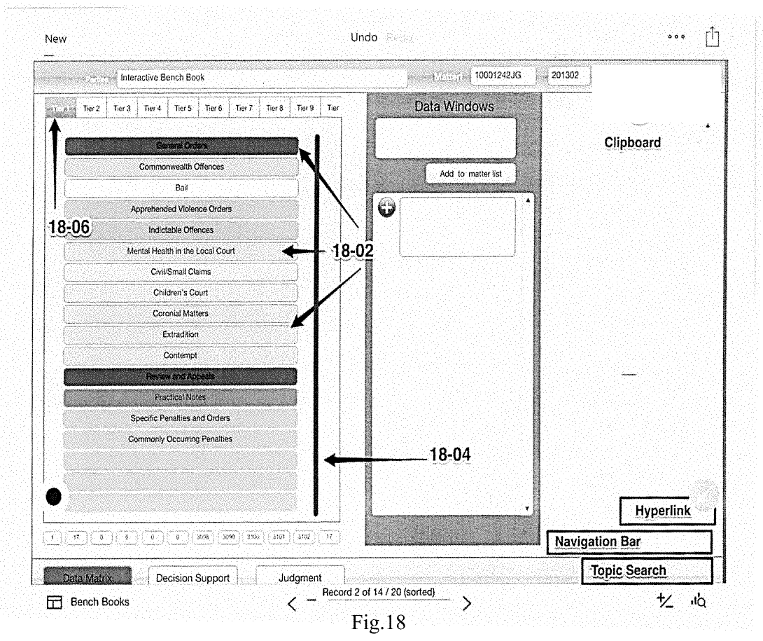

FIG. 18 shows the Data Matrix mode of a Judicial Decision Support System. This Judicial Decision Support System contains the data resources which facilitates the discharge of a Judges (or Magistrates) judicial function—that is, the discharge of their duties in deciding cases and applying the law in court in relation to legal issues and problems which arise in matters which proceed before that judicial officer. The database relevant to the discharge of a judges judicial function is large, and includes (but is not limited to) many types of matters, from commercial disputes, sentencing defendants, criminal law proceedings, family law proceedings, personal injury proceedings, and administrative law proceedings, depending on the jurisdiction of the court over which the judge presides.

In one example, the primary topics relevant to the judicial function of a Judge of Magistrate are contained in a Data Matrix, called in this example the Interactive Bench Book. By way of illustration, a “Bench Book” is a common professional term for a book which contains the data and resources frequently used by a Judge when presiding over a specific court.

18-02 illustrates the primary nodes within the Data Matrix relevant to the Interactive Bench Book contained in the visual display.

A node is a topic. Every node within computer system 100 has at least 1 Data Window linked to it. In Data Matrix mode there are frequently more than one Data Window associated with each node.

Nodes may share Data Windows—that is, one Data Window may be linked to more than 1 node within decision system 100.

The number of topic nodes is not restricted by the size of the visual display as there is a directional arrow or scroll bar which allows the user to bring into the visual display other primary topics or nodes. The scroll bar is illustrated in 18-04.

The primary nodes are located at Tier 1 of the decision system 18-06. The decision maker may scroll though those nodes to select the node for which they require assistance.

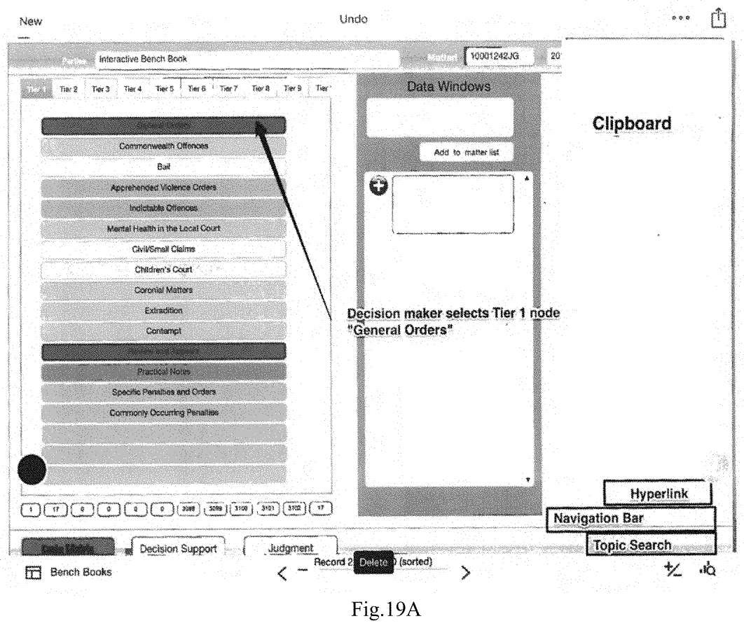

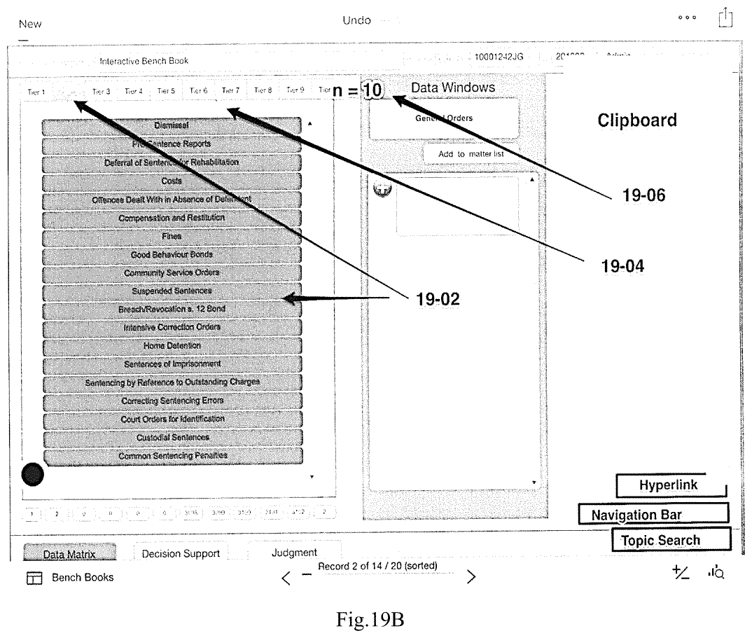

When the node for which user 101 requires assistance is selected, computer system 100 operates to update the computer screen 102 to bring the downstream nodes (or sub-nodes) relevant to the selected node into the visual display. FIG. 19A shows the decision maker having selecting a Tier 1 node. That causes computer system 100 to bring into the visual display the sub-nodes relevant to the selected primary node topics.

Those sub-nodes are in the computer system 100 shown as Tier 2 nodes in FIG. 19B. They are the downstream nodes relevant to the primary node and when the primary node is selected by user 101 these Tier 2 downstream topic nodes are brought into the visual display to replace the nodes in Tier 1 as shown in FIG. 19B.

Computer system 100 has a vast capacity in relation to the number of Tiers of data that may be included in the Data Matrix. The plasticity or depth of the decision system 100 will be determined by the volume of data contained in the decision system, the complexity of both the data and the decision trees relevant to solving problems related to that data, and the extent of the inter-relationships between the nodes within and between the Data Matrix and Decision Assistance modes.

The number of Tiers of nodes in the Data Matrix reflects the complexity of the decision system and is identified by an index “n” 19-04. That index is a measure of the depth or complexity of the decision system. It is referred to as the Depth Index of the decision system. In one example, the Judicial Decision Support system used as one example in this patent application, the Decision Support System has a depth index of 10 19-06.

Every Tier in the Data Matrix takes the decision maker deeper into the primary node—providing increasingly greater specialisation and increasingly greater focus on the complexity of the subject area of that node. The decision maker may delve as deeply as they may require into the complexity of the subject area depending on the specific problem they are using the decision system 100 to solve.

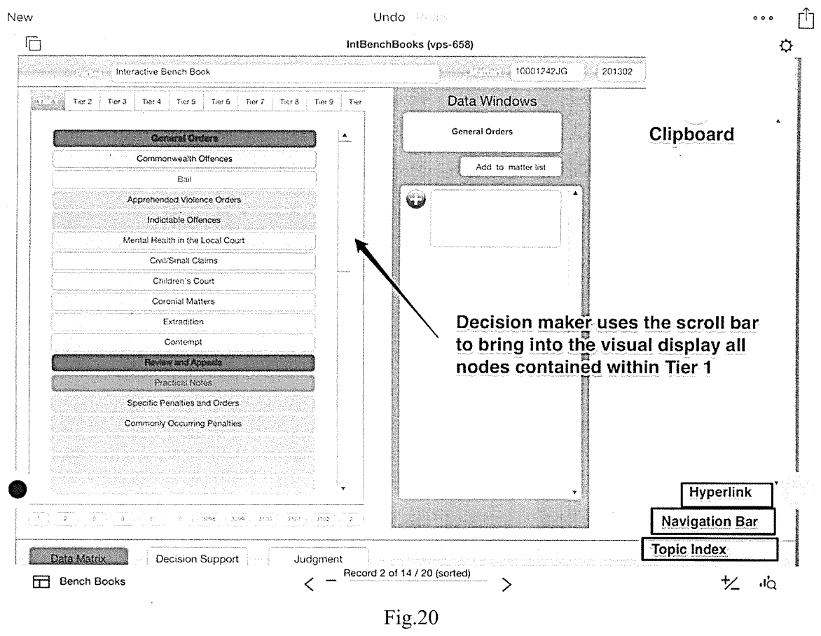

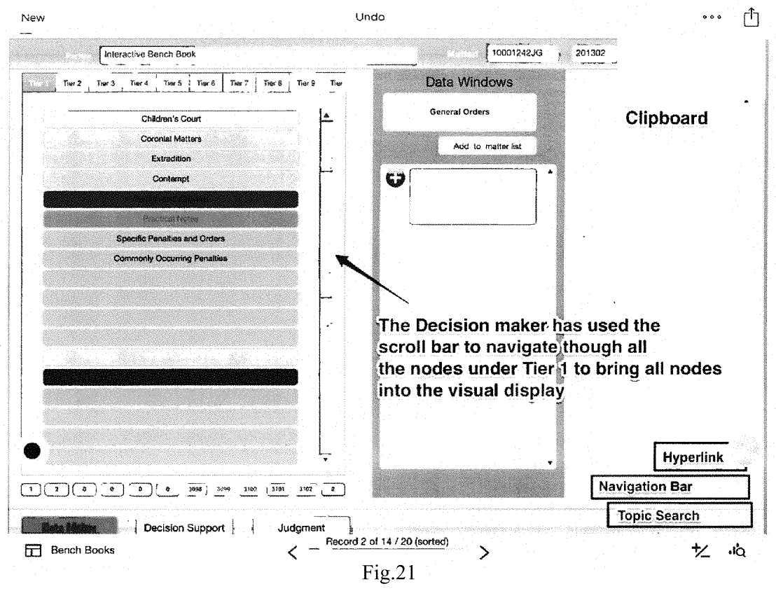

FIGS. 20 and 21 illustrates the use of the scroll bar by the decision maker to navigate though all of the nodes within the primary node Tier of decision system 100 and illustrates that the number of nodes within any Tier of the decision system is not limited by the size of the computer screen 102.

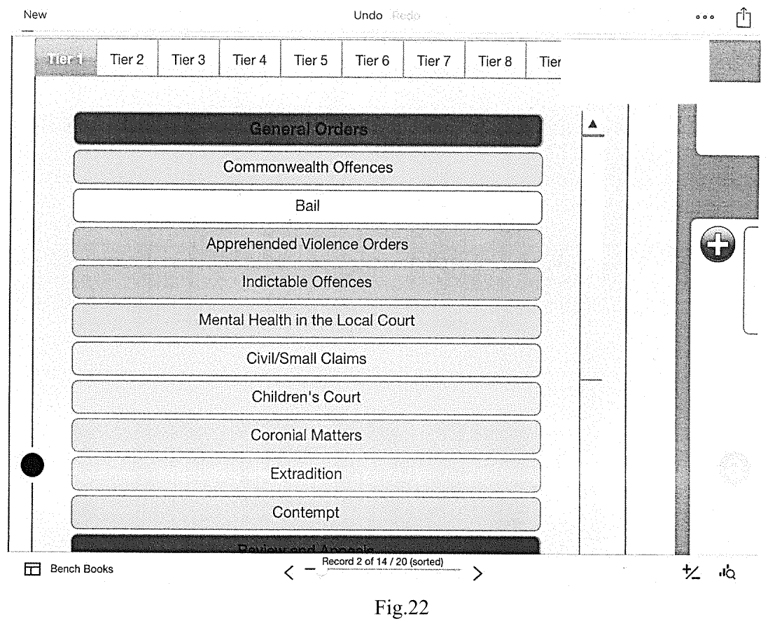

FIG. 22 illustrates that the visual display may be resized by the decision maker to any size (for example to fit the screen or to any proportion of the screen) to enable more accessible visual access to the interface to the program.

The re-scaling of the visual display may be activated by the decision maker using their fingers to rescale the size of the contents of the screen using the capacitative function available on computer screens and tablet devices or by other means available from computer system 100 on non-capacitative computer screens.

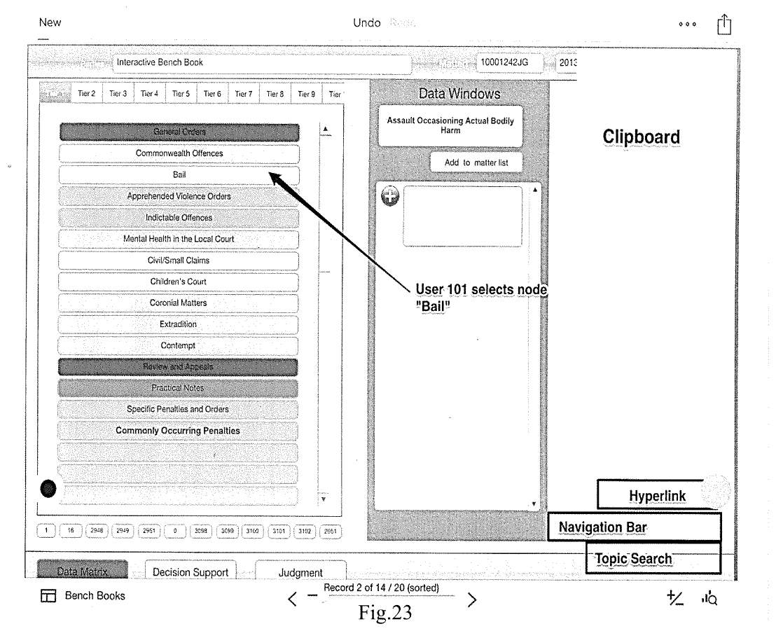

FIG. 23 illustrates a computer screen visual display after the decision maker selects a node from Tier 1. In one example, the decision maker selects a primary node, Bail, from Tier 1 of computer system 100 23-02

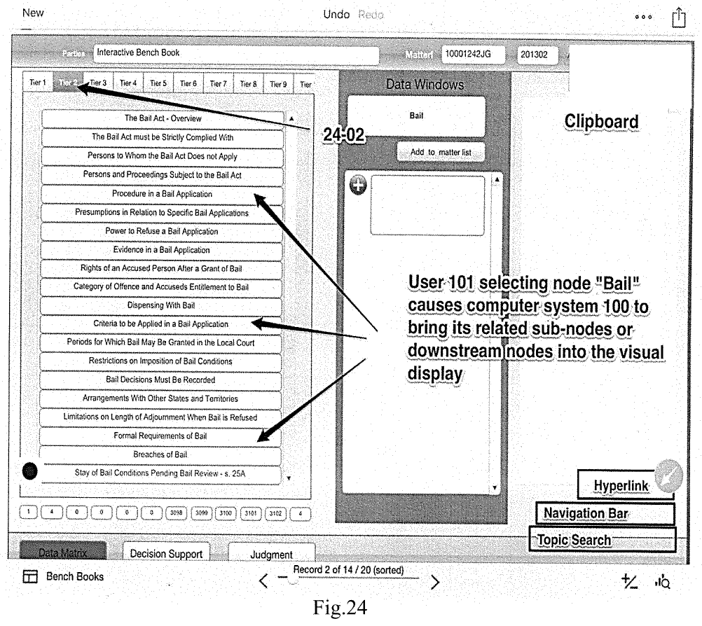

FIG. 24 illustrates the visual display of the downstream (Tier 2) nodes displayed by computer system 100 when the primary (Tier 1) node of “Bail” is selected 24-02. The selection of the Tier 1 node “Bail” brings its related sub-nodes (or downstream nodes) into the visual display.

Those Tier 2 nodes replace the Tier 1 primary topic nodes in the visual display. The nodes in Tier 2 which appear when a Tier 1 node is selected are the sub-topic nodes relevant to the upstream topic node selected.

The Tier 2 nodes which appear when a Tier 1 node is selected by the user are the downstream topics or related subtopic nodes to the Tier 1 topic selected.

Accordingly, and by way of illustration, when the primary node “Bail” is selected, all key sub-nodes relevant to Bail are brought into the visual display.

The Tier 2 button is highlighted 24-02 so the decision maker is made aware of their location in the sub-nodal hierarchy relevant to the primary node 24-02. That highlighted Tier 2 button shows the decision maker how far (or how deeply) into the Data Matrix they are in relation to the topic nodes in the display before them.

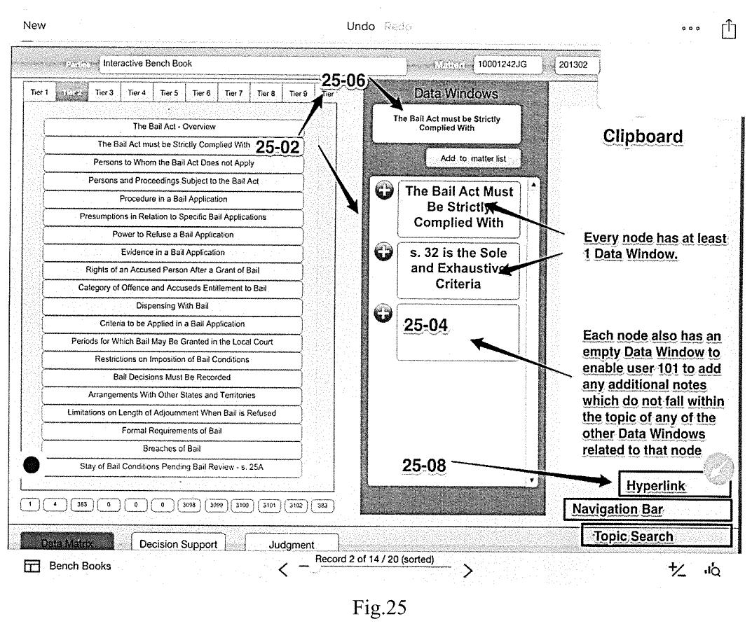

FIG. 25 illustrates that every node—whether primary or secondary (or further subsidiary) node—has at least one data window 25-02.

A Data Window may be shared by more than one node within computer system 100.

Every node also has an empty Data Window in addition to containing Data Window or Windows containing content so that a decision maker may add any additional notes they may wish in respect of that node topic 25-04.

Each Data Window or Windows relevant to a topic node appear in column 2 Data Windows when the node is selected 25-06.

As noted, every node in the Data Matrix has at least 1 Data Window. Each Data Window within computer system 100 (whether in Data Matrix, Decision Assistance, or Report or Judgment mode) may contain different types of information, including (but not limited to) text, graphics, images, intra-links (being links to other items within the Data Matrix), inter-links (links to other items in related decision support computer 100 systems within a network) and hyperlinks. When the user selects a hyperlink embedded in the Data Window, the hyperlink can open the document or resource (ie pdf, graphic, any image or other resource) within a window within computer system 100 in the visual display. Alternatively if user 101 selects the user may follow the hyperlink outside computer system 100 to the resource to which the hyperlink relates on the world wide web.

25-08 illustrates hyperlink button which remains in the persistent display. The decision maker may select the hyperlink embedded in the Data Window and by pressing the hyperlink button 25-08 may open that hyperlinked resource (which may include but is not limited to a document, images, videofile, or audiofile) within a window within computer system 100 and the decision maker may use the copy facility to remove data from a hyperlinked resource to insert into a Data Window within computer system 100.

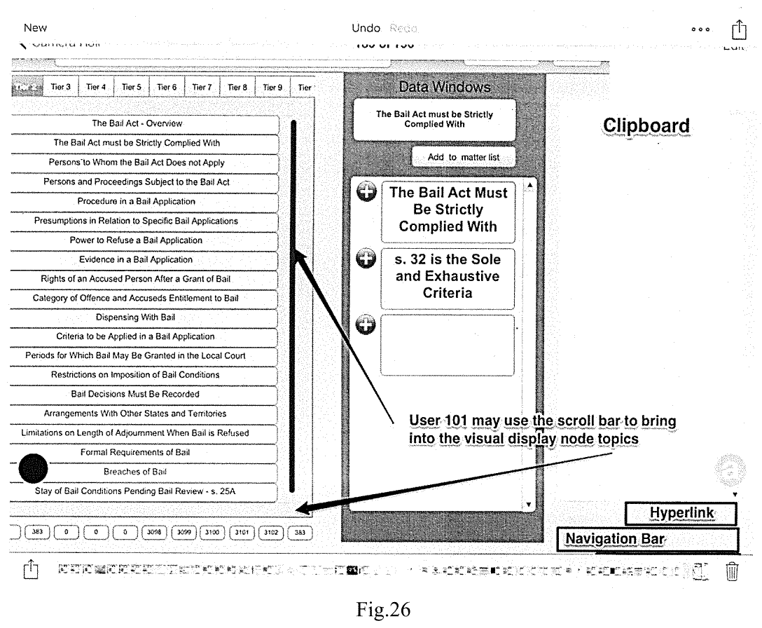

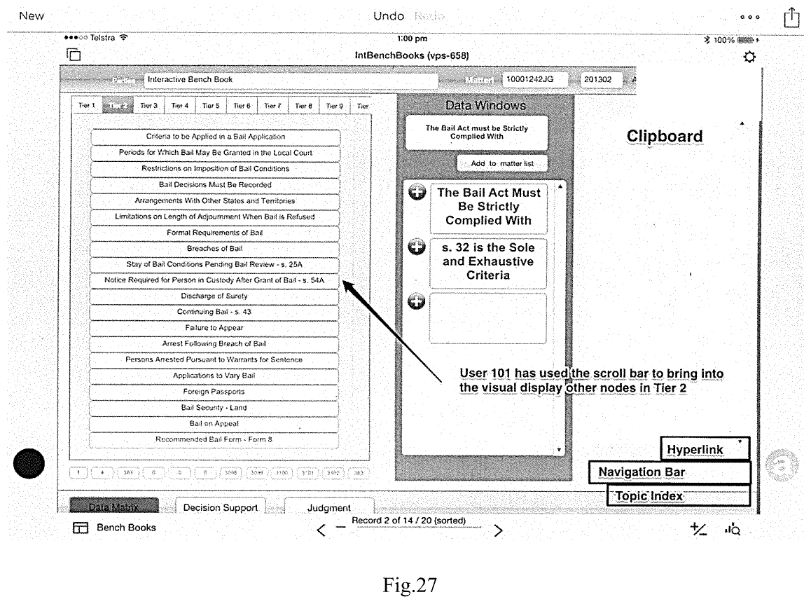

FIGS. 26 and 27 illustrate that the number of Tier 2 nodes (sub-nodes for the primary node “Bail”) are not restricted by the size of the visual display i The decision-maker may use the scroll bar function to peruse all Tier 2 sub-nodes relevant to “Bail”.

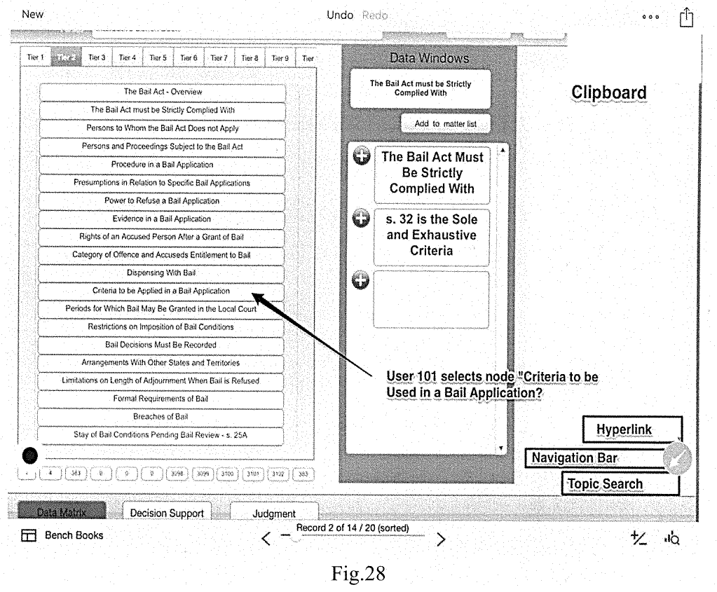

FIG. 28 illustrates a decision maker selecting a further sub or downstream node in relation to the “Bail” node. In this example, the user selects the topic node “Criteria to be Applied in a Bail Application”.

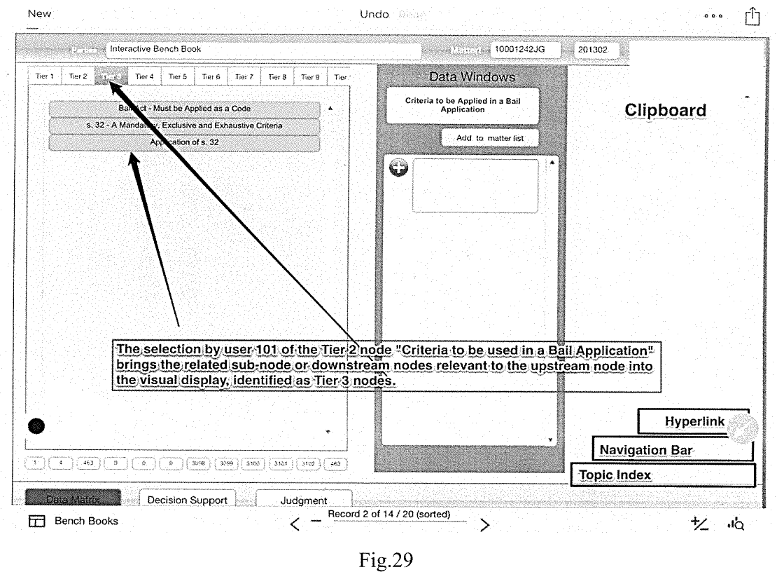

FIG. 29 illustrates that when the decision maker selects the Tier 2 node “Criteria to be Applied in a Bail Application” illustrated in FIG. 28, there are 3 downstream sub-nodes relevant to that topic which computer system 100 then brings into the visual display.

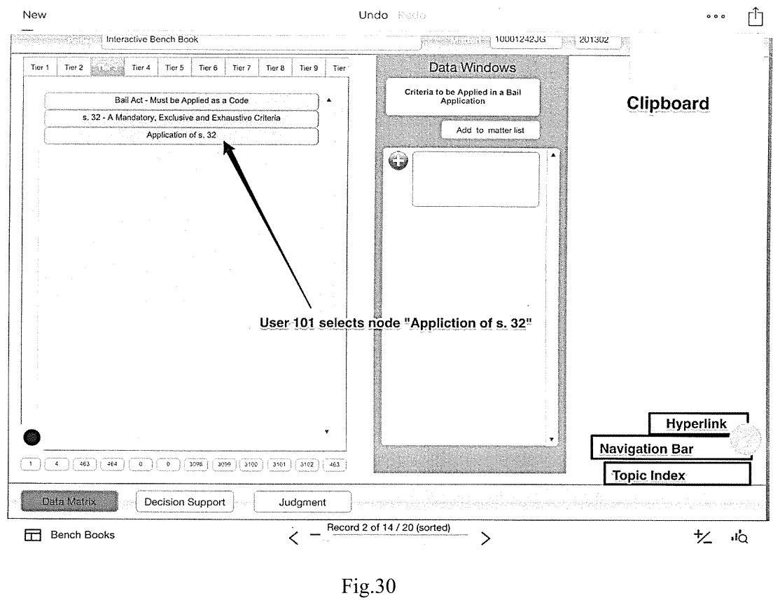

By further example, and to illustrate the decision maker moving further into the Data Matrix, the particular problem the decision maker may require assistance with in one example is the Application of s 32 of the Bail Act. This section applies in relation to the law which is applicable to the question of whether a person will be considered likely to appear in answer to their Bail conditions. FIG. 30 illustrates the decision maker selecting the node “Application of s. 32”

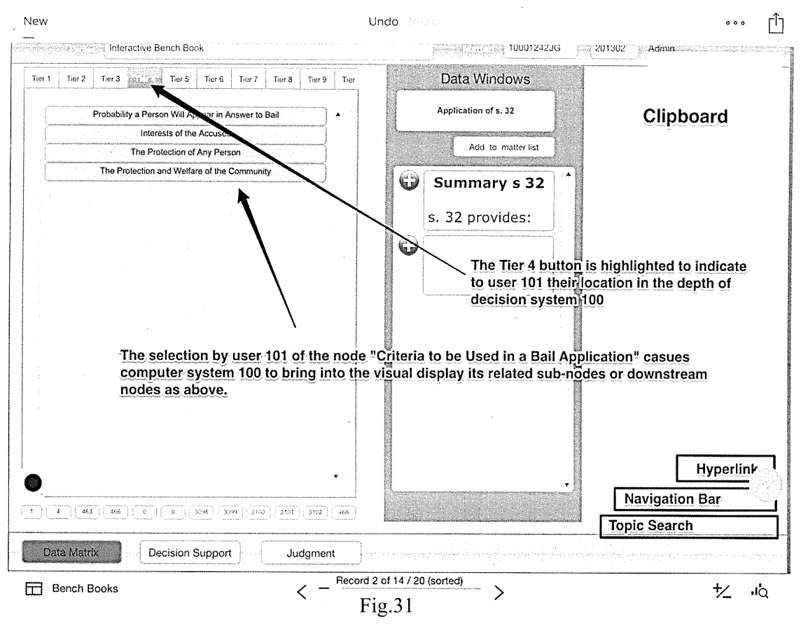

FIG. 31 illustrates computer system 100 updating the screen display when activated by user 101 in selecting “Application of s. 32” and displays the screen display in which computer system 100 identifies the matters that relate to the Application of s. 32 of the Bail Act 718.

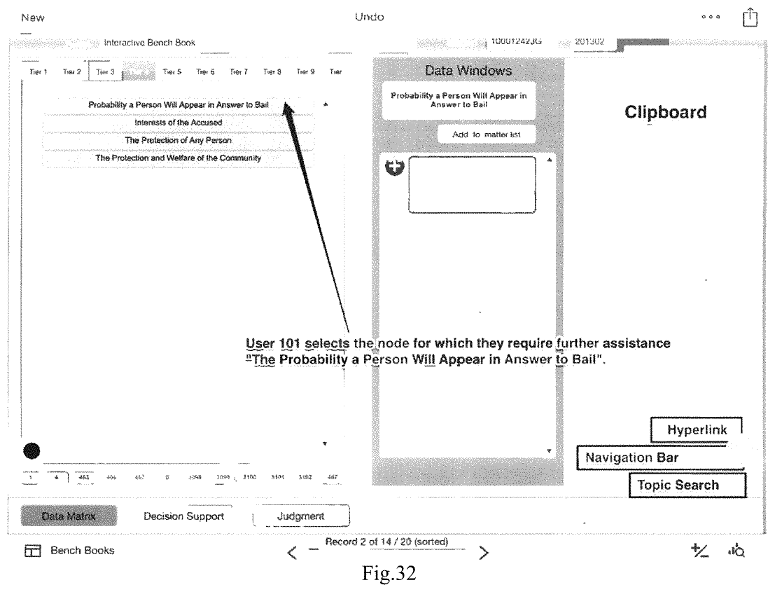

FIG. 32 illustrates the user selecting the node in respect of which they require further assistance—the Tier 4 node “The Probability a Person Will Appear in Answer to Bail” 32-02.

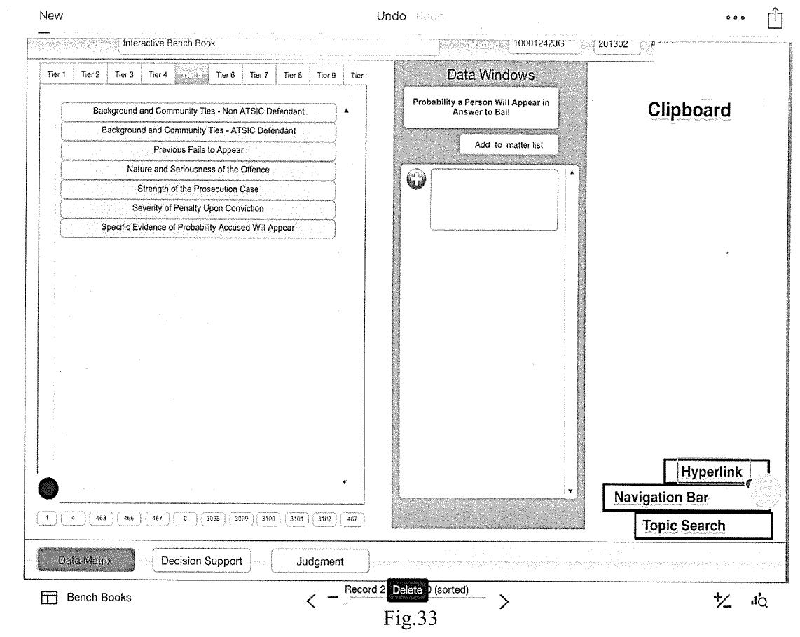

FIG. 33 illustrates computer system 100 displays the legal topic nodes a Judge is required to consider in determining whether a person is to be considered or concluded to be probable to appear in answer to Bail.

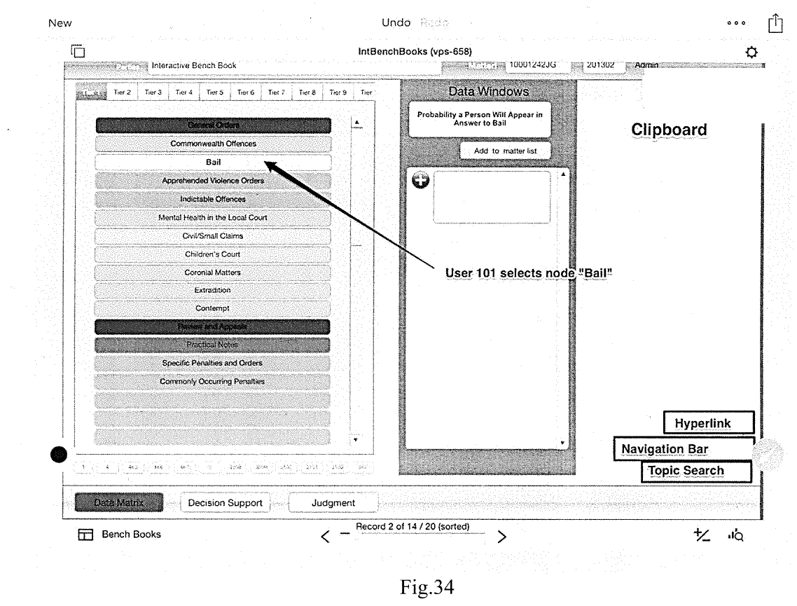

FIG. 34 provides a further illustration of the use of the Judicial Officers Decision System in Data Matrix mode.

The decision maker wishes to know the procedure in a Bail Application.

FIG. 34 illustrates the decision maker selecting the topic node Bail From Tier 1 nodes.

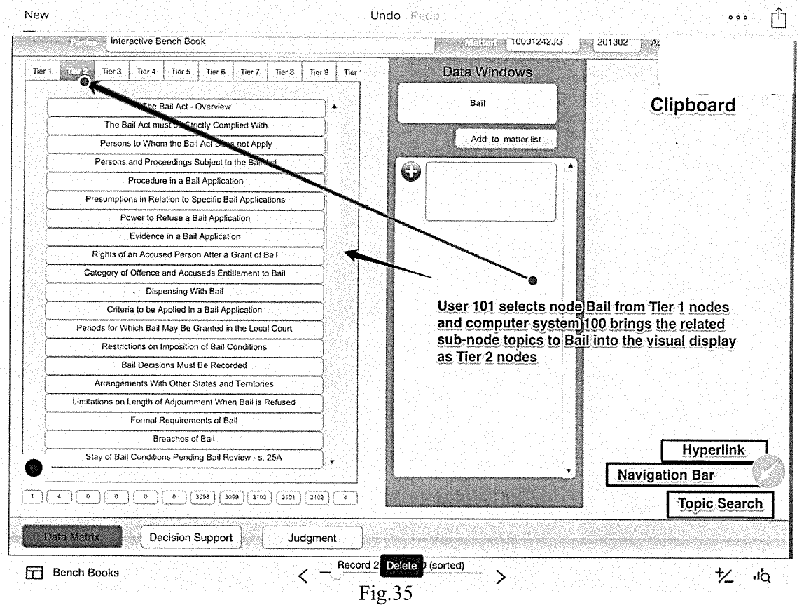

FIG. 35 illustrates that the selection of the Bail node within computer system 100 brings into the visual display the sub-topic nodes relevant to the node Bail.

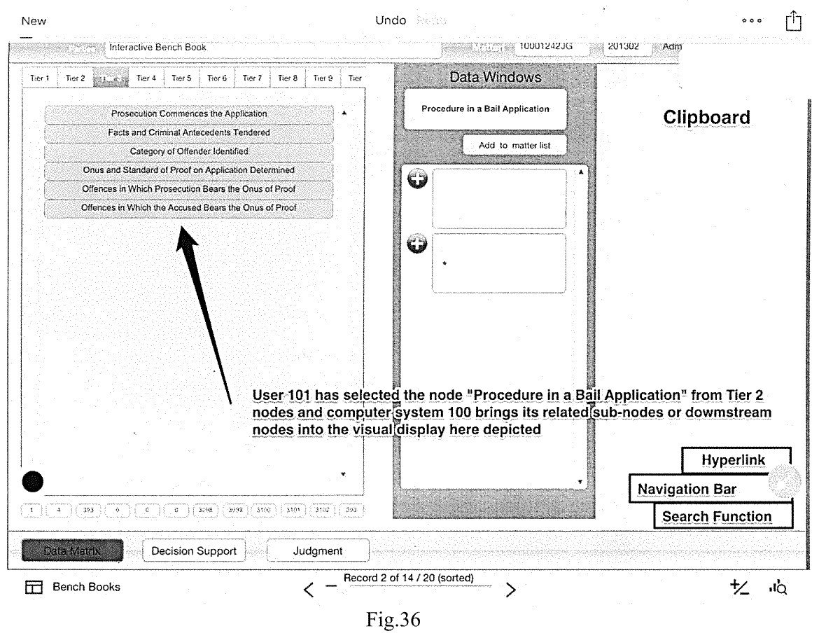

FIG. 36 illustrates the decision maker selecting the node “Procedure in Bail Application” and the selection of that node causes computer system 100 to bring into the visual display the Tier 3 sub-nodes relevant to the node “Procedure in a Bail Application”—as illustrated, those nodes being:

Prosecution Commences the Application

Facts and Criminal Antecedents Tendered

Categories of Offender Identified

Onus and Standard of Proof on Application Determined

Offences in Which Prosecution bears the Onus of Proof

Offences in Which the Accused Bears the Onus of proof

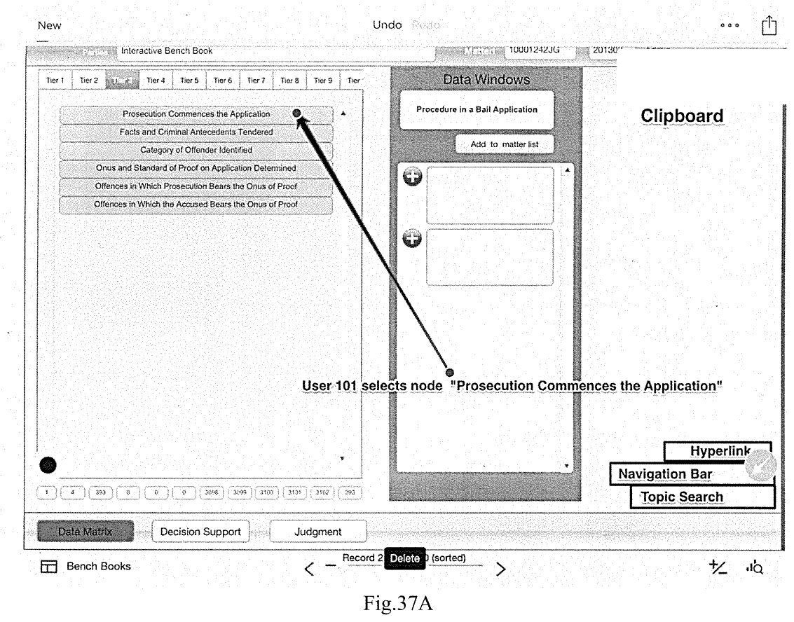

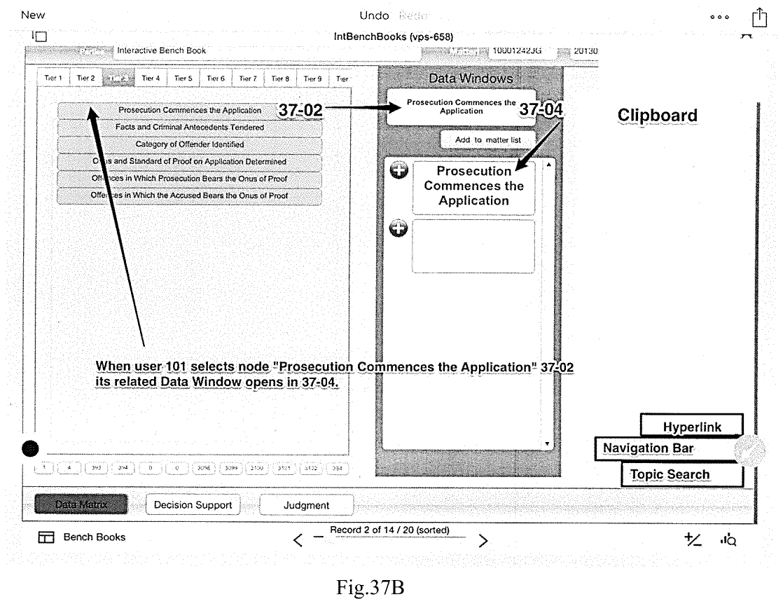

FIG. 37A illustrates the decision maker selecting the first Tier 3 node—that is, the first sub-node relevant to the upstream Topic Procedure in a Bail Application. The decision maker selects “Prosecution Commences the Application” 37-02 and that opens the Data Window relevant to that topic in FIG. 37B.

The Heading in the Data Window 37-04 is the same as the topic node which has been selected by user 101.

Every Data Window within computer system 100 (whether in Data Matrix, Decision Support or Report mode) may contain plural different types of resource information, including but not limited to:
(1) Intralinks—links between specific content in the Data Window to other Data Windows within that same decision system
(2) Interlinks—links between specific content in the Data Window and specific Data Windows within other distinct (but related) Decision Systems (such as a related Evidence Decision Support System, or a Complex Sentencing Decision System); and
(3) Hyperlinks—links between specific content in a Data Window and to sites on the World Wide Web, such that a specific document or image etc may be opened in a window within the computer program or at the election of the decision maker the decision maker may be taken outside the decision system to the website containing the hyperlinked resource.

There can be any number of Data Windows related to a topic node in the Data Matrix.

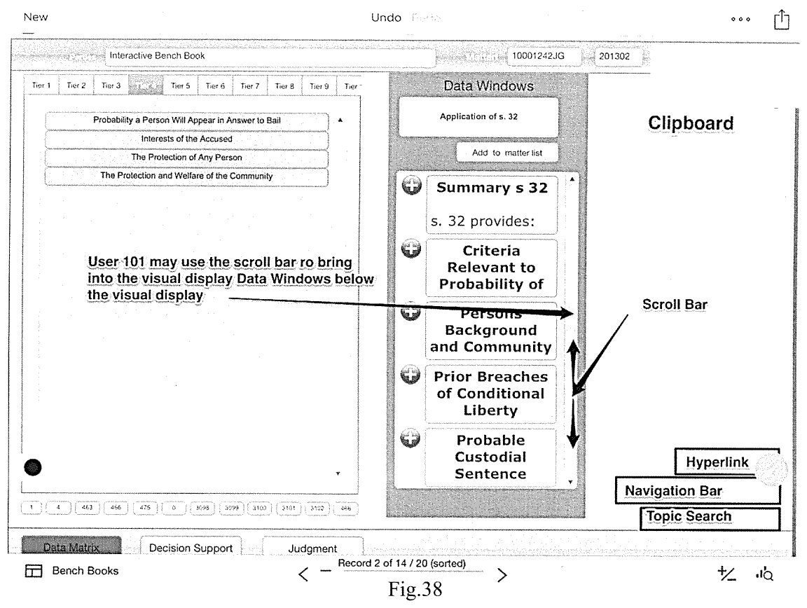

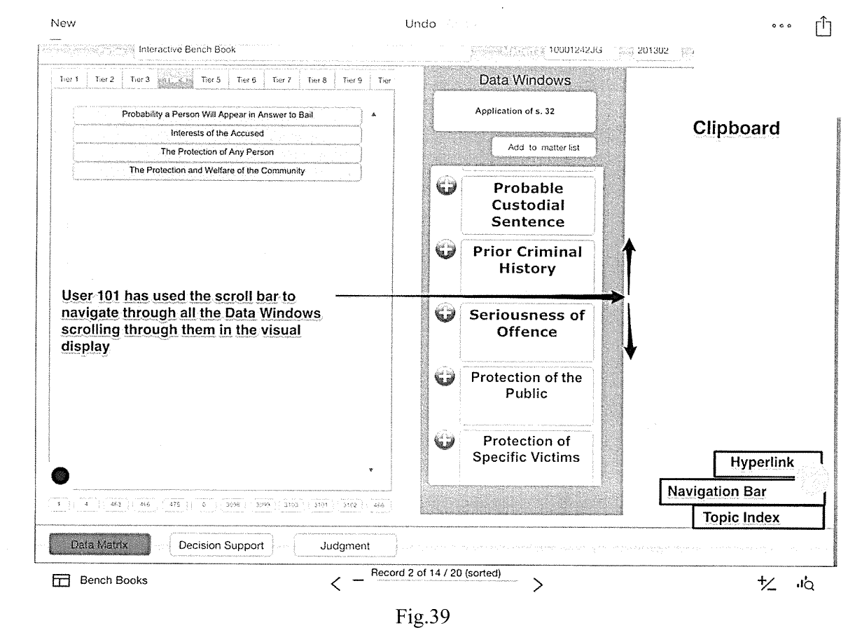

The decision maker may use the scroll bar in the Data Windows column to bring into the visual display of the computer screen 102 all of the Data Windows linked to that topic node. This is illustrated in FIGS. 38 and 39. These Data Windows relate to the node Application of s. 32 and provide a summary of the topics which may be selected and explored in greater detail by the user—progressing further into the Data Matrix as required—by selecting any of the nodes in FIG. 38. The decision maker may use the scroll bar to bring into the visual display those Data Windows shown in FIG. 39.

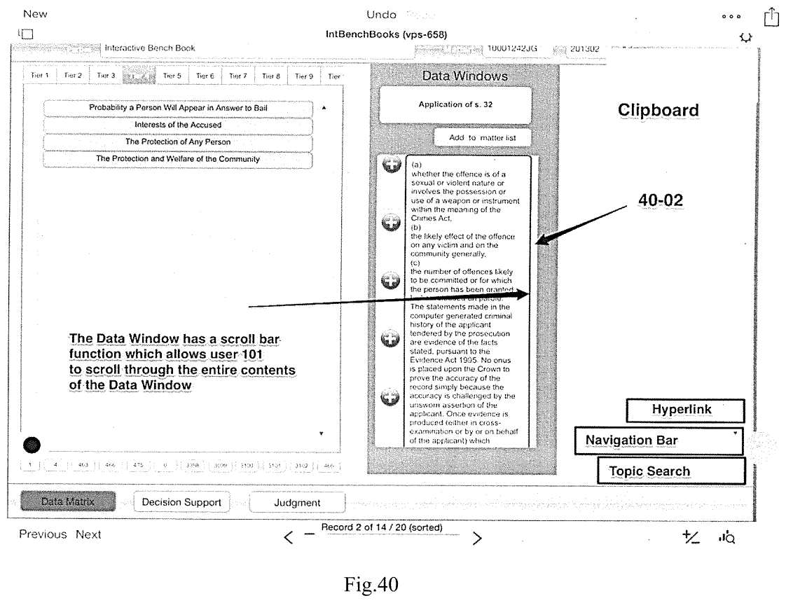

FIG. 40 illustrates how a Data Window may be opened by the decision maker by selecting it.

The Data Window has a scroll bar function 40-02 which permits the amount of Data in the Data Window to exceed the size of the visual display and which may be brought int the visual display by the decision maker utilising the scroll bar function.

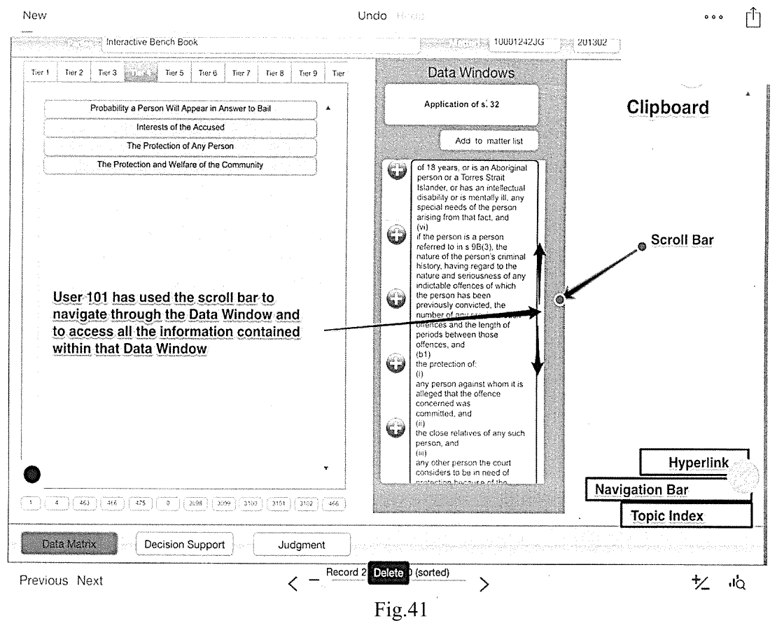

FIGS. 40 and 41 illustrates the location of the scroll bar function and its use by the decision maker to read the contents of the Data Window.

All Data Windows in computer system 100 (whether in Data Matrix, Decision Support or Report mode) have the scroll bar facility enabling the Data Window to store more information than may appear immediately in the visual display and will allow the user to scroll through the data contained in the Data Window.

The decision maker may re-size the Data Window to permit more ready access to the contents of the Data Window when the Data Window appears in the Data Windows column or the Clipboard.

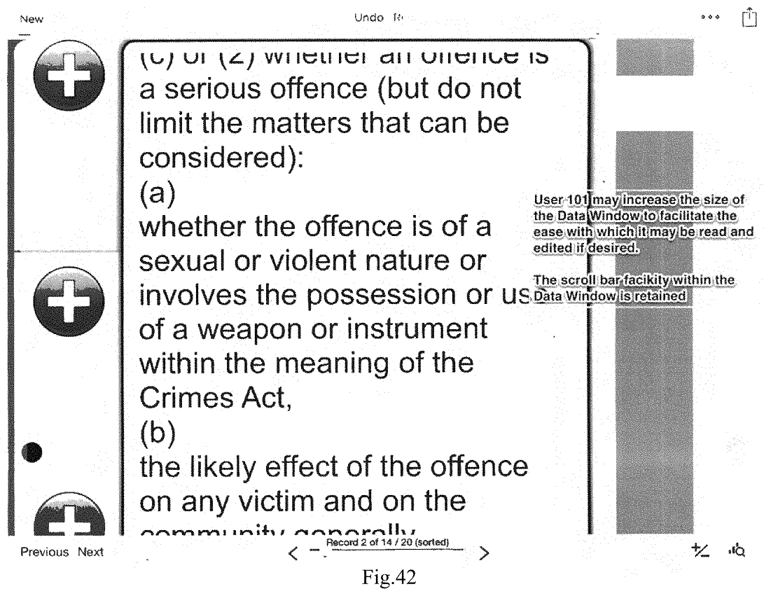

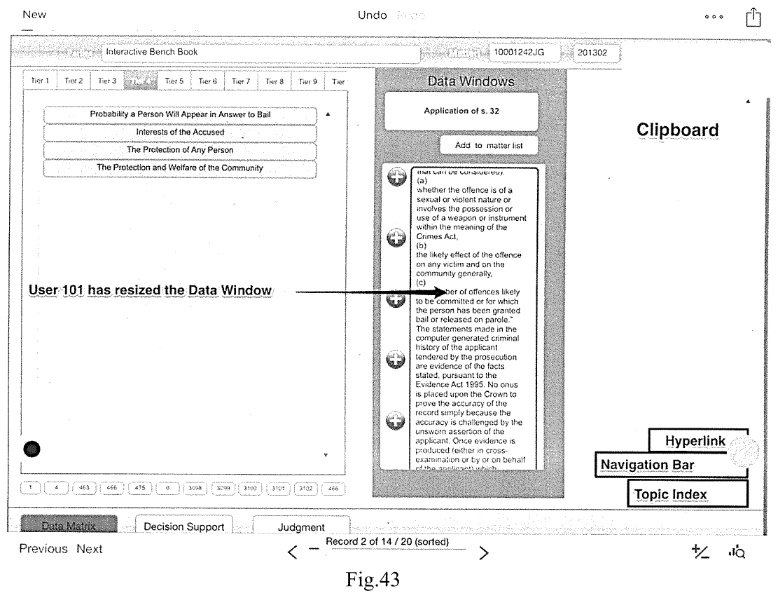

FIGS. 42 and 43 illustrates the decision maker re-sizing the Data Window. This may be done by a re-sizing button on a computer screen or by the decision maker using their fingers on the screen in a computer or tablet computing environment and the Data Window may be resized to the scale the decision maker considers useful. When the Data Window has been re-sized the Data Window retains the use of the scroll bar facility.

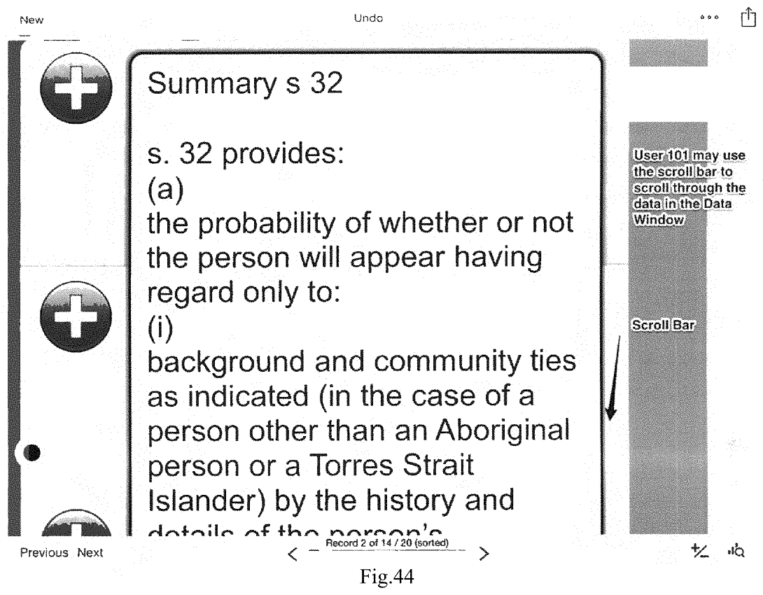

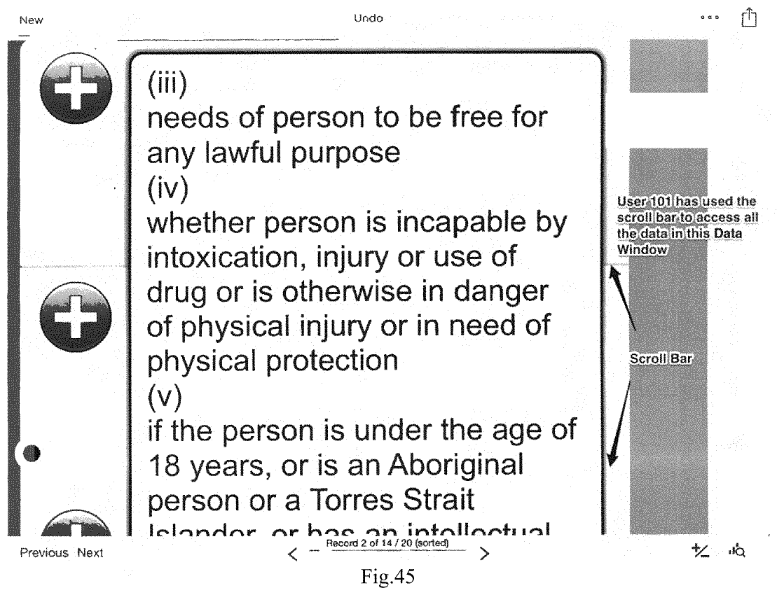

FIGS. 44 and 45 illustrates the decision maker using the scroll bar facility to scroll through the data contents of a resized Data Window.

If the decision maker considers the data contained in any Data Window of use in the problem the decision maker is seeking to solve, the decision maker may copy the entire contents of the Data Window to the clipboard by activating the Add to Clipboard Button relevant to that Data Window.

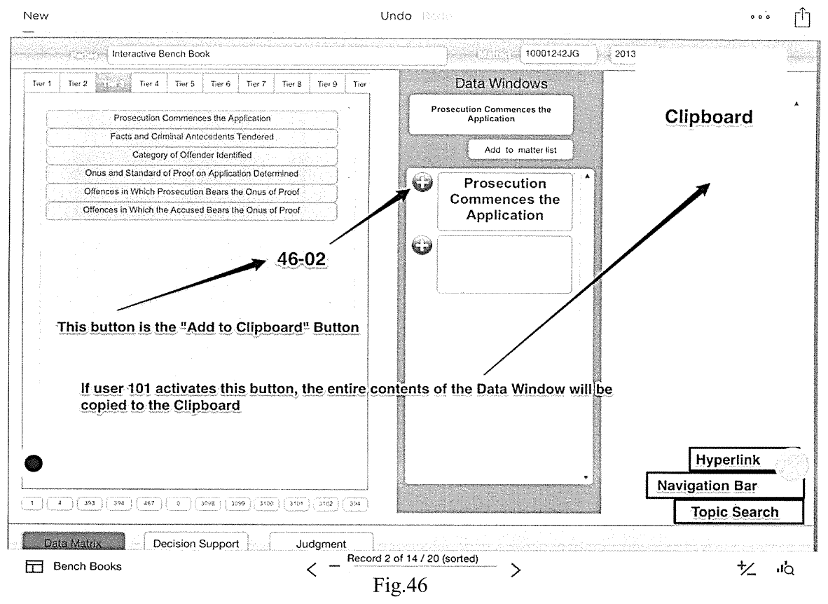

FIG. 46 illustrates a decision maker selecting the Add to Clipboard button and copying the Data Window onto the Clipboard.

Activating the Add to Clipboard Function 46-02 will copy the entire Data Window selected to the Clipboard, being column 3 of the visual display.

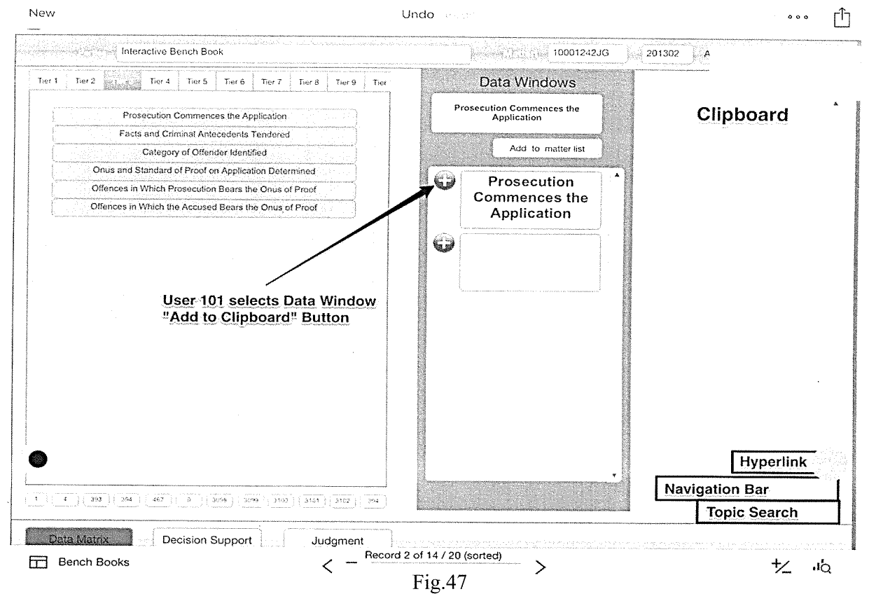

By way of one illustration, FIG. 47 illustrates a decision maker selecting the Data Window “Prosecution Commences the Application” by activating the Add to Clipboard Button.

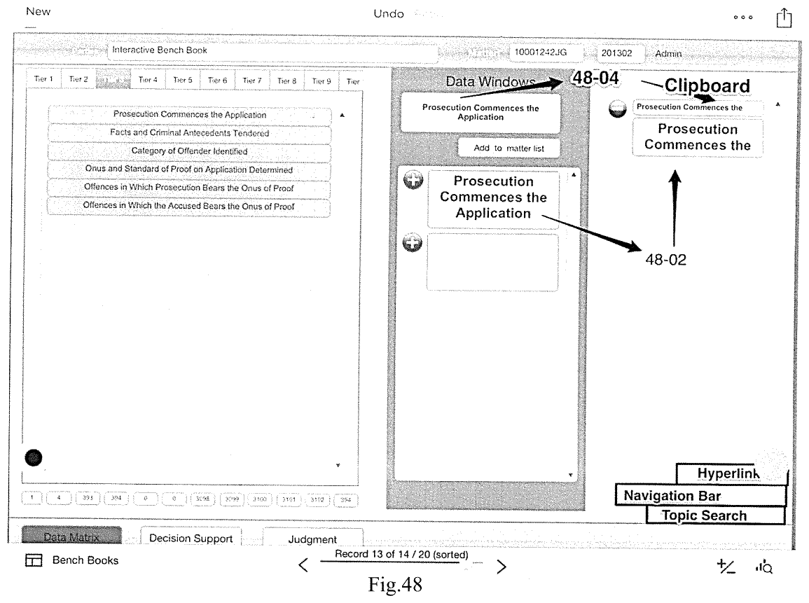

FIG. 48 illustrates the Data Window now added to the Clipboard 48-02. The Topic of the Data Window is preserved under a separate heading on the Clipboard for ease of recognition, editing and management by the decision maker 48-04. The Data Window retains its scroll bar function and all data is copied when the Add to Clipboard function is activated.

The decision maker may not wish to utilise all the data in a particular data window. The decision maker may open a Data Window and using a cut and paste function select only that data which is of assistance to them in relation to the specific problem they are trying to solve.

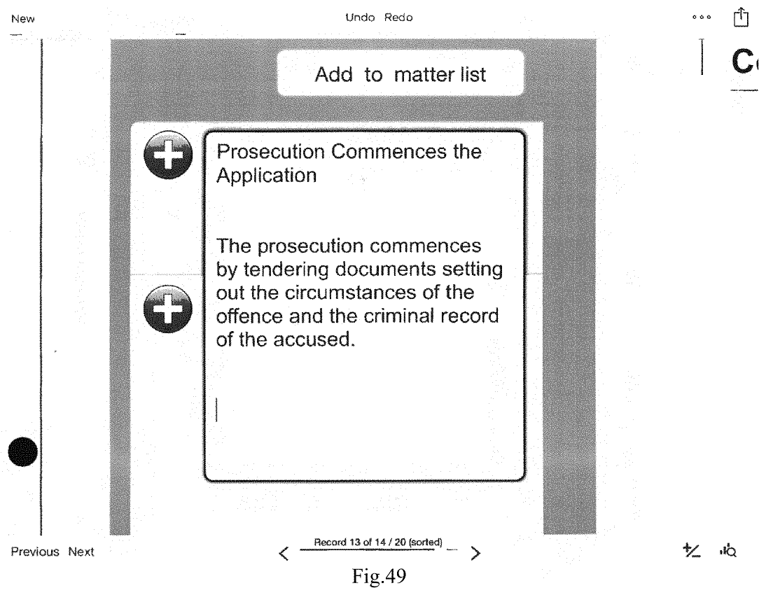

FIG. 49 illustrates the decision maker re-sizing a Data Window from the Data Window column.

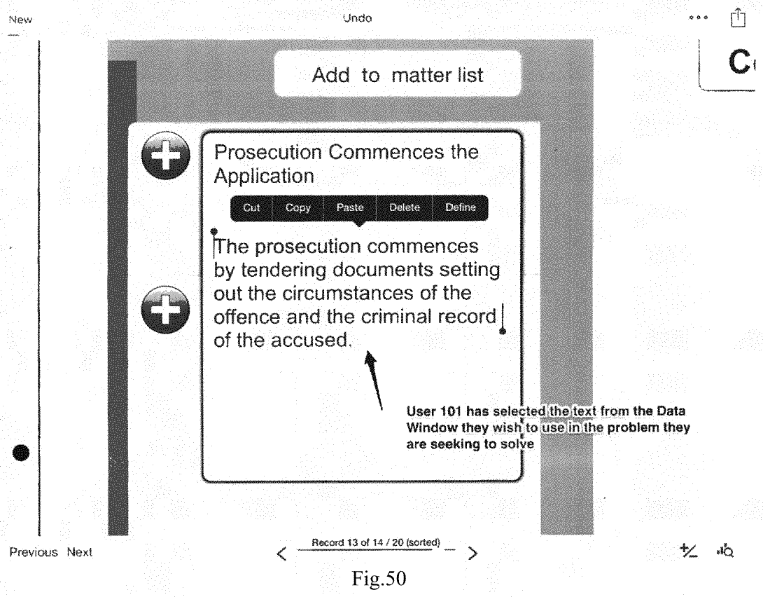

FIG. 50 illustrates a decision maker using the cut and paste function to select part of the data in that Data Window.

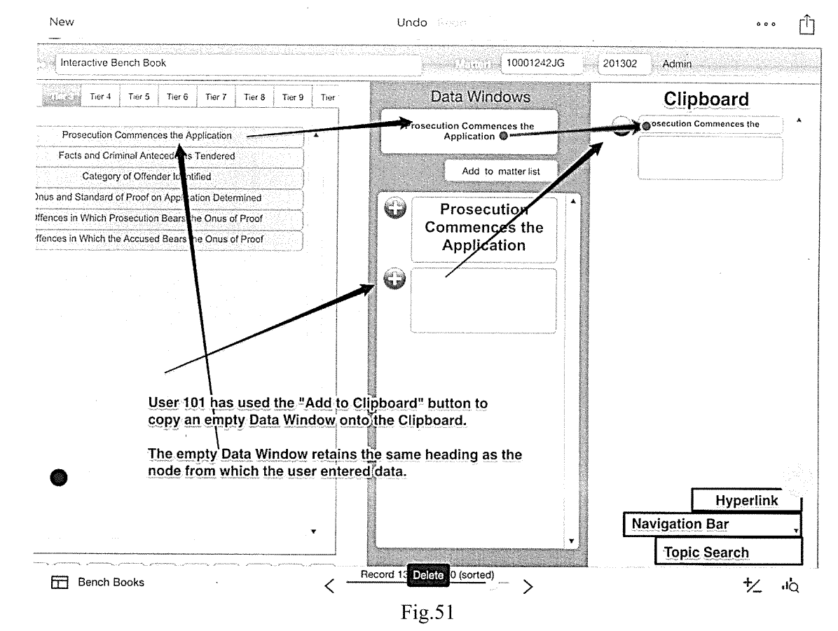

FIG. 51 illustrates the decision maker using the Add to Clipboard function to copy an empty Data Window onto the Clipboard.

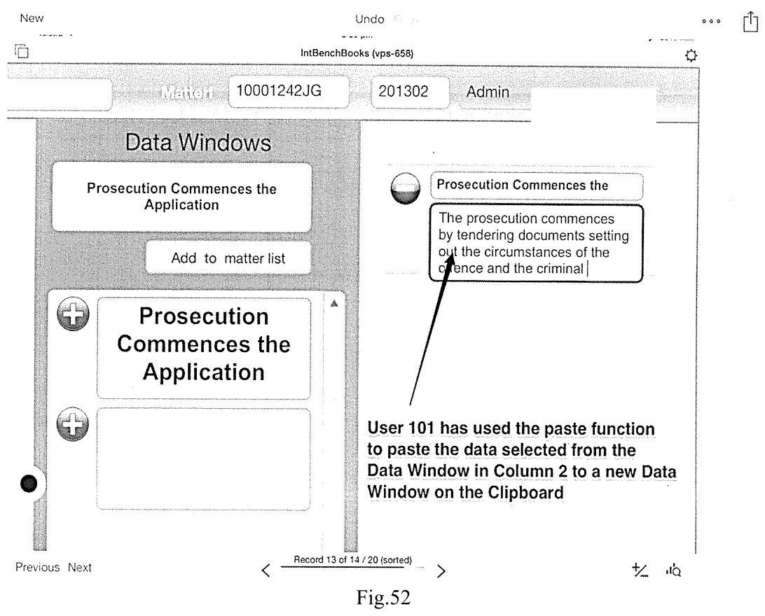

FIG. 52 illustrates the decision maker using the paste function to copy the selected part of the Data Window in the Data Windows Column to the empty Data Window in the Clipboard.

This permits the decision maker to use only that part of the content of a Data Window which is relevant to the problem they are seeking to solve.

The Data Window on the Clipboard into which the decision maker has copied part of the contents of the Data Window in the Data Windows column retains the Heading within the Data Window of the Clipboard from that from which the Data Window was originally copied—that being the heading displayed in relation to the node from which it was selected.

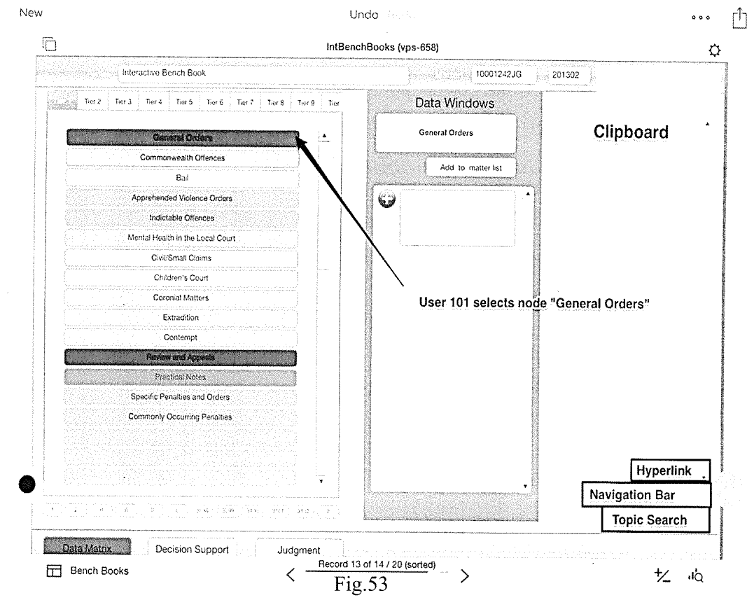

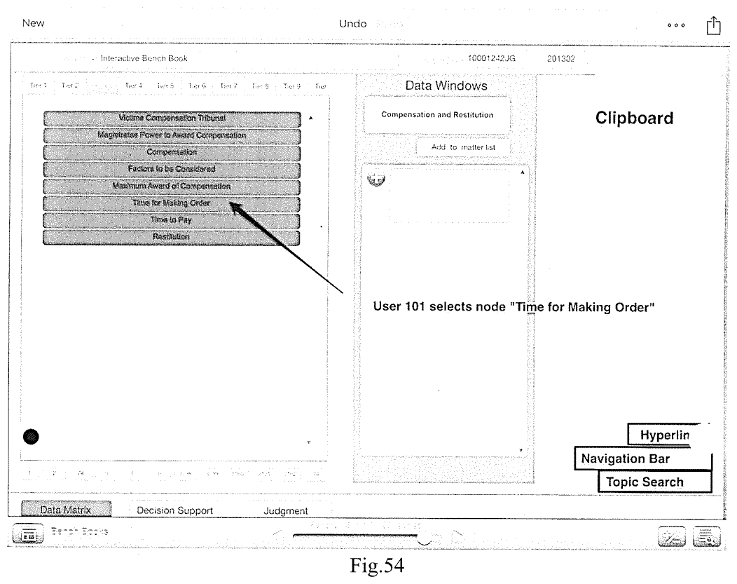

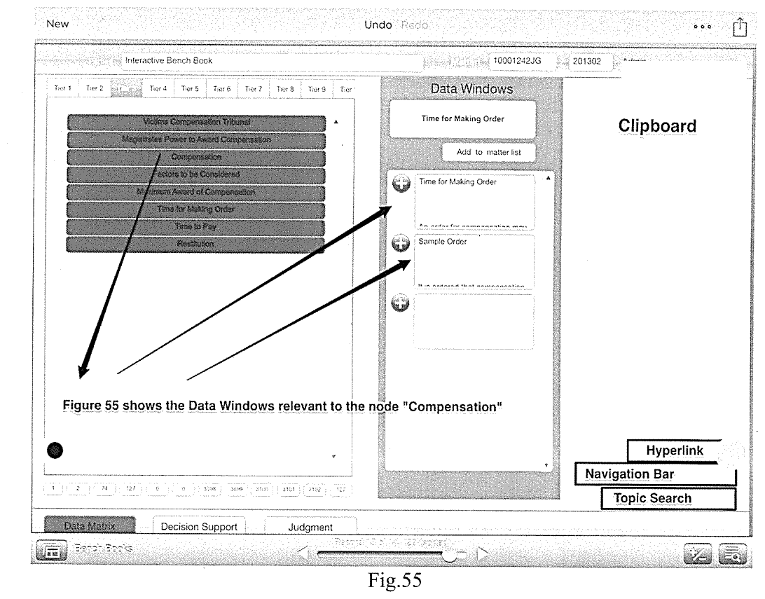

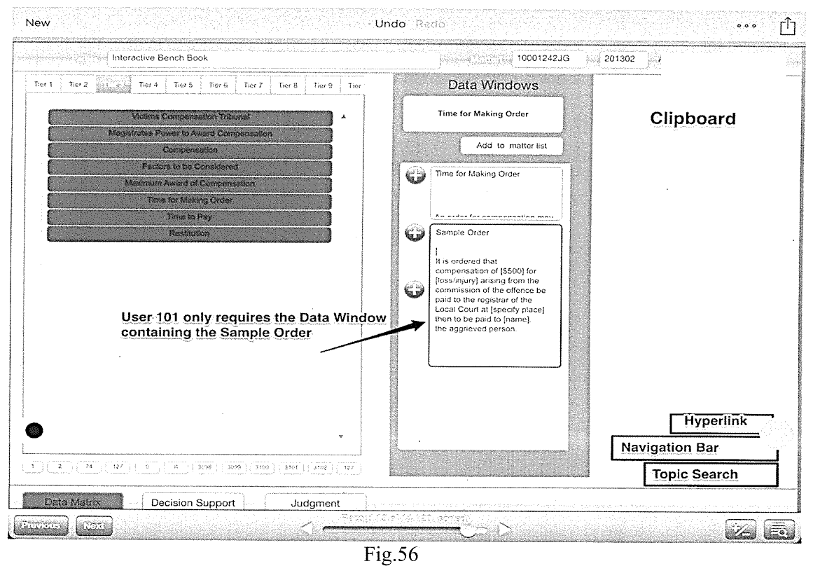

By way of further example, FIGS. 53-56 illustrate the decision maker is accessing the Data Matrix in relation to the Tier 1 node of General Orders, then the Tier 2 node Compensation and Restitution, and then the Tier 3 node Time for Making Order. The Data Window relevant to the node “Time for Making Order” contains different Data Windows relevant to that node and the decision maker may for example only require the draft or sample order.

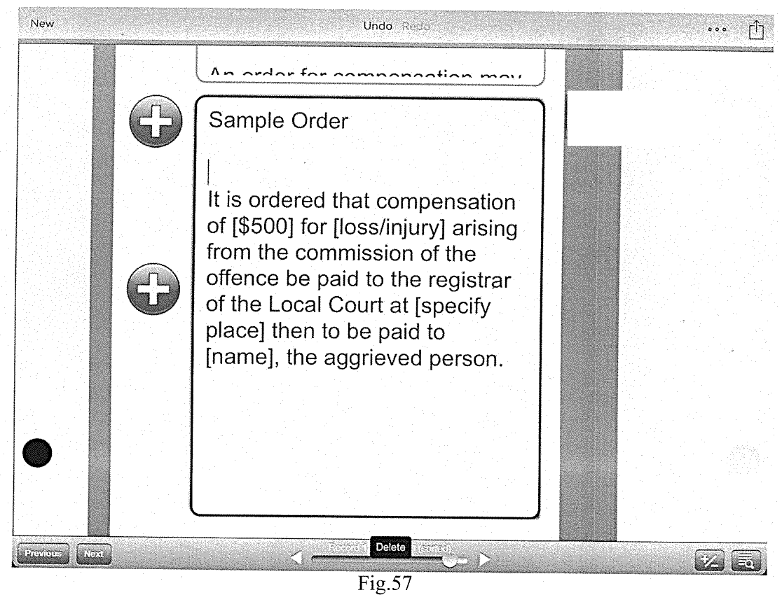

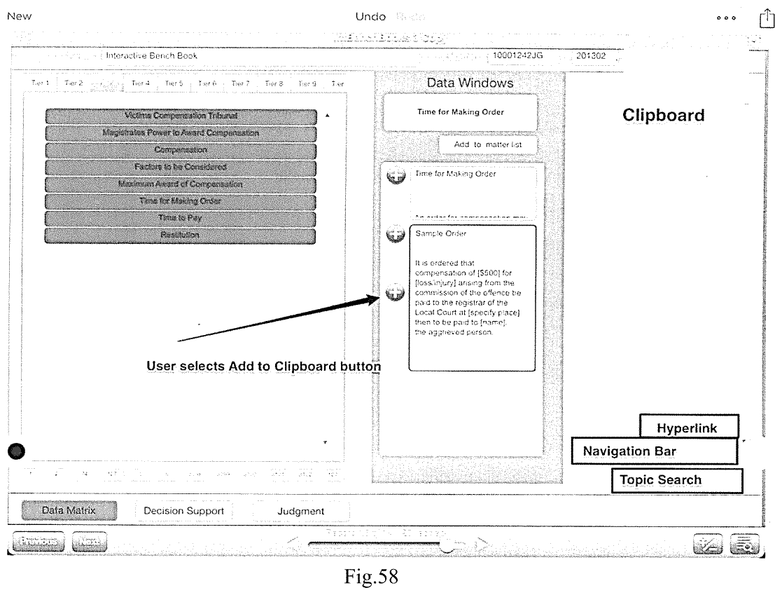

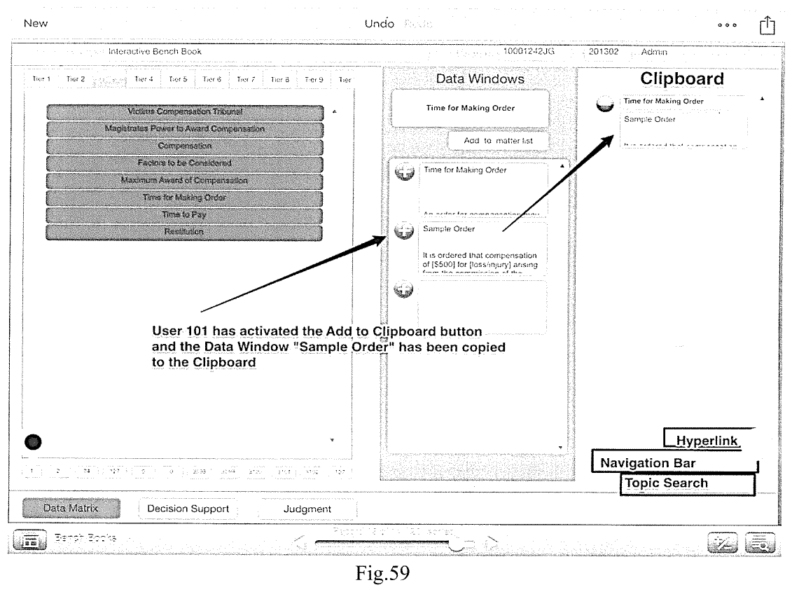

FIGS. 57 and 58 and 59 illustrates the decision maker opening the Data Window, “Sample Order”, resizing that window to read its content clearly, then uses the Add to Clipboard button to copy that Data Window onto the Clipboard.

The method provided by computer system 100 may be used by a decision maker to provide extremely rapid access to the information the decision maker may require to discharge their function—in one example, their judicial function.

A further illustration of the access the computer program 100 provides to the decision maker is found in access to common penalty nodes in the Data Matrix.

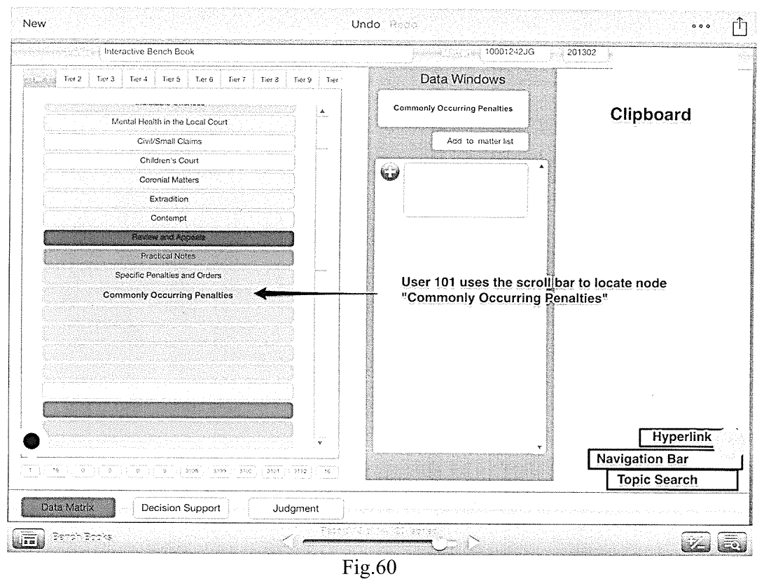

FIG. 60 illustrates the decision maker scrolling through the nodes in Tier 1 of the decision system to the node “Commonly Occurring Penalties”.

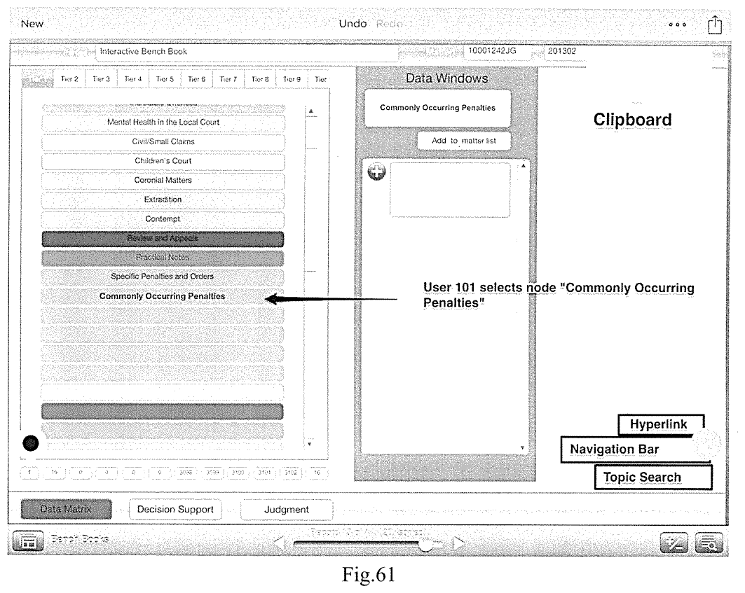

FIG. 61 illustrates the node “Commonly Occurring Penalties” being selected by the decision maker.

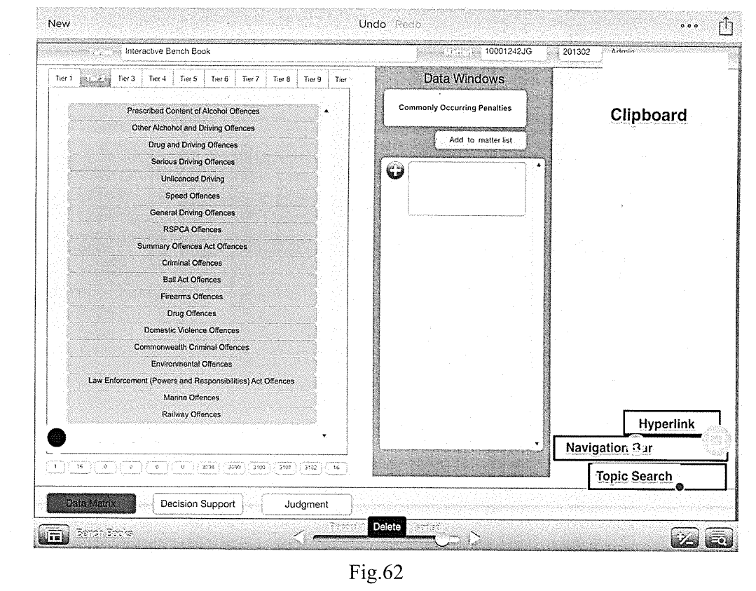

FIG. 62 illustrates the visual display when the node “Commonly Occurring Penalties” in FIG. 61 is selected.

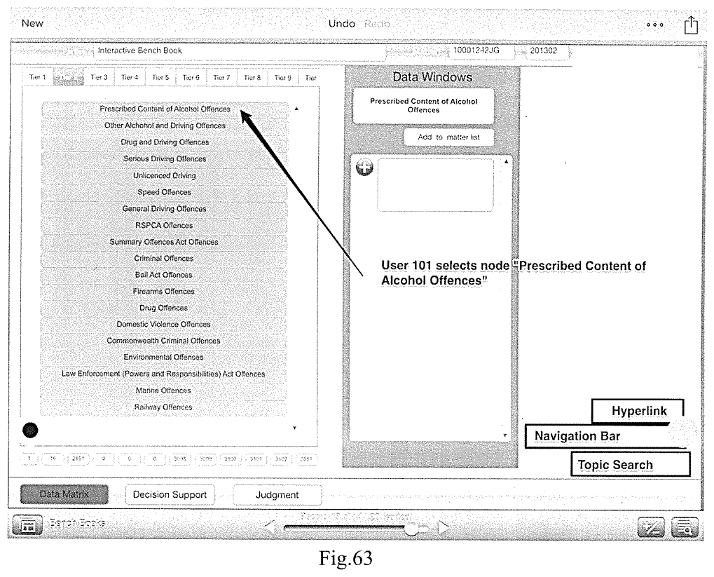

FIG. 63 illustrates the decision maker selecting the node “Prescribed Content of Alcohol Offences”.

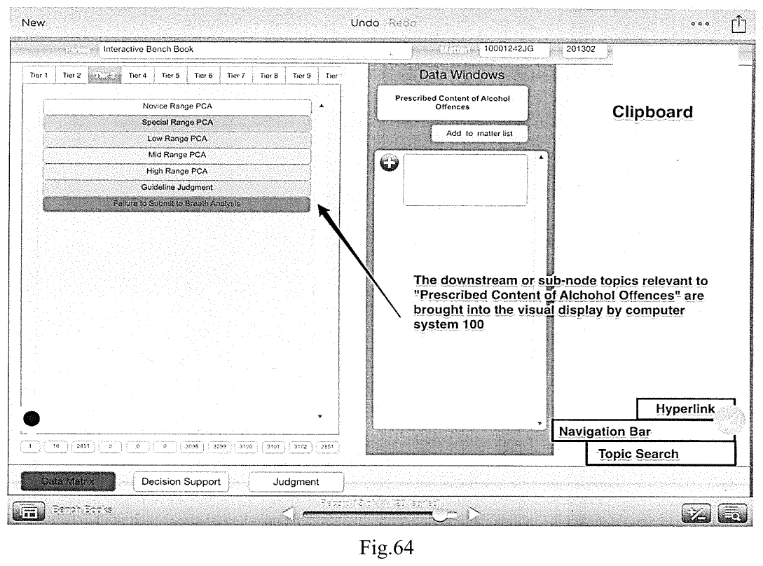

The downstream or sub-nodes relevant to the node “Prescribed Content of Alcohol Offences” appear in. FIG. 64 as Tier 3 topic nodes.

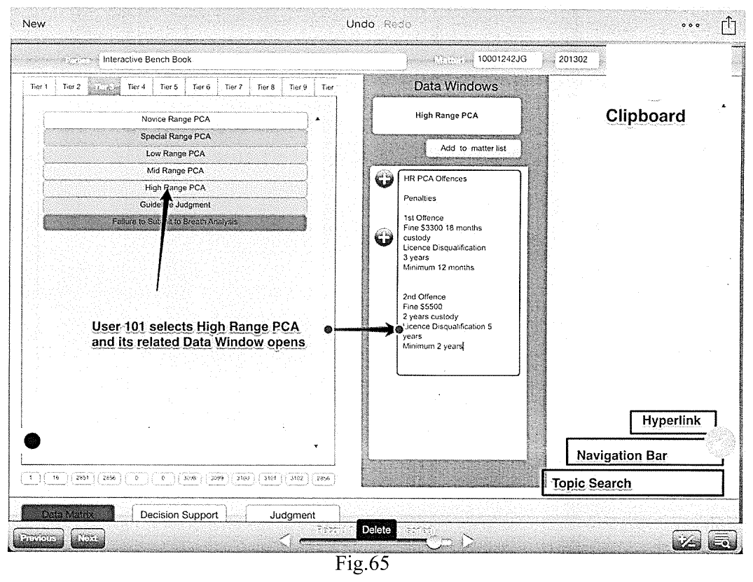

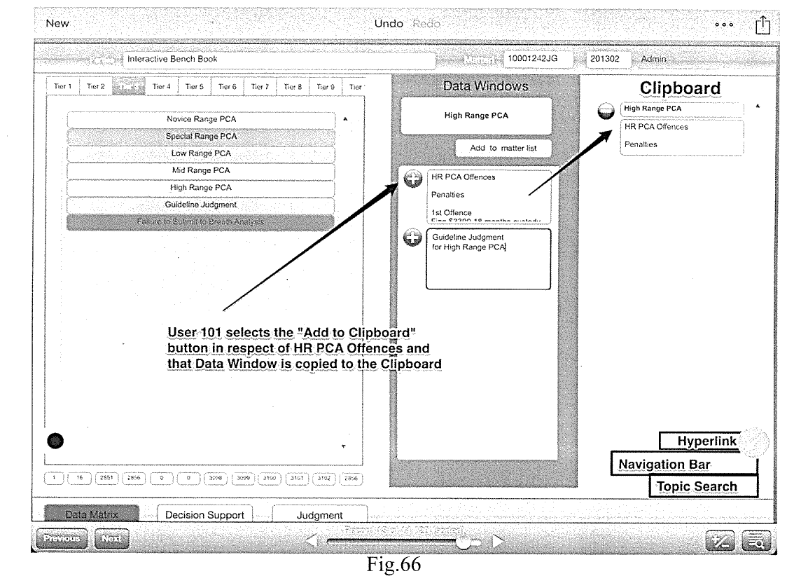

FIGS. 65 and 66 shows the decision maker selecting the node “High Range PCA” and the Data Window relevant to that node appears in Data Windows, and that Data Window may be selected by the decision maker activating the “Add to Clipboard” button.

There may be different Data Windows with different types of data relevant to the node “High Range PCA”. They may include (but are not limited to) the statutory charge provisions and the driving disqualification periods which apply to that offence.

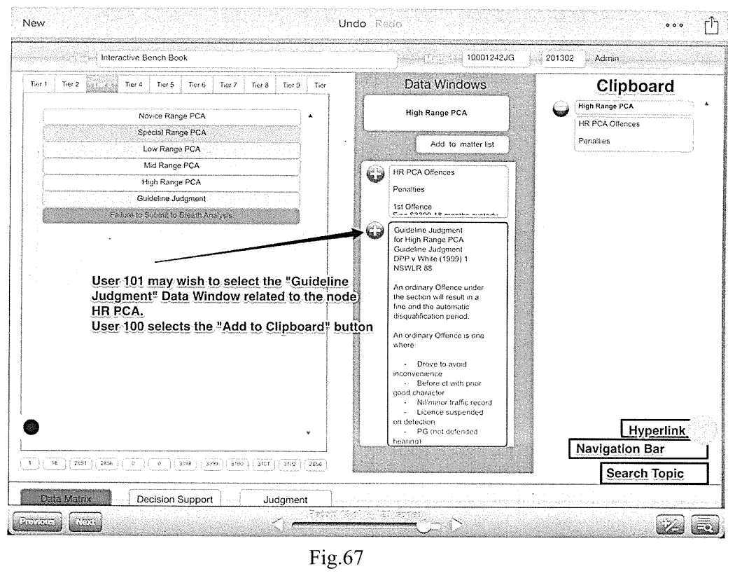

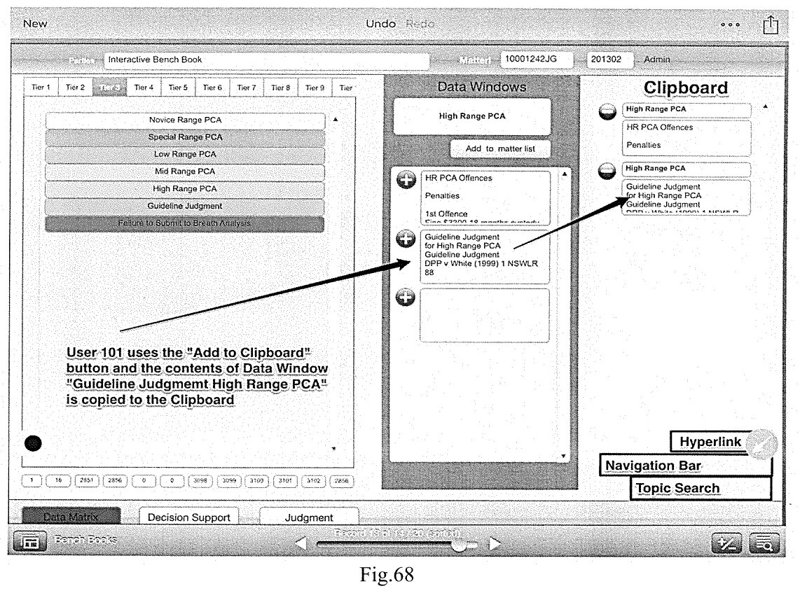

By way of further illustration, a decision maker may wish to gain the assistance of what is known as a “Guideline Judgment”—a decision of a superior court which contains a convenient statement of the approach of that court to its subject area. FIGS. 67 and 68 illustrate the decision maker selecting the “Guideline Judgment” node from the Tier 3 nodes and using the Copy to Clipboard function, copy that Data Window to the Clipboard.

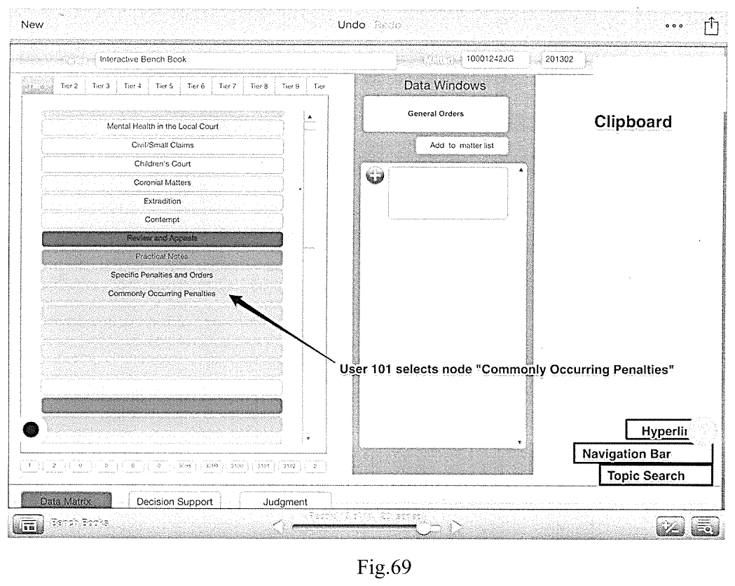

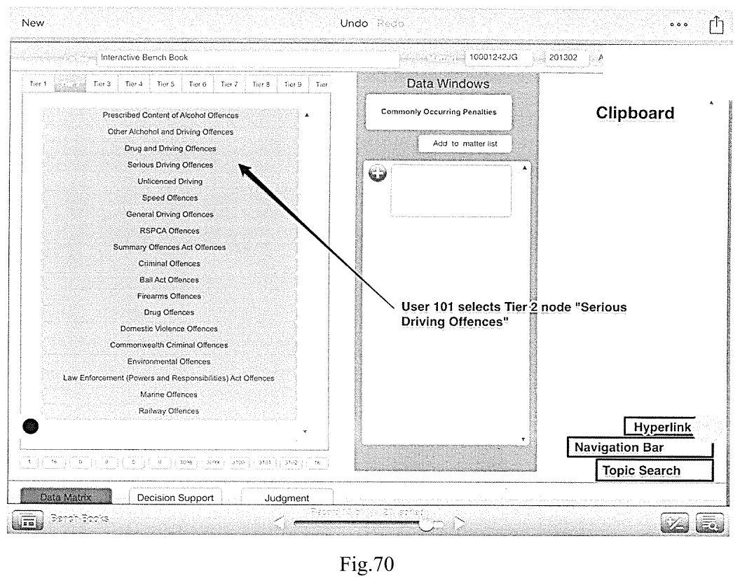

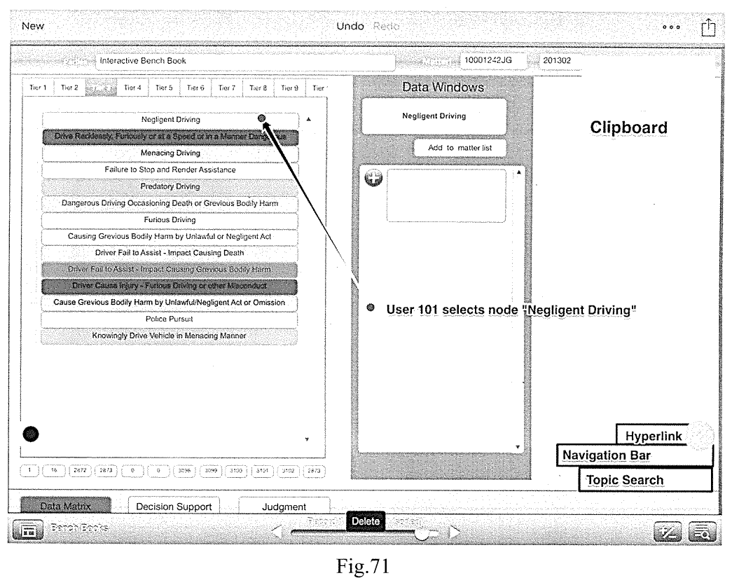

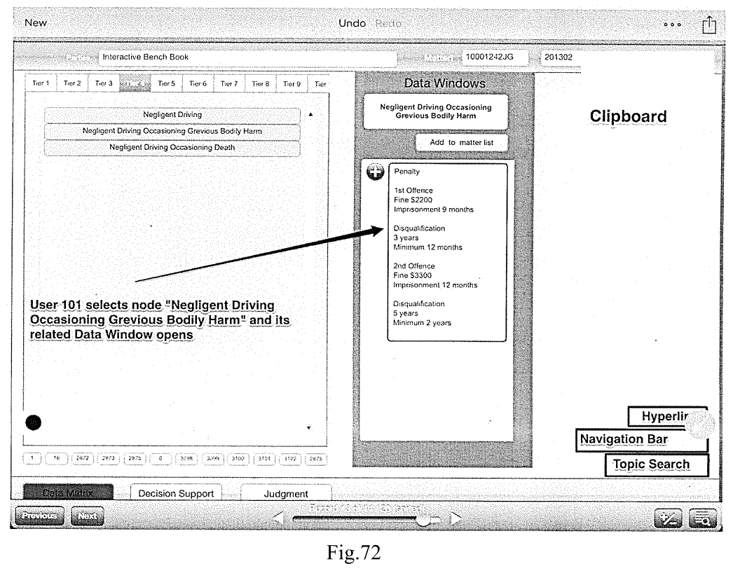

By way of further example, FIGS. 69-72, show a decision maker selecting Tier 1, Commonly Occurring Penalties, then Serious Driving Offences, then Negligent Driving and the specific offences and penalties relevant to Negligent Driving causing Grievous Bodily Harm. This illustrates the very rapid access computer system 100 provides a decision maker to the data relevant to the exercise of their judicial function.

The Topic Search function in Data Matrix mode allows the user to type in a topic or part thereof. When selected, it will open a window with all nodes containing the search terms in the visual display and the decision maker may then select the applicable node and computer system 100 will take the decision maker to that node.

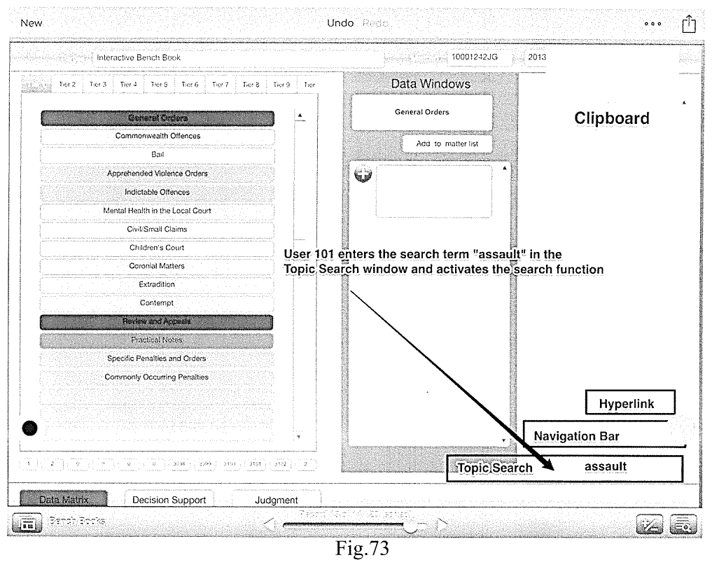

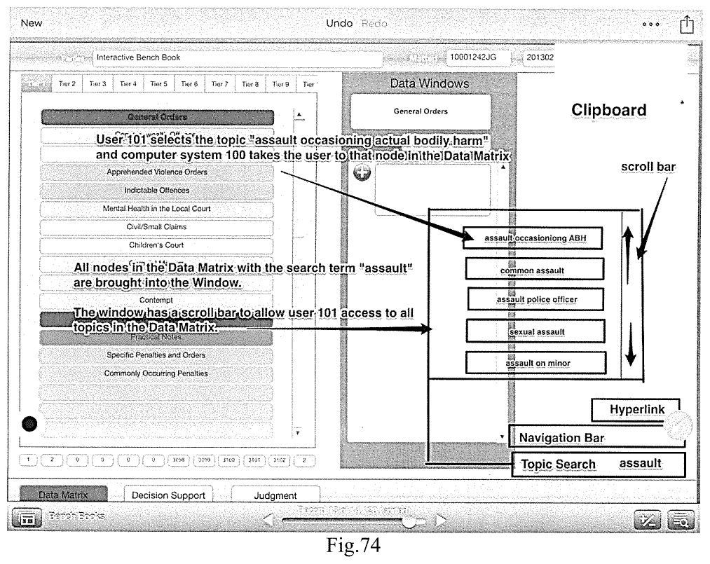

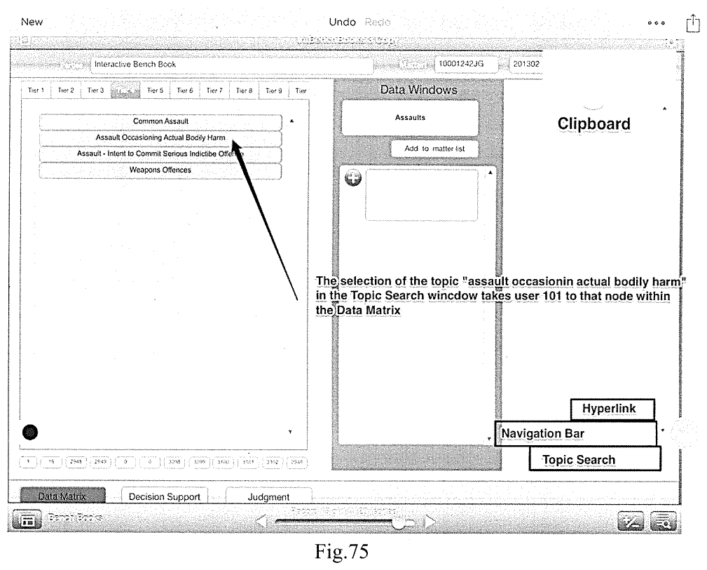

FIGS. 73, 74 and 75 illustrate the decision maker using the search function in relation to the term “assault”, selecting the appropriate node, and computer system 100 then updating the visual display to take user 101 to the selected node.

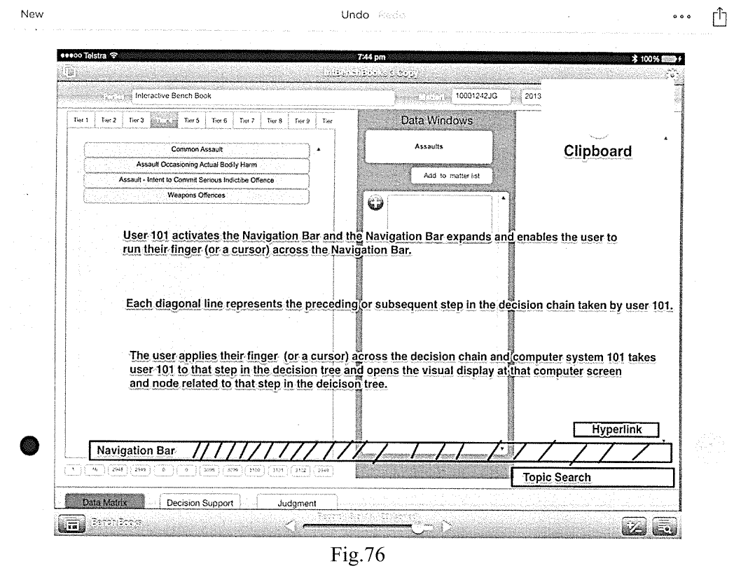

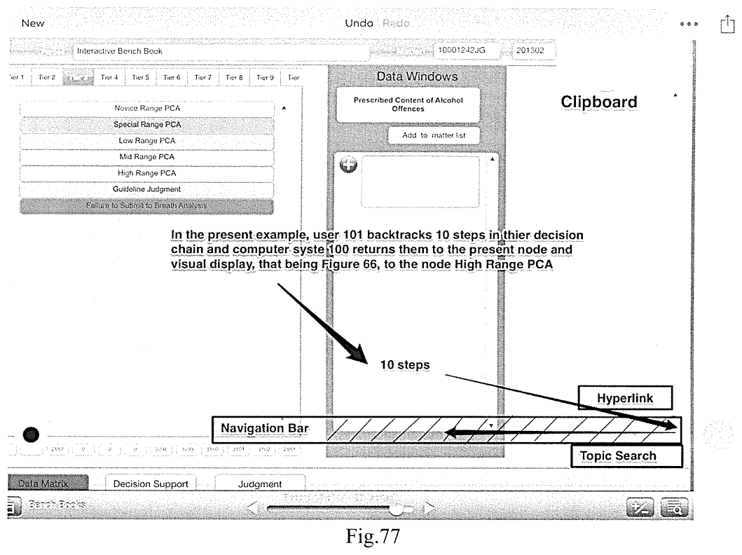

FIGS. 76 and 77 illustrates the decision maker using the navigation bar to retrace the steps the decision maker has taken though computer system 100.

The navigation bar traces the steps taken by the decision maker and when the decision maker activates the navigation bar computer system 100:
1. saves in electronic copy the history of the decision steps taken by user 101 to that point in the decision tree followed.
2. the decision maker may stop the navigation function at any point in the decision steps previously taken and re-commence moving through the decision tree from any prior historical point.

The decision maker may move interchangeably between the Data Matrix and Decision Assistance modes (as well as the Report mode) and the navigation function will record sequentially all steps taken whether user 101 is using computer system 100 in Decision Assistance or Data Matrix mode.

The Data Matrix mode provides a highly efficient structure for the provision of large amounts of data which a decision maker may wish to be able to rapidly and conveniently access. It further offers the convenience of providing all data relevant though the one computer system. In one example, all Bench Books for Judges relevant to one jurisdiction may be contained as the primary node topics in Tier 1—for example, the Sentencing Bench Book, the Criminal Trials Bench Book, the Family Law Bench Book, the Evidence Bench Book etc may all be accessed from within computer system 100.

Decision Assistance Mode

At any stage during the operation of the computer program the decision maker may select Decision Assistance Mode by selecting the Decision Assistance Button.

When selected the program activates Decision Assistance Mode.

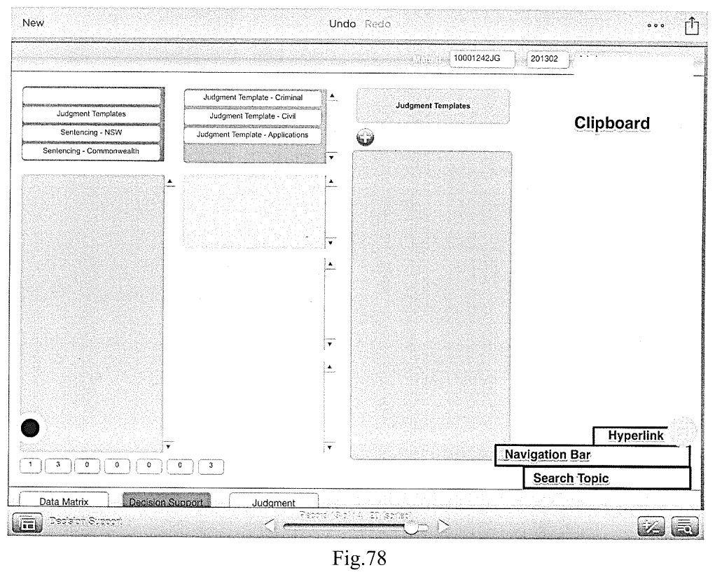

FIG. 78 illustrates the visual display when the decision maker has activated Decision Support mode.

Decision Assistance mode provides the decision maker with the benefit of an experts decision chain in respect of problems falling within or related to the subject matter (nodes) of the Data Matrix.

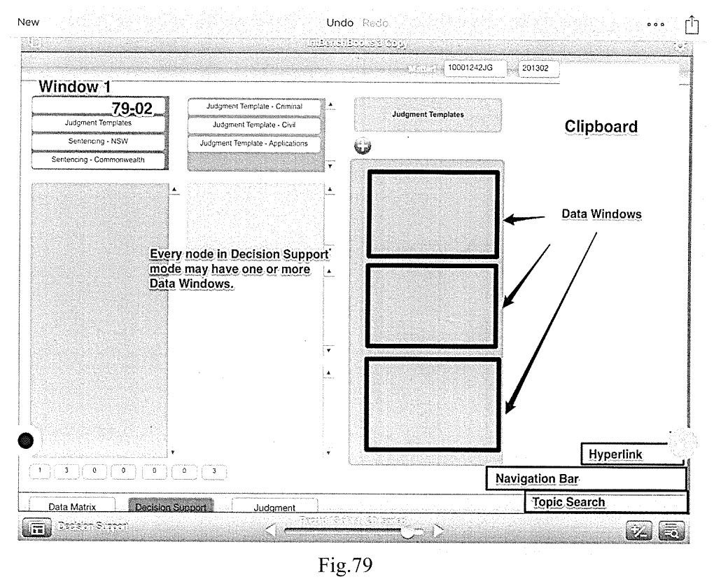

FIG. 79 illustrates computer system 100 when Decision Assistance mode has been activated.

There are 6 windows in Decision Support mode topic areas, comprising columns 1 and 2 of the visual display of computer system 100 in FIG. 79.

Window 179-02 contains the following nodes:
1. the nodes contained in Tier 1 of the Data Matrix mode. That is all of the subject areas of the nodes contained within the Data Matrix. All of these nodes in the present example are contained in Window 1 of Decision Assistance mode.
2. the nodes which are relevant to the decision maker solving problems or making decisions in respect of the nodes contained in the Data Matrix. These nodes include expert decision trees providing the decision maker with the decision support assistance they require to solve problems arising in respect of the nodes in the Data Matrix and such additional nodes will in the present example include (but will not be limited to) judgment templates, expert guidance to the problems the judge will be required to solve in relation to the laws of evidence, expert guidance in relation to the law on any topic relevant to the subject area of the Data Matrix, including but not limited to criminal offences and defences, children's law, contempt of court, expert guidance in respect of matters related to criminal procedure, and expert guidance on how to (inter alia) approach applying the law on sentencing an offender.

All nodes in Decision Support mode have at least 1 Data Window linked to them and in addition a blank or empty Data Window to enable the decision maker to add any notes they wish which may not fall within the Data Windows topics linked to that topic node.

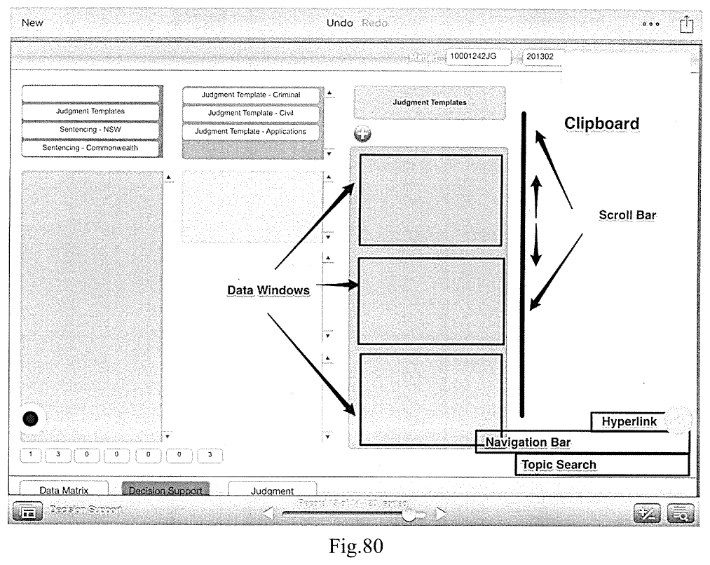

The illustration used in this patent application use one data window in Decision Assistance mode but computer system 100 may use any number of Data Windows linked to one node and the Data Windows column (being column 3 of the visual display of computer system 100 in Decision Support mode) will have a scroll bar facility so that the visual display will not restrict the number of Data Windows that may be linked to one node. See FIG. 80.

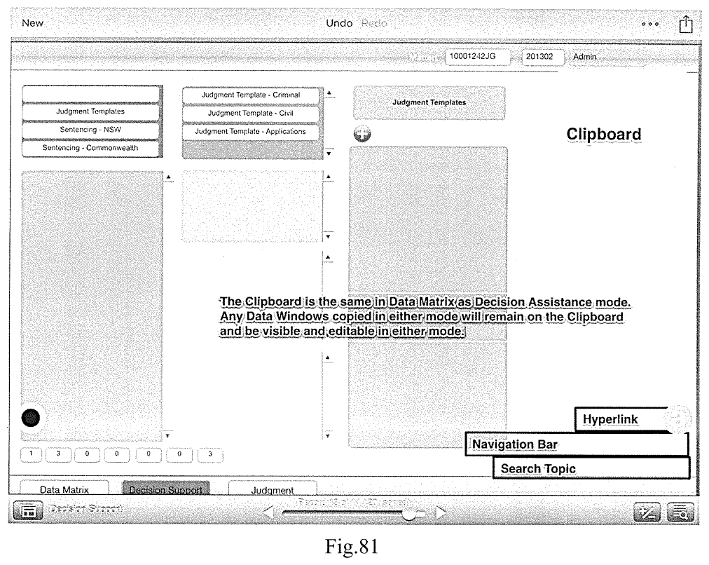

The Clipboard is illustrated in FIG. 81.

The Clipboard in Decision Assistance mode is the same as the Clipboard in Data Matrix mode.

Whether the user has entered Data Windows onto the Clipboard in Data Matrix mode or whether the user has entered Data Windows onto the Clipboard in Decision Assistance mode, the Clipboard in either mode will display the Data Windows which have been copied onto it from either mode.

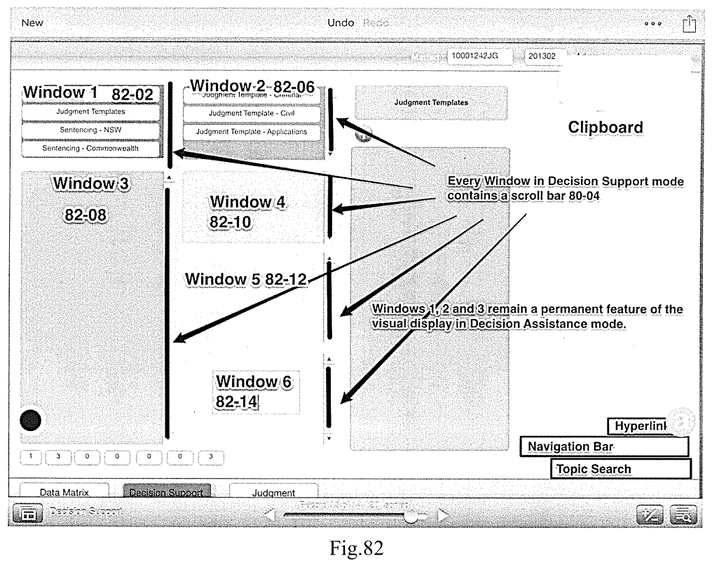

FIG. 82 illustrates:

Window 1 82-02 contains
1. The primary node topics contained in Tier 1 of the Data Matrix. In the Judicial Officers example, there may be different categories of problems which may arise in relation to the Tier 1 nodes in the Data Matrix.
2. Any additional nodes relevant to the discharge of the decision makers functions. These may include evidence problems, how to approach sentencing an offender under State and Commonwealth Law, how to approach applying the law relevant to criminal offences or defences, dealing with applications in respect of specific legal applications (ie appeals against decisions of the authority to suspend a persons driving licence etc), applying general law and statutory causes of action (ie how to approach Sale of Goods Act claims, contract claims etc).

Each Window in the visual display of computer system 100 contains a scroll bar 80-04.

That means that the size of the Window in the visual display does not restrict the number of nodes the Window may contain.

Window 2 contains the specific topics for which decision support is available in computer system 100 in respect of each of the nodes in Window 1 82-06.

Window 3 contains the decision steps relevant to each topic for which decision support is available in Window 2. 82-08

Window 3 contains a scroll bar 80-04 so the number of steps in the expert decision tree is not restricted by the size of the visual display.

Window 3 is a larger window in the visual display present in this mode of computer system 100 as that larger window size will allow the decision maker a larger view of the steps in the decision tree or chain relevant to the specific problem.

Windows 1, 2 and 3 remain a permanent feature of the computer screen display in Decision Assistance mode within computer system 100. Each of these windows contains a scroll bar so that the number of nodes they may contain is not limited by the size of the visual display.

Window 4 82-10 contains those sub-node topics relevant to the first step in the expert decision tree contained in Window 3.

There may be more detailed or specialised information applicable to a Window 4 node and the user may proceed deeper into the expert decision chain or decision tree by selecting that node topic in Window 4 and computer system 100 will then cause the downstream nodes to the Window 4 node to open in Window 5 82-12. Window 6 is 82-14.

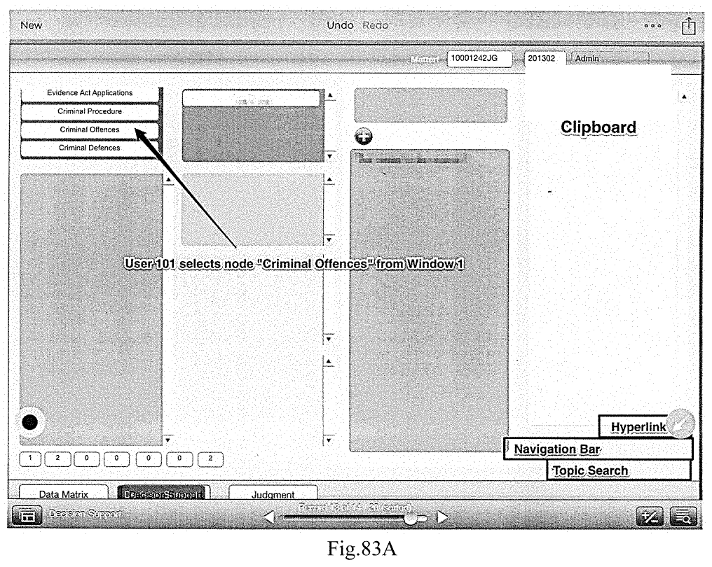

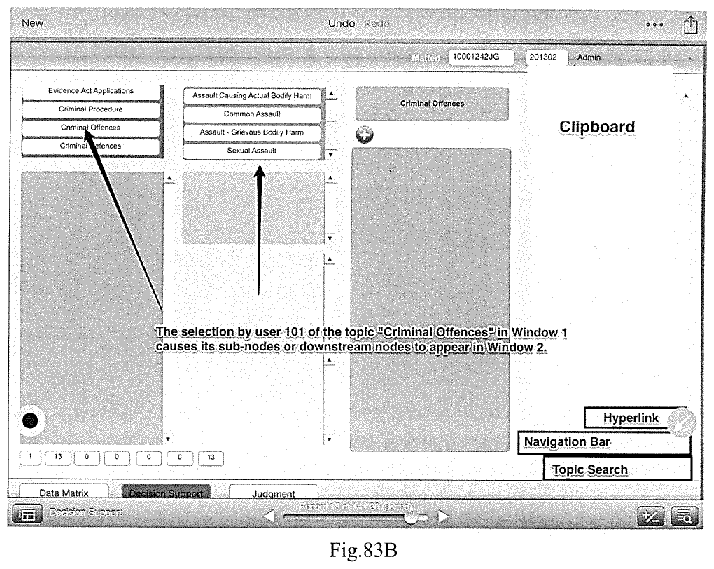

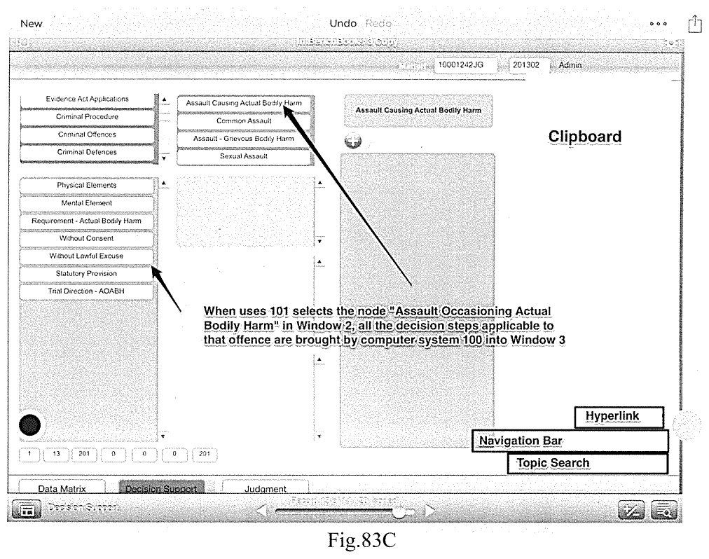

Diagram 83A shows a decision maker selecting the node “Criminal Offences” in Window 1. As illustrated in FIG. 83B, computer system 100 then causes all sub-nodes or downstream nodes relevant to the node “Criminal Offences” to appear in Window 2. The decision maker scrolls through those nodes for which decision support is available and selects “Assault Occasioning Actual Bodily Harm”. That node is selected and the decision steps applicable to that offence are then brought into the visual display by computer system 100 in Window 3—see FIG. 83C.

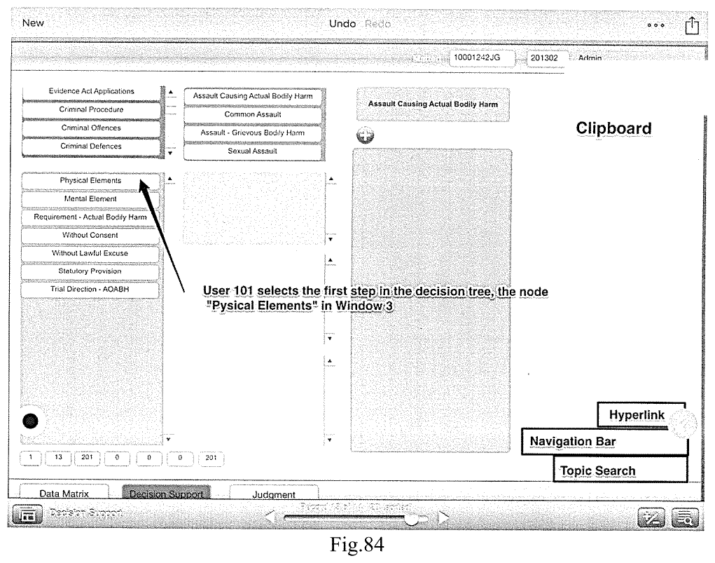

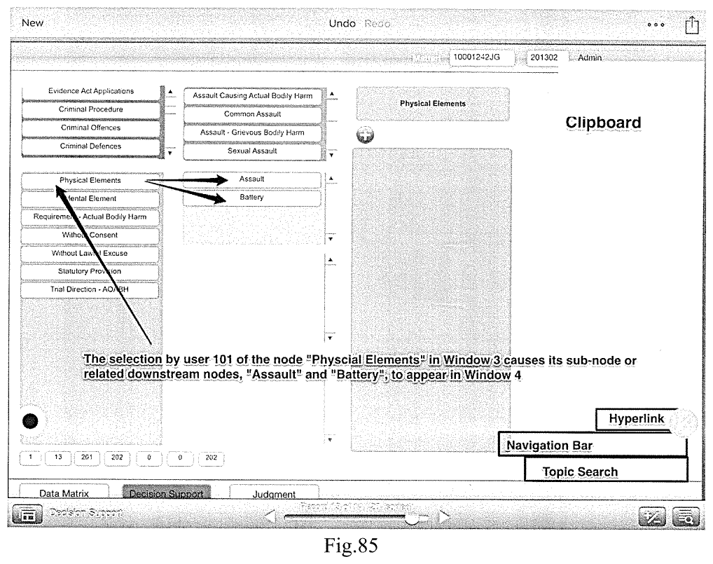

The decision maker selects the first step or node in the expert decision chain, “Physical Elements”. See FIG. 84. That causes the downstream nodes relevant to “Physical Elements” to appear in the visual display—those nodes being “Assault” and “Battery” in FIG. 85.

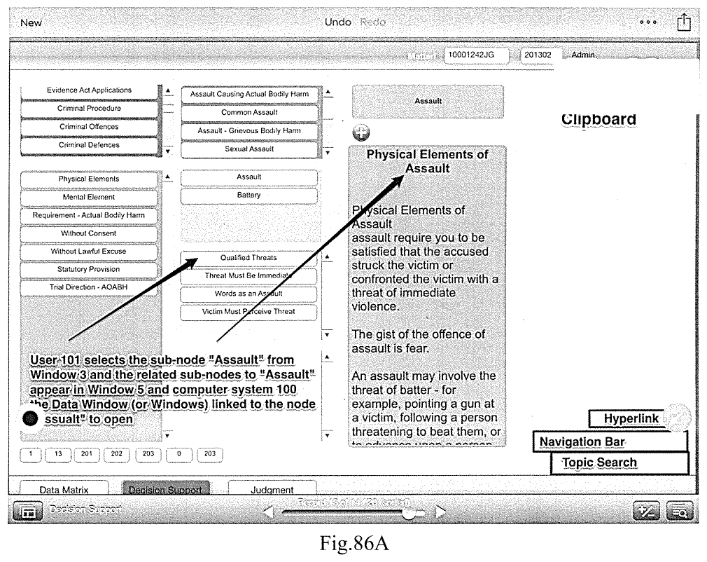

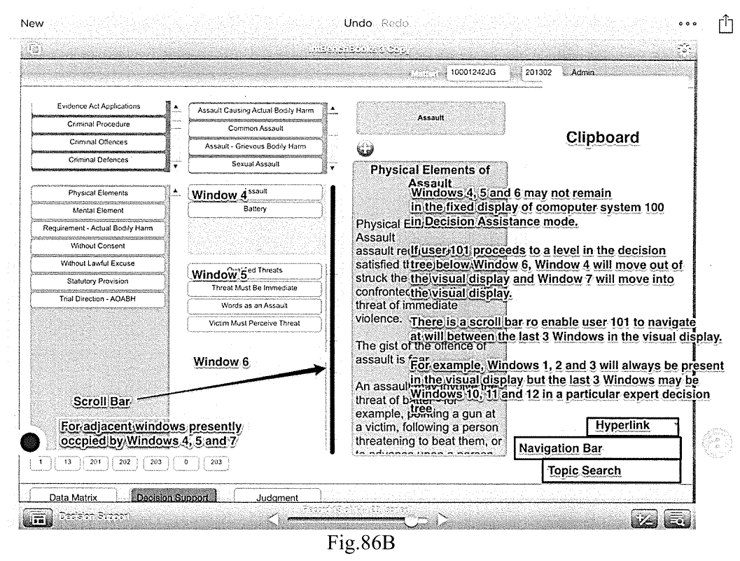

The decision maker selects the node “Assault” from Window 4 and as FIG. 86A illustrates the computer system 100 causes:
1. The Data Window relevant to the node “Assault” to open in the visual display; and
2. the related downstream nodes to the node “Assault” to open in Window 5.

When that selection is made, computer system 100 brings into the visual display the related sub-node topics in Window 5.

If further and more specialised assistance is required from computer system 100, the decision maker selects the node relevant in Window 5 and its related sub node or downstream nodes are brought into the decision system in Window 6.

Windows 4, 5 and 6 may not remain part of the fixed display in Decision Assistance mode. If the decision maker wishes to proceed further into the expert decision chain, and selects a node in Window 6, computer system 100 changes the display by moving Window 4 out of the visual display and showing Window 7 in the visual display of the computer screen 102. In other words, the last 3 Windows used by the decision maker will remain in the visual display of computer system 100 in the location of Windows 4, 5 and 6 if the decision maker has progressed that deeply into the decision tree contained within computer system 100. Windows 1, 2 and 3 do not change and remain in the visual display at all times in the operation of computer system 100.

By way of further example, in a highly complex problem, the visual display of computer system 100 in Decision Support mode may display Windows 1, 2 and 3, and Windows 9, 10 and 11. User 101 may use the scroll bar adjacent to the location of Windows 4, 5 and 6 to move the Windows in and out of the visual display at that location as may be appropriate.

The visual display of computer screen 102 will always display Windows 1, 2 and 3, and the last 3 windows used by the decision maker in the location originally displayed by Windows 4, 5 and 6.

The scroll bar relevant to the locations of Windows 4, 5 and 6, allows the user to move the different Windows from windows 4, 5 and 6 through to the last Window—Window “n”-used by the decision maker when utilising and following the expert decision chain which commences in Window 3. See FIG. 86B.

The inventor notes that a problem would be extraordinarily complex to require more than 6 Windows for its resolution but those further steps are readily available to the user within computer system 100.

In one example, a Magistrate or Judge may have legal problems to solve in respect of the data contained in the Data Matrix concerning (inter alia):

How to Apply Sentencing Law

Civil Law Applications

Civil Law Motions

Applying the Evidence Act

Approaches to Applying Criminal Law Offences

Approaches to Applying Criminal Law Defences

Appeals to the Local Court

Applications to the Local Court

Contempt in the Local Court

etc

These subject areas will be contained in Window 1 in addition to the nodes contained in Tier 1 of the Data Matrix mode.

A Judicial Officer may be provided with guidance in the form of (inter alia) judgment templates for Criminal and Civil proceedings, Application proceedings, may require assistance in approaching complex sentencing matters and in the steps involved in applying the law in relation to various criminal offences and defences as well as commercial and other causes of action.

Decision Assistance provides that decision support.

It is noted that similar issues arise in respect of any professional decision system. This is not a decision support system related only to legal problems but may apply in respect of (inter alia) medical and veterinary diagnosis and treatment, agricultural processes, building and architectural processes, teaching and instruction processes, and information manuals for large companies which have various processes employees must initiate in respect of certain matters (ie grievance procedures and training modules)

Decision Support is a meta-function of this decision support system.

Decision Support provides a mechanism which allows a replication of historical patterns, that being defined as the steps in the decision chain taken by a high level expert (or a decision chain prepared by an expert panel) in the resolution of a problem or problems which may arise in respect of the topics in the Data Matrix by way of structured paradigm of steps prepared by that expert (or panel of experts).

The steps in the decision chain may be prepared by 1 person, or a number of persons.

Computer system 100 will provide the expert decision tree to all persons seeking to solve the problem to which that expert decision tree is addressed. By way of one example, a medical general practitioner in a remote African village will be able to access the Infectious Diseases diagnostic decision tree of a leading Harvard medical professor. That decision chain will be of extremely high quality and will incorporate knowledge, experience and updated medical information that a general practitioner would not in the usual course have available to them. Indeed, the expert decision chain may be a collaborative project involving (for example) Harvard, Yale, Johns Hopkins, the University of Johannesburg, and the European University experts. It would be widely available to all medical practitioners seeking to solve infectious diseases problems and would greatly improve the quality of diagnosis and treatment offered—particularly in the context of less resourced medical environments, for example, in relation to the provision of medical information to the poor, the 3rd world, and to remote and poorly resourced regions. Experts could make available Decision Assistance though computer system 100 in relation to any subject area—for example, obstetrics and cardiology, and so on. The best practice treatment for Aids, for example, could be designed in a computer system 100 and made available to practitioners providing medical support for this condition throughout the 3rd world.

Accordingly, this decision support system greatly facilitates an unrestricted number of high level experts developing and maintaining a decision system in respect of a specific problem, and provides that decision chain to all persons seeking to solve a problem on the topic of the decision chain. That means that the decision chain will be of extremely high quality and widely available to all persons who are engaged in endeavouring to solve similar problems.

It is noted that this invention provides a computer system which will allow collaborative efforts of a potentially large number of experts in devising and updating a decision tree. That will mean that the power of such a decision tree—which may be made available to all persons seeking assistance with the subject of the decision tree—will be considerably greater than any decision system that may be able to be prepared by one person. The top 20 specialists in, for example, oncology in the United States (or the world) are, by way of example, likely to produce a collaborative decision system in computer system 100 than any one of them alone would be likely to generate and the diagnostic benefits of having that collaborative diagnostic decision system available to other medical practitioners, including general practitioners, hardly needs explanation.

The inventor says that the applications of this decision support system are significant and obvious.

In one example, lawyers who are engaged in highly complex legal problems will be able to avail themselves of the approach to such problems taken by the best legal practitioners (such as senior judges) and high level academic experts in respect of that problem or legal topic. Such a problem could involve but is not restricted to complex conveyancing issues, complex evidence issues, complex sentencing issues and problems arising in respect of the application of the law in specific topic areas such as contract, commercial law, property law and equity.

It is also to be noted that the decision system 100 also confers a high level of transparency in relation to the approach decision makers take to solving problems.

Apart from recording the steps a decision maker takes as they progress though a problem, the Navigation function permits the steps in that decision to be recorded and saved as a separate data file. That data file may be saved and emailed to another user (user 102) and may be loaded into user 102 decision system 100. User 102 may transparently retrace the steps taken by user 101 and user 102 will be able to follow every step user 101 took in deciding or solving the problem or coming to user 101's conclusion or diagnosis.

By way of example, that will mean that a general practitioner of medicine may email (or otherwise electronically transfer) their diagnostic decision process to a medical specialist when referring a patient. That medical specialist will be able to comprehensively review the basis on which the general practitioner determined the patient has a particular condition and why they determined a particular treatment (such as a specific drug) was appropriate. That will make it considerably easier for the specialist to review the patients history and in rendering very transparent the diagnostic approach of the general practitioner any errors are much more likely to become apparent when the patients diagnostic chain is reviewed at any level—whether it be a senior registrar reviewing a junior doctors diagnosis and treatment of a patient at hospital level, or a specialist practitioner reviewing a patients records. In the inventors view, there are very high levels of advantage in the use of decision system 100 in terms of the overall quality of service offered to patients, both in terms of the decision support offered by their treating practitioner who has the benefit of an expert decision chain in relation to the problem, and in the process of review of the diagnosis and treatment afforded to the patient.

The Decision Support mode also confers a high level of transparency in respect of the approach decision makers take to solving particular problems.

The Decision system records the decision steps which have been taken and that data is able to be retained, filed and if necessary forwarded to other practitioners or sources.

It would also significantly enhance transparency in the provision of medical professional services to patients and be a very valuable tool in exposing any error rapidly and allowing remedial activity to occur.

It is also a valuable teaching tool, in professional and any learning context, because it will expose the reasoning process which the student or learner has employed in the resolution of the problem.

In one example, the decision maker, a magistrate or judge, may wish to avail themselves of the steps an expert in sentencing law would take when engaged in sentencing a defendant appearing before the court.

Sentencing law falls within the subjects contained in the Data Matrix in one example.

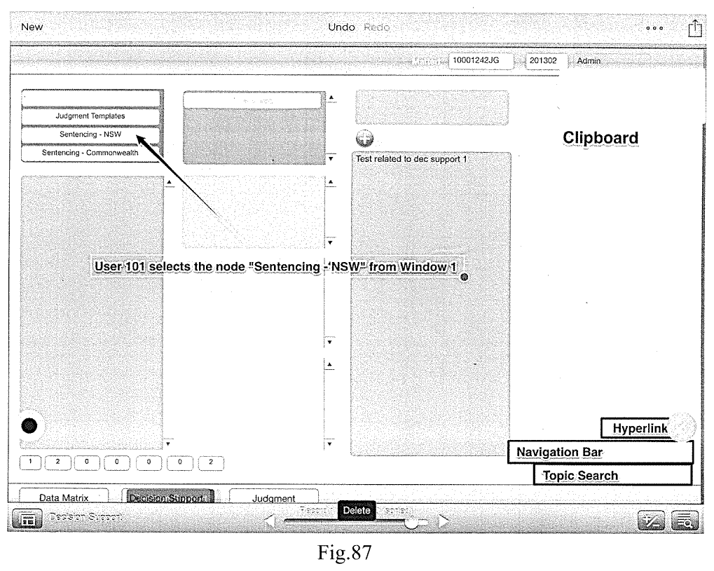

FIG. 87 illustrates the decision maker opening Window 1 on Decision Assistance mode and selects Sentencing—NSW.

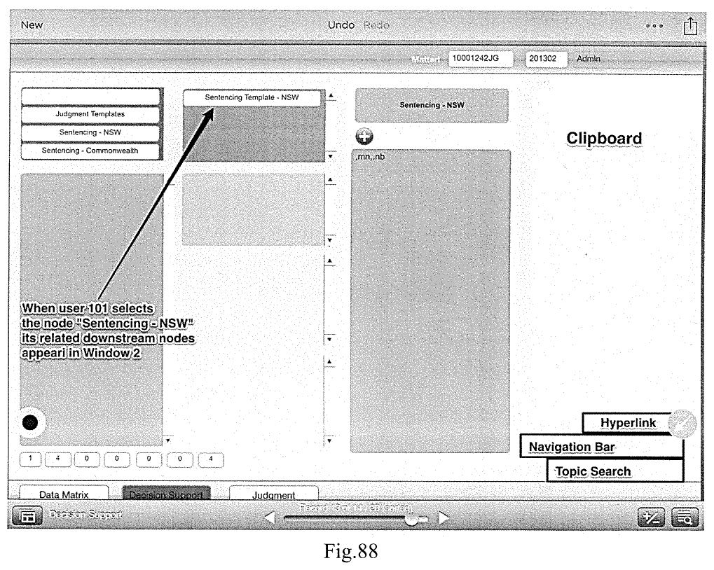

FIG. 88 illustrates the sub-nodes relevant to Sentencing—NSW appear in FIG. 88.

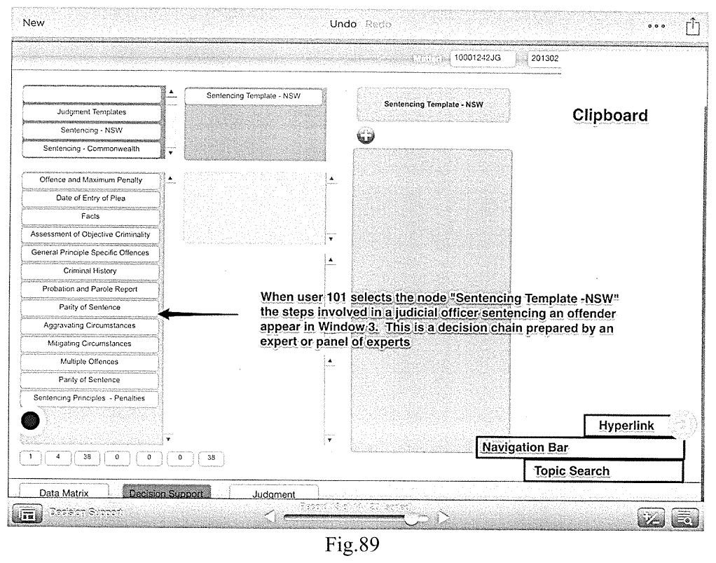

FIG. 89A illustrates the decision maker selecting Sentencing Template—NSW.

The steps involved in a judicial decision maker approaching sentencing an offender then appear in Window 3. See FIG. 89B.

A decision maker may wish to proceed though the steps sequentially.

Every node has at least one related Data Window.

By way of example, a decision maker may proceed though the decision chain to the node Aggravating Circumstances.

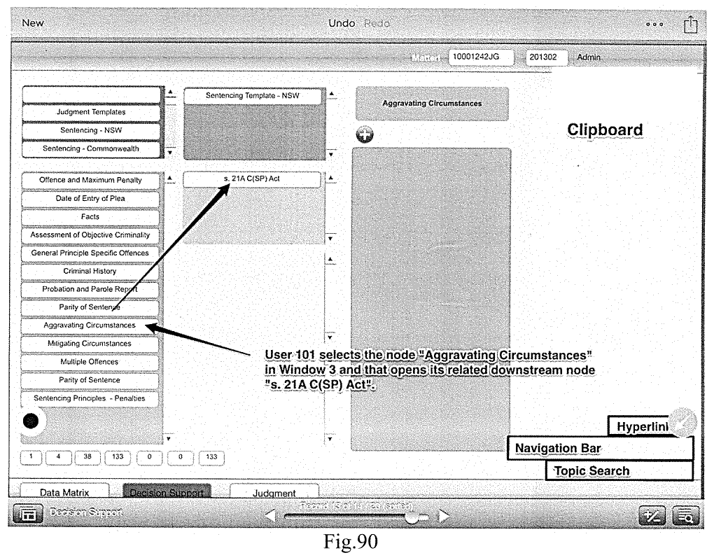

Selecting the node Aggravating Circumstances will cause computer system 100 to update the visual display and bring into the visual display the relevant statutory principle—in this example, s. 21 Crimes (Sentencing and Procedure) Act—in the downstream node in Window 4. See FIG. 90.

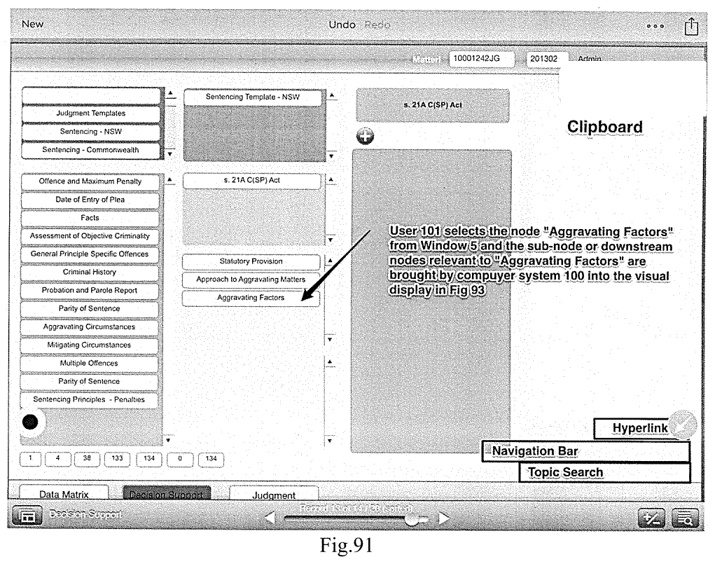

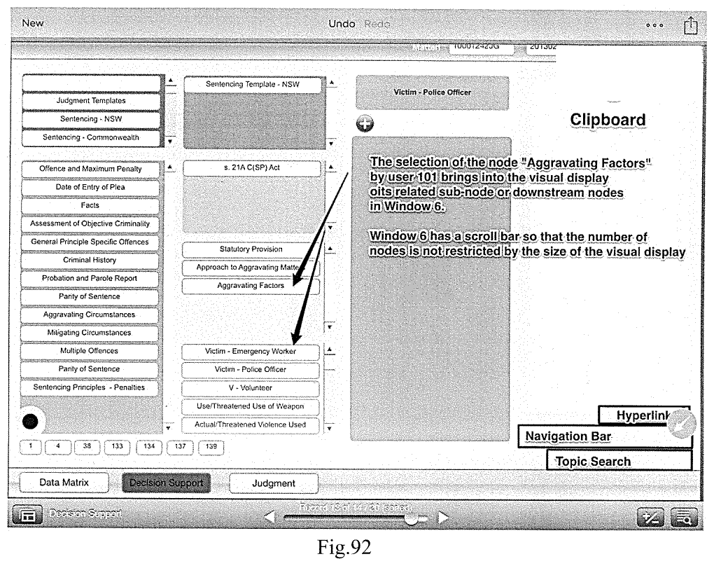

The decision maker selects the node “s. 21 Crimes (Sentencing Procedure) Act in Window 4 and its downstream related sub-nodes appear in Window 5. See FIG. 91. They are

Statutory Provision

Approach to Aggravating Matters

Aggravating Factors

FIG. 91 illustrates the decision maker selecting the node “Aggravating Factors” and the sub-node topics relevant to Aggravating Factors is brought into Window 6. See FIG. 92.

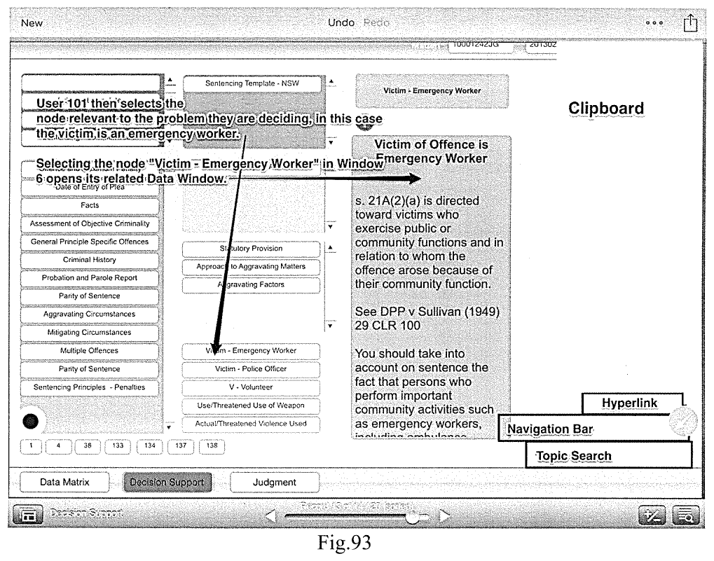

The decision maker then selects the particular matter relevant to the circumstances before them for sentence—on one example, the victim of the assault is a emergence worker—and that node is selected. See FIG. 93.

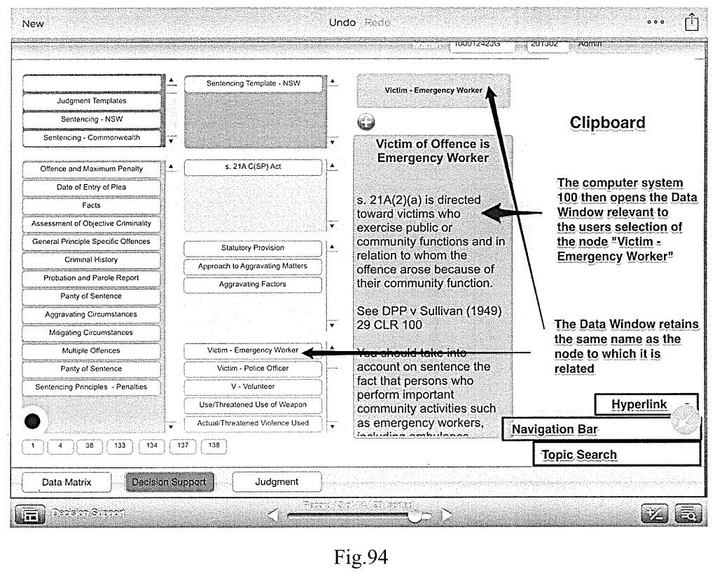

The Data Window relevant to the law applicable to setting the appropriate sentence when the victim of the offence is an emergency worker then appears in its related Data Window. See FIG. 94.

The decision maker may open that data window, and edit its contents.

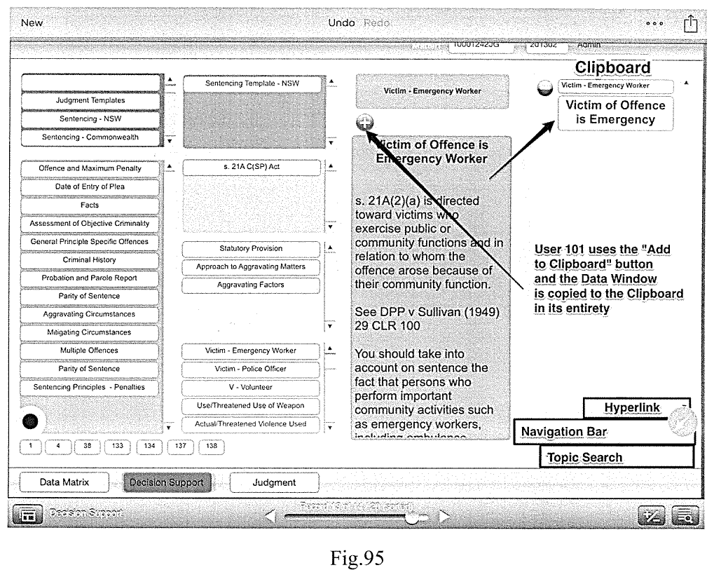

The decision maker may move the entire Data Window to the clipboard by activating the “Add to Clipboard” button. See FIG. 95.

The Data Window in FIG. 95 is then copied to the Clipboard.

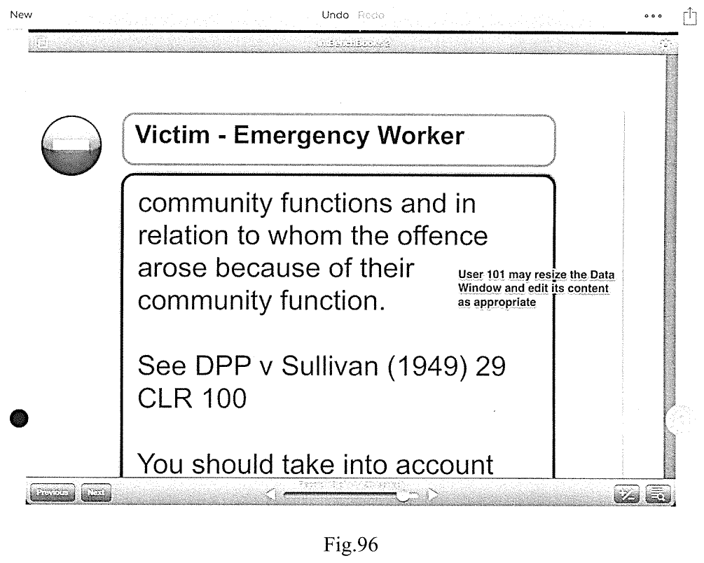

FIG. 96 shows the decision maker re-sizing that Data Window and editing its content by reference to the specific facts before the judicial decision maker—that is, the details of the victim of the present offence, the nature and extent of their injuries, and how the emergency worker victim came to be injured.

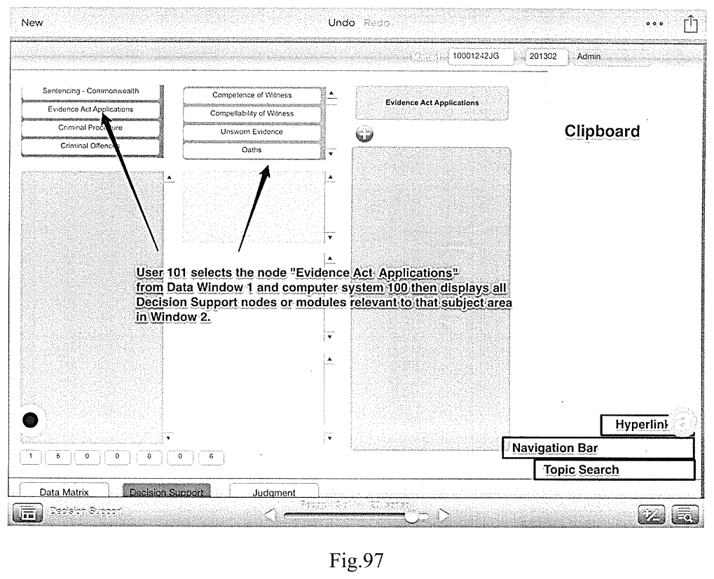

By way of further example, the decision maker may wish to obtain assistance in solving an Evidence problem that may arise in the course of a hearing. The decision maker scrolls though the topic nodes in Window 1 and selects “Evidence Act Applications”. See FIG. 97.

Computer system 100 then displays the following topic nodes in Window 2.

Competence of Witness

Compellability of Witness

Unsworn Evidence

Oaths

Leading Questions

Unfavourable Witness

Court Control of Witness

Reviving Memory

Documents—Cross Examination

Relevance

Hearsay Evidence

Opinion Evidence

Admissions

Admissibility—Judgments

Admissibility—Convictions

Tendency Evidence

Coincidence Evidence

Credibility Evidence

Character Evidence

Identification Evidence

Client Legal Privilege

Legal Professional Privilege

Sexual Assault Privilege

Public Interest Immunity

General Discretion to Exclude

Discretion—Illegally Obtained

Discretion—Prejudicial Evidence

Standard of Proof

Burden of Proof

Each of these nodes has an expert decision chain or tree which when selected will appear in Window 3. Each of the topics or problems for which decision assistance is available may be referred to as a module.

When each of the steps in the decision chain is selected, its related sub-node topics will appear in Window 4, and when the sub-topic nodes in Window 4 are selected, its downstream or sub-topic nodes appear in Window 5 and so on

Each node has its own Data Window or Windows and the user may copy and paste them to the Clipboard, either in whole or in part.

The following figures illustrate a worked illustration of a judicial decision maker using computer system 100 to provide decision assistance to the decision maker/judge when discharging their judicial function in making legal decisions during the course of a matter proceeding before them and illustrates how decision system 100 assists them in preparing a written judgment whilst a hearing is proceeding before them. This illustrates that decision system 100 provides decision support in relation to processes and procedures (such as judgment writing) as well as problems.

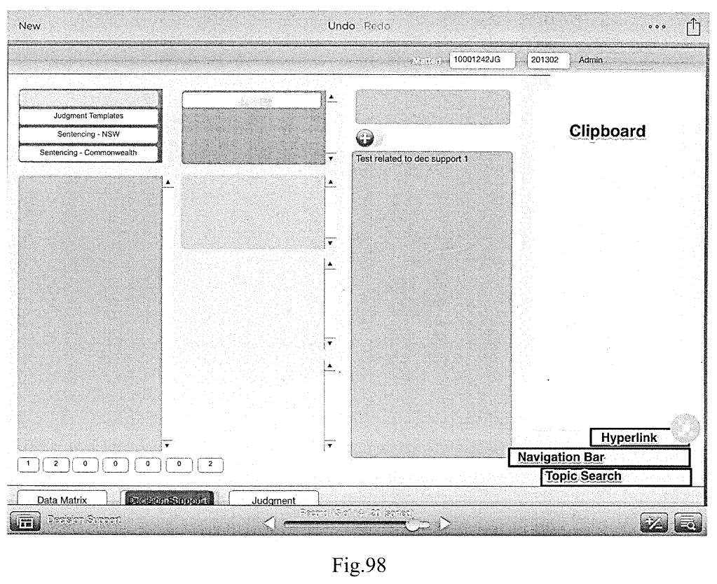

FIG. 98 illustrates user 101, a Judge, opening computer system 100 in Decision Assistance mode.

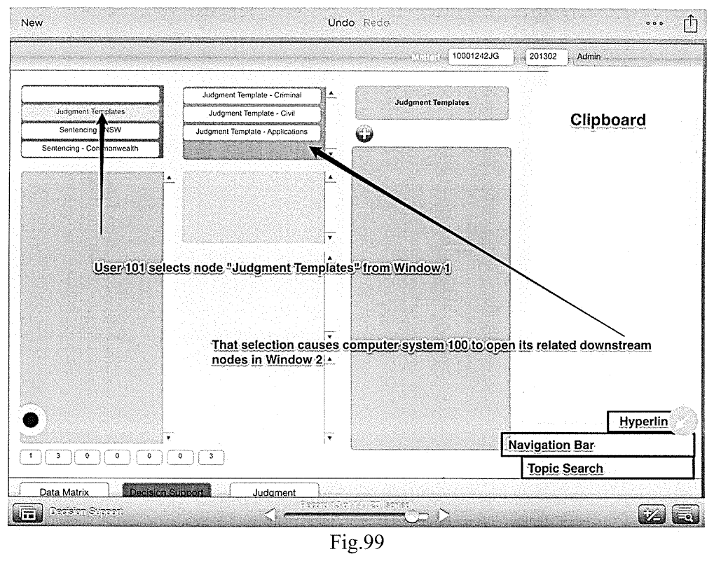

FIG. 99 illustrates user 101 selecting Judgment Templates from Window 1.

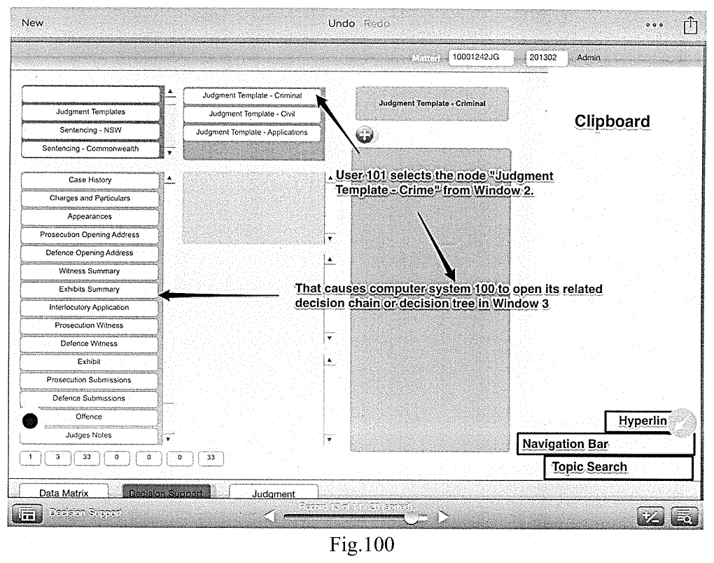

FIG. 100 illustrates user 101 selecting Judgment Template—Criminal from Window 2.

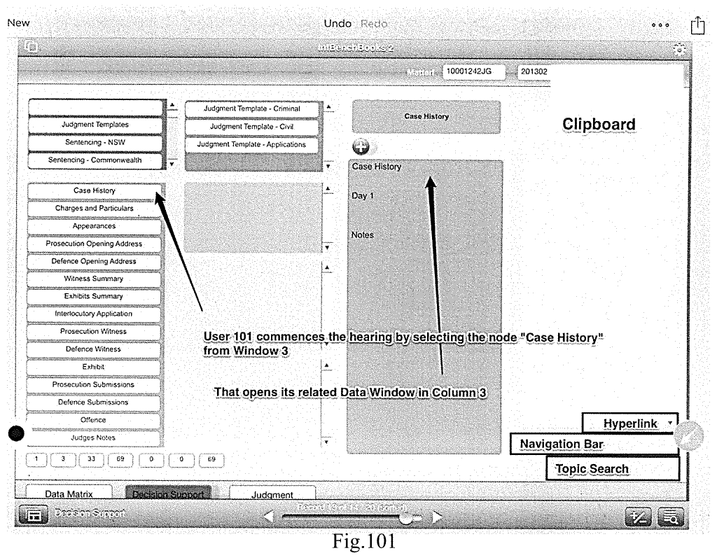

FIG. 101 illustrates uses 101 commencing the hearing by selecting Case History from Window 3, the decision steps in the conduct of a criminal trial.

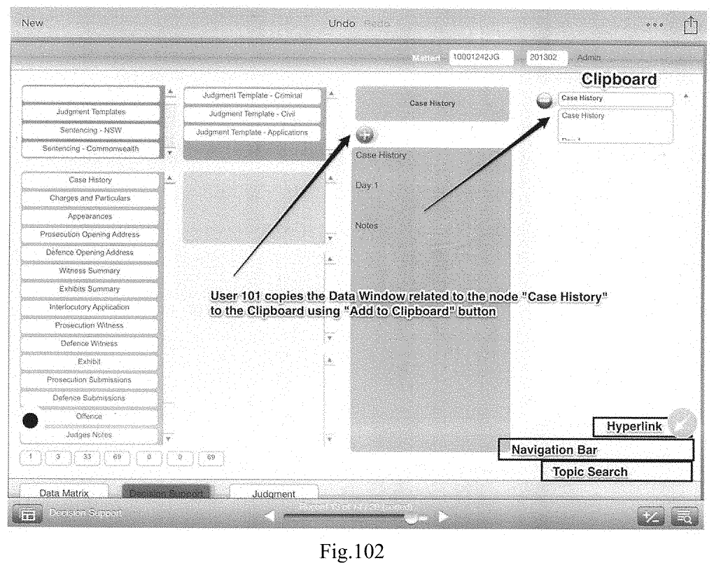

FIG. 102 Illustrates user 101 selecting “Add to Clipboard” function in relation to the Case History Data Window.

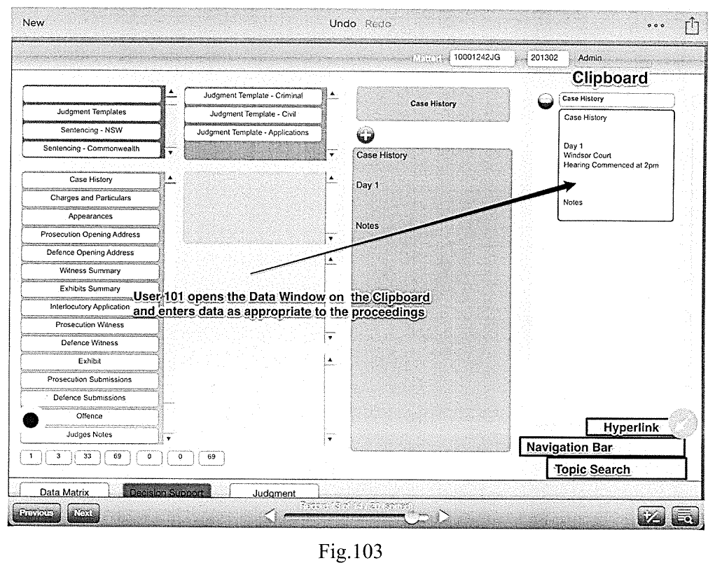

FIG. 103 illustrates user 101 opening Data Window “Case History” and adding the information relevant—ie Day 1, Windsor Court. Matter commenced at 2 pm, and any other matter which may be applicable.

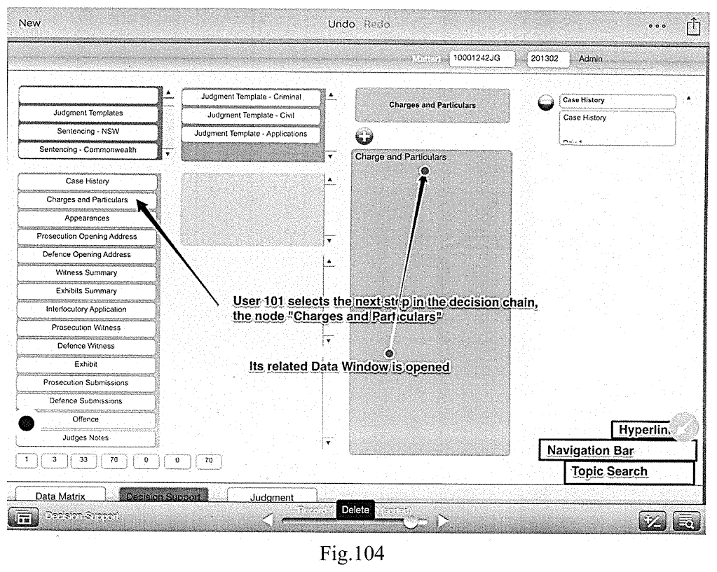

FIG. 104 illustrates user 101 selecting the next step in the decision chain in Window 3, Charges and Particulars.

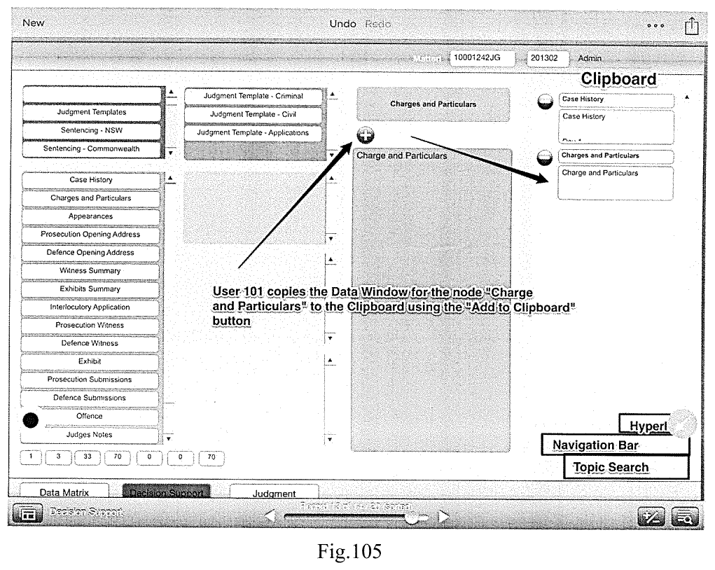

FIG. 105 illustrates the user 101 selecting the “Add to Clipboard” function and copying this Data Window to the Clipboard.

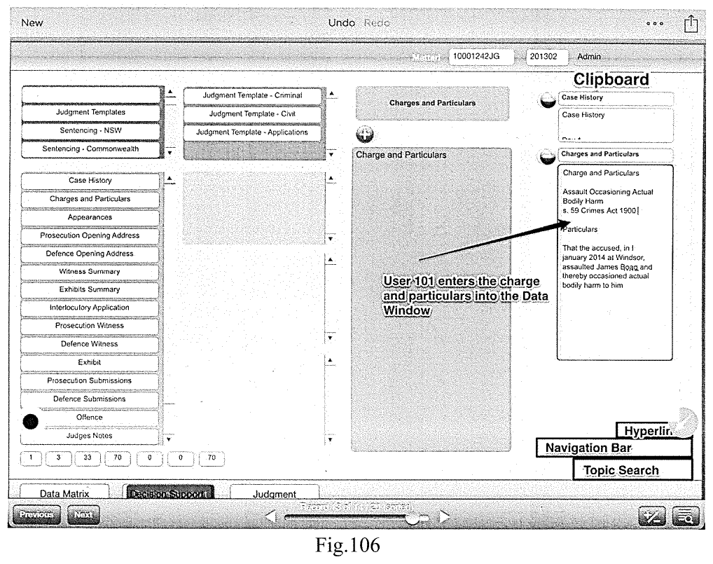

FIG. 106 illustrates user 101 entering the charge and the particulars into the Data Window.

The charge before the judge in this example are Assault Occasioning Actual Bodily Harm. That charge is entered into the Data Window and the particulars of that offence are added into that Data Window.

The judge then wishes to know the penalties that apply to the charge before the court.

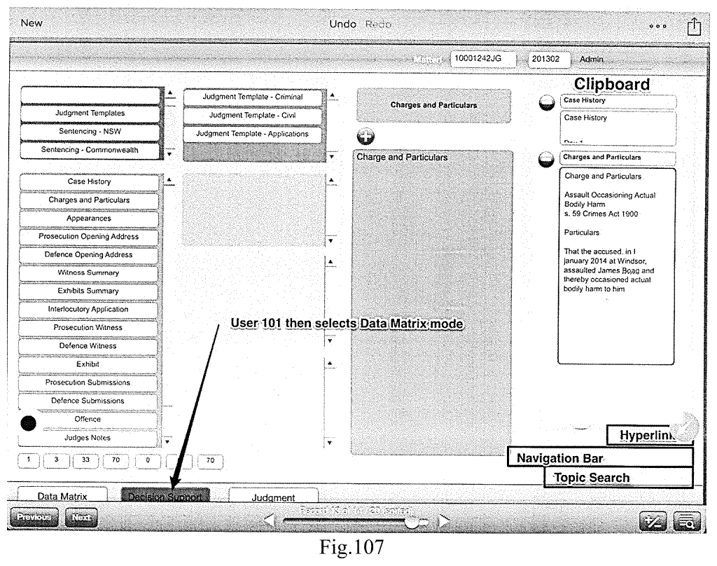

FIG. 107 illustrates user 101 selecting Data Matrix mode.

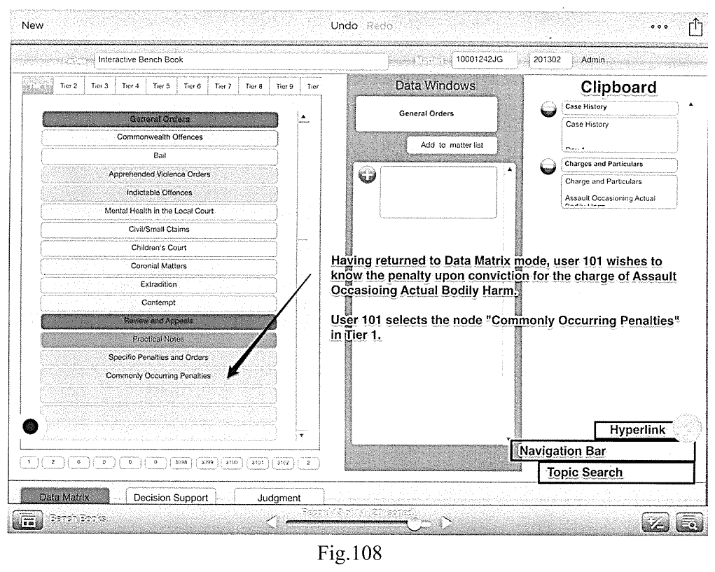

FIG. 108 illustrates user 101 selecting the node “Commonly Occurring Penalties” at Tier 1.

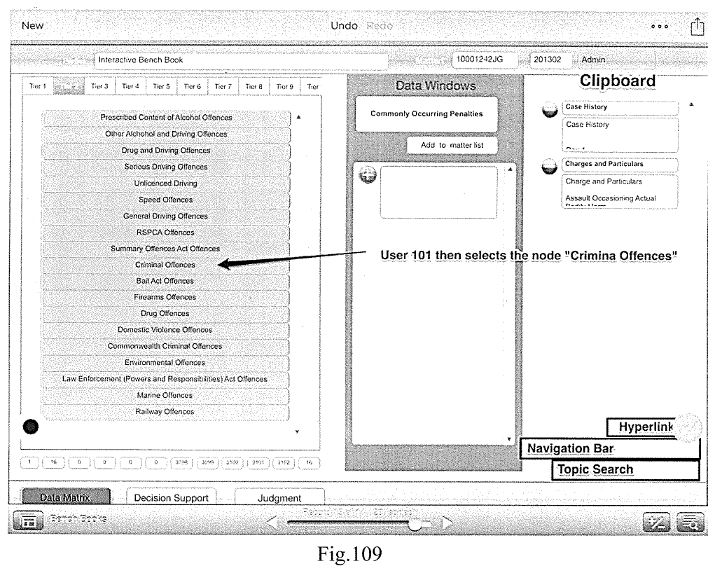

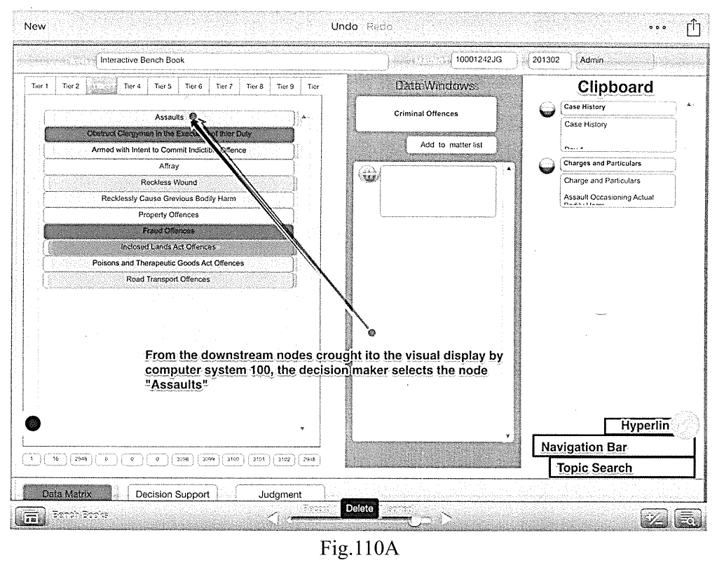

FIG. 109 illustrates user 101 then selecting the Criminal Offences node at Tier 2.

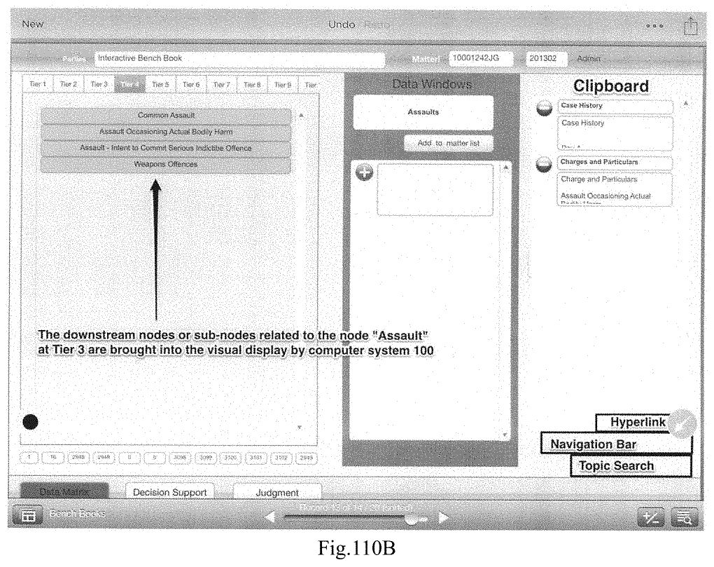

FIG. 110A “Assaults” is brought into the visual display by computer system 100 and is selected by user 101.

FIG. 110B then illustrates the downstream nodes related to the node “Assault”.

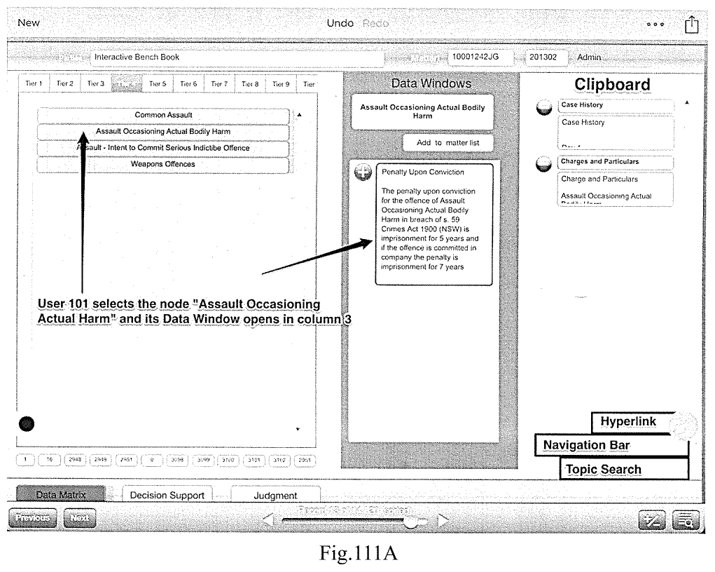

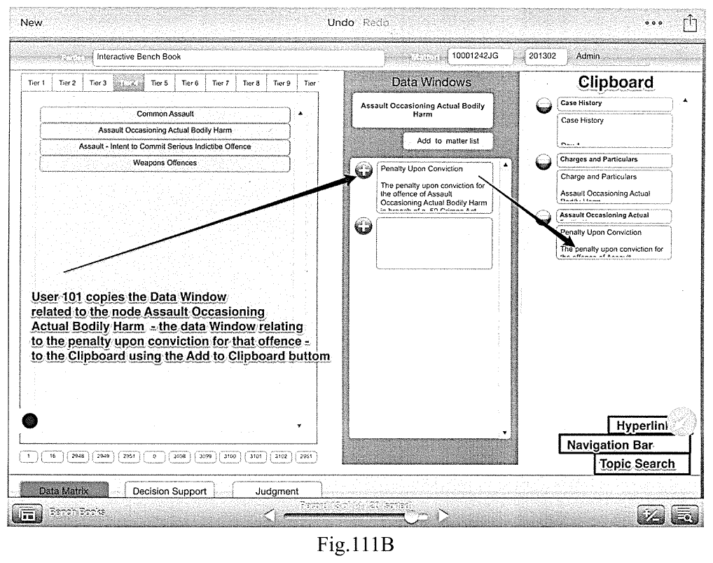

FIGS. 111A and 111B illustrate user 101 Selecting the Data Window Assault Occasioning Actual Bodily Harm and copying it to the Clipboard.

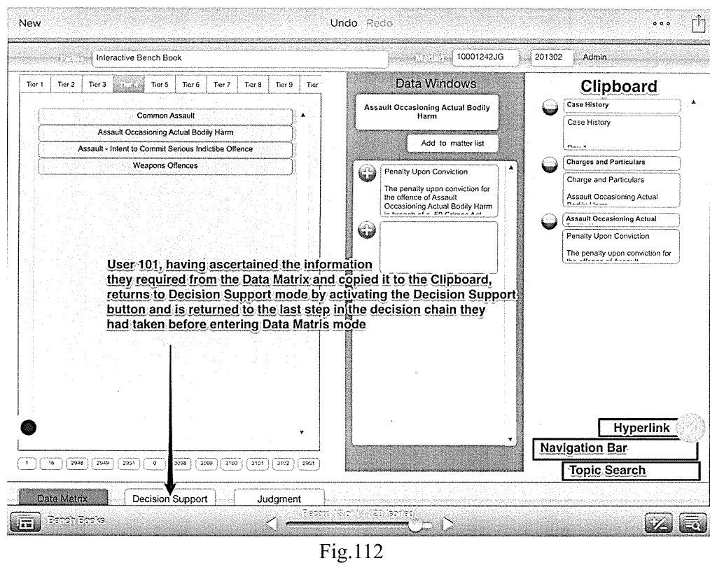

FIG. 112 illustrates the judge then selecting decision Assistance mode.

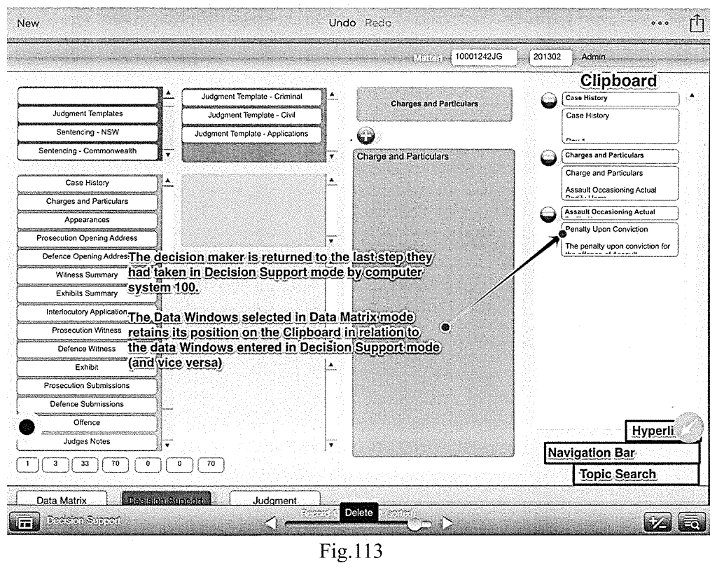

FIG. 113 illustrates the how the Data Window selected in Data Matrix mode retains its position in the Data Windows in the Clipboard relative to the Data Windows selected in Decision Assistance mode.

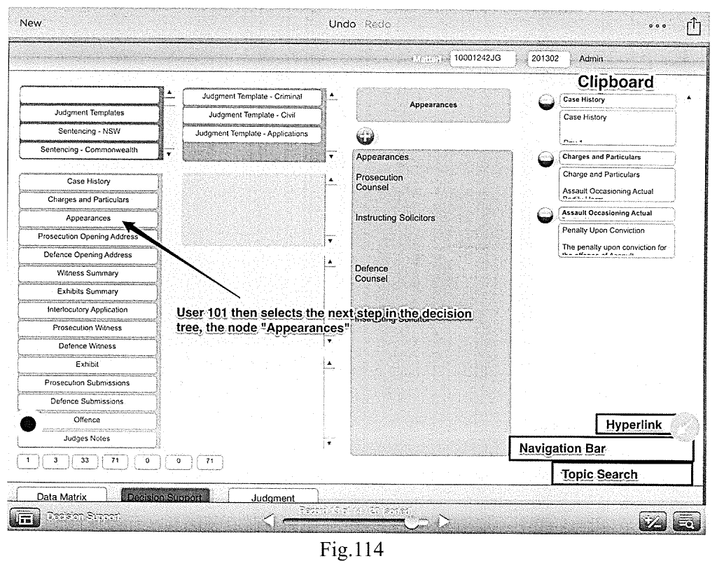

FIG. 114 illustrates the judge then selecting the node “Appearances”.

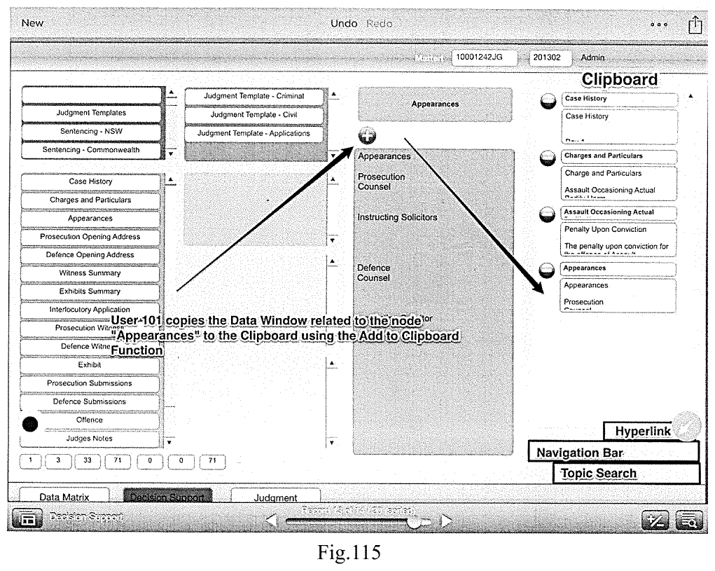

FIG. 115 illustrates the judge selecting the Data Window for the node “Appearances” and copying it to the Clipboard.

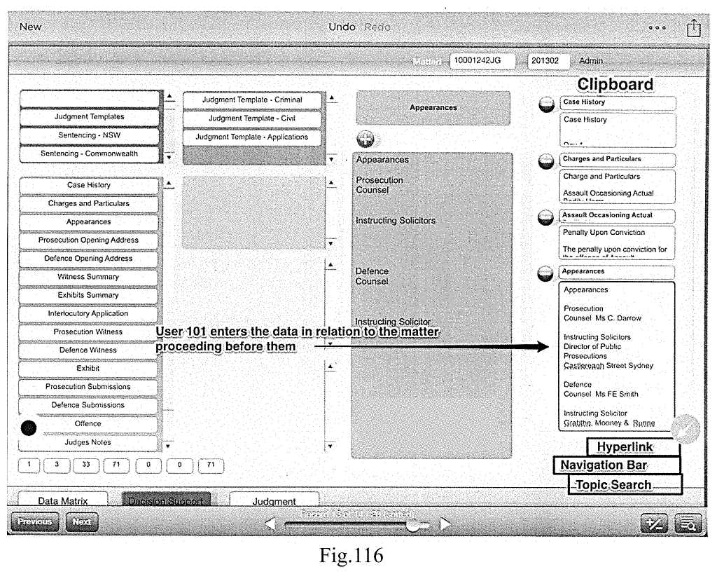

FIG. 116 illustrates the judge opening the Data Window for “Appearances” in the Clipboard and entering the data relevant from the matter proceeding before the judge.

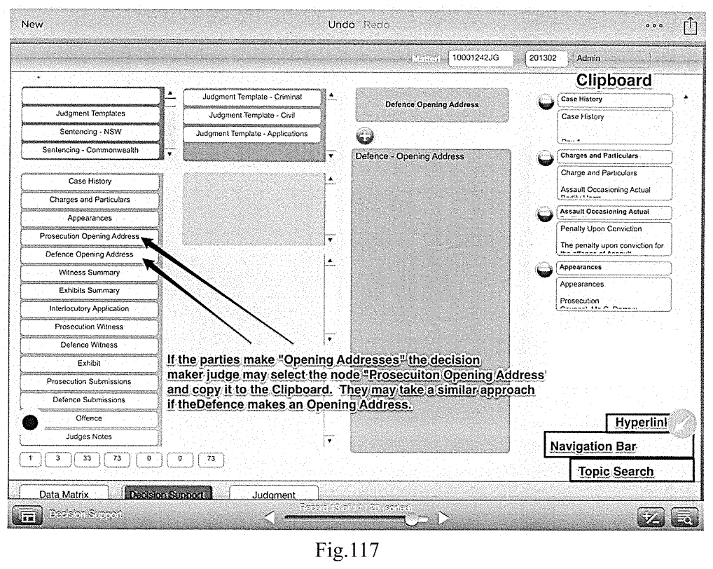

FIG. 117 illustrates that if the parties make opening addresses, the judge may select the “Opening Addresses” node from the decision chain in Window 3.

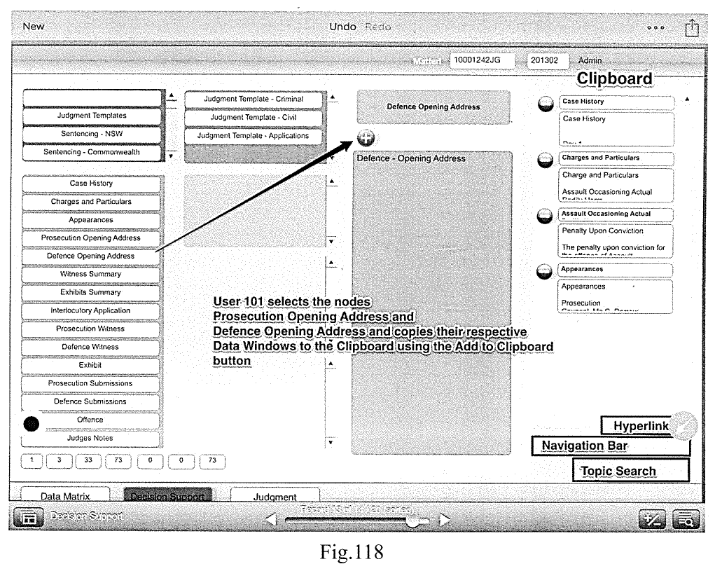

FIG. 118 illustrates the judge copying the Data Window relevant to the node “Opening Addresses” the Clipboard and the judge may then make their notes as appropriate.

The same procedure applies if the Defence makes an Opening Submission.

The transcript may be linked to this Data Window by a hyperlink. This would allow the judge to open the written transcription of the proceedings of the Opening Submissions in a window within computer system 100 for the assistance of the judge when writing their judgment.

The first witness is called.

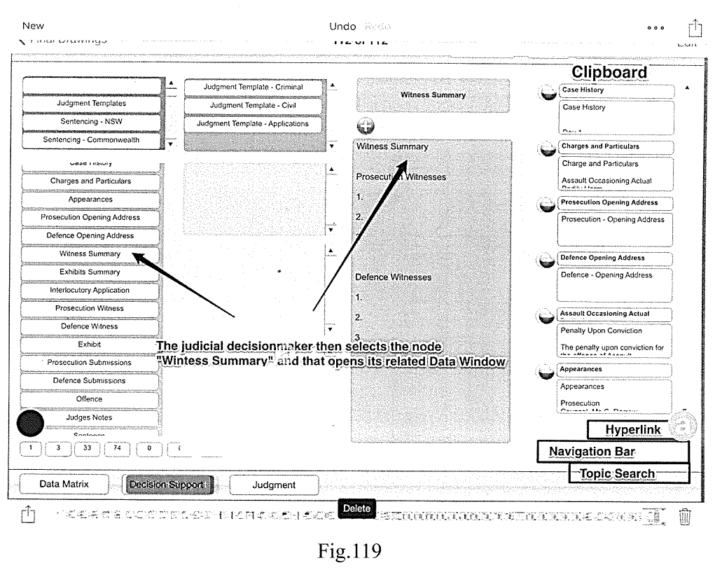

FIG. 119 illustrates the judge selecting the “Witness Summary” node from the Decision chain in Window 3.

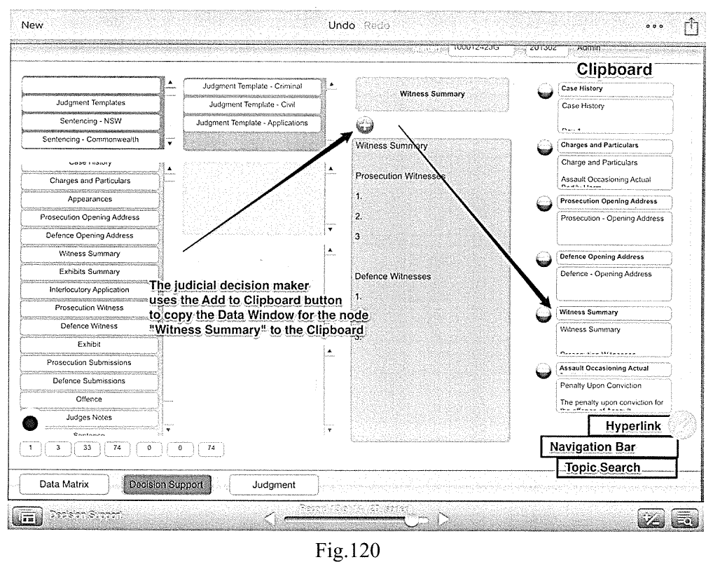

FIG. 120 illustrates the judge copying the Data Window relevant to the node “List of Witnesses” onto the Clipboard.

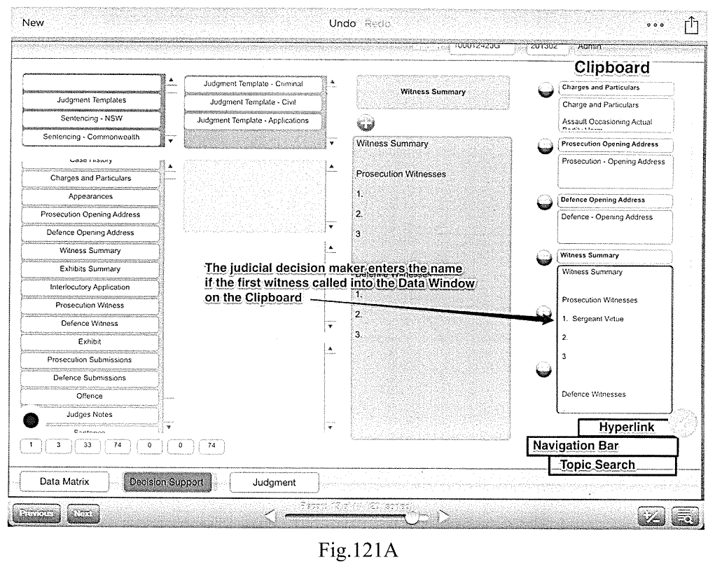

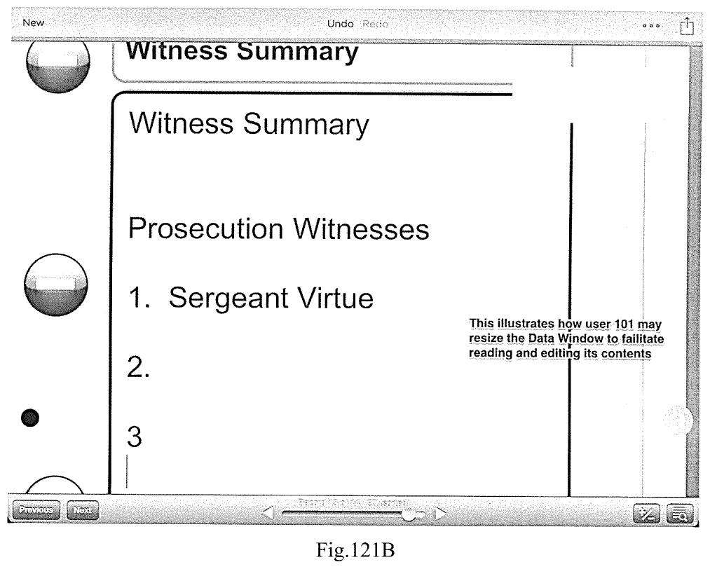

FIG. 121A illustrates the judge entering the name of the first witness. FIG. 121B illustrates how user 101 may resize the Data Window to facilitate reading and editing that Data Window.

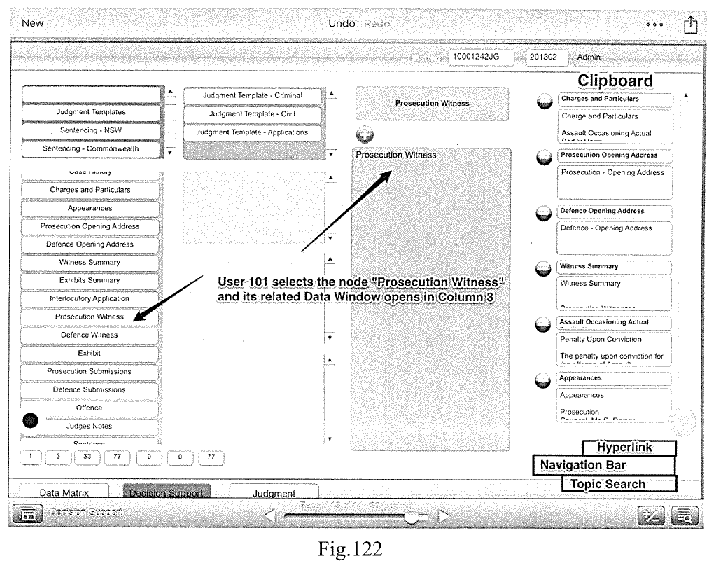

FIG. 122 illustrates the judge then selecting the node “Prosecution Witness” from the steps in the decision chain in Window 3.

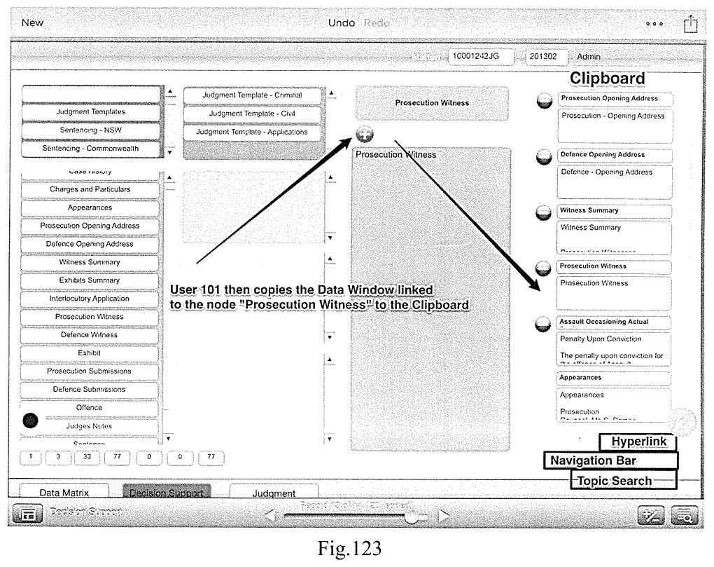

FIG. 123 illustrates the judge then copying the Data Window relevant to the node “Prosecution Witness” in Window 3 to the Clipboard.

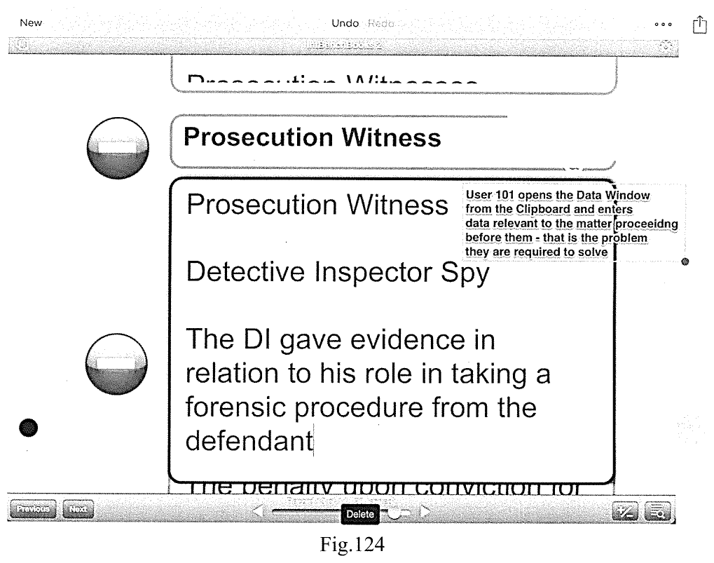

FIG. 124 then illustrates the judge opening the Data Window in the Clipboard, and entering that witnesses name and making notes in respect of that witnesses evidence.

The Data Window may have added to it a hyperlink allowing the transcription of that witnesses evidence able to be opened in a window within computer system 100 for the

convenience and reference of the judge and that window may also have added a hyperlink to the audio recording of the witnesses evidence.

This process is continued for the evidence of all witnesses called.

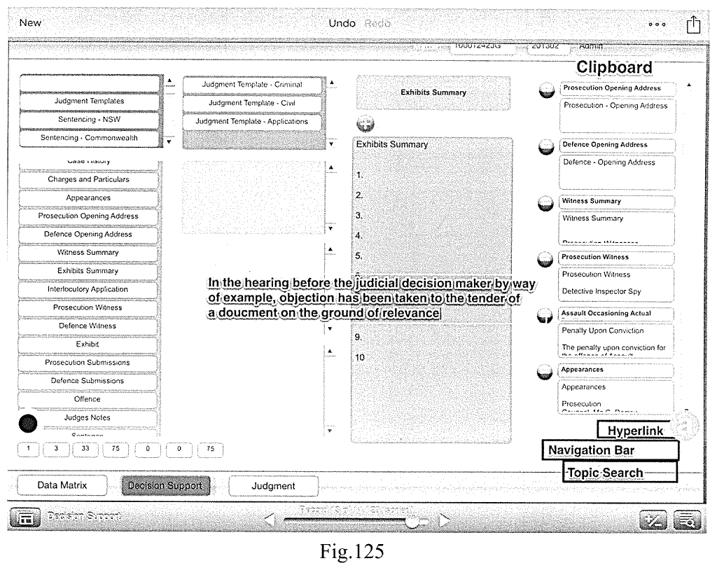

FIG. 125 illustrates the operation of computer system 100 in respect of the tender of a document.

Objection maybe taken to the tender of the document on the grounds of relevance.

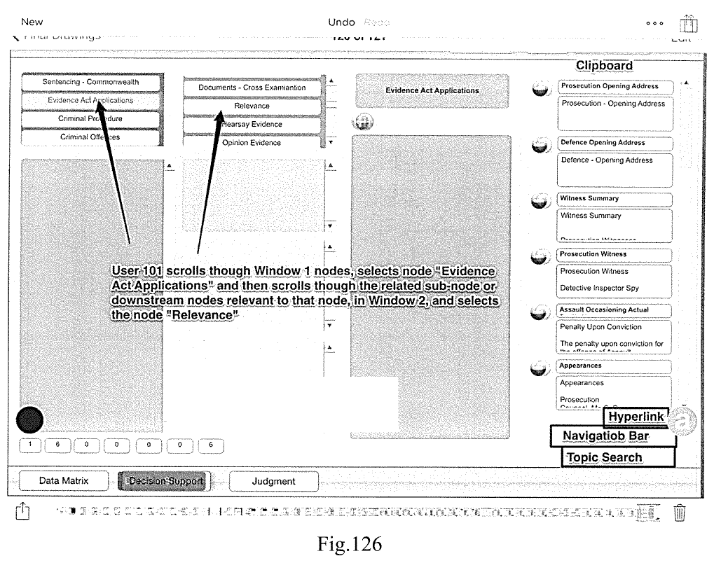

FIG. 126 illustrates the judge returns to Window 1 of the Decision Support mode and selects the node, “Evidence Act Applications” and the judge then scrolls though the nodes relevant to “Evidence Act Applications” which appear in Window (2) and selects the node “Relevance”.

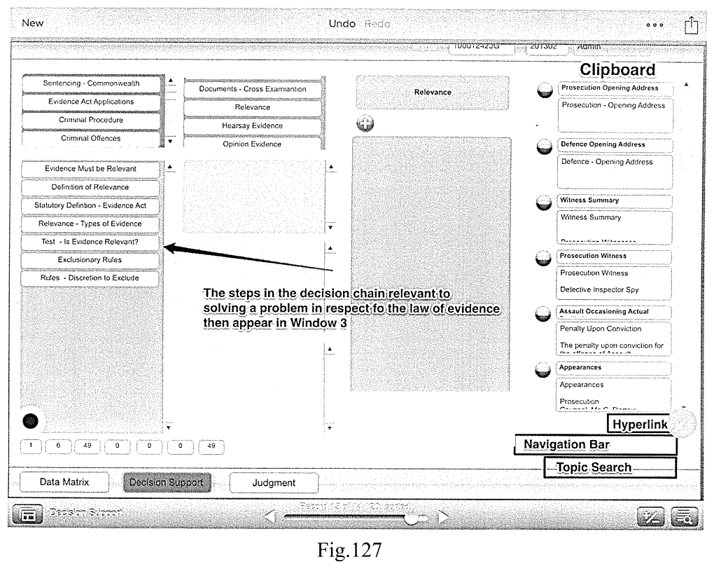

FIG. 127 shows the steps in the decision chain relevant to solving a problem in respect of the law of relevance then appear in Window 3.

The judge may follow the steps in that decision chain sequentially or may proceed directly to the node which addresses the specific aspect of the problem which presents itself.

The judge may by way of one example want to know the definition of the word “relevance” as a legal construct.

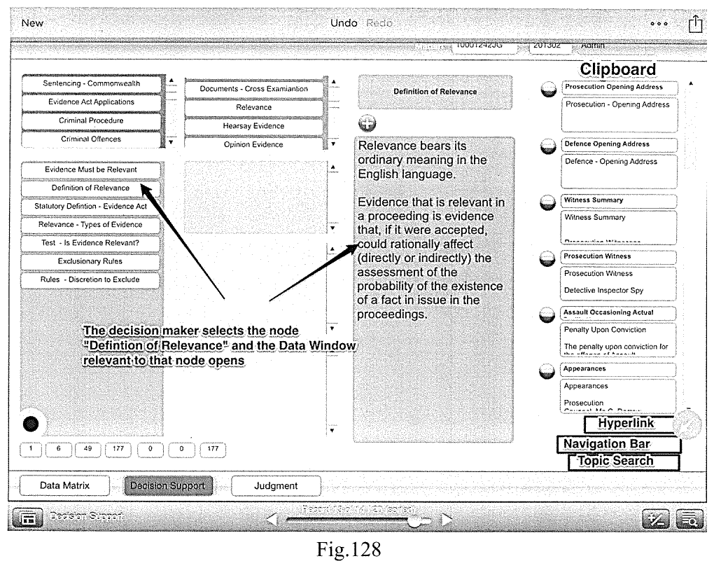

FIG. 128 illustrates the decision maker selecting the node “Definition of Relevance” and the Data Window relevant to that node opens.

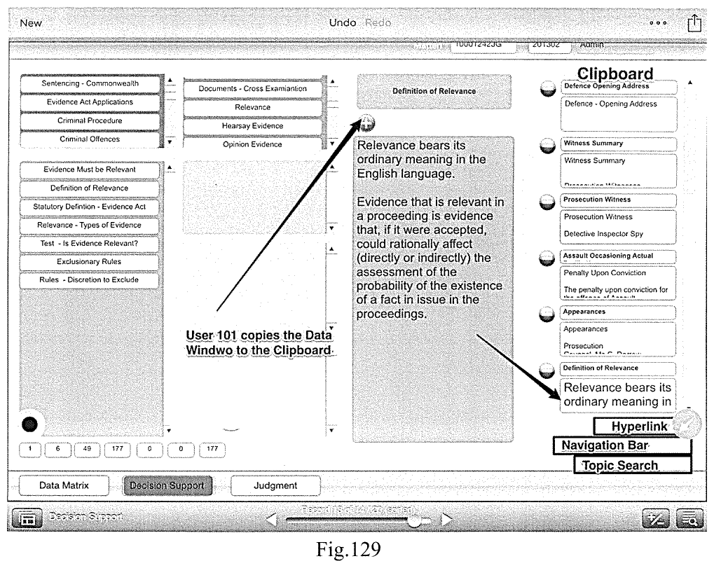

The Decision maker may copy that Data Window to the Clipboard by selecting the “Add to Clipboard” function and the Data Window then appears in the Clipboard. See FIG. 129.

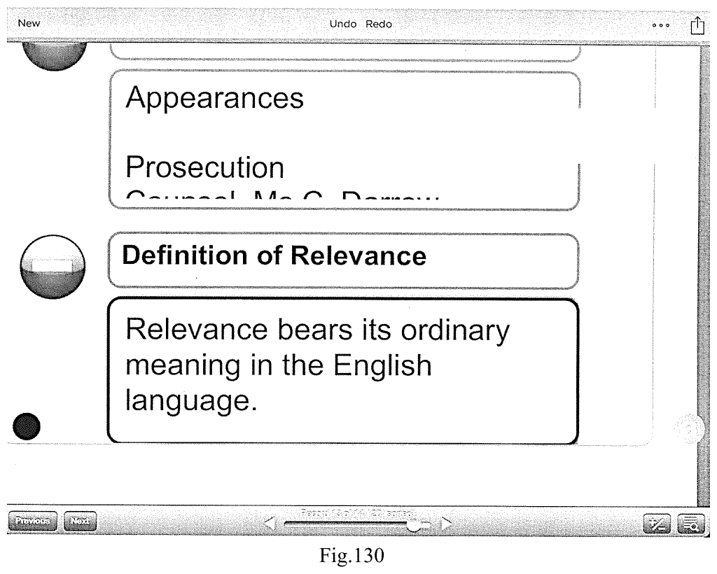

FIG. 130 illustrates the decision maker opening and editing that Data Window on the Clipboard as is appropriate by reference to the specific problem in the matter before the judge.

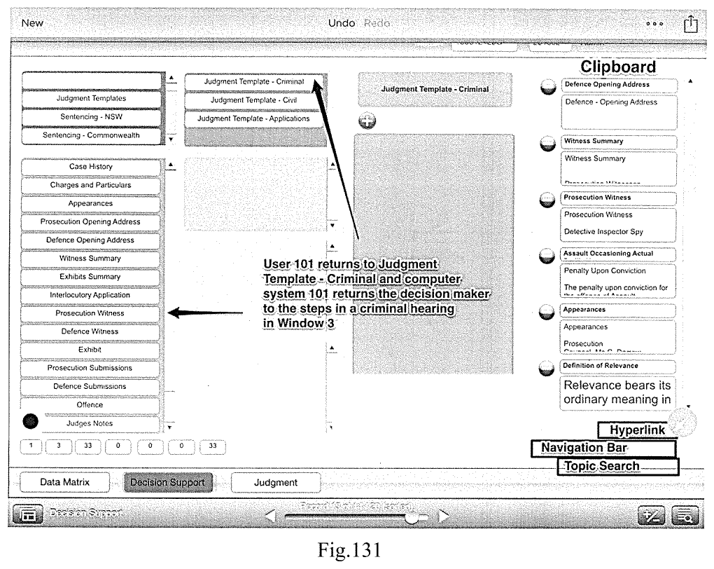

If the document is held by the judge decision maker to be relevant and admissible, FIG. 131 illustrates the judge returning to the node “Judgment Template—Criminal” in Window 1.

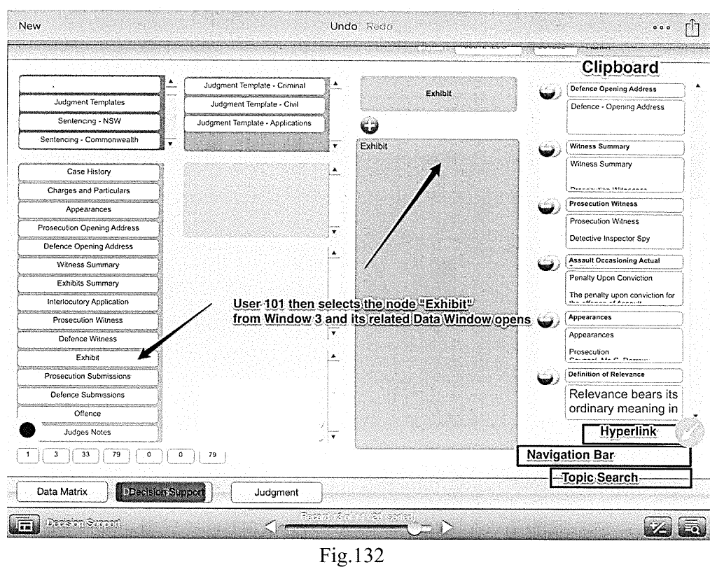

FIG. 132 illustrates the judge then selecting the node “Exhibit” from Window 3 and its Data Window opens.

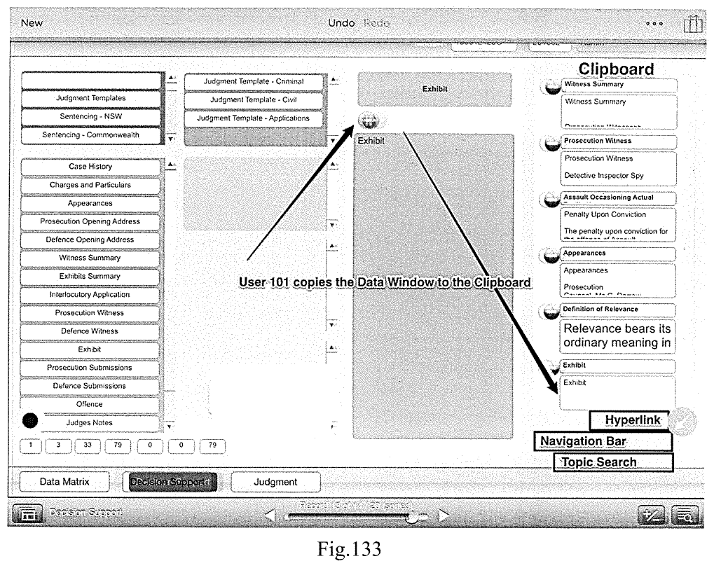

FIG. 133 illustrates the judge copying the Data Window “Exhibit” onto the Clipboard.

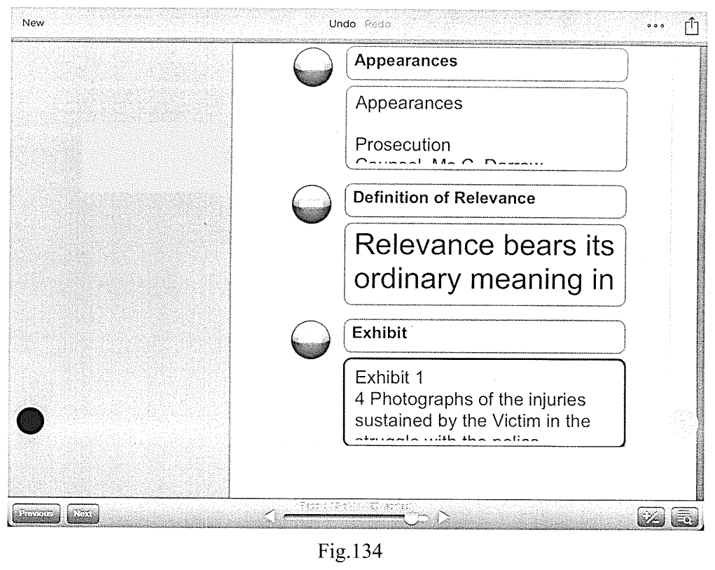

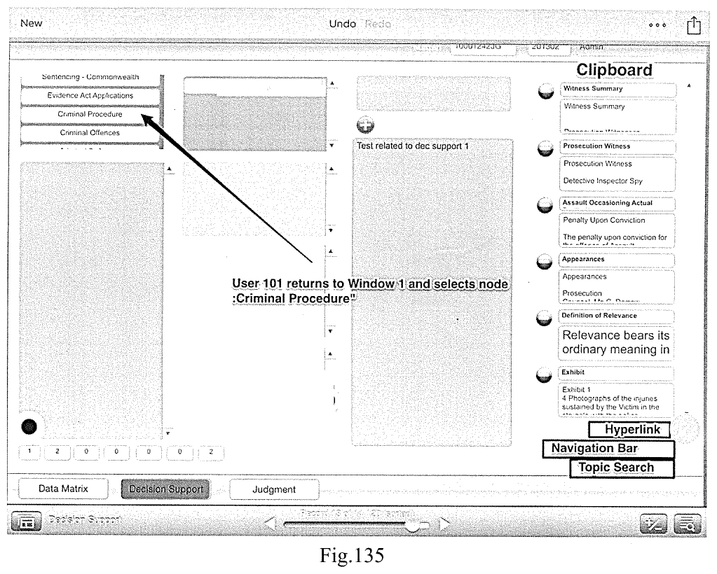

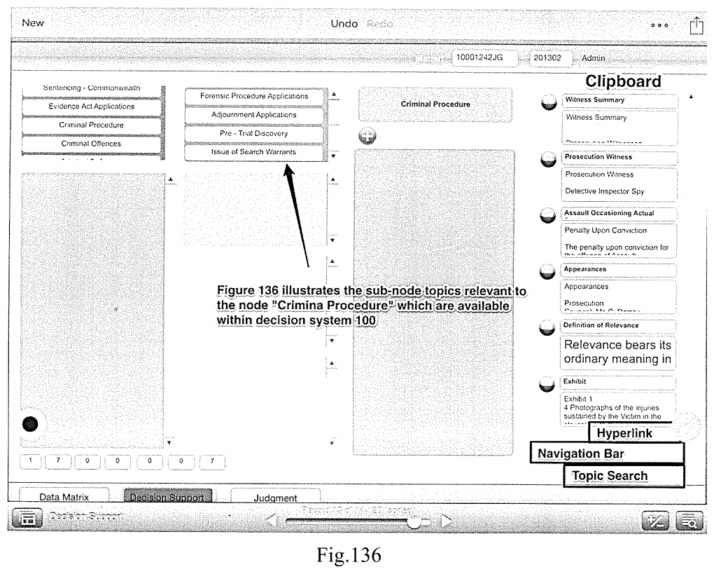

FIG. 134 illustrates the judge then opening the Data Window in the Clipboard and entering the details of the Exhibit and any notes the Judge may have of the exhibit—in respect of its importance in the proceedings, or in relation to the specific part of the exhibit that is of significance in the proceedings.

A hyperlink may be included in the Data Window allowing a graphic picture of the exhibit to be opened within a window in computer system 100 for the assistance of the Judge.

If an interlocutory application is made during the proceedings, such as the defence making what in Australia is referred to as a “Prasad Application”, the decision maker may return to Window 1 and select the node “Criminal Procedure”. See FIG. 135.

FIG. 136 illustrates the sub node topics available within computer system 100 in relation to the node “Criminal Procedure”.

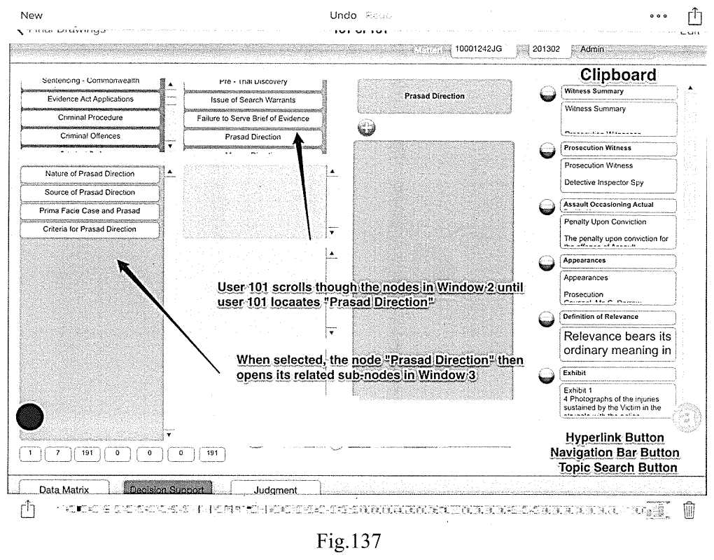

FIG. 137 illustrates the decision maker scrolling though these sub-node topics relevant to the node “Criminal Procedure” until the decision maker locates “Prasad Direction”.

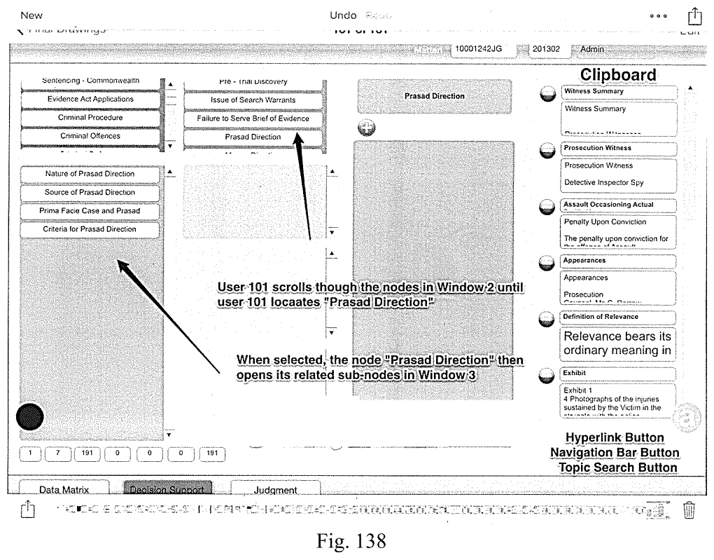

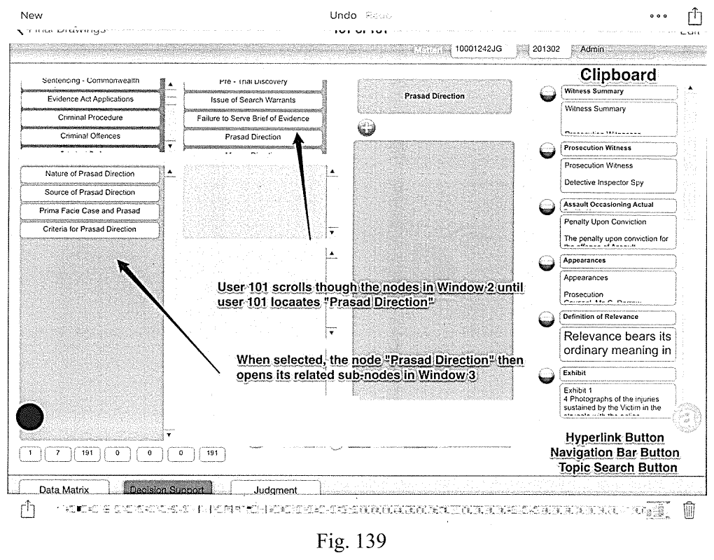

FIG. 138 illustrates the Decision Maker selecting the node “Prasad Application” and its related sub-node topics—those topics relevant to the decision tree necessary for a judge to determine whether an application for a Prasad Direction should be made. FIG. 139 illustrates the decision step nodes—appear in Window 3.

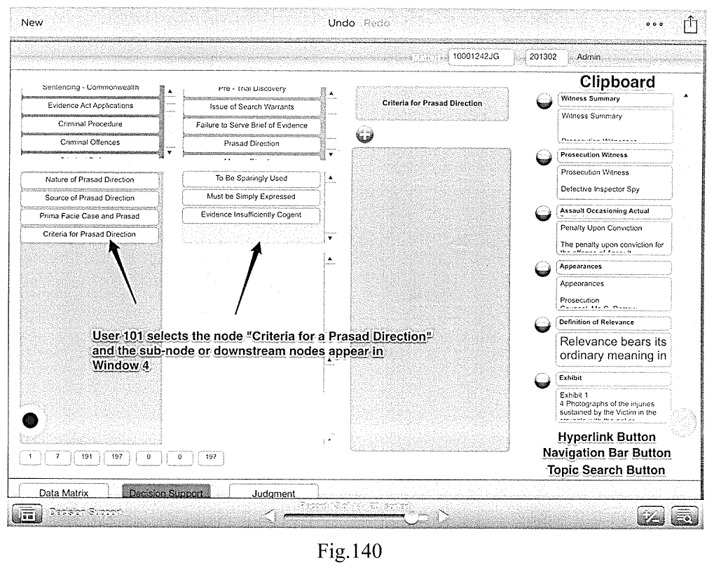

The decision maker may proceed though each step in the decision chain and may select the node “Criteria for Prasad Direction”. That is illustrated in FIG. 140.

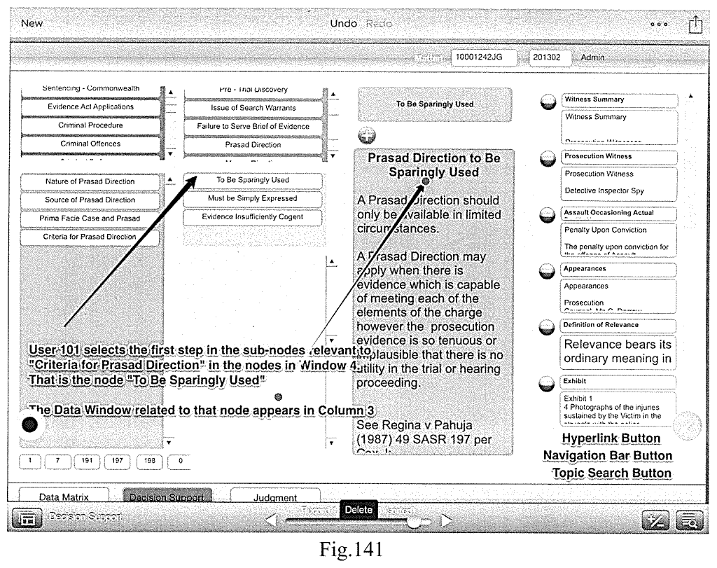

The decision maker may select the first step in the criteria to be applied and the Data Window relevant to that node opens. This is illustrated in FIG. 141.

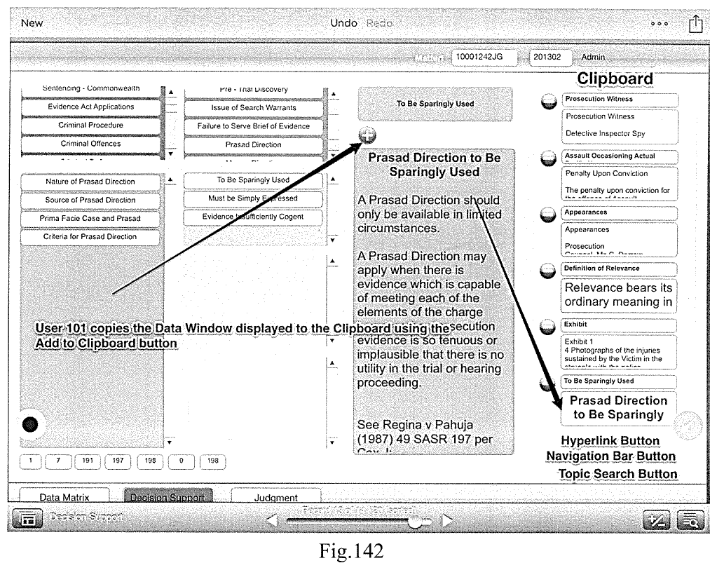

FIG. 142 illustrates the decision maker copying the Data Box relevant to the node “To Be Sparingly Applied” into the Clipboard.

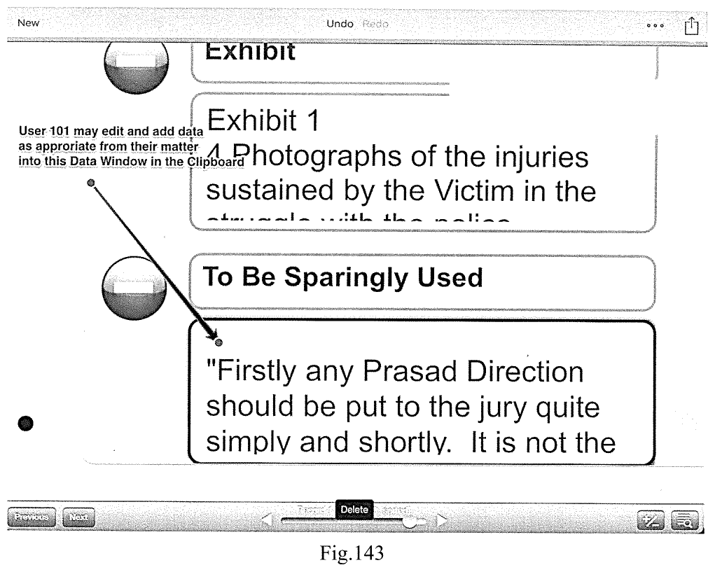

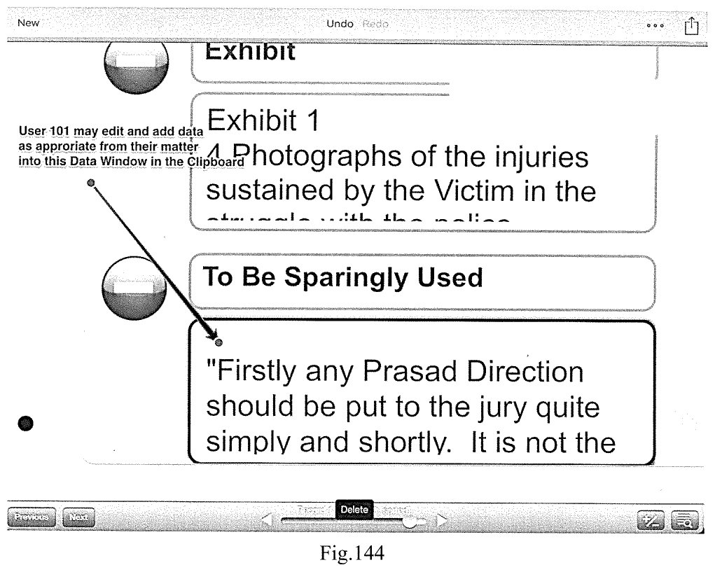

FIGS. 143 and 144 illustrates the decision maker resizing the Data Window to edit its contents and add data relevant to the matter proceeding before the judge.

The judge then makes a determination of whether the Prasad Direction should be made. More complex legal problems may require computer system 100 to take the decision maker deeper into the decision system, into Windows 6, 7 etc to “n” as may be appropriate.

This example illustrates how computer system 100 operates to provide the decision maker with access to that level of decision support relevant to the specific problem the decision maker is endeavouring to solve. More complex problems will progress further into the decision tree than will less complex problems. It is noted that computer system 100 provides user 101 with that level of detail and complexity necessary to solve the specific problem before that user.

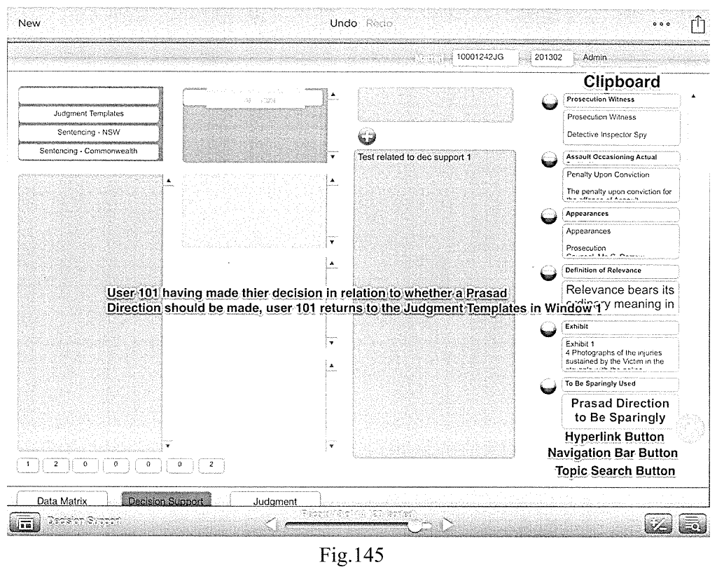

Having made a decision on the Application for a Prasad Direction that the hearing will continue, the decision maker returns to the Judgment Templates in Window 1, illustrated in FIG. 145.

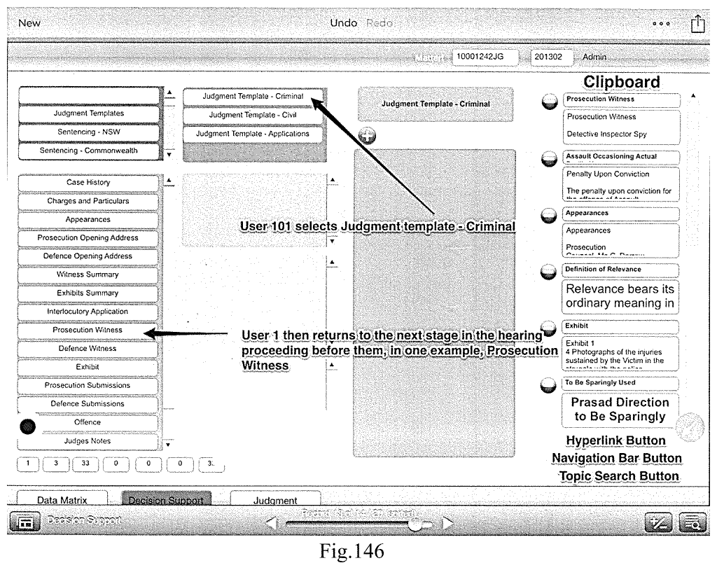

The decision maker then selects Judgment Template—Criminal in Window 2, as illustrated in FIG. 146, and then returns to the hearing by selecting the node relevant to the next stage in the hearing, in one example, Prosecution Witness.

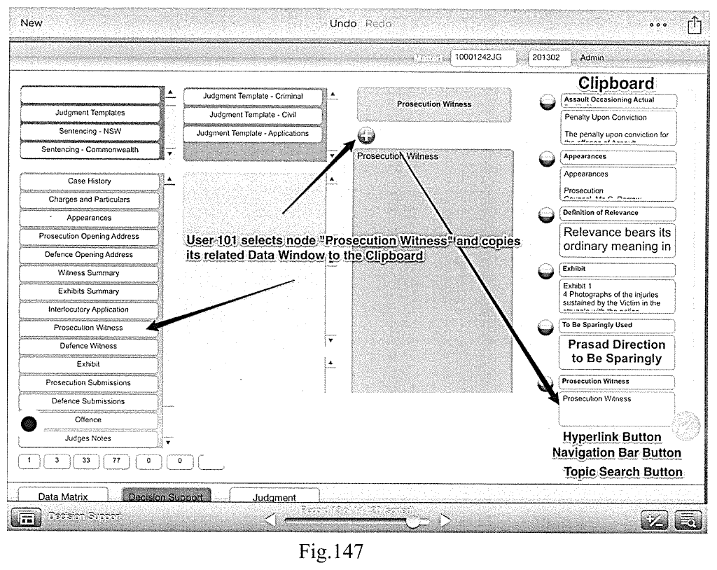

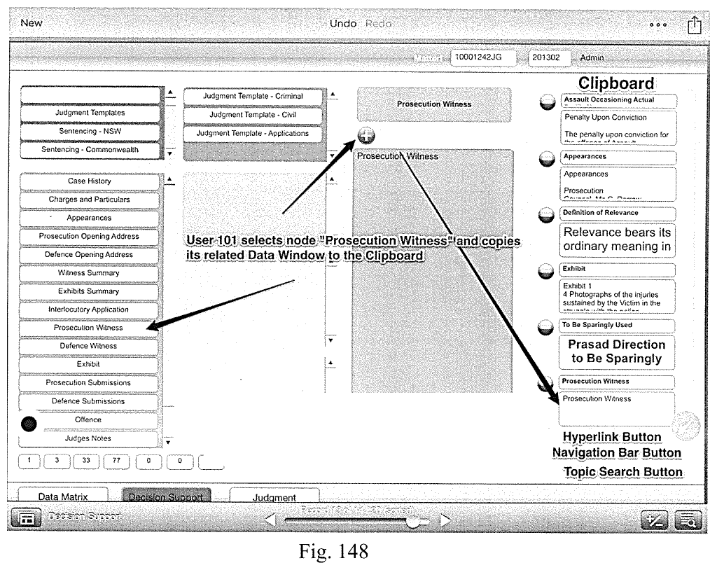

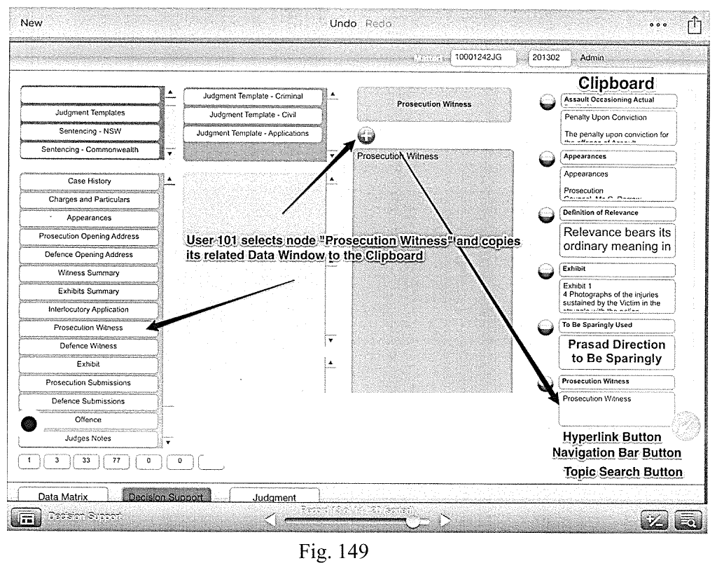

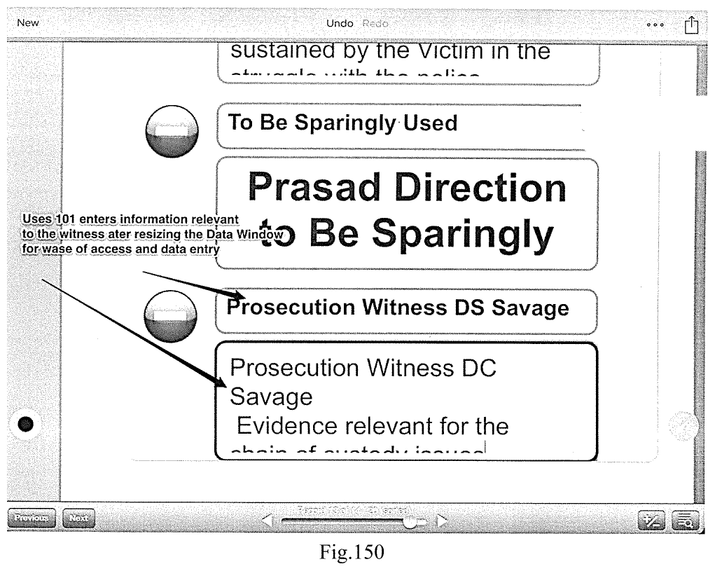

FIG. 147 illustrates the decision maker selecting the node “Prosecution Witness”, its Data Box appearing in FIG. 148, and that Data Window copied to the Clipboard in FIG. 149, and in FIG. 150, the decision maker enters details of the witness and notes of the witnesses evidence.

The decision maker judge continued though the hearing and after the decision maker has heard the testimony of all witnesses, ruled on evidential issues and applications that have arisen during the hearing with the decision support assistance provided by the computer, the judge will hear the parties submissions and enter their notes of the parties submissions into computer system 100 by selecting the parties submissions.

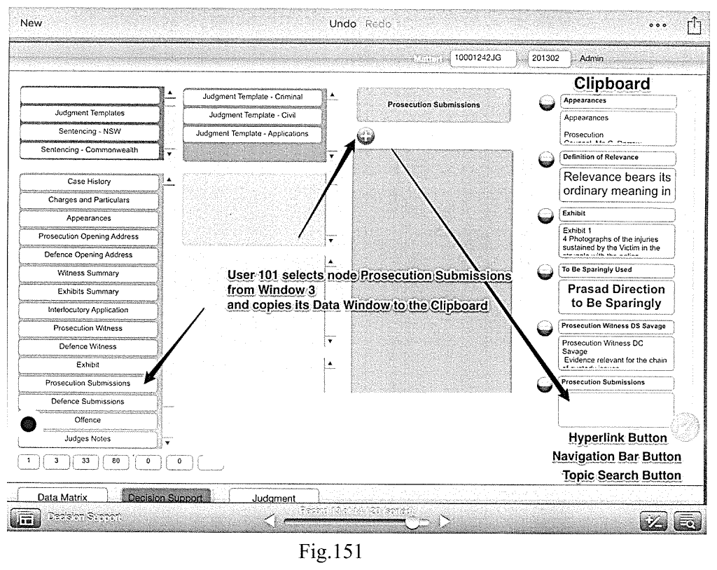

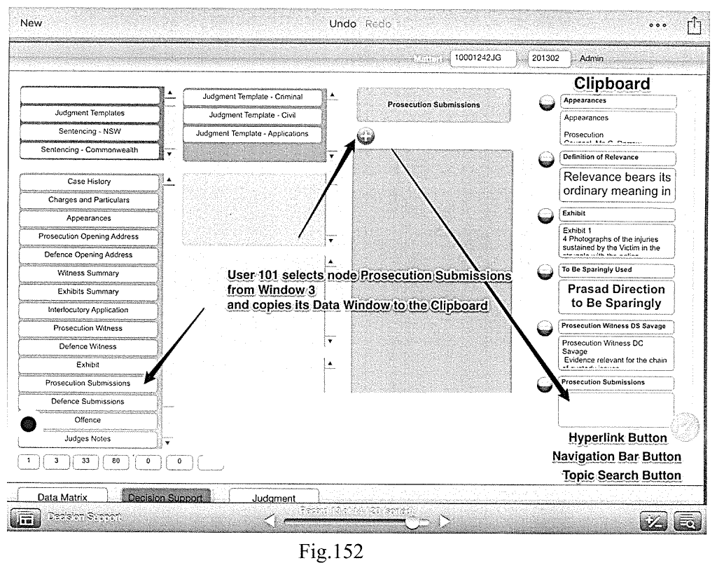

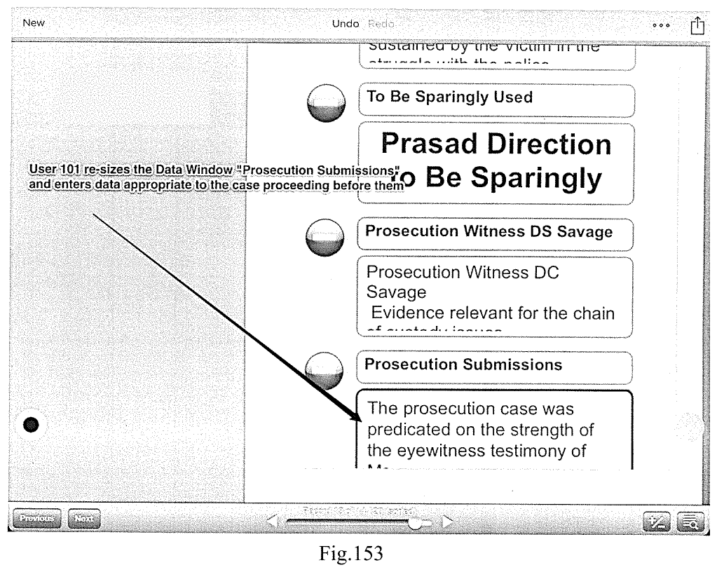

FIG. 151 illustrates the decision maker selecting the node “Prosecution Submissions” from Window 1 of Decision Assistance mode. FIG. 152 illustrates the Data Window relevant to that node being copied onto the Clipboard and FIG. 153 illustrates the judge opening that Data Window and entering the judges notes in relation to the submissions. It is noted that a hyperlink to the transcription of the submissions may be entered into this Data Window and opened from within computer system 100 by the user for their assistance when the transcription is provided.

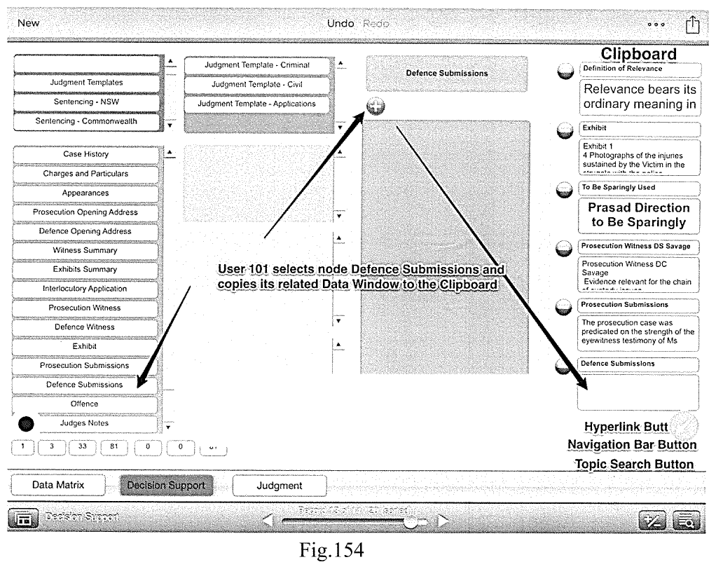

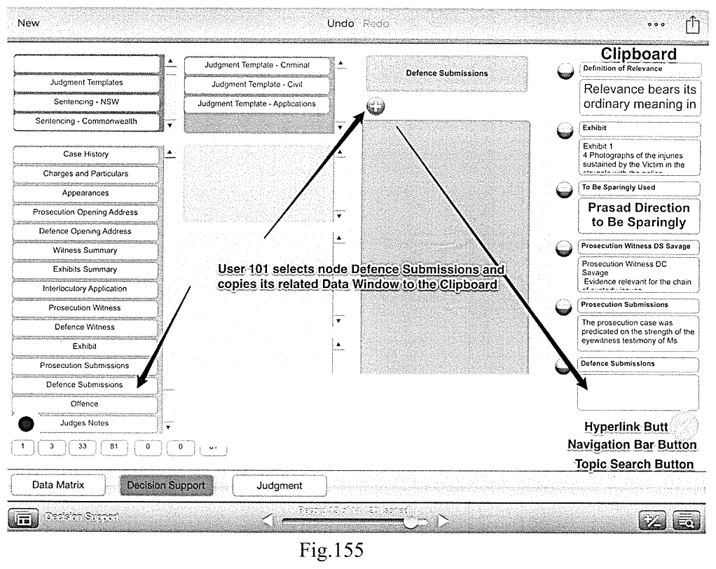

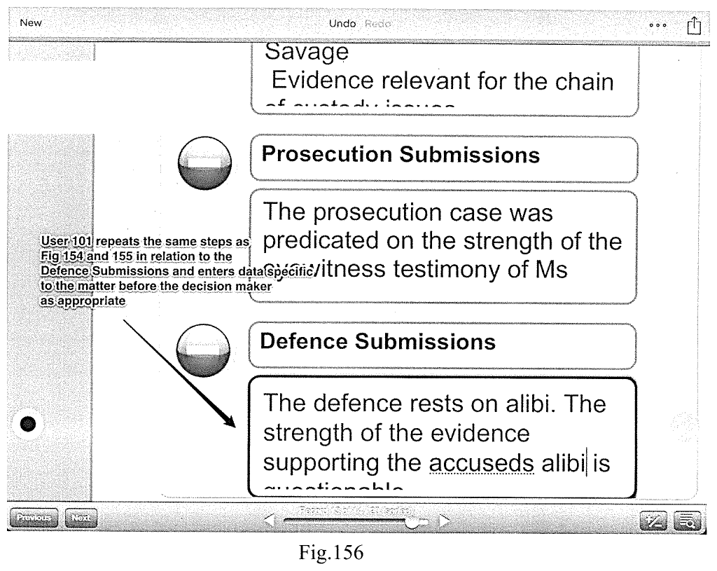

FIGS. 154, 155 and 156 illustrates the decision maker taking the same steps in relation to the submissions made on behalf of the defendant.

The hearing has then finished and the judicial decision maker then must decide whether the offence has been proven to the standard required by law and whether the defendant should be convicted.

That step in the decision chain requires the judge to identify the elements of the offence “Assault Occasioning Actual Bodily Harm”, and to apply those legal principles to the facts found.

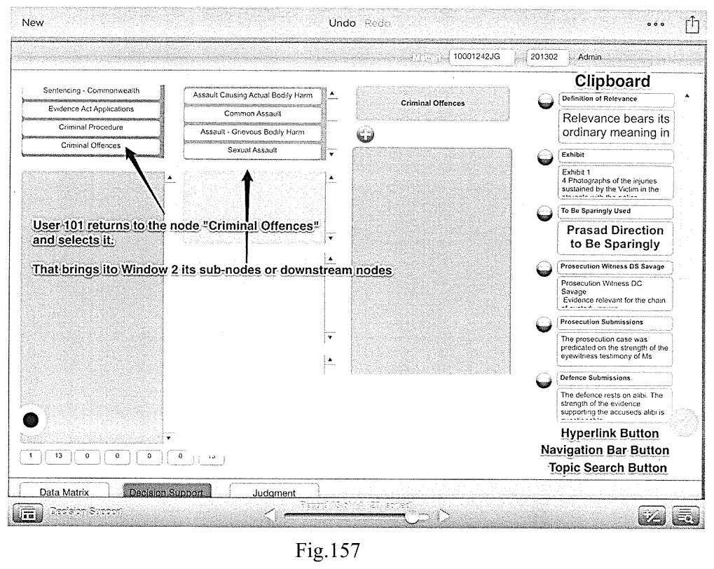

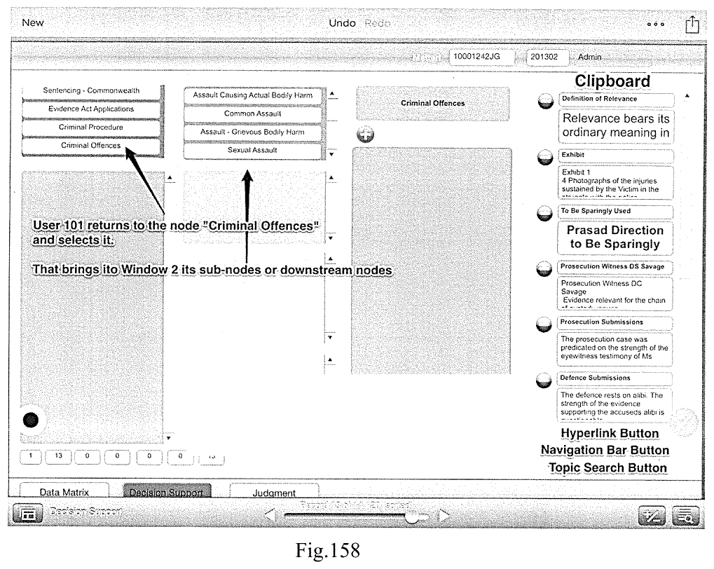

FIG. 157 illustrates the decision maker returning to Window 1 and selecting the node “Criminal Offences”. The list of offences for which decision support is available in computer system 100 appears in FIG. 158 in Window 2.

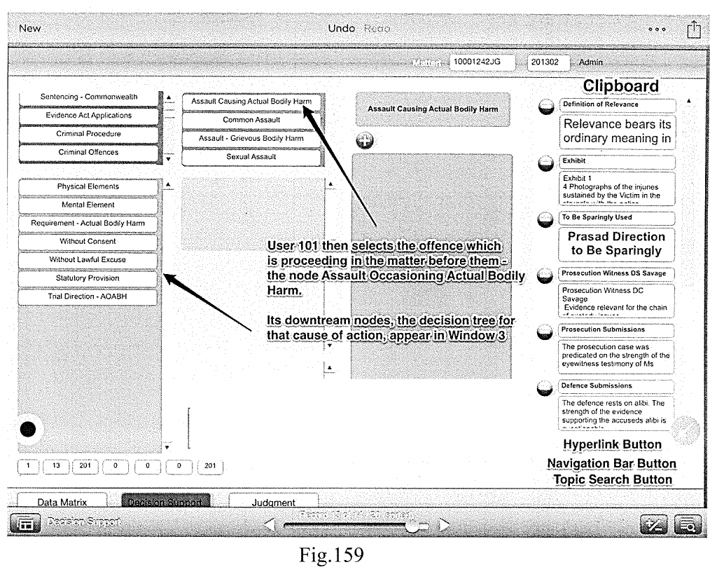

FIG. 159 illustrates the decision maker/judge then selecting the offence which is before that decision maker in the present trial, the node “Assault Occasioning Actual Bodily Harm”

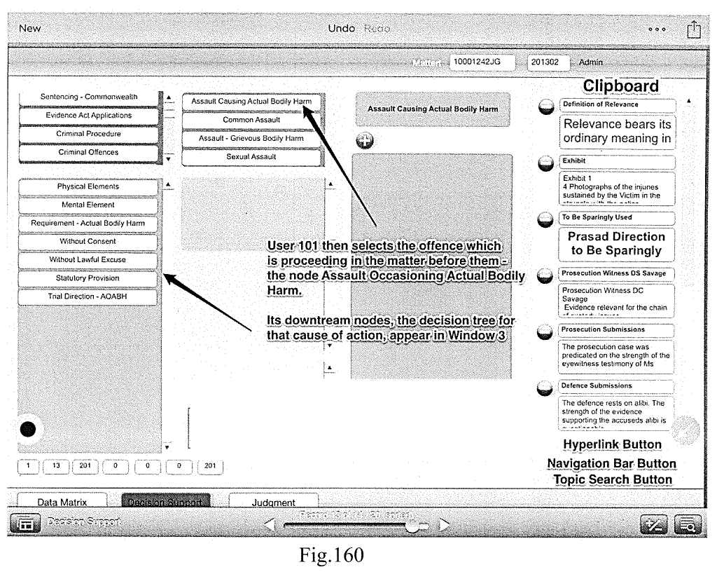

FIG. 160 illustrates the judge selecting the node “Assault Occasioning Actual Bodily Harm” and computer system 100 then brings the elements of that offence into the visual display.

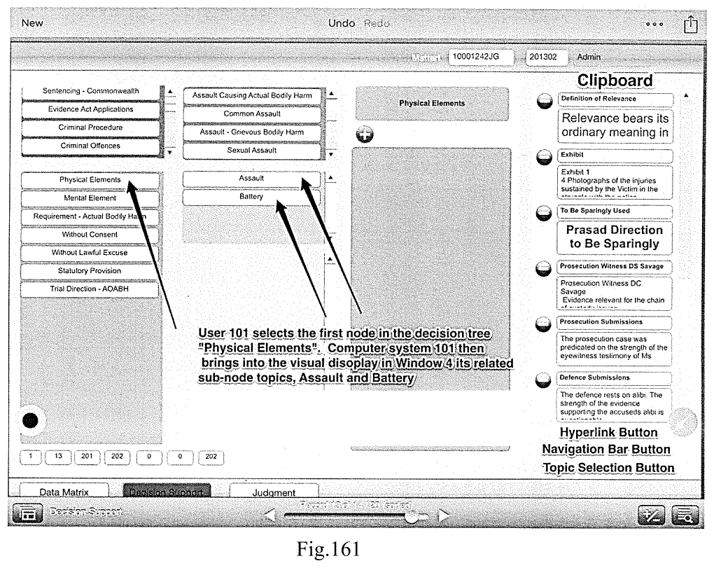

FIG. 161 illustrates the, decision maker selecting the node of the first step in the decision chain, Physical Elements.

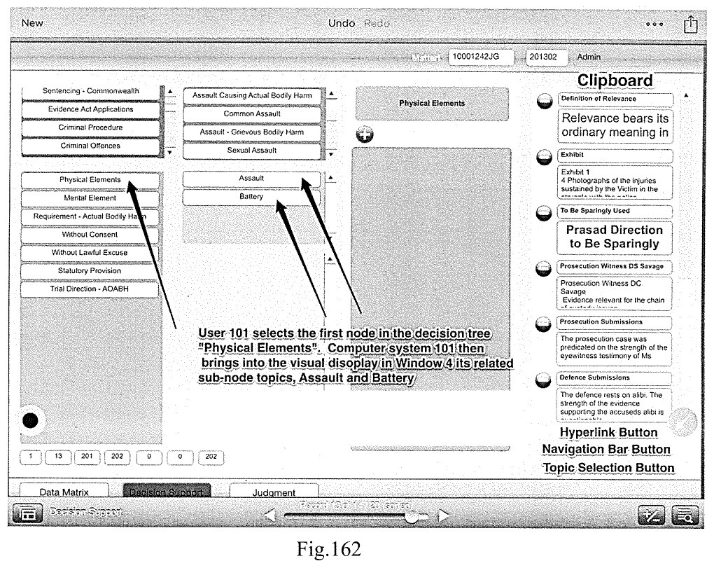

FIG. 162 illustrates computer system 100 bringing the sub-node topics relevant to the node “Physical Elements” into the visual display.

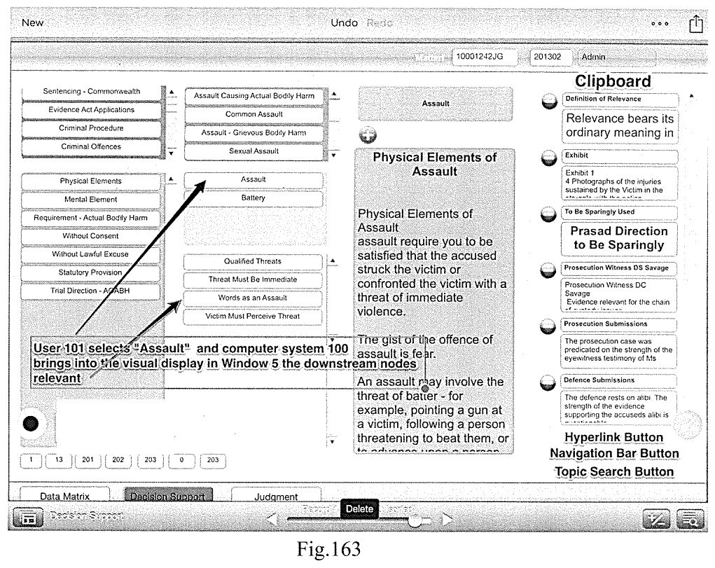

FIG. 163 illustrates the decision maker selecting the node Assault and computer system 100 then brings the Data Window relevant to the node Assault into the visual display in column 3. The selection of the node “Assault” also brings into the visual display the sub-node (or downstream) node topics relevant to the node “Assault”, in the present example, being

Qualified Threats

Threat Must Be Immediate

Words as Assault; and

Victim Must Perceive Threat.

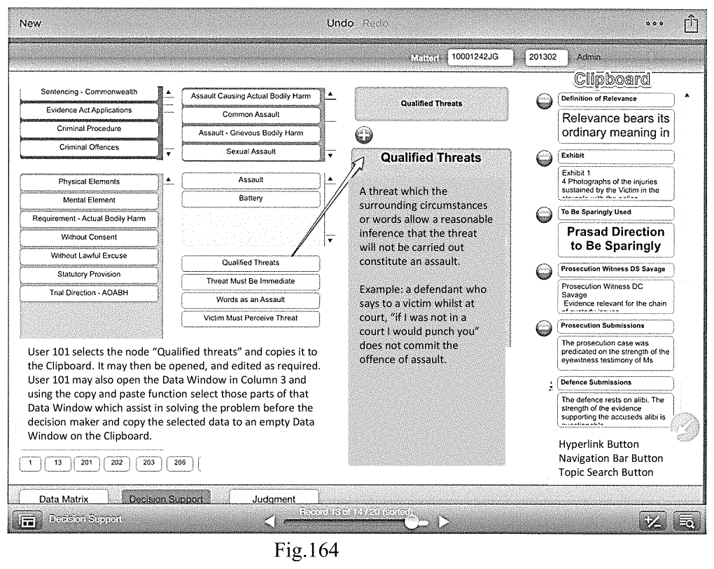

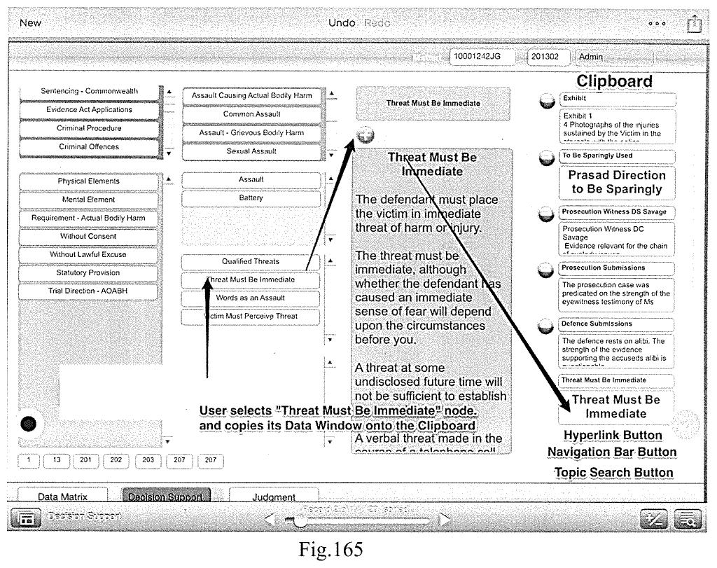

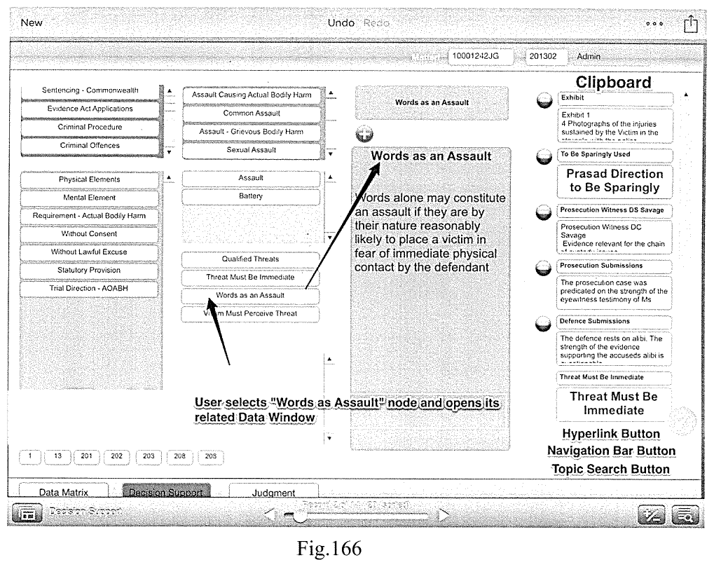

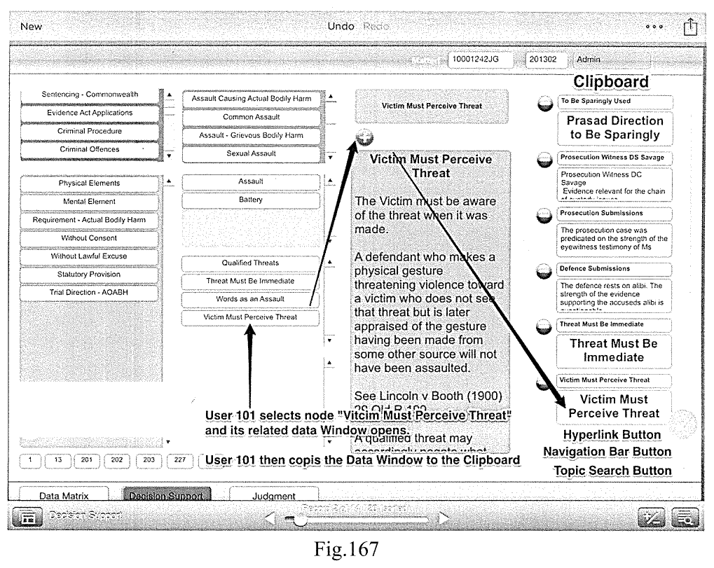

FIGS. 164, 165, 166 and 167 illustrate the user selecting each of the above Window 4 topics and computer system 100 bringing into the visual display the Data Windows relevant to those nodes.

The Decision Maker may copy all or any of the Data Windows (or using the copy and paste function) any part of those Data Windows which may be relevant to the matter proceeding before the judge, onto the clipboard.

The decision maker may progress though the steps in the expert decision chain.

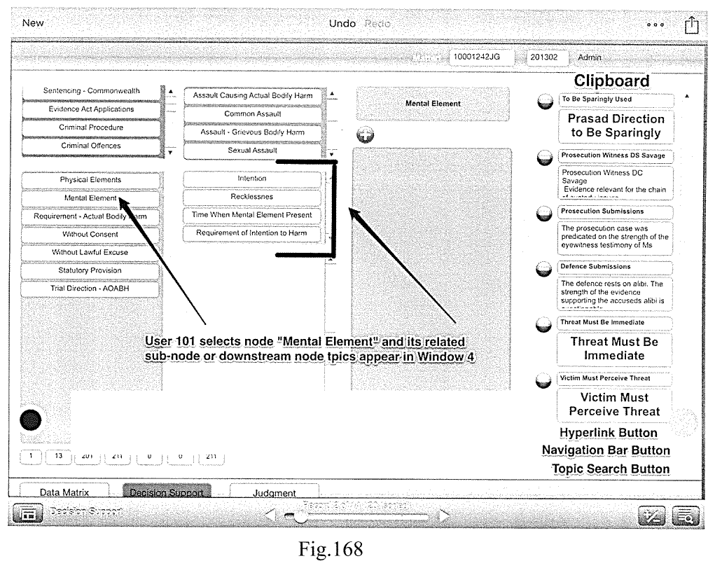

In one example, in FIG. 168, the decision maker judge selects the Mental Element node in Window 3.

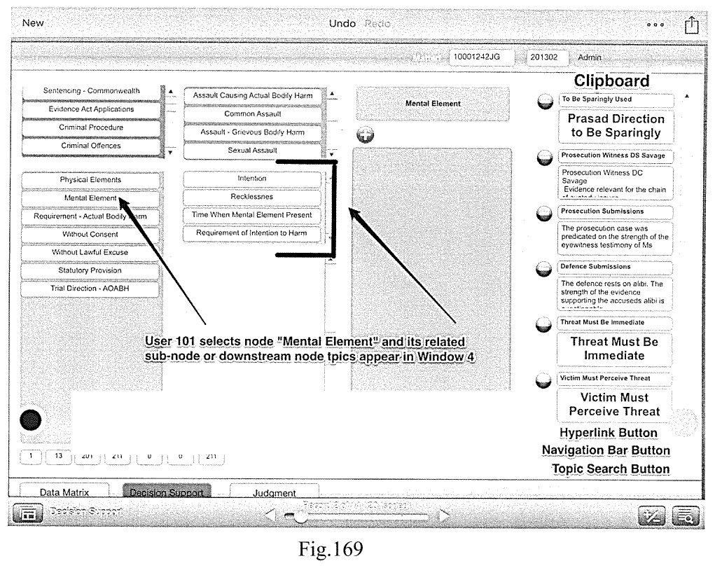

FIG. 169 illustrates computer system 100 brings into the visual display the downstream nodes relevant to the node Mental Element in Window 4.

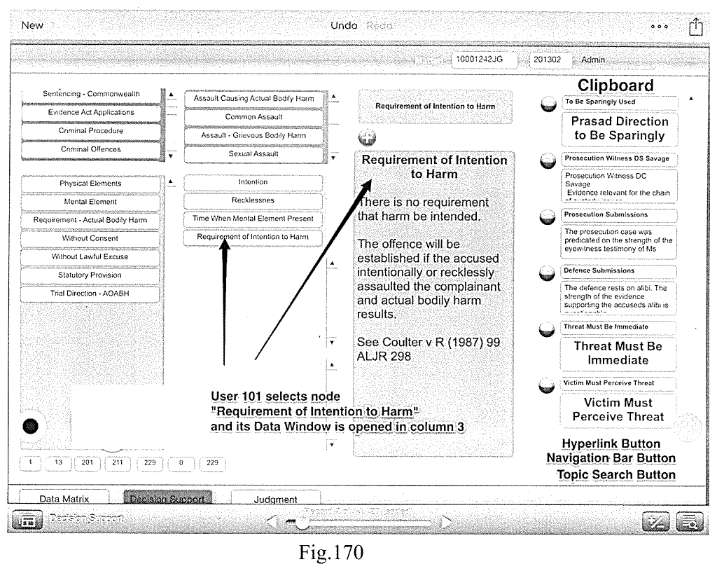

By way of further example, FIG. 170 illustrates the user selecting the Window 4 node “Requirement of Intention to Harm” and illustrates how computer system 100 brings the Data Window relevant to that node into the visual display of the computer screen in Column 3.

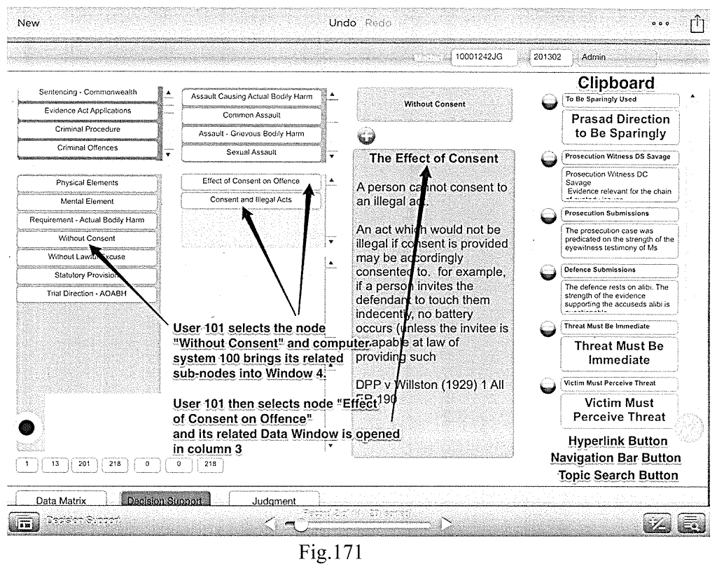

FIG. 171 illustrates the decision maker selecting the next step in the decision chain in Window 3, “Without Consent”, and the computer system 100 bringing its related sub-node topics into the visual display in Window 4.

FIG. 171 illustrates the decision maker selecting the node “Effect of Consent on Offence” and illustrates how computer system 100 then brings the Data Window relevant to that node topic into the visual display.

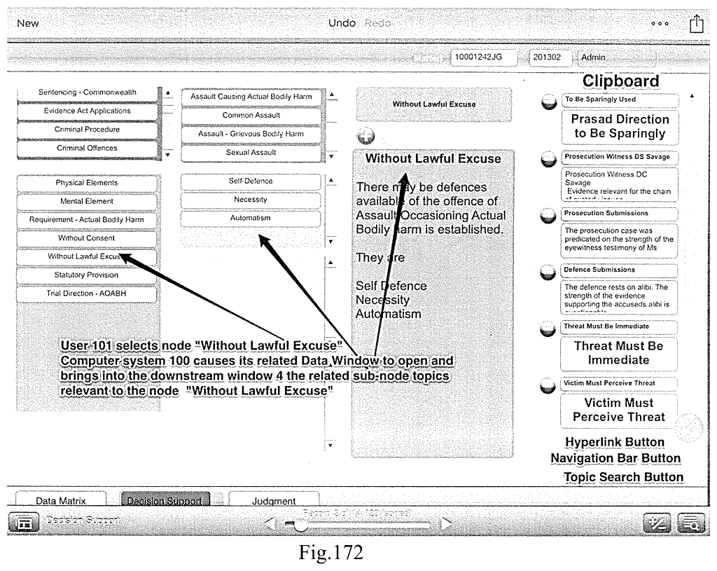

FIG. 172 illustrates the decision maker selecting the node “Without Lawful Excuse” from Window 1 of the visual display.

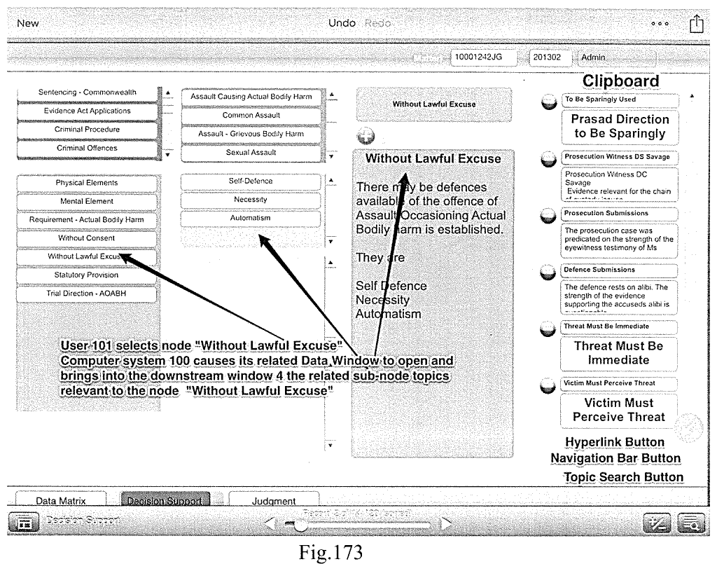

FIG. 173 illustrates the selection of the node “Without Lawful Excuse” causes computer system 100 to perform 2 functions.
1. Computer system 100 causes the Data Window relevant to the node “Without Lawful Excuse” to open in Column 3. This provides the decision maker with a summary of the defences which are available to the offence of Assault Occasioning Actual Bodily Harm.”
2. Computer system 100 causes the defences which are available to the decision maker to open in its related sub-node or downstream window. The defences so displayed are
   Self-Defence
   Necessity
   Automatism

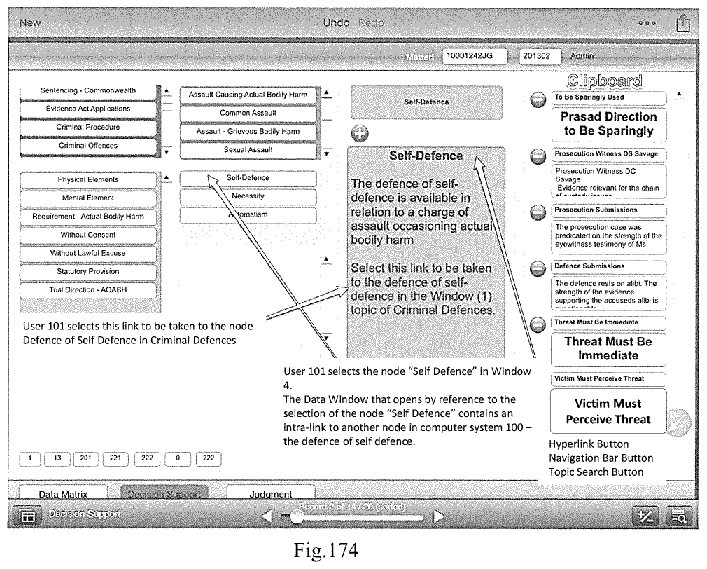

FIG. 174 illustrates the decision maker selecting the defence of self-defence, raised on the facts before the judge in the hearing.

The selection of the node “Self-Defence” causes computer system 100 to open the related Data Window to the node self-defence.

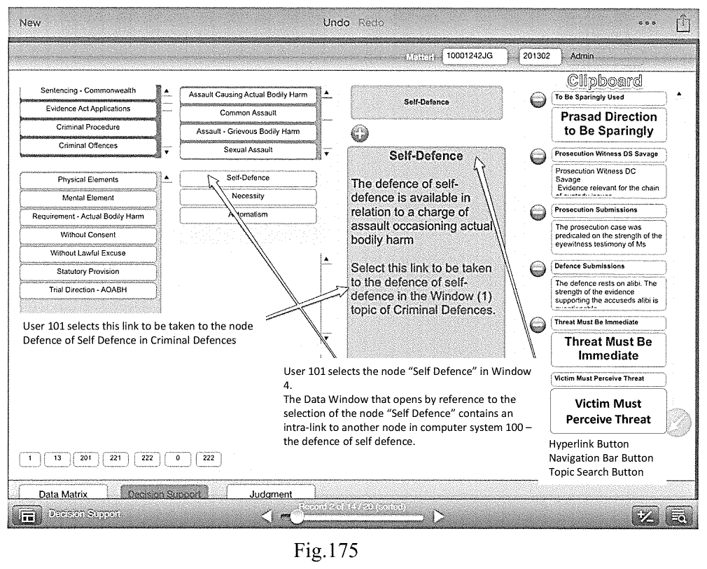

FIG. 175 illustrates that the Data Window informs the judicial decision maker that the defence of Self-Defence is available in relation to a charge of “Assault Occasioning Actual Bodily Harm” and contains a link which takes the decision maker to the defence of Self-Defence within computer system 100.

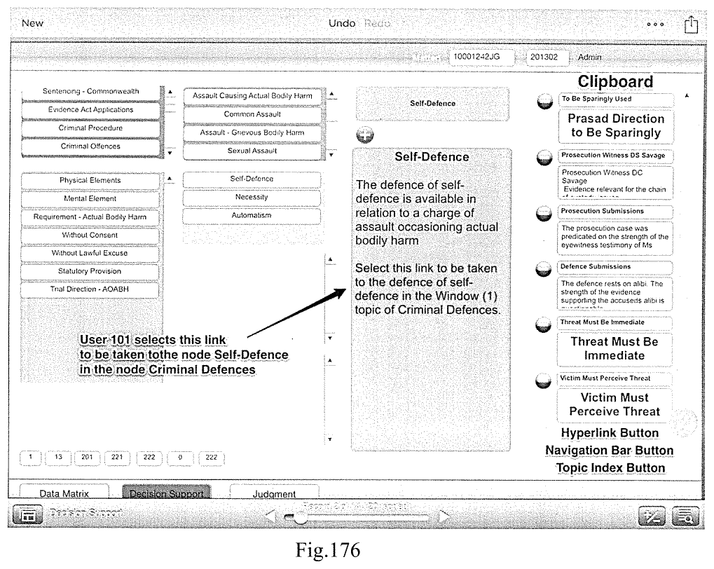

FIG. 176 illustrates the decision maker selecting the link in the Data Window Self-Defence in the decision chain relevant to Assault Occasioning Actual Bodily Harm.

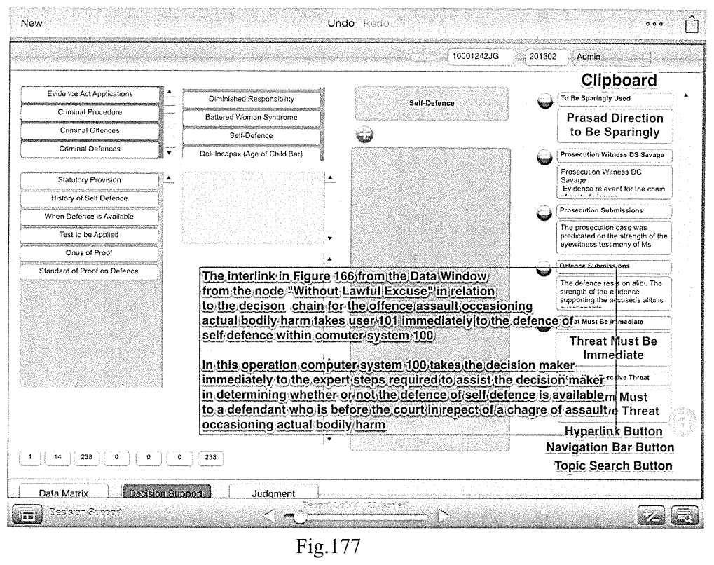

FIG. 177 illustrates computer system 100 taking the decision maker to the node Criminal Defences in Window 1, and its related sub-node “Self Defence” in Window 2, and opens the steps in the Decision Chain relevant to the defence of Self-Defence in Window 3.

In this way, computer system 100 takes the decision maker immediately to the expert steps required of a judge to determine whether or not the defence of self-defence is available to a defendant who is before the court in respect of a charge of Assault Occasioning Actual Bodily Harm.

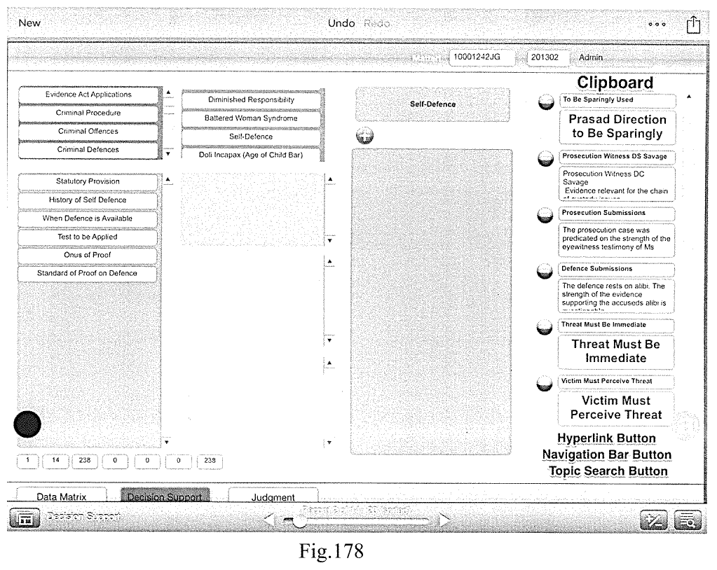

FIG. 178 illustrates the steps an expert has prepared to guide a user 101 in respect of how such a user approaches applying the law in respect of the defence of self defence.

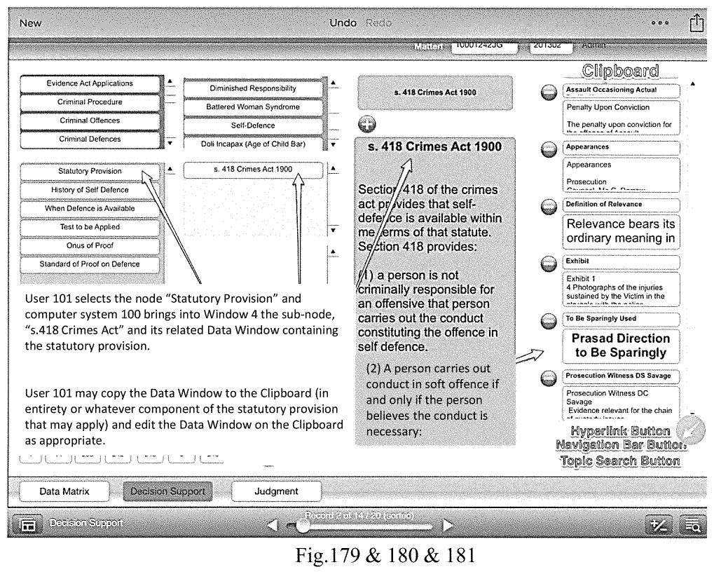

FIG. 179 illustrates the decision maker judge selecting the node Statutory Provision from Window 3.

FIG. 180 illustrates computer system 100 bringing into the visual display in Window 4 the related sub-node or downstream node “s. 418 Crimes Act 1900”.

FIG. 181 illustrates the decision maker selecting that node and illustrates computer system 100 bringing that node into the Data Window in Column 3, where it may be copied in whole (or parts selected and copied by the user) onto the Clipboard.

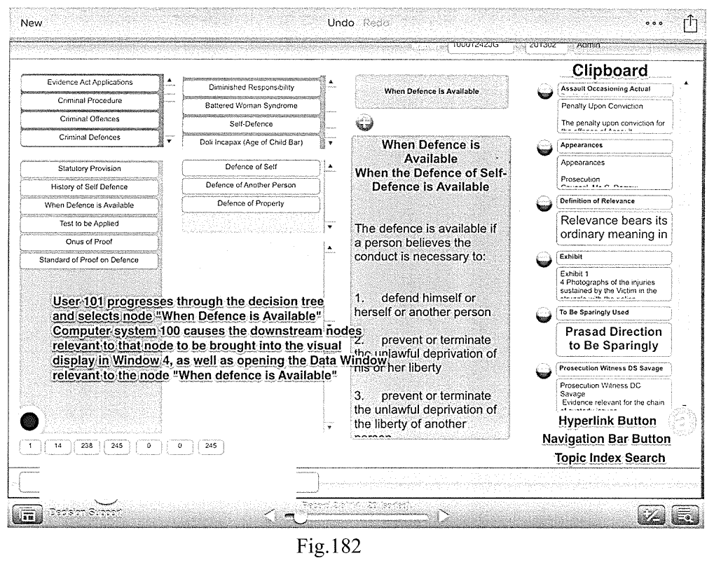

FIG. 182 illustrates the decision maker progressing though the decision steps in Window 1, selecting the next step, the Window 3 node, “When Defence is Available”.

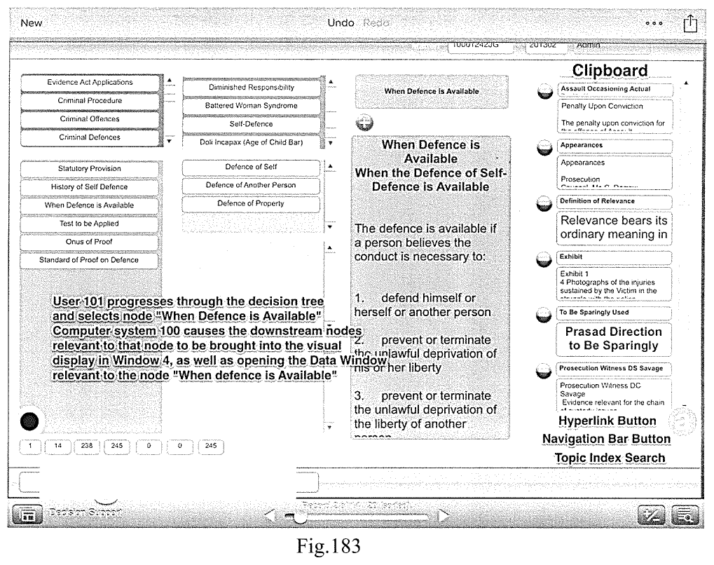

FIG. 183 illustrates the selection of the node “When Defence is Available” opens the Data Window relevant to that node, which provides a summary of the circumstances when the defence is available and also opens the sub-nodes or downstream nodes relevant to each of the defences available which, when in turn selected, will allow the decision maker to explore each of those nodes in increasing level of their detail and complexity in their further related downstream nodes as the particular problem confronting the decision maker judge may require.

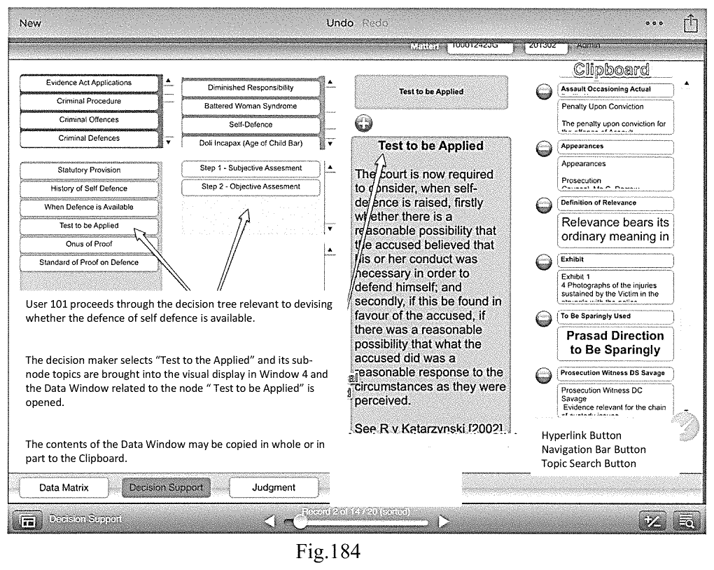

By way of further illustration, the decision maker, having been satisfied with the assistance provided by computer system 100 that the defence is available to the defendant, the decision maker returns to the decision steps contained in Window 3 and FIG. 184 illustrates the decision maker selecting the node “Test to be Applied”.

The selection of the node “Test to be Applied” causes the visual display to be updated by: 1. Opening the Data Window in Column 3 relevant to the node “Test to be Applied: and

2. Opening in Window 4 the sub-node or down stream nodes taking the decision maker more deeply into that topic.

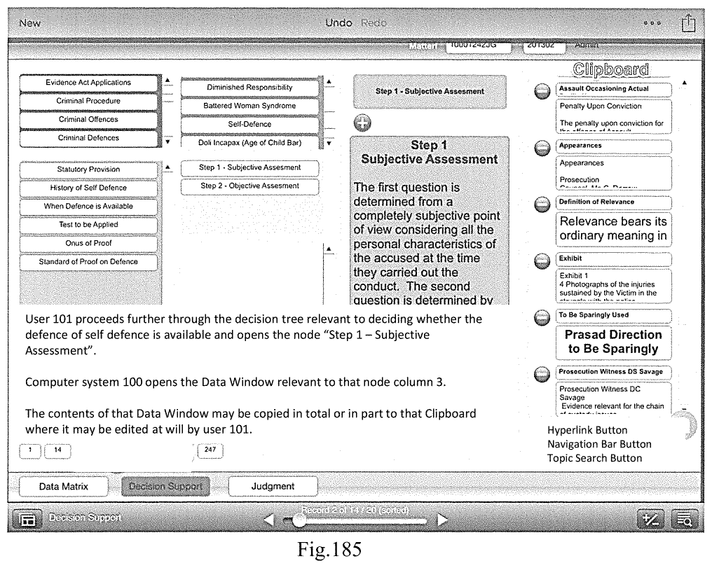

FIG. 185 provides a further example of the decision maker selecting the Window 4 node “Step 1—Subjective Assessment”. That causes computer system 100 to update the visual display and open the Data Window relevant to the node “Step 1—Subjective Assessment” to open in Column 3 of the computer screen.

The decision maker judge many copy the entire Data Window to the Clipboard using the Copy to Clipboard button or may open the Data Window and using the select and copy function copy part of the contents of that Data Window to the Clipboard. The Data Window headed “Step 1—Subjective Assessment” may be edited and data relevant to the matter proceeding before the decision maker may be added.

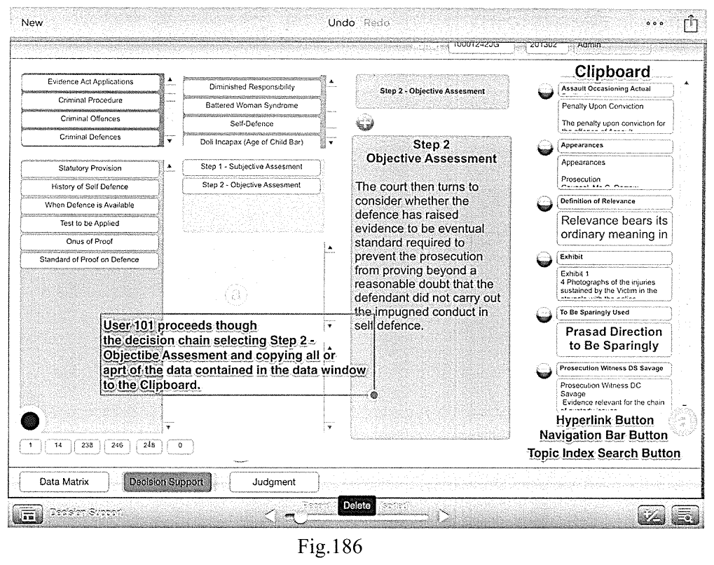

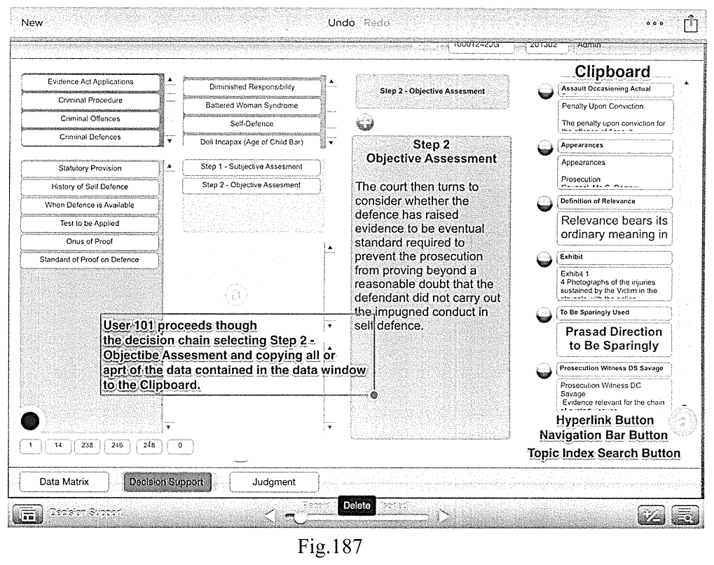

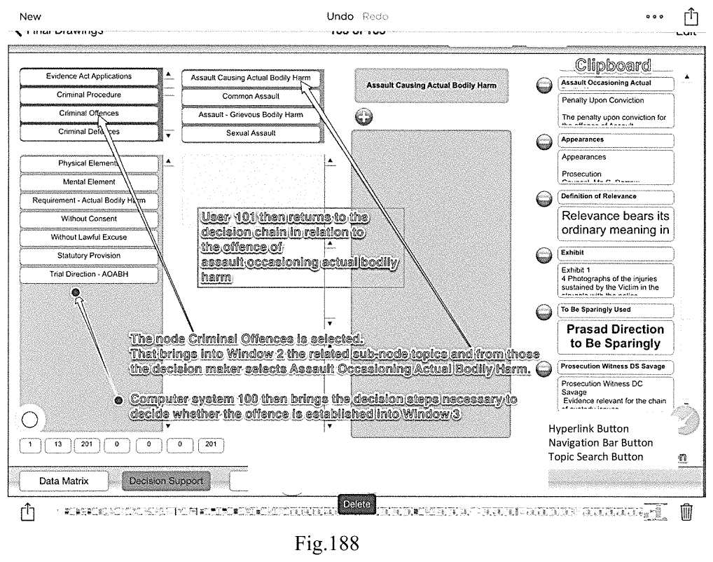

The decision maker may then proceed to the next step within that step in the decision chain, selecting the node “Step 2—Objective Assessment.” FIG. 186 illustrates how the selection of this node causes its related Data Window to open in Column 3. That Data Window may be copied in whole or in part to the Clipboard by the user in the same steps described in respect of FIG. 187. FIG. 188 illustrates the decision maker having copied the Data Window “Step 2—Objective Assessment” onto the Clipboard.

The decision maker may progress though the expert steps contained within computer system 100 in Window 3 of the visual display.

Every time a node is selected the computer screen is updated to display the decision steps relevant to that point in the decision tree. The decision maker may progress through the decision chain to the depth of Window “n” which as previously described in this Application computer system 100 may bring into the visual display of the depth of the decision tree used by user 101 so requires.

The decision maker may then return to the nodes relating to the elements of the offence before the decision maker. FIG. 188 illustrates the decision maker selecting from Window 1 the node “Criminal Offences”. Computer system 100 brings its related topics into Window 2.

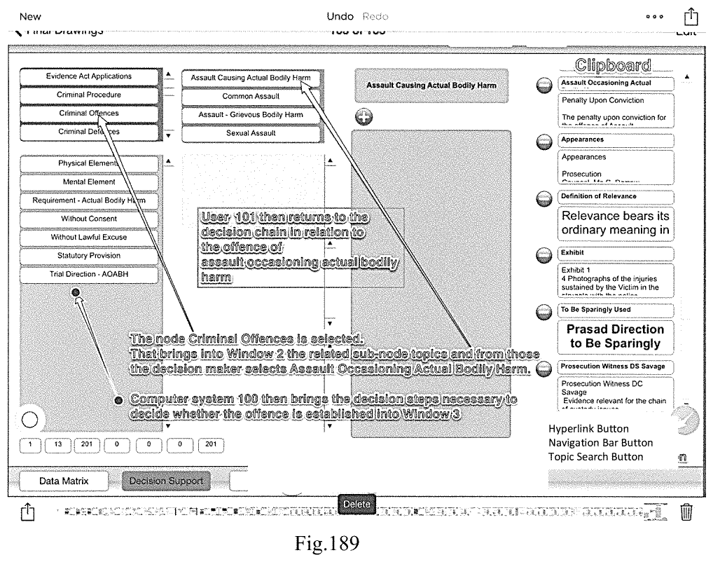

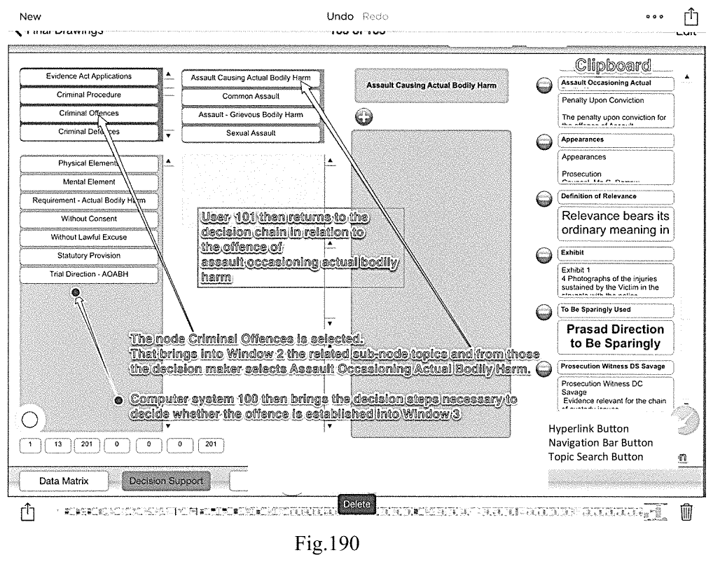

FIG. 189 illustrates the decision maker then selecting the node “Assault Occasioning Actual Bodily Harm” which causes computer system 100 to update the computer screen to bring into the visual display the nodes in Window 3, those being the expert steps in the decision chain relevant to that node.

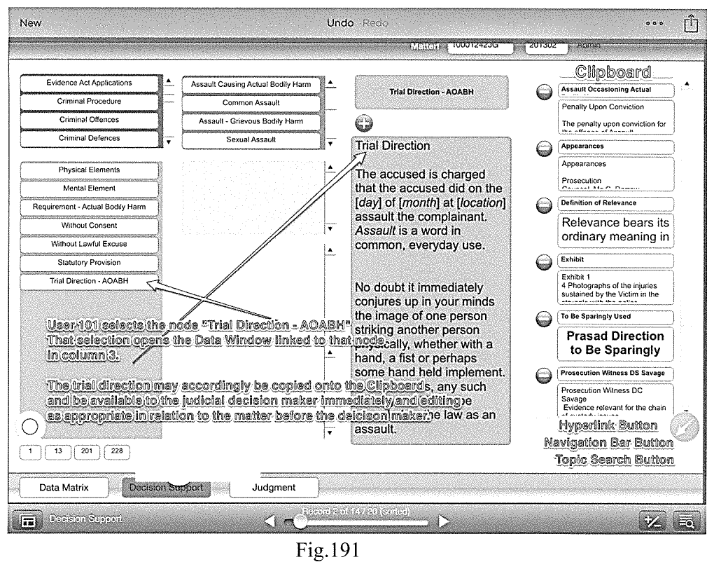

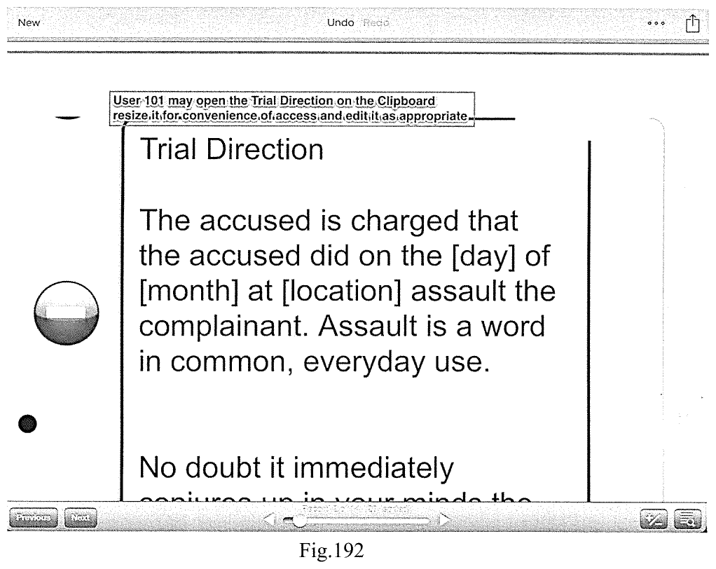

FIG. 191 illustrates the decision maker selecting the node “Trial Direction”. That causes computer system 100 to update the visual display by causing the Data Window related to Trial Direction—AOABH to appear in Column 3.

The Data Window Trial Direction—AOABH may be copied onto the Clipboard using the Add to Clipboard button by the decision maker. It may be opened and edited by the decision maker on the Clipboard.

Computer system 100 in the manner described in this example provides a decision system providing high level support for a judicial decision maker writing a judgment. It provides support for
1. the process the judicial officer needs to follow in the conduct of the hearing
2. expert decision support in respect of legal problems the judge will be required to decide during the course of the hearing; and
3. expert decision support for how the judge will be required to approach the application of the law—in one example provided, in respect of the elements of the offence assault occasioning actual bodily harm, and the potential availability of a defence of self defence thereto.

This decision support system will raise the quality of decision making across the body of professionals, who share common task functions, such as, in one example, judges, who are as a group applying the same law to the cases proceeding before them. Having high quality decision trees available in respect of the many different legal problems they apply on a day to day basis will mean there will be a significant increase in the quality of decision making across this professional field and an increase in the consistency and predictability of the application of the law—both highly desirable attributes in any legal system. Further, the increase in the quality of decision making will reduce the costs associated with variable and wring decision making across the legal system. It will generate a reduction in the number of appeals, and the very high cost to the public purse of providing the court resources to hear and determine such appeals as well as the very substantial costs to litigants associated with correcting judicial error arising from misapplication of the law—that is, poor quality decision making.

The system 100 provides a very high level of support for a decision maker in relation to the criteria to be applied to those specific steps necessary to comprehensively work their way through a legal problem.

As illustrated, the responses provided by the decision maker to the criteria, information, directions and questions within Decision Assistance mode will take the Decision Maker through divergent routes through the topics contained within the Data Matrix, depending upon the particular circumstances confronting the decision maker. They are supported throughout the decision process by the high quality expert decision tree contained in Decision Assistance mode. The system 100 responds to the input in respect of the specific item by directing the user 101 to a related downstream node and the computer screen 102 is updated in response.

The purpose of Decision Support is to allow a decision maker to be guided through the decision chain relevant to solving their problem by their responses to a serious of questions, information, directions, and criteria designed by the expert or experts who have designed the decision assistance module.

By way if illustration, in relation to a Decision Support topic in the illustrated Data Base, a Sentencing Module may have been prepared by a Supreme Court Judge who has had 30 years experience, and has an established jurisprudential reputation. This way, the historical patterns stored in Decision Assistance mode in the sentencing module between

the nodes within the network are made by that Supreme Court Judge. This decision assitance module may of course be collaboratively designed, wither by groups of judges, and academics, and senior practitioners.

There may well be designed a court specific or accredited Decision Support System using computer system 101 on specific topics, and industry standard decision support systems which insurance, due diligence and other prudential requirements require be used or complied with.

Further, the experience, knowledge and expertise of in one example a senior judicial officer (or expert panel) in respect of their approach to sentencing law may be available in Decision Assistance mode to any person, whether Judge, Magistrate, practicing lawyer, student or layperson, who has access to this decision support system. Computer system 100 may be designed with laypersons in mind, and the language and accessibility of the system designed accordingly. By way of one example, computer system 100 could easily be designed to provide laypersons with a resource to assist them if they had to go to court to be sentenced for an offence. At present, clients pay legal practitioners very substantial sums of money—to attend court on their behalf and make submissions in respect of pleas of guilty before sentence. There are in busy courts sometimes 5 minutes available for a plea to be made by a practitioner and there is insufficient time for the practitioner to make a quality plea which puts before the Magistrate all the matters relating to the offender which are legally relevant on the plea, and assuming the practitioner representing the offender has themselves a high sentencing decision chain—that is, is skilled and knowledgeable about this area of the law. Computer system 100 could readily be designed so take a user—a layperson who is to appear before the court for sentence—through the steps the Judge has to consider, and computer system 100 could invite such a user to enter the information the Judge must take into account at the various stages of the sentencing decision chain as illustrated in this patent application. The layperson user could print the report and hand that information to the judge at court when their matter proceeds to sentence. Such a report would greatly assist the judge—they would then have before them the information from the offender which addressed all the decision criteria the judge should consider in imposing sentence and these are in effect highly specialised written submissions relevant to sentence that the layperson has been assisted in providing without having to go though a legal practitioner. The very high costs of the provision of legal services is and should be a concern to the community. There are very substantial numbers of persons within the community who cannot afford to pay a solicitor and who may not qualify for legal assistance and they simply have to turn up at court and under the significant time constraints, and the fact that Judges cannot without the risk of compromising their judicial function, give an offender legal advice on how to best present their plea. Computer system 100 may well offer a very significant contribution to giving lay people access to the law and thereby to their rights. It is trite to say that if members of the community cannot afford to access their legal rights they do not in real terms have them—what they have is privileges which money can buy. Further, it may be used in one example as an adjunct to the provision of legal services. For example, the legal aid authorities may direct a user to complete the steps in a computer system 100 on sentencing so that the resultant report could be provided to the legal aid lawyer at court. That would save potentially hours of the lawyers time in taking instructions before court or at court and provide that lawyer with a comprehensive foundation for their submissions. Computer system 100 could also be made available in computer terminals at the Court House to enable users to complete their report for sentence at Court before their matter goes before the Judge.

It is clear that any government process or steps in any complex application could be made available to the community through computer system 100. There would be significant savings in relation to the provision of public servants, who currently staff counters to assist people in making complex applications, and the time public servants currently take in providing such assistance. That expert function would significantly be incorporated into decision system 100. The assertion is not made that computer system 100 would remove the need for some assistance by public servants but the assertion is made that it will significantly support the decision assistance functions those public servants currently provide and will result in significant cost savings to government.

The design of a decision system 100 as a laypersons guide to sentencing illustrates how computer system 100 will operate to democratise knowledge. It may be designed using any complex database of knowledge in the Decision Matrix and may be designed to me as sophisticated in its language and the level of specific instruction contained in Decision Assistance mode as the user base to which it is directed is appropriate.

By way of one example, an instruction and/or user manual may be provided for the operation of a motor vehicle. The Data Matrix would contain the detailed information in respect of the component parts of the vehicle (that large amount of data now provided in small print and detailed manuals) and instruction steps on how to operate the vehicle, or how to identify or diagnose the nature of problems with the operation of the vehicle, contained in Decision Support mode.

The Decision Support mode also has a Search Function in its permanent display. That function allows the user to enter a term or terms and computer system 100 then brings into the visual display a list of all the nodes within Decision Support mode which contain that term or terms. User 101 may then select the desired node and computer system 100 will take the user to that location within Decision Support.

Such a search function permits a user to go to any stage in a decision tree that may be of convenience to them in relation to the specific problem they are required to solve.

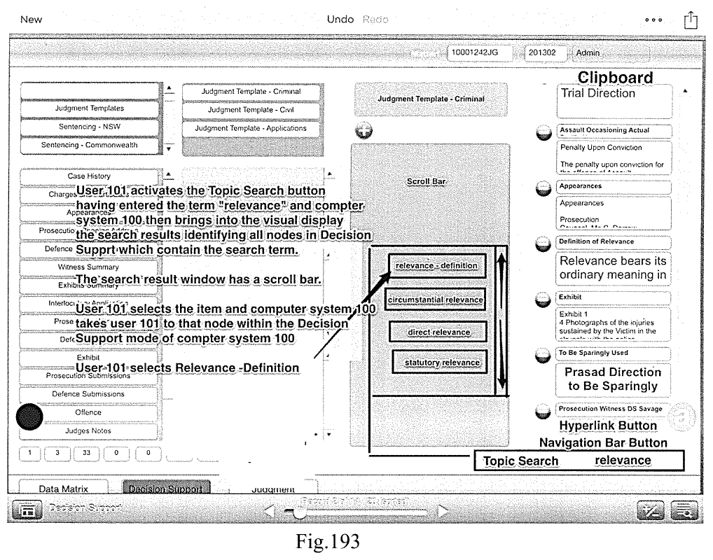

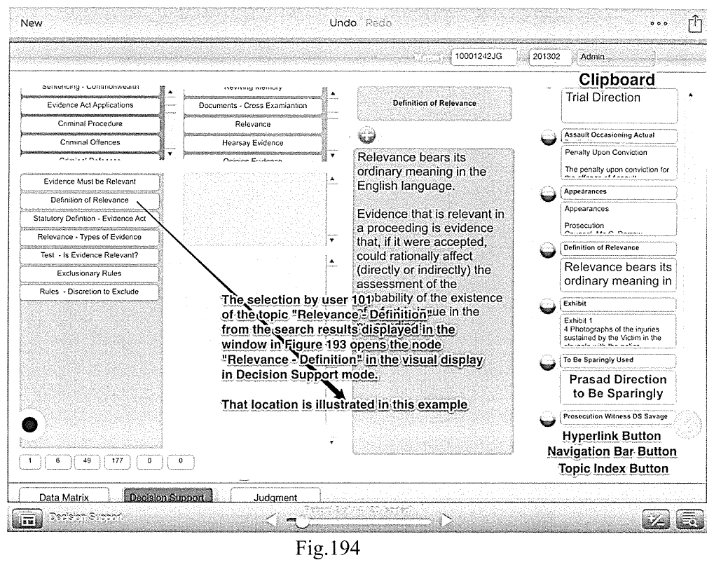

FIGS. 193-194 illustrate the operation of the search function in computer system 100.

Computer system 100 also has a navigation function. That operates whether user 101 is using computer system 100 in Data Matrix or Decision Assistance mode. The Navigation system records any steps taken whether in Data Matrix or Decision Assistance mode. The Navigation System, when activated by user 101, allows user 101, by sliding their finger backwards or forwards across the Navigation Bar (or by using any device to control input) to retrace the steps they have previously taken and to re-commence their steps though the decision tree in a different direction from some earlier node.

Computer system 100 makes an electronic record of the steps taken in the decision tree by the user to the point at which the Navigation bar is activated by user 101 and stores that decision chain. A new record commences from the point at which user 101 starts to take a different step through decision system 100. User 101 may retrieve and re-load any prior decision chain into computer system 100 at any time during their use of the decision system and the decision chains will be saved until deleted by user 101.

The Navigation function works in the same way whether the decision maker is in Data Matrix or Decision Assistance mode of computer system 100. When activated the Navigation system allows the user to retrace the steps they have taken though the decision tree and to recommence steps taking a different path from any previous step. Computer system 100 will save an electronic copy of any decision chain if the decision maker alters the steps they have taken and all decision chains will be available to the user until they have been deleted from computer system 100.

The Clipboard Function

The clipboard function is always present whether the computer system is operating in Data Matrix, Decision Assistance or Report (or Judgment) Writing modes.

In Data Matrix, Decision Assistance, or Judgment Writing modes, the Data Windows may be opened by the user and data edited or entered by user 101.

The Data Windows in Clipboard mode will contain the sequentially entered Data Windows entered by user 101 in Data Matrix or Decision Assistance mode.

All Data Windows will have a scroll bar so that the amount of data they contain are not limited to the size of the window in the visual display.

Each Data Window may be opened, resized and edited at any stage of the decision process.

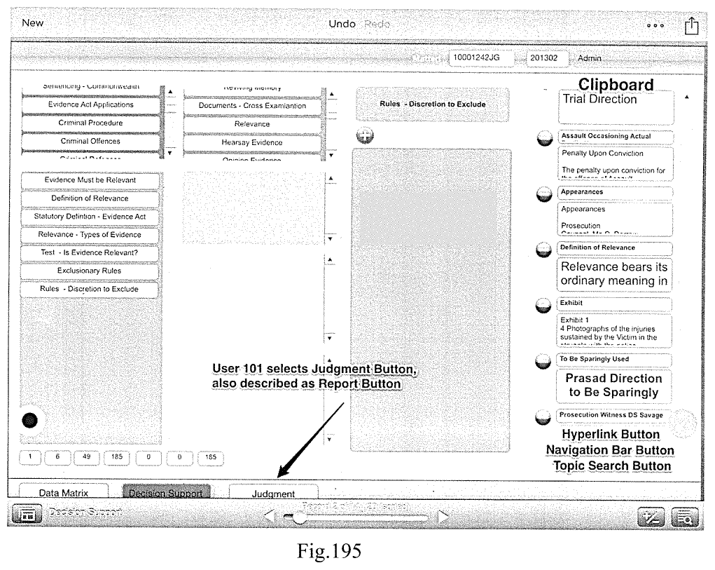

FIG. 195 illustrates a user opening computer system 100 in Report or Judgment mode by the user selecting Report (or Judgment) mode from the permanent display when in Data Matrix or Decision Support mode. User 101 may select the different modes of computer system 100 as may be convenient.

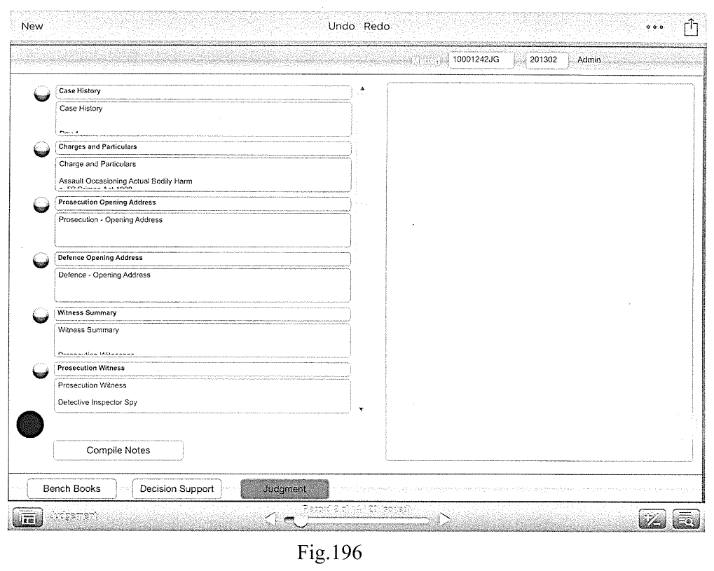

FIG. 196 illustrates computer system 100 in Report or Judgment mode.

Each Data Window may have different content contained within it, including but not limited to graphics, pictures, audio recordings, links to other Data Windows in the instant and other linked computer system 100 decision systems and hyperlinks to resources on the world wide web. The user may chose to open a hyperlink within a window within computer system 100, from which data may be selected, copied and pasted into a Data Window on the clipboard, or the user may elect to use the hyperlink to go to the site or resource on the wide wide web to which the hyperlink relates.

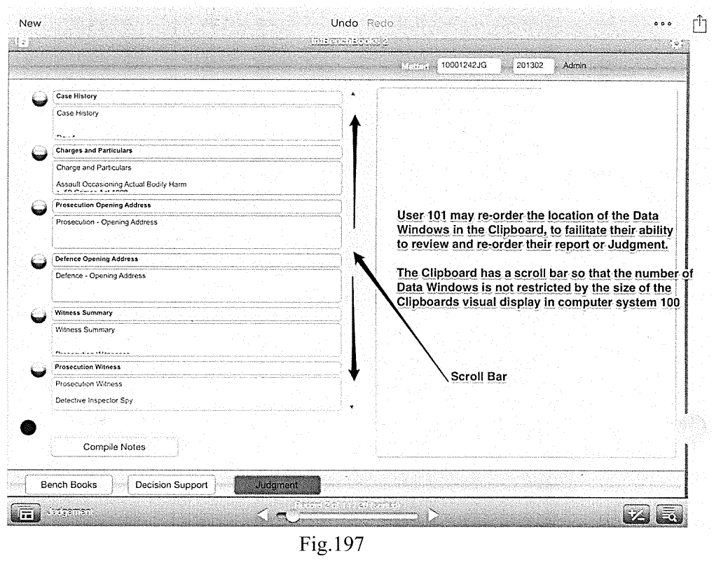

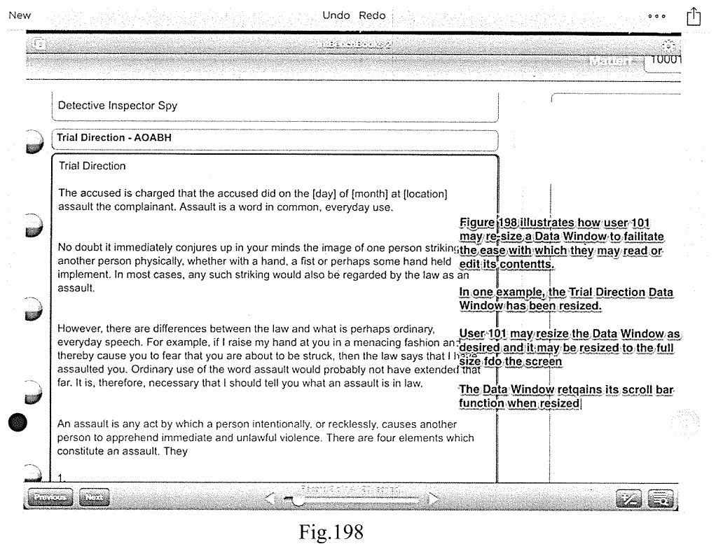

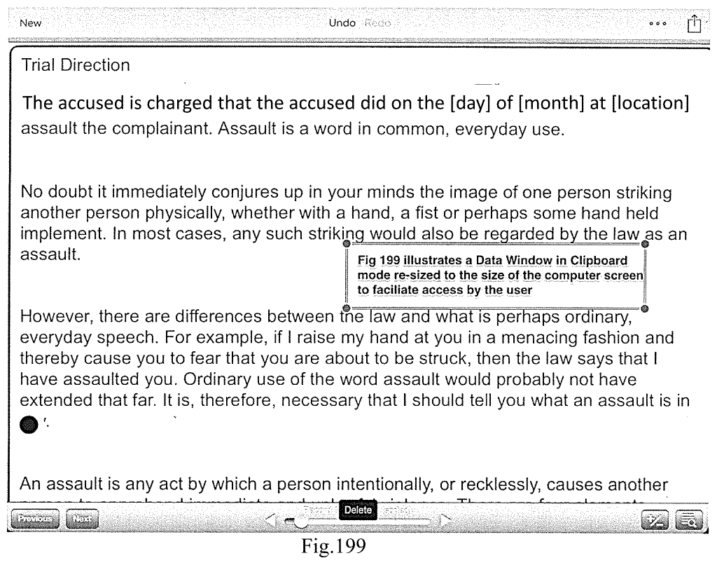

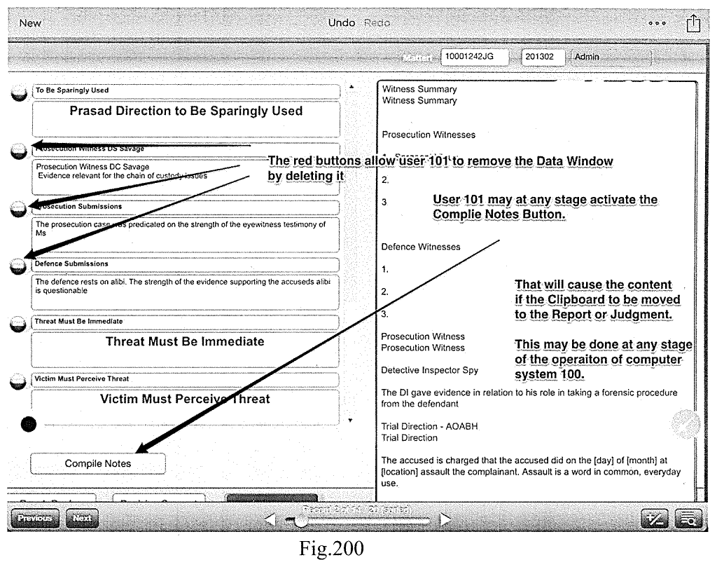

User 101 may re-order the location of the Data Windows on the Clipboard in Report mode. That will facilitate the preparation of the final report (or judgment). See FIG. 197. The Clipboard has the same content as it does when used by user 101 in Data

Matrix and Decision Support modes and any changes made by user 101 to the Clipboard in Report or Judgment mode will remain with the Clipboard if user 101 returns to either Data Matrix or Decision Support modes.

The Clipboard has a scroll bar so the number of Data Boxes it contains is not limited by the size of its visual display or screen display.

User 101 may at any stage in the use of computer system 100 activate the “Compile Notes” button in Report (or Judgment) mode. Computer system 100 will automatically copy the contents of the Clipboard into the Report or Judgment Window.

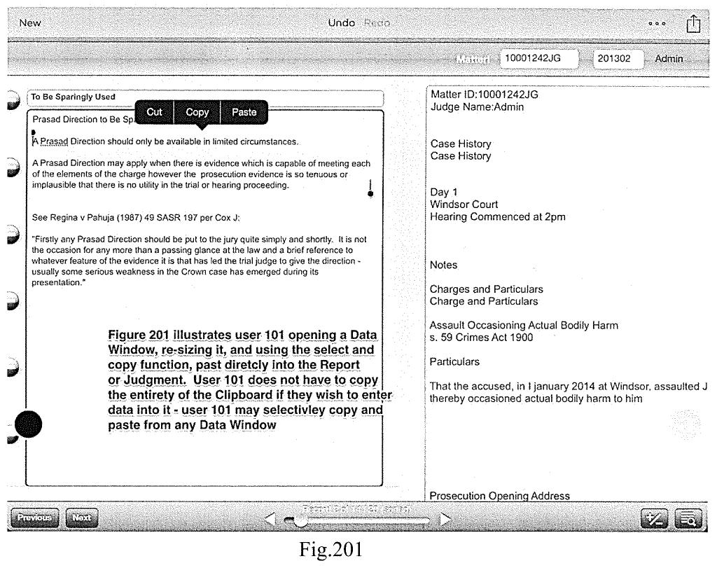

FIG. 201 illustrate user 101 may also open any Data Window on the Clipboard and select and copy the contents (or part thereof) into the report or Judgment using the select, copy and paste function.

User 101 may at any stage during the operation of computer system 100 open the Report or Judgment in Report (or Judgment mode) and edit its content.

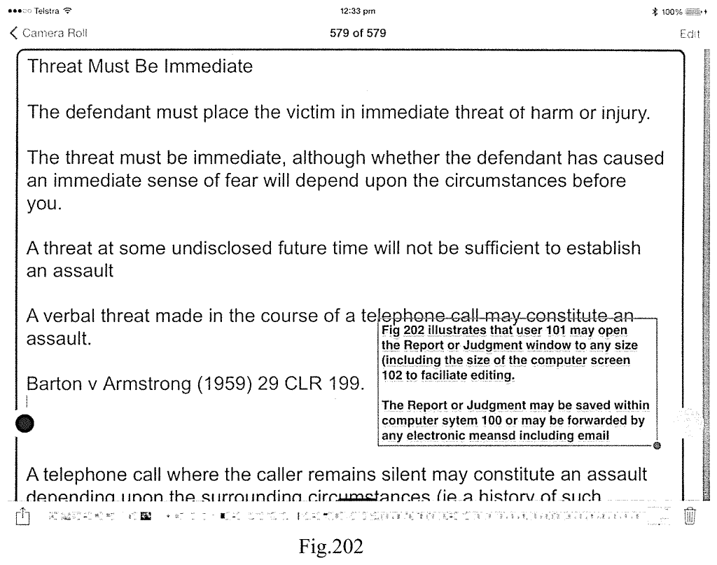

FIG. 202 illustrates user 101 opening and resizing the Report or Judgment Window. The Report or Judgment Window may be resized to the size selected by user 101 and it may be resized to fit the full size of computer screen 102.

The Report (or Judgment) Window has a scroll bar and the content of the window is not restricted by the size of the visual display.

FIG. 201 illustrates user 101 using the scroll bar facility to scroll though the Report (or Judgment).

User 101 may at any time export the content of the judgment or report by email (or any form of electronic transmission).

### Detailed Description of Third Embodiment

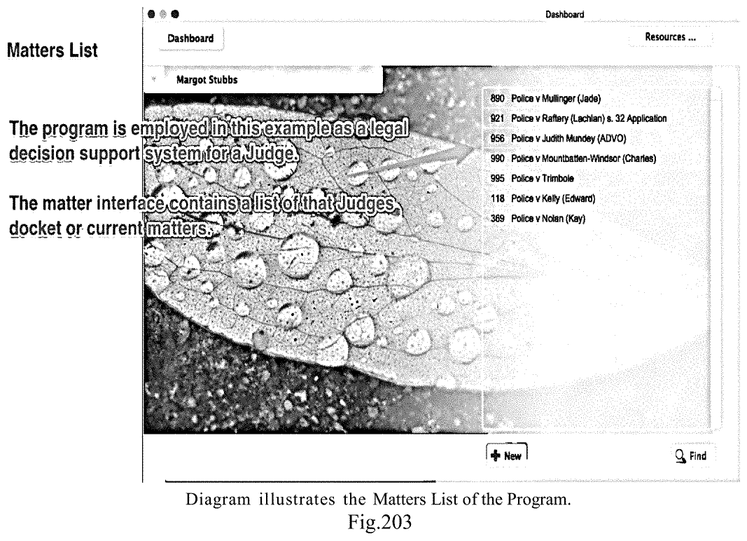

With reference to FIGS. 203-257 there are illustrated screenshots of a third embodiment of the control system operating as a decision support system for a matter.

### Definitions

The following terms have the following meanings in the description which follow:

Algorithm

The term algorithm is used to describe a process or set of rules to be applied in a complex problem-solving operation whereby a User is required to perform a complex decision making task on a particular topic which requires specialist knowledge and experience to generate an outcome which involves plural and complex steps. It does not embrace a process which operates independently of the User and cannot generate a decision outcome without iteration with the User. It is not intended to convey an automated equation.

Clipboard

A window comprising 1 or plural data windows which may be generated by the system and in which the User may describe by reference to the tags contained in the system or allocate such description as the User may deem appropriate.

Data Matrix

An organised data base comprising linked data modules on a specific topic organised in a linked hierarchy of increasing complexity which the User may access at any stage of the operation of the decision support system and which may contain relevant data from any source including plural resources captured and stored by the user to assist them in their decision making function, an example being conference papers delivered and collected from conferences attended etc and any plural resources compiled or collected by the User in relation to their decision functions as a judge and including plural types of resources such as hyperlinks, video presentations, and other multi-media resources

Matter

A Matter is a proceeding where a User is required to make complex decisions across the progress of a proceeding which is designed to produce an ultimate decision result in the form of a or Report (in the In-Use illustration a Judgment) wherein the Matter requires the User's application of specialist sets of rules at different points during the progress of the matter as well as a decision outcome based on the Users application of the said correct rules during the process and in the preparation of the Report.

In the legal example of a court case proceeding in a Hearing or Trial, the specialist rules are complex, and may traverse may different types of rules, such as the law of evidence, the law of practice and procedure, the law in relation to a very wide range of causes of action, such as Negligence Law, Contract Law, Fraud Law, Misrepresentation Law, Conveyancing Law, Family Law, each f which has many different subsets of laws the user may be required to apply. In relation to criminal offences, the Law of Assault, which may embrace many different subsets of this category, such as common assault, assault of occasioning actual bodily harm, assault occasioning grievous bodily harm, indecent assault, sexual assault, assaults in company, as well robbery, dishonestly obtain Property by deception, property damage, damage by fire etc.

A Matter in a legal context of a Hearing or Trial requires a Judge to perform 2 general functions, firstly to review, consider, compare, contrast, evaluate and interrelate items of evidence in the Hearing to come up with findings of fact in respect of the issues they are required to address in the decision algorithm. The User/Judge must then apply the facts to the relevant legal principles ensuring all of the applicable law is identified, and applied correctly to the facts, and this is the process resulting in the ultimate decision out come of a Matter being the Judges generation of their Judgment which contains their conclusions about which of the parties will succeed in the Matter. It is noted that there are also post-Judgment decisions which to be made involving application of identified and accurate legal principles such as may arise in respect of applications the parties may make in relation to Costs etc.

Third Party Inputs

Any item of data collected in a data window during the course of a matter by the user comprising items of evidence from witnesses, documents tendered, legal submissions made by legal practitioners during the Matter, and collected into data windows by the user and further including resources/items extracted from the data matrix and decision support algorithms.

Broadly, the third embodiment relates to a control system in the form of a decision support system for a matter; the matter of indeterminate duration; and during the course of the matter third-party inputs are received consecutively and periodically for the duration of the matter; The decision support system allowing replication of an expert's approach to a complex problem; said system comprising:

a primary clipboard into which data is entered by the user into one or more data windows;

multiple sub-clipboards which are generated from the primary clipboard and wherein

all sub-clipboards contain plural data windows;

a database of public information comprising data modules for selective input into a Data Matrix relevant to the matter;

preconfigured specialist algorithms assembled by an expert knowledgeable in decision-making in relation to the matter; the specialist algorithms contained in a Decision Support mode of the decision support system;

an input system by which the third-party inputs comprising each item of data presented to the User as a matter progresses are consecutively and periodically assembled by a user at first instance on to the primary clipboard;

an input system by which the user selects portions of the public information and portions of the preconfigured specialist algorithms and assembles them at first instance onto the primary clipboard;

and wherein the selections and assembly made in response to all items of data received during the matter are entered onto the primary clipboard;

thereby to assemble a primary clipboard of public information portions, specialist algorithm portions and third-party input portions in a continuous format so as to present a chronological record of all items of data and decisions made by the user as the matter progresses with the assistance of the specialist algorithms and data modules relevant to the matter and as selected by the user as the matter progresses.

In preferred forms the items of the data on the primary clipboard are

tagged by the user with tags during the course of the matter or by the User when the user is preparing their decision or report.

In preferred forms the tag is a categorising device by which an item of data is characterised according to a criterion selected by the user from the Decision Support System.

In preferred forms Every item of data entered into the Primary Clipboard is assembled by the User as the matter progresses.

In preferred forms every item of data able to be edited by way of both imported and user generated multiple tags to enable the content of the Primary Clipboard to be filtered into the Sub-Clipboards in accordance with the tags selected by the User.

In preferred forms the User generates a specialist display of the system in which the User may populate a Report (for example a judgement in a legal matter).

In a preferred form the display presents the selected decision support algorithm, as well as a sub-clipboard into which the User may select any sub clipboard containing the fact findings the User has made which are relevant to the selected decision support algorithm; and which display allows the user to assemble their Report (in the In-Use example a Judgment) by copying relevant data windows (or data from data window) from the decision support algorithm into the Report (or Judgment) Clipboard and then to populate that data window in the Report or Judgment Clipboard with data from the sub-clipboard in the adjacent display of the program containing the Users findings of fact.

In a preferred form the display further allows the User to so work sequentially though the topics from the expert decision algorithm on which the User must make an outcome decision.

In a preferred form the system thereby generating a Report (or Judgment) as the ultimate outcome of the process of applying the expert decision topics to the facts across the matter.

In a preferred form being those facts identified as relevant to every decision in the decision algorithm through the assistance provided in other modes of operation of the system.

Preferably the decision support system further incorporates further Plural integrated tagging functionality.

Preferably the tagging functionality includes colour tagging data windows.

Preferably the tagging functionality linking data windows to documents and other media saved into the decision system and generation of a chronology of all items of significance in the matter.

Preferably an item of data may be tagged with more than one tag thereby categorising the item of data according to more than one criterion.

In a preferred form all items of data which are tagged by a tag according to a particular criterion may be assembled by the system, on command of the user, into a sub clipboard or independent data window in a compilation mode of the system's operation.

Preferably the system extracts from the field of data collected by the user onto the Primary Clipboard during the Matter into Sub-Clipboards containing all data windows related to the Users selected tagging criteria.

Preferably the system allows a user to select one or more sub clipboards in a particular visual display and from those sources open and compare multiple data windows to view them at the same time.

Preferably then each independent data window displays links to related documents which a user may also bring into that specialist display; items of data tagged according to a different criterion so that the user is enabled to select one or more sub clipboards in a particular way to view more than one independent data window from multiple Sub Clipboards at the same time.

In preferred forms each independent data window displays items of data tagged according to a different criterion in each independent data window.

Preferably the matter relates to a legal investigation in the form of a court case.

In an alternative preferred form the matter relates to an investigation process relating to a medical investigation.

In an alternative preferred form the matter relates to a scientific investigation.

In an alternative preferred form the matter relates to an insurance investigation.

In a preferred form a user may invoke steps in the specialist algorithm in order from less complex to more complex-only to the level needed for the particular matter.

In a preferred form the criterion for a tag may be determined by the user or automatically imported, as part of a specialist decision algorithm which is imported into the decision system by the user.

In a preferred form Tags may be imported by the user.

In a preferred form the system comprises the primary clipboard which allows all data relevant to the matter from plural third-party sources from the system in data matrix and decision support mode to be input into the primary clipboard through a system of data windows which permits a user a powerful system to manage, link, and compare all of the data on the primary clipboard in any configuration selected by the user utilising the tagging function in each data window.

In a preferred form the system allows an expert decision algorithm to contain tags which will be automatically incorporated into the tagging functionality of the system which relates to the plural factual matters the user has to decide to correctly apply the law and wherein the user will also be able to add their own plural tags as they may consider appropriate.

In a preferred form the system has colour functionality for each data window.

In a preferred form the user may attach a colour to each data window to operate as a reminder to the user when reviewing the items in the primary clipboard in accordance with a user selected criteria by reference to the colours.

In a preferred form the data window in the selection of the chronology button permits the user to incorporate the event in the data window into a timeline which will be generated during the conduct of the matter.

In a preferred form the chronology will be available to the user in every mode of the system.

In a preferred form there are at least 6 visual interfaces/modes of operation comprising Matters, Data Matrix, Decision Support, Compilation, Judgment and Chronology

in a preferred form the specialist algorithms are assembled by an expert knowledgeable in decision-making in relation to the matter contained in Decision Support mode of the Program.

In a preferred form the system is modular and permits the user to generate and export from the system their own data matrix modules and their own decision support modules to be available for incorporation by other users of the system into their respective decision support algorithm lists or their data matrix, of public information.

FIG. 203: Illustrates the Matters List of the Program. Example Screen displays the matters which a User/Judge has outstanding/part heard/or waiting the completion of Judgment on their current docket.

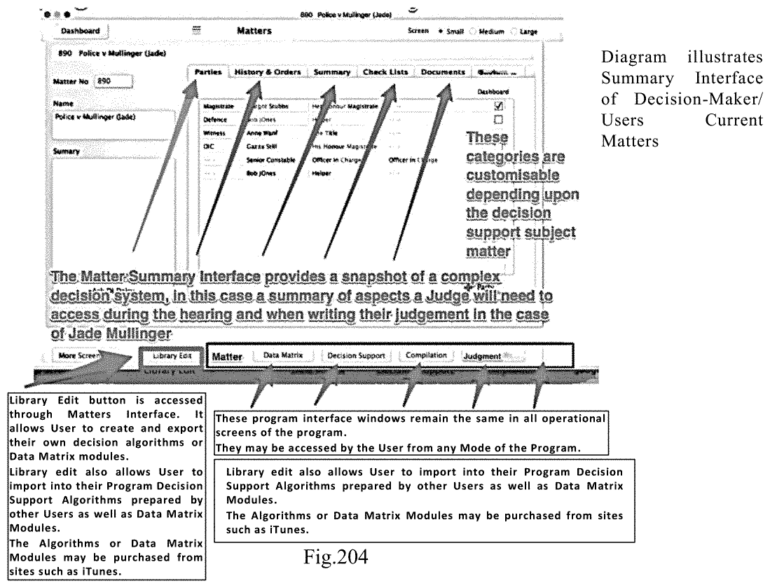

FIG. 204: Illustrates a Summary interface of Decision-Maker/Users key matters occurring in the progress of a Matter to which the User/Judge may wish to have immediate access to during the progress of the Matter.

Example: a complex trial or hearing may have multiple parties (including 2nd and 4rd cross-defendants etc), many Interlocutory Orders (decisions a Judge must make during the progress of a matter which can impact upon other decisions a Judge may have to make later in the hearing or trial), summaries of key occurrences in the hearing or trial, Check Lists of things that have to be done as the Hearing or Trial progresses, and a document management system which allows documents to be incorporated into other aspects of the Users management of the content of the Data Windows in other interfaces within the Program.

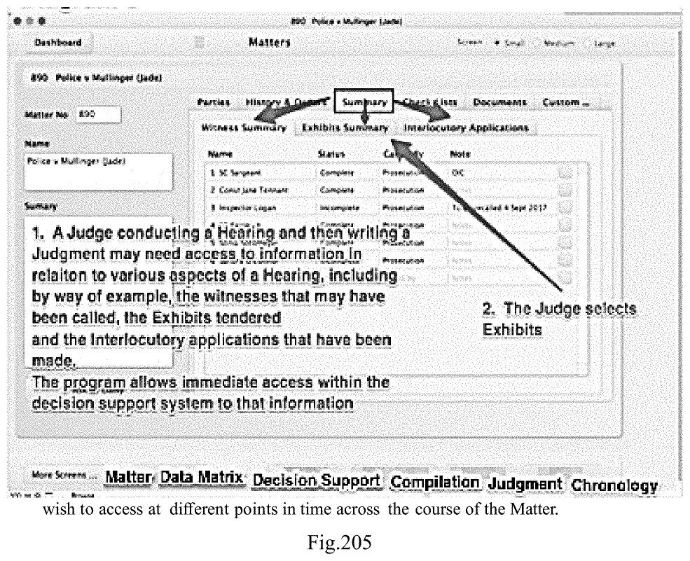

FIG. 205: Illustrates the plural summary aspects of a Matter that the Program allows the User/Judge to access in the Matters Summary Interface at different points in the progress of the Matter (Hearing or Trial) or when User is writing their Judgment which may be selected and brought into the visual display in Matters mode of the Program.

Example: It allows the Judge/User to immediately see a list of the Witness who have been called, to access a Summary of information relevant to their evidence, all the Exhibits tendered, and any Interlocutory Orders or Decisions they have made across the progress of the Matter.

It is noted that these are all matters to which a User/Judge frequently has to refer to during the course of a Matter, both during the course of a Hearing or Trial or when the User/Judge is writing their Judgment.

The User/Judge has access to 6 modes of the operation of the program in all visual displays of the Programs operation, being Matter, Data Matrix, Decision Support, Compilation, Judgment and Chronology.

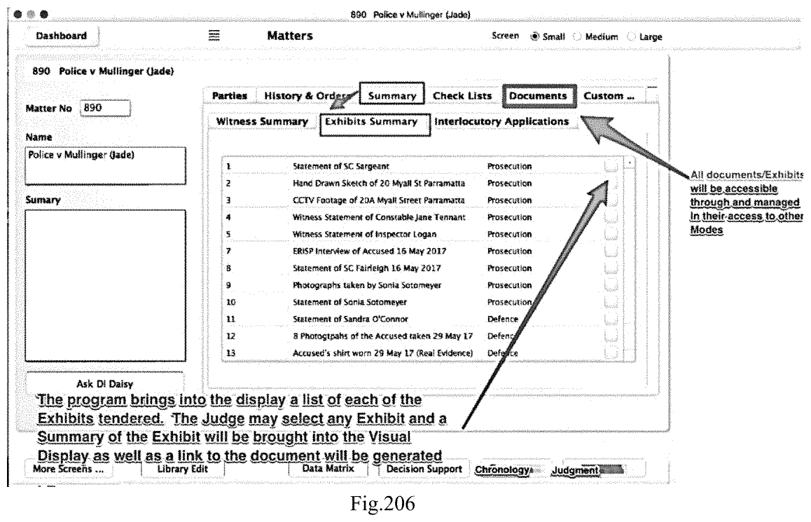

FIG. 206: Illustrates the Summaries of Key items a User/Judge may have ready access in the Matters Interface of the Program. The User/Judge may enter any document or multi-media document into the Program in the Documents interface of the Matters display of the Program and this will allow any documents or media (ie video footage etc) to be linked to any Data Window in other modes of the operation of the Program.

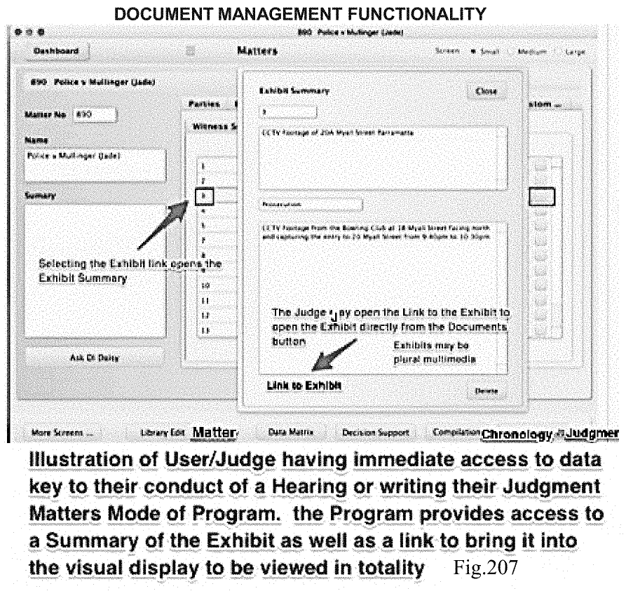

FIG. 207: Illustrates User/Judge having immediate access to many documents to which constant referral is likely to occur across both the conduct of the matter as well as when the User/Judge is writing their Judgment.

Example: Program allows Judge to immediately access a Summary of all Exhibits tendered as well as to like the Summary to the Exhibit itself, and allows the User/Judge to bring the Exhibit directly into the visual display via the link to the Exhibit contained in the Summary Window.

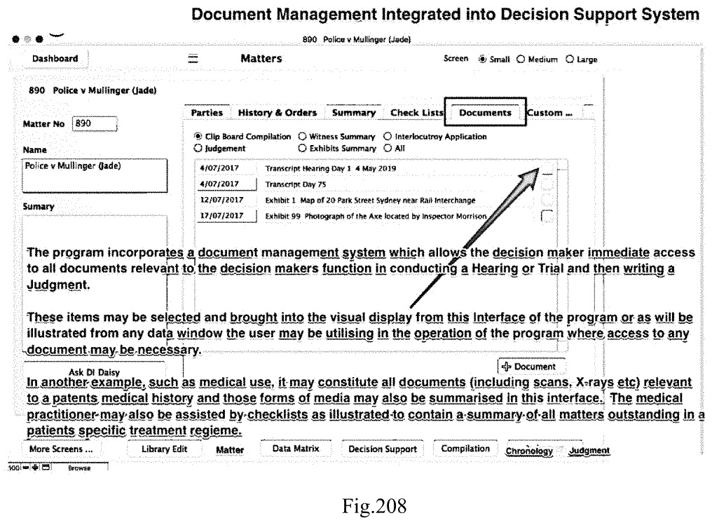

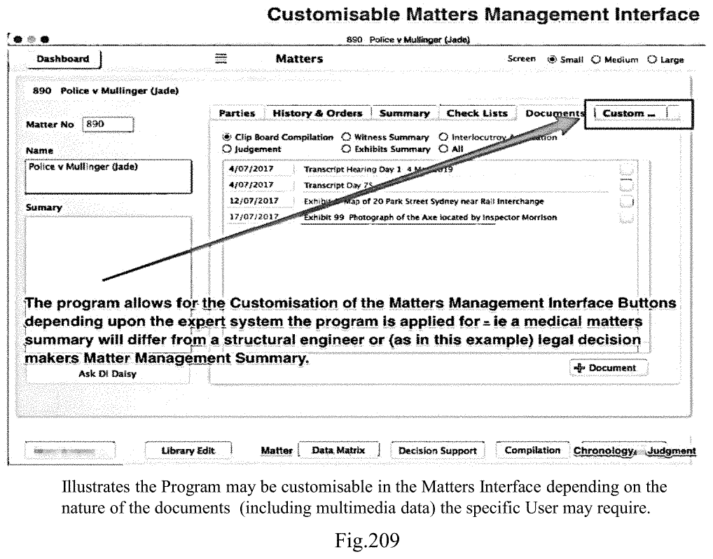

FIG. 208: Illustrates the Programs incorporation of all documents (including multi-media documents) relevant to the Matter into the Program through the Matters Interface and which will allow all documents to be linked to the evidence of any party in any data window or to any other item of Evidence in Tagging and Compilation mode.

The Program contains a Check List function to enable a record to be kept of items the User/Judge may have to do during the course of the conduct of a Matter.

FIG. 209: Illustrates the Program is customizable in respect of the type of Summary Matters the specific User of the Program may require.

Example: a medical user may have Summary items recording consultations, a Summary of all specialist referrals, a Check List, etc.

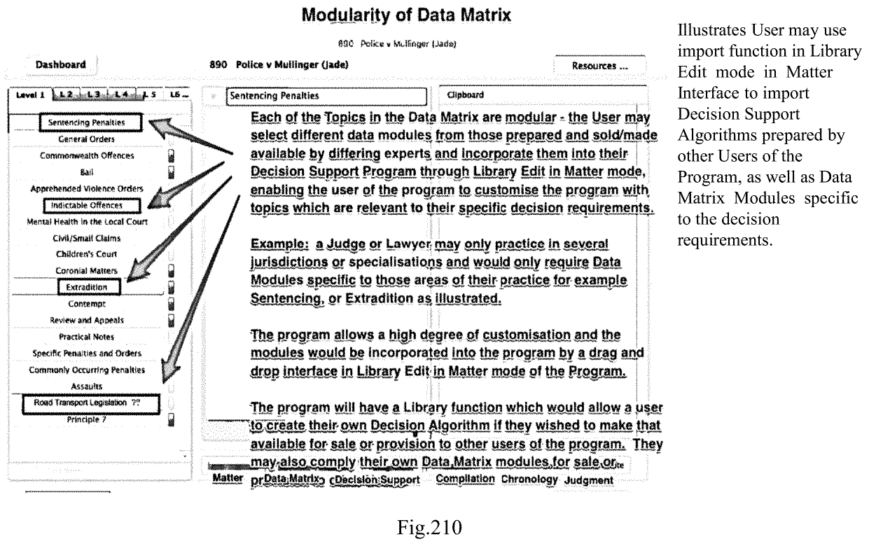

FIG. 210: Illustrates User may use Import function in Library Edit mode in Matter Interface to import Decision Support Algorithms prepared by other Users of the Program, as well as Data Matrix Modules specific to the Users decision requirements. which have been prepared by other Program Users.

The Library Edit function operates in relation to Data Modules prepared in Data Matrix as well as in Decision Support.

The Program also allows the User to generate their own Decision Support (and Data Matrix modules) which they can use Library Edit to export to be made available to other Program Users.

These Algorithms and Data Matrix modules are able to be downloaded from 3rd party sites (such as iTunes etc) making expert decision support modules widely available.

The Program also makes available individual modules generated by the Program available operate alone as a Run Time.

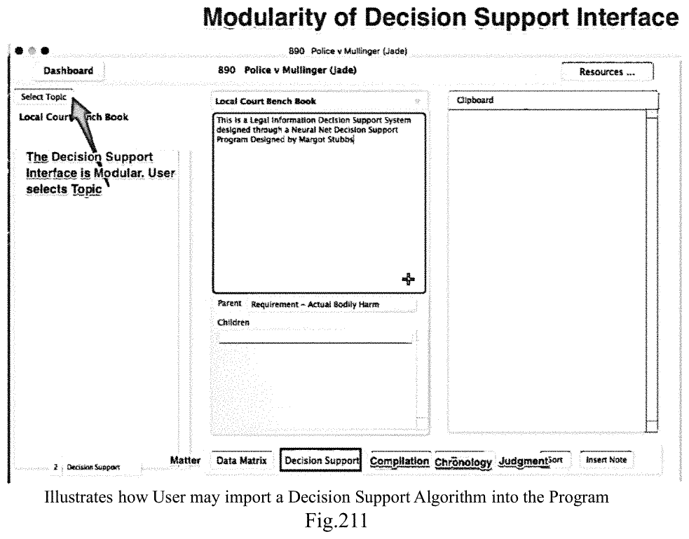

FIG. 211: Illustrates how User may import a Decision Support Algorithm into the Program

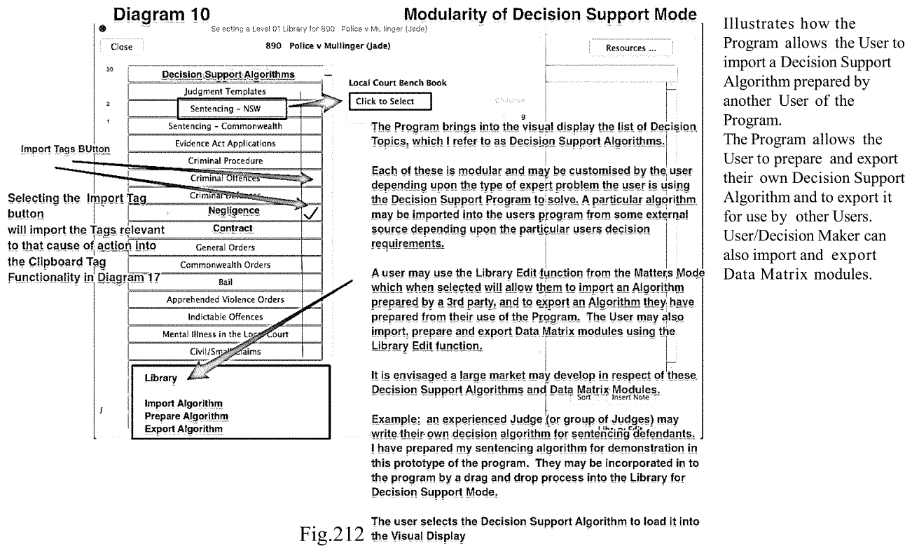

FIG. 212: Illustrates how the Program allows the User to import a Decision Support Algorithm prepared by another User of the Program.

The Program allows the User to prepare and export their own Decision Support Algorithm and to export it for use by other Users.

The same functionality may be used to import modules into the Data Matrix mode prepared by other Users and for the User to prepare and export their won Data Matrix modules.

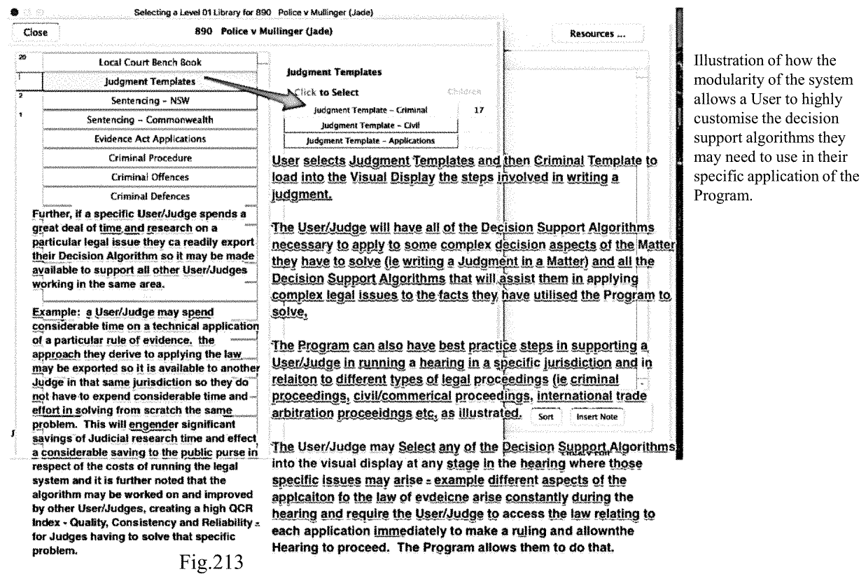

FIG. 213: Illustrates how the Modularity of the Decision Algorithms and Data Matrix modules allows a User to highly customise and specialise their Program to their specific decision making needs. The Program will provide a system which will significantly increase the may in which expert system support algorithms are able to be generated and made available to persons engaged in making decisions in respect of specific types of Matters (ie legal decision makers, medical decision makers, engineers, etc).

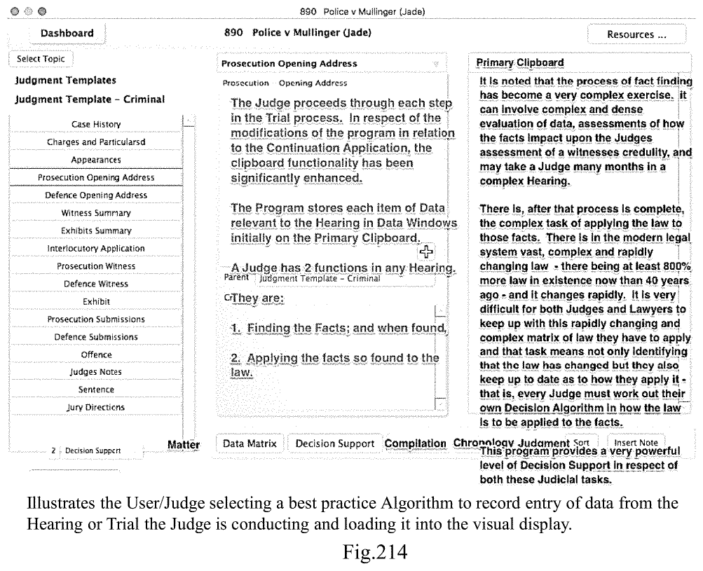

FIG. 214: Illustrates the User/Judge selecting a best practice Algorithm to record entry of items of data from the Hearing or Trial the Judge is conducting and loading it into the visual display in Decision Support mode. It is noted that a User/Judge may have different data entry algorithm depending upon the type of Hearing they may be conducting as illustrated in the previous diagram—Criminal Hearing, Civil Hearing, Sentencing Hearing, Coronial Enquiry etc.

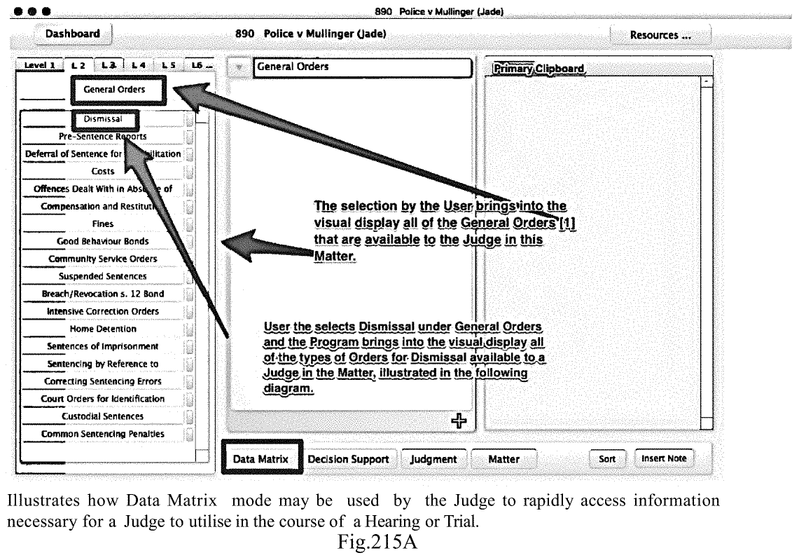

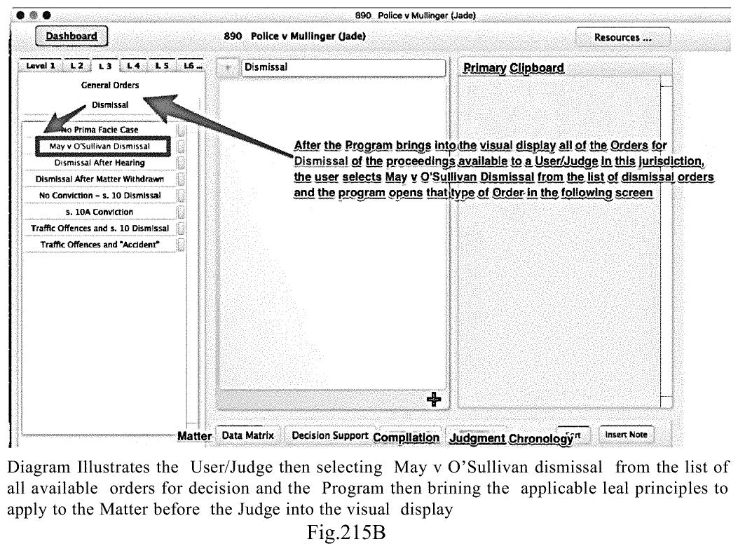

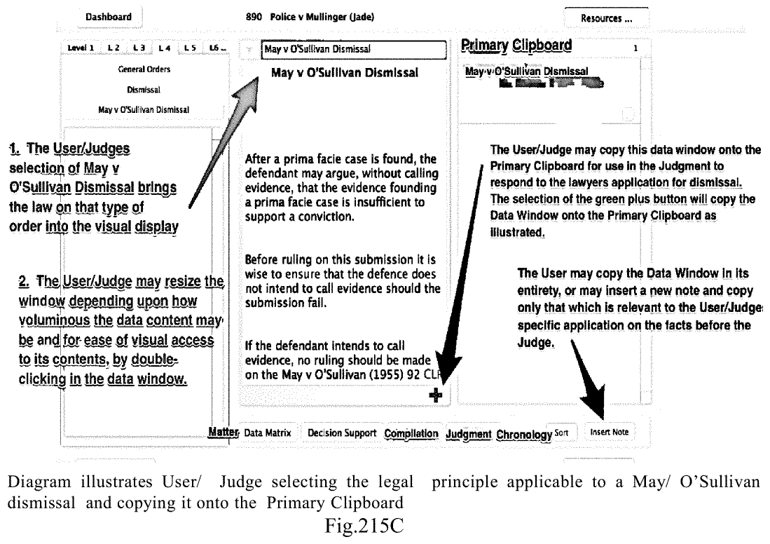

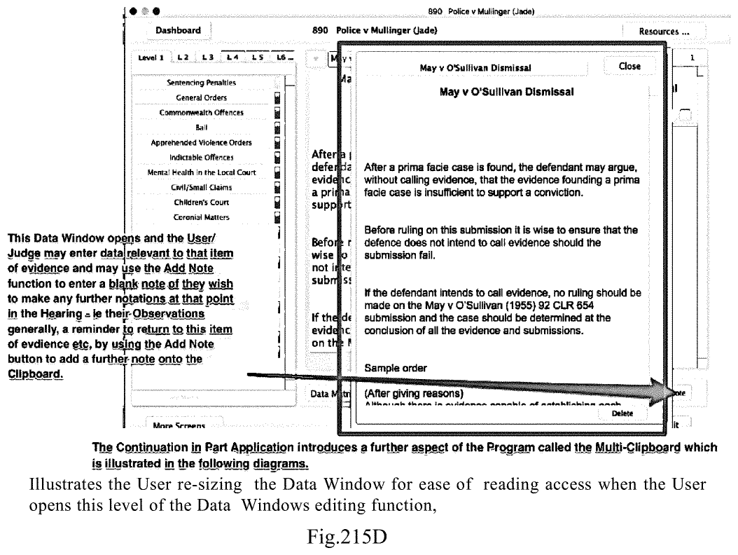

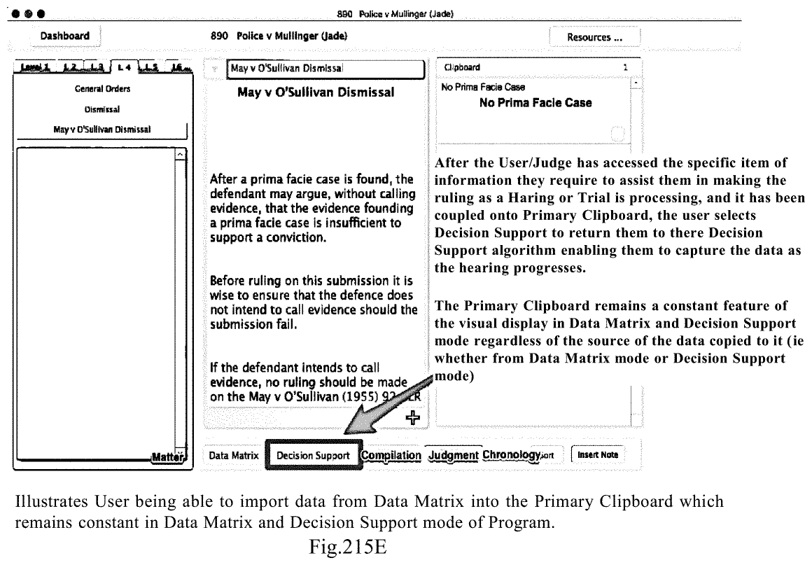

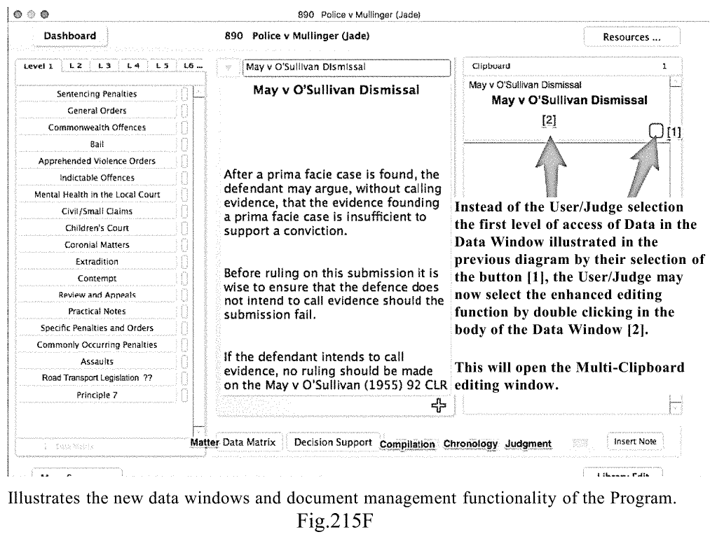

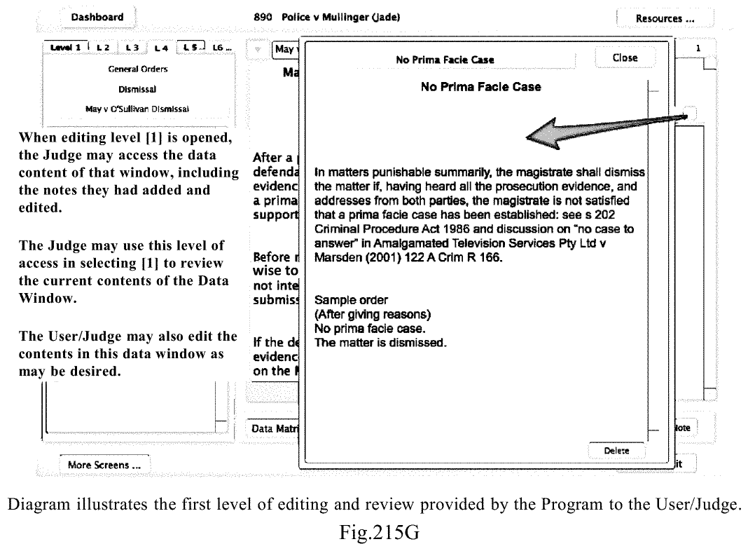

FIG. 215A: Illustrates how Data Matrix mode may be used by the Judge to rapidly access information necessary for a Judge to utilise in the course of a Hearing or Trial

Example: during a Trial a lawyer may make an application for a Dismissal Order in accordance with the legal rule May v O'Sullivan. The User switches from Decision Support mode to Data Matrix mode, selects General orders. The Program brings into the visual display all the Dismissal Orders available to the User, and from that list, the User selects Dismissal Orders, and then May v O'Sullivan Dismissal Orders.

FIG. 215B: Illustrates the User/Judge then selecting May v O'Sullivan dismissal from the list of all available orders for decision and the Program then brining the applicable leal principles to apply to the Matter before the Judge into the visual display

FIG. 215C: Illustrates User/Judge selecting the legal principle applicable to a May/O'Sullivan dismissal from this Data Window and copies it onto the Primary Clipboard.

FIG. 215D: Illustrates the Program allowing the User to resize any window for ease of visual access depending on the volume of Data contained within the window. tTe User may rescale windows to any proportion of screen size.

FIG. 215E: Illustrates User/Judge returning to Decision Support mode after having extracted the data relevant to a May v O'Sullivan Dismissal from the Data Matrix mode and after copying it to the Primary Clipboard.

That data window containing the May v O'Sullivan Dismissal Order remains on the Primary Clipboard and the User then selects Decision Support mode to be returned to the previous Decision Support algorithm the User had been applying before going to Data Matrix mode to collect the information required to be used by them in making a decision (whether the Matter should be dismissed on the legal application of May v O'Sullivan) and the User then proceeds through the Matter collecting data and making other decisions with the support of the decision algorithms as issues rearing expert determination arise.

FIG. 215F: Illustrates the new data windows and document management functionality of the program.

There are 2 editing windows available to a User/Judge that may be accessed from any Data Window.
[1] will open a window to display the text content of the window. This was in the initiating application.
[2] will open an enhanced document management window which allows the user powerful control over correlating, linking, editing, and managing all of the data collected in the Primary Clipboard so that data may be extracted on any criteria the User/Judge may select to significantly enhance the ability to organise, manage, compare, contrast and ultimately make their findings of fact.

FIG. 215G: Illustrates the first level of editing and review provided by the Program to the User/Judge.

Any data window on the Primary Clipboard may be opened and edited by the User/Judge at any mode of the operation of the This will be used when they are reviewing the content of the Data Window and they may edit its content in this level of the display should they wish to do so.

The following diagram illustrates the editing and data windows management functionality of the Continuation in Part.

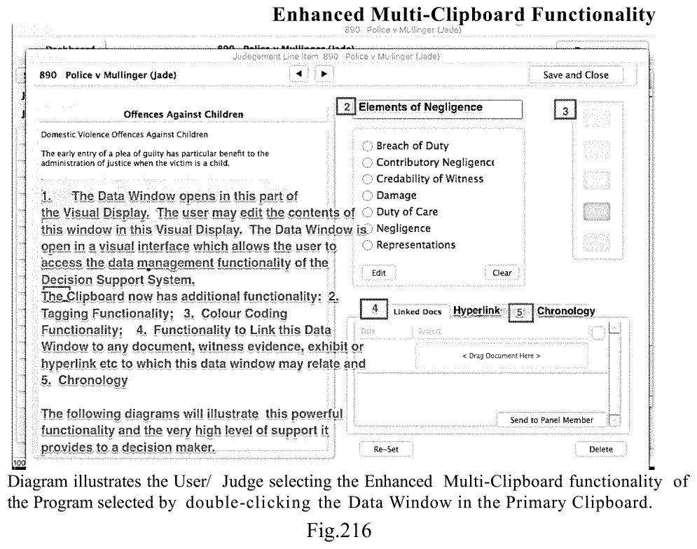

FIG. 216: Illustrates the User/Judge selecting the Enhanced Multi-Clipboard functionality of the Program, which is selected by double-clicking in the Data Window in the Primary Clipboard. this editing function is also available from any Data Window in any mode off operation of the Program.

This window contains the editing functionality for the Data Window open to the User in selection mode [1] and also contains powerful tools for managing the data to assist the User/Judge in their fact-finding activities when selection mode [2] is utilised.

The User/Judge may return to this mode of editing the Data Window at any stage in the utilisation of the program, wither when the evidence is unfolding before them during the Matter (Hearing or Trial), after court has finished and when reviewed the evidence and when preparing and writing their Judgment.

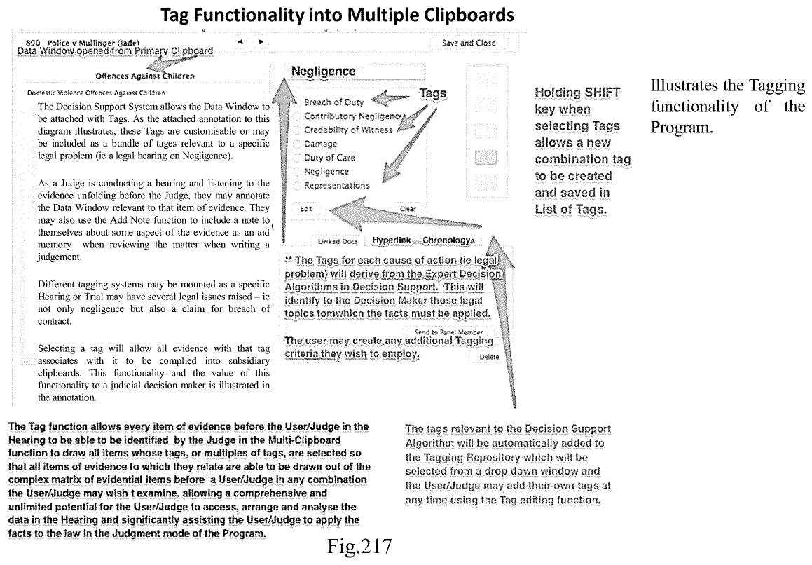

FIG. 217: Illustrates the Tagging functionality of the Program.

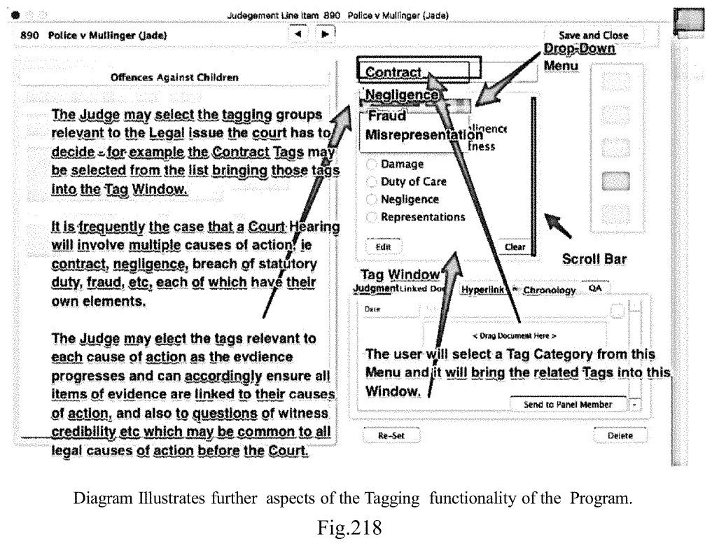

FIG. 218: Illustrates further aspects of the Tagging functionality of the Program. Tag groups relevant to the criteria which a User/Judge has to make findings of fact may be selected. Example selecting Tag Group Negligence will bring into the display the tags (inter alia) Duty of Care, Breach of Duty, Damage and Causation.

The Judge may select as many tags as they may require for each Data Window and the User/Judge may select a multi-Tag by holding SHIFT key when making their selection. a Malt-Tag containing each of the said tags will be created, attached to the Data Window, and saved in the Tag List and new tags may be created by the User at any time during the operation of the Program.

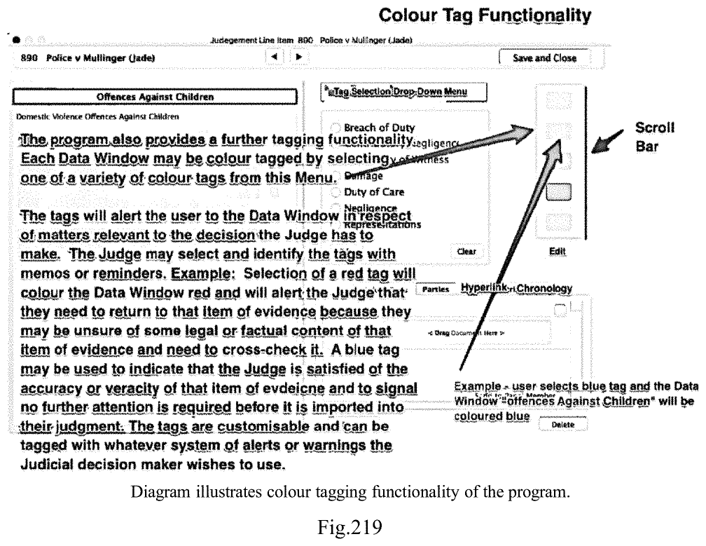

FIG. 219: Illustrates colour tagging functionality of the program. The User/Judge may select a colour as the background colour for that Data Window.

The tags are customisable to the User/Judges selection and may indicate by way of example selecting Red indicates to the User/Judge that they have a concern about this item of evidence. A Blue Tag may signify the Judge accepts that item of evidence and it is ready to be imported into the Judgment without further need for review.

The colour coding of a Data Window assists the Judge intuit it minimises the risk that the problem or issue in the Data Window that needs further attention will not be overlooked.

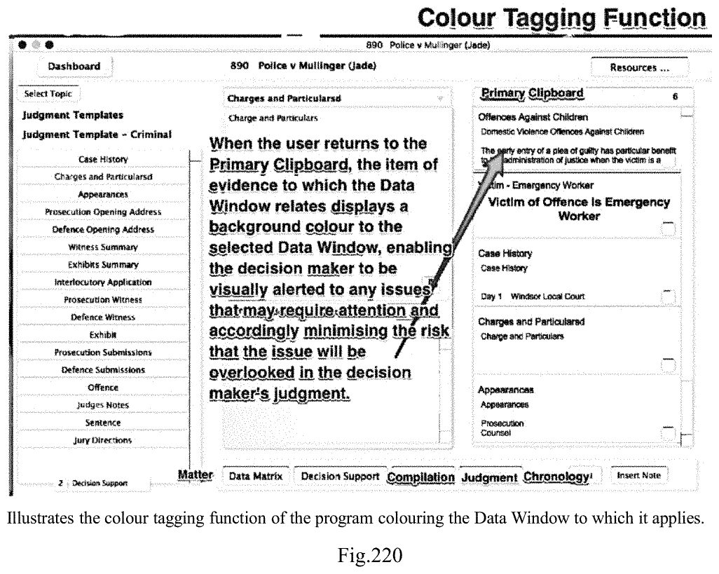

FIG. 220: Illustrates the appearance of the colour tagging function in a data window to which it is applied.

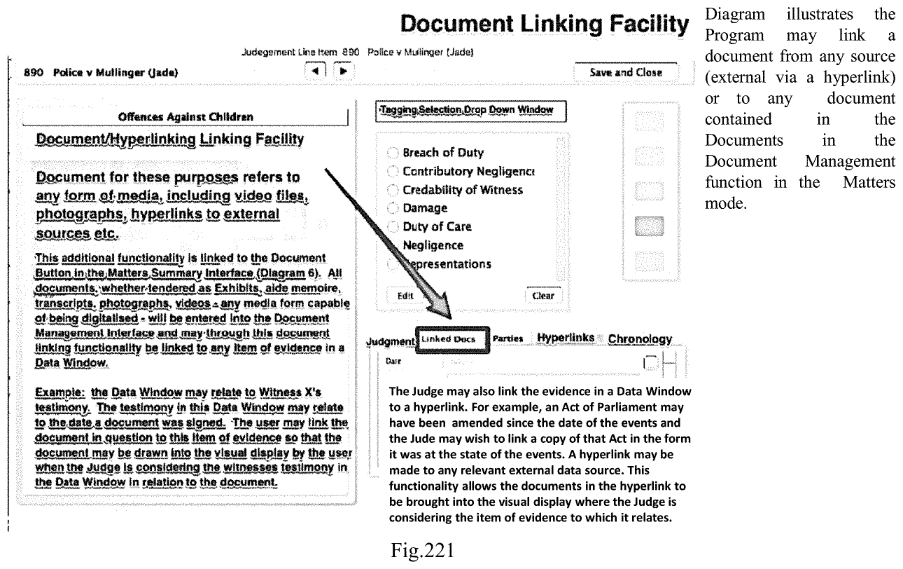

FIG. 221: Illustrates the Program allows the User/Judge to link the content of the Data Window to any external data source via hyperlink, or to any document (including multi-media etc) contained in the Documents section of the Matters interface. Multiple items may be linked.

Once linked, the User/Judge may open into a window in the visual display any linked document when reviewing any evidence in any Data Window which relates to that document or item.

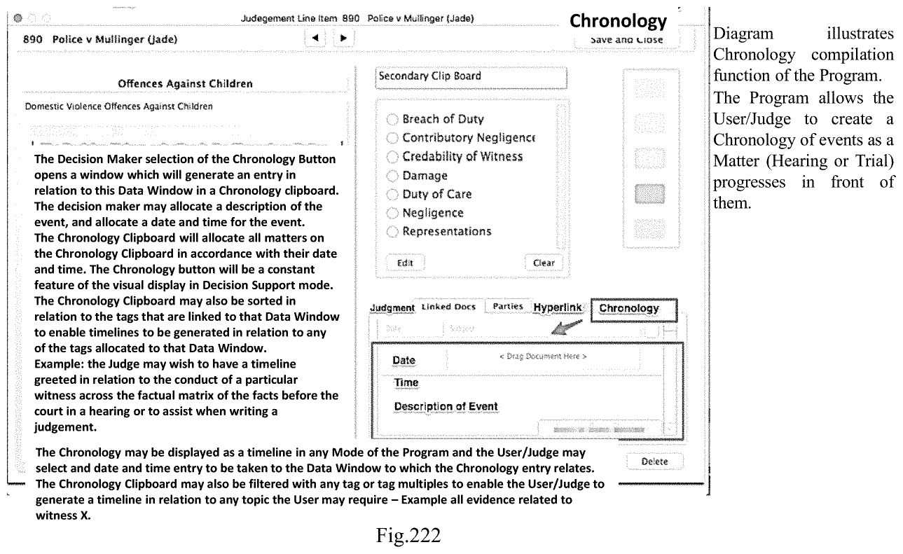

FIG. 222: Illustrates Chronology (Time Line) compilation function of the Program. The Program allows the User to generate a highly detailed Chronology of events as a Matter (Hearing or Trial) progresses before the User/Judge.

A Chronology is a critical document in the management of any litigation, during both the course of the Hearing or the Trial, and in assisting the User/Judge greatly in preparing Judgment.

A User/Judge (or User conducing any investigation or enquiry) will frequently need to refer to the Chronology of events in the matter and the Chronology accordingly is available in every mode of operation of the Program.

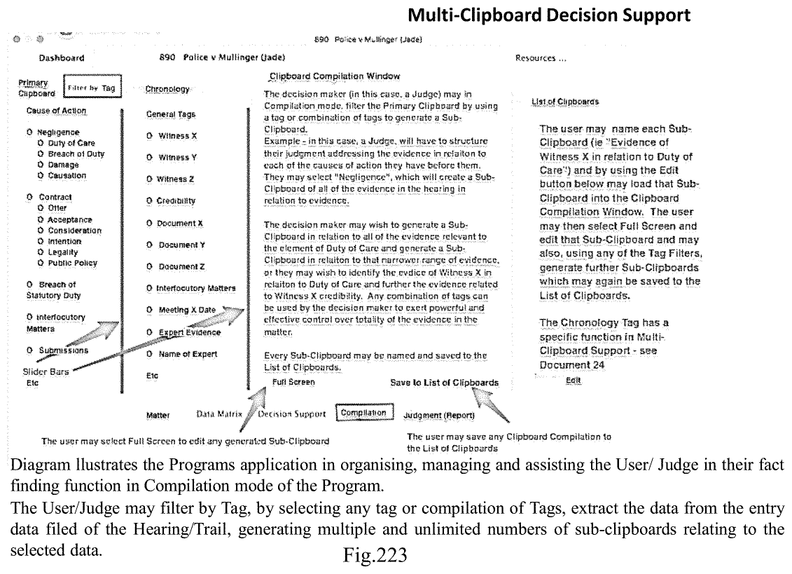

FIG. 223: Illustrates the Programs application in organising, managing and assisting the User/Judge in their fact finding function in Compilation mode of the Program.

The User/Judge may Filter by Tag, by selecting any tag or compilation of Tags, extract the data from the entry data filed of the Hearing/Trial, generating multiple and unlimited numbers of sub-clipboards relating to the selected data. Such Sub-Clipboards so generated by the User/Judge will be saved to a List of Clipboards and will be available to the User/Judge to bring into the visual display.

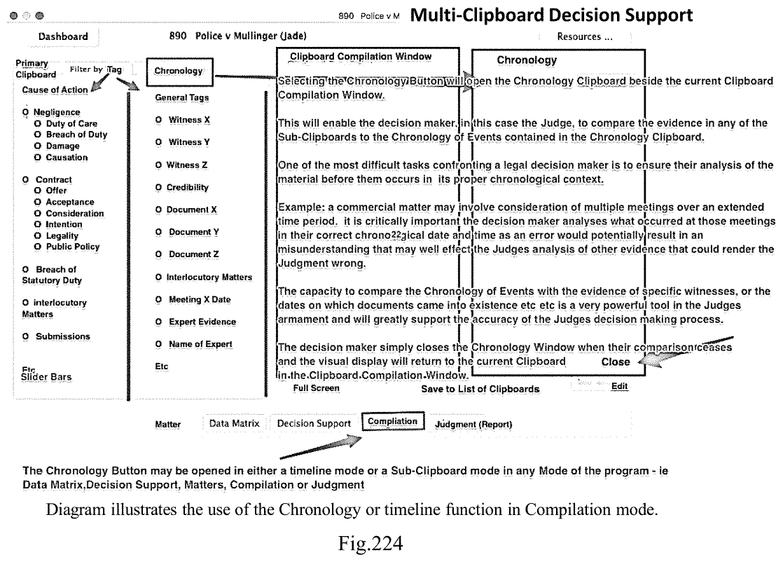

FIG. 224: Illustrates the use of the Chronology (Timeline) function in Compilation mode.

Chronology or timeline function in Compilation mode.

The User may be significantly assisted when extracting data from the Primary Clipboard to be able to see the timeline of events (Chronology) to assist them is selecting the tags they wish to utilise to extract data into a particular sub-Clipboard so the Chronology or timeline function is also available in this interface of the Program.

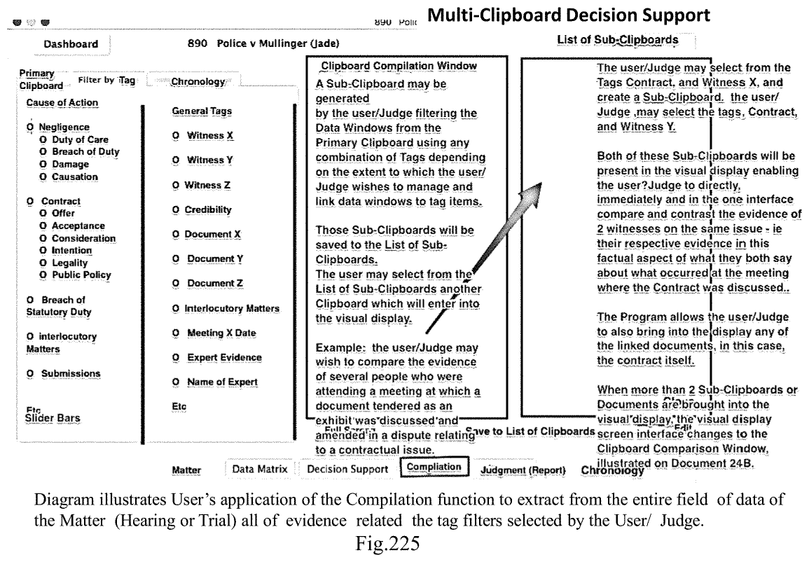

FIG. 225: Illustrates User's application of the Compilation function to extract from the entire field of data of the Matter (Hearing or Trial) all of evidence related to the tag filters selected by the User/Judge.

This functionality is also applicable to any persons condign investigations in respect of complex factual investigations to allow the User a powerful system for comparing and contrasting competing evidence and ensuring they do not miss any data critical to their decision.

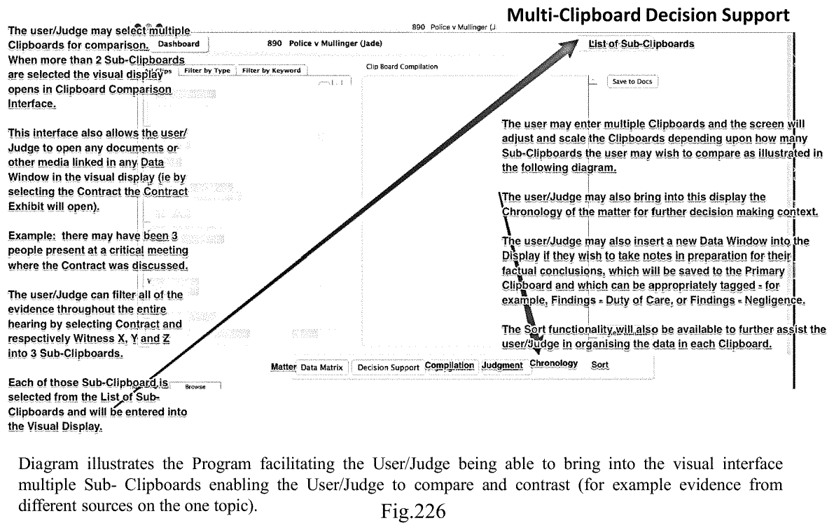

FIG. 226: Illustrates the Program facilitating the User/Judge being able to bring into the visual interface multiple Sub-Clipboards enabling the User/Judge to compare and contrast (for example evidence from different sources on the one topic) Example: different witnesses evidence one one issue the User/Judge has to decide.

Example: there may be 10 witnesses who gave evidence about what occurred at a specific meeting, and the Program allows each of that witnesses evidence on that topic to be brought into the visual display, as illustrated in the following diagram.

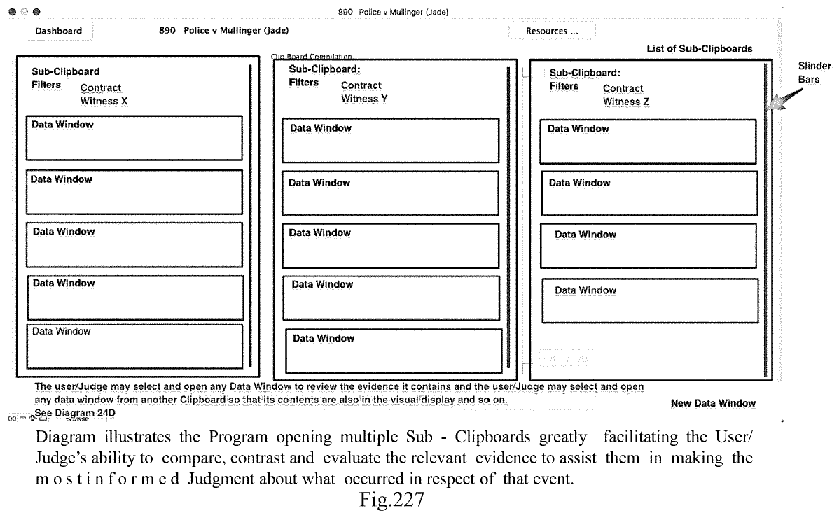

FIG. 227: Illustrates the Program opening multiple Sub-Clipboards greatly facilitating the User/Judge's ability to compare, contrast and evaluate the relevant evidence to assist them in making the most informed Judgment about what occurred in respect of that event.

The User/Judge may resize the Sub-Clipboards and open their Data Windows to assist the Users fact-finding function.

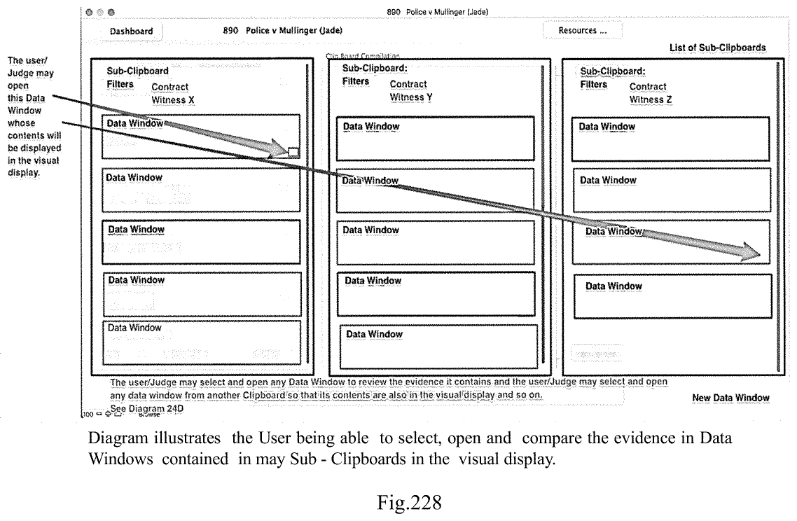

FIG. 228: Illustrates the Program allowing the User/Judge to select any data window from any of the Sub-Clipboards which will allow the User to directly and immediately compare the evidence contained in that data window.

Example: User extracts all evidence given by 3 key witnesses about a contract. The User may open the data window relevant to that issue and compare 1 or more of the witnesses evidence which is gathered in the visual display

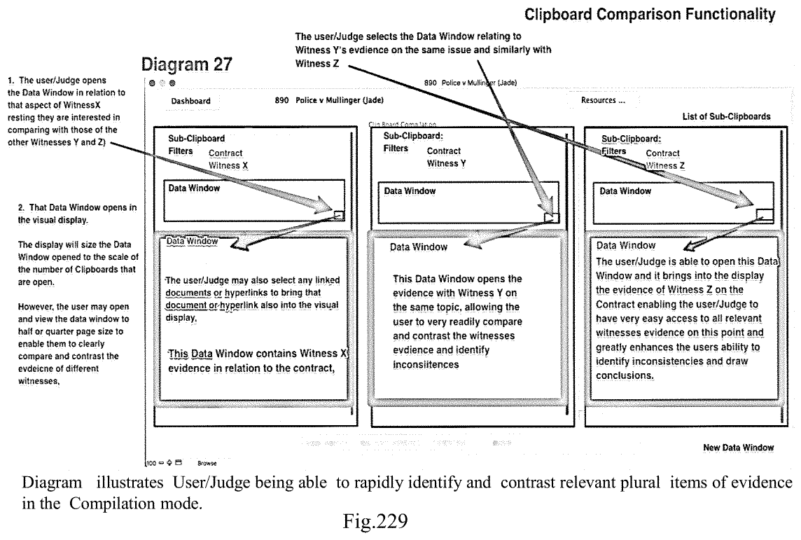

FIG. 229: Illustrates User/Judge being able to rapidly identify and contrast relevant plural items of evidence in the Compilation mode.

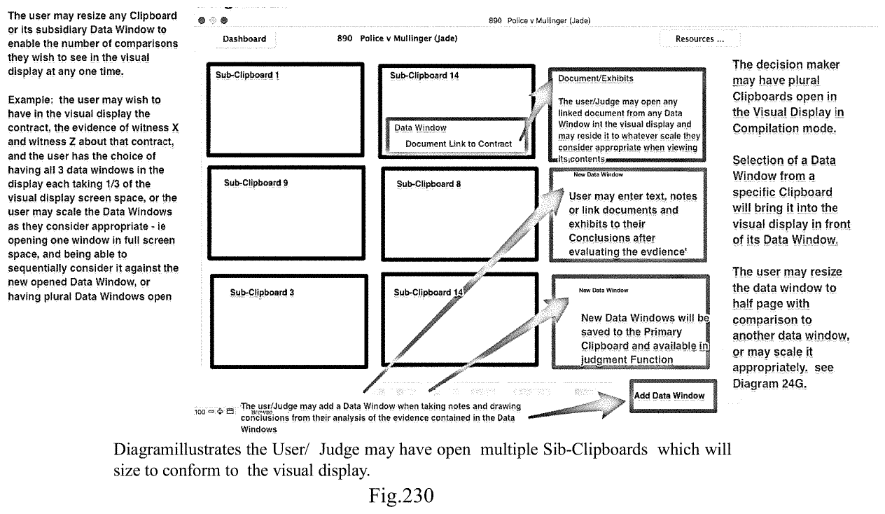

FIG. 230: Illustrates the User/Judge may have open multiple Sib-Clipboards which will size to conform to the visual display.

Any Data Window from any Sub-Clipboard may be selected and brought into the visual display and resized as the User may require, and directly be used to compare to any other Data window or windows from any other Sub-Clipboard.

The User may also select and open in the visual display any linked document—example the contract about which the Judge has selected the related witnesses evidence.

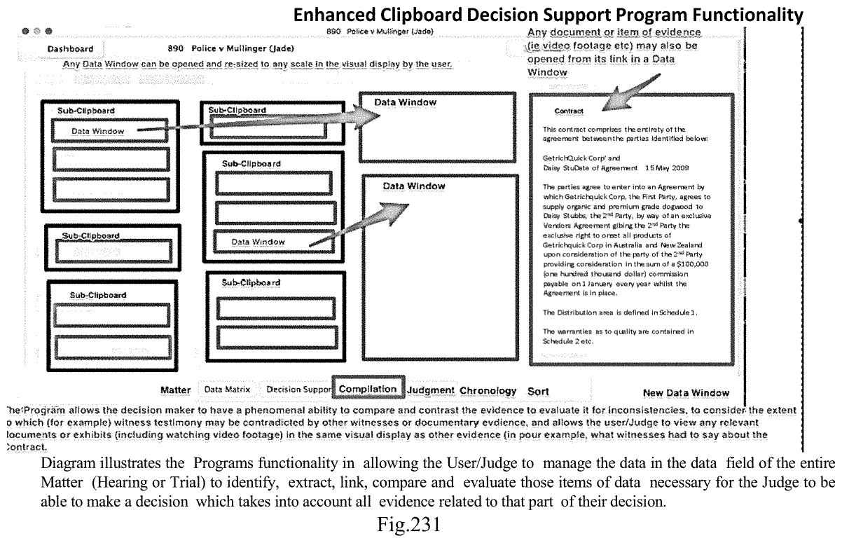

FIG. 231: Illustrates the Programs functionality in allowing the User/Judge to manage the data in the data field of the entire Matter (Hearing or Trial) to identify, extract, link, compare and evaluate those items of data necessary for the Judge to be able to make a decision which takes into account all evidence related to that part of their decision.

The User/Judges conclusions are able to be entered into a New Data Window reflecting their conclusions of fact and the reasons substantiating that conclusion based on their thorough analyses of all items of related evidence in the data field of the Primary Clipboard as well as ensuring they do not omit any evidence relevant to their decision on that issue.

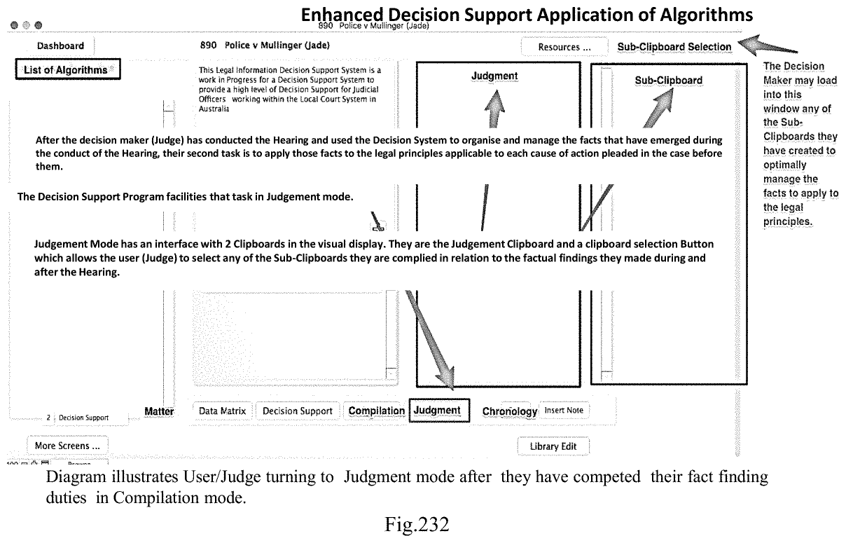

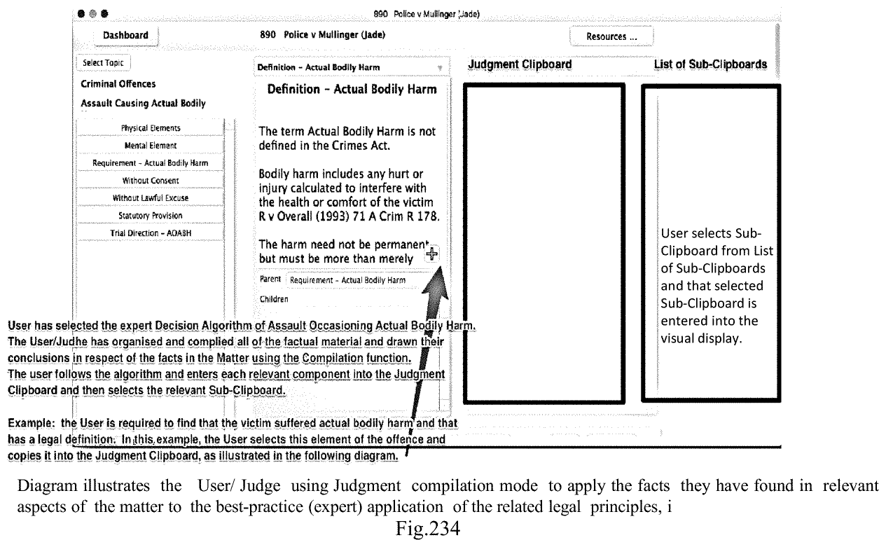

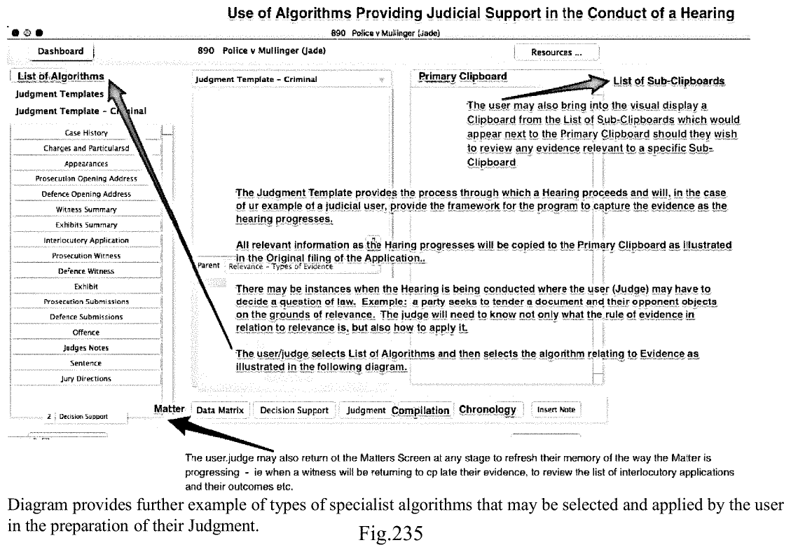

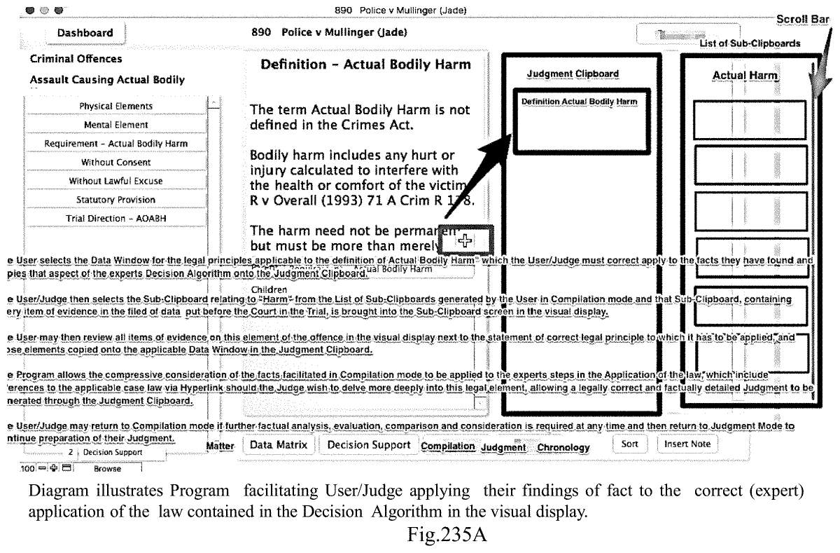

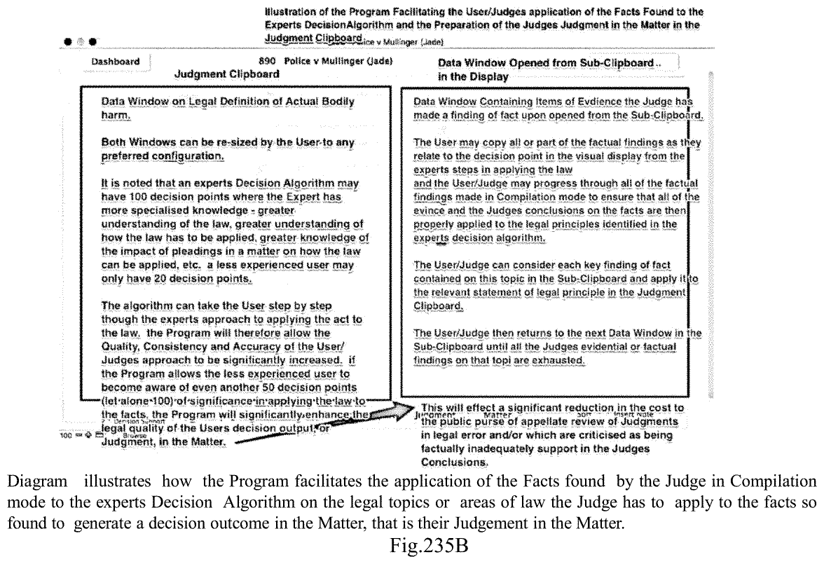

FIG. 232: All fact-finding in sub-clipboards-fact-finding completed-now go into judgement mode—with reference to FIG. 232)—user selects a list of algorithms in FIG. 234—and loads the algorithm they need to apply into the visual display (illustrated in FIG. 235A—report assembly function).

Illustrates User/Judge returning to Judgment mode after having competed their fact finding function about deciding the facts and drawing their conclusions in respect of competing evidence using the Compilation mode of the program.

A Judgment Clipboard opens in the visual display and the User may bring into an adjacent window in the Visual display any of the Sub-Clipboards generated in the User/Judges application of the Compilation mode.

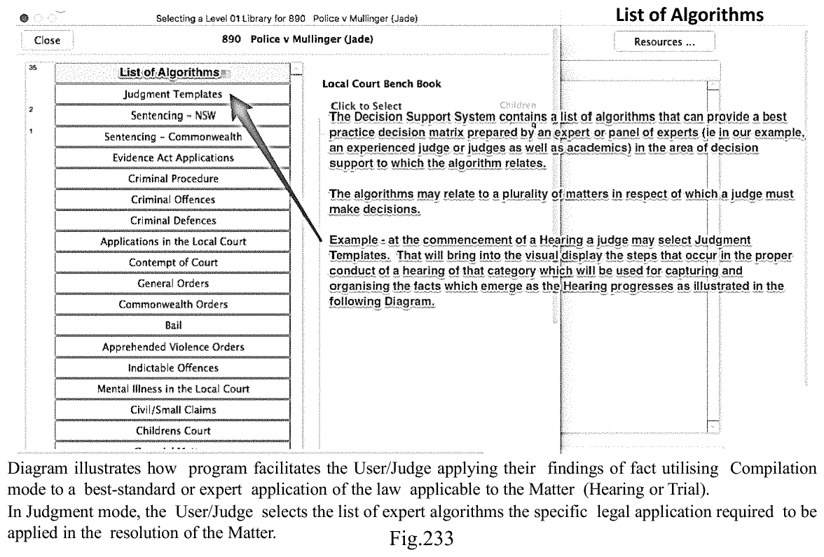

FIG. 233: Illustrates how program facilitates the User/Judge applying their findings of fact utilising Compilation mode to a best-standard or expert application of the law applicable to the Matter (Hearing or Trial).

In Judgment mode, the User/Judge selects from the List of expert algorithms the specific legal application required to be applied in the resolution of the Matter.

FIG. 234: Illustrates the User/Judge using Judgment compilation mode to apply the facts they have found in relevant aspects of the matter to the best-practice (expert) application of the related legal principles, in this example, the expert approach to applying the law relating to Assault Occasioning Actual Bodily harm selected from their lost of Algorithms.

FIG. 235: Provides further example of types of specialist algorithms that may be selected and applied by the user in the preparation of their Judgment.

In instant example the Judge/User may select a best practice algorithm for the structure of a Judgment.

FIG. 235A: Illustrates Program facilitating User/Judge applying their findings of fact to the correct (expert) application of the law contained in the Decision Algorithm in the visual display.

The User/Judge proceeds through the experts decision steps, opens the experts Data Windows in that decision process to which the facts before the User/Judge relate, copies that Data Window into the Judgment Clipboard, opens the Sub-Clipboard findings if fact to which that legal issue relates, and the Program brings into the visual display the data window containing the expert application of the law and allows the User/Judge to proceed though their findings of fact and copy the relevant conclusions into the Judgment data window to which those facts relate.

FIG. 235B: Illustrates how the Program facilitates the application of the Facts found by the Judge in Compilation mode to the experts Decision Algorithm on the legal topics or areas of law the Judge has to apply to the facts so found to generate a decision outcome in the Matter, that is their Judgment in the Matter.

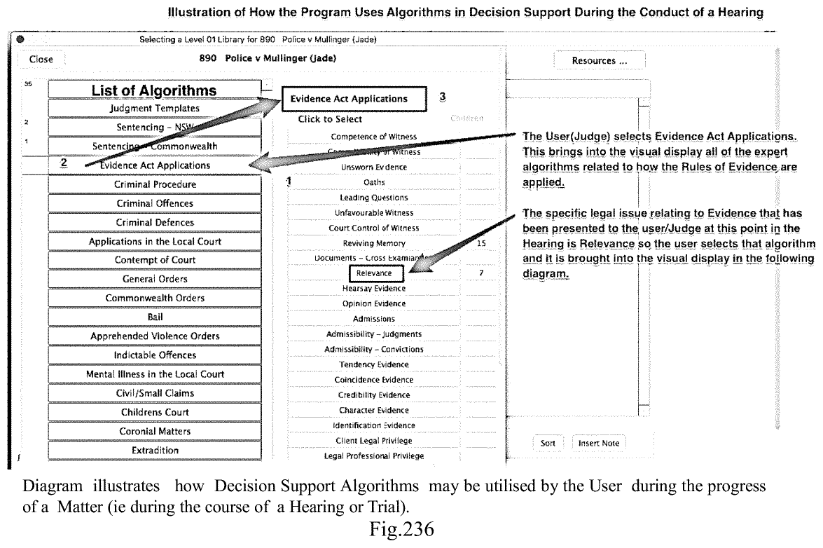

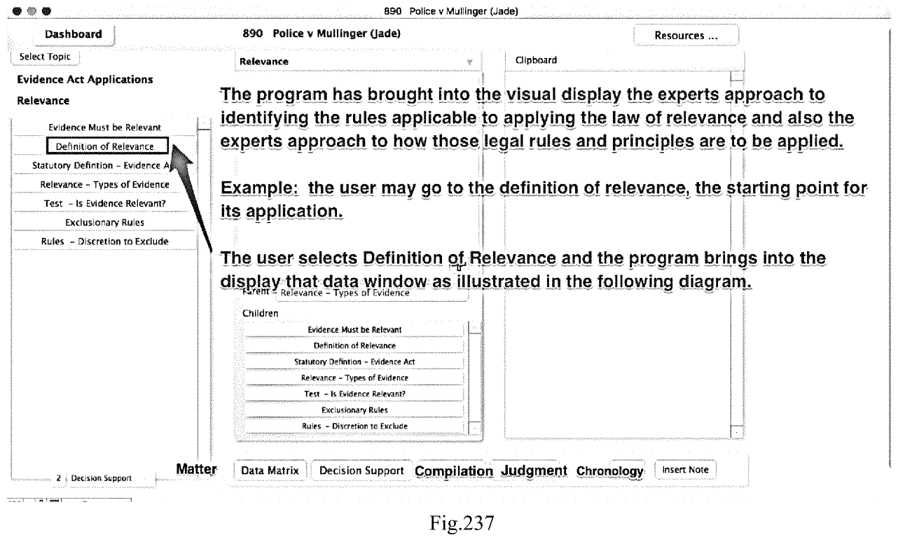

FIG. 236: Illustrates how Decision Support Algorithms may be utilised by the User during the progress of a Matter (ie during the course of a Hearing or Trial).

Example: it is very frequently the case that a User/Judge has to make a ruling during the conduct of a Matter in relation to the law of Evidence.

User selects Evidence Law from the List of Algorithms when an evidential ruling (ie decision on the application of the law to the facts before the Judge) is required, and will then select the topic in the law of evidence that is being argued by the lawyers.

The User/Judge selects that topic and it is loaded into the visual display in the following diagram.

FIG. 237: Illustrates the User/Judge bringing into the visual display an expert decision support algorithm to assist them make a legal decision relating to an aspect of the progress of the matter before them.

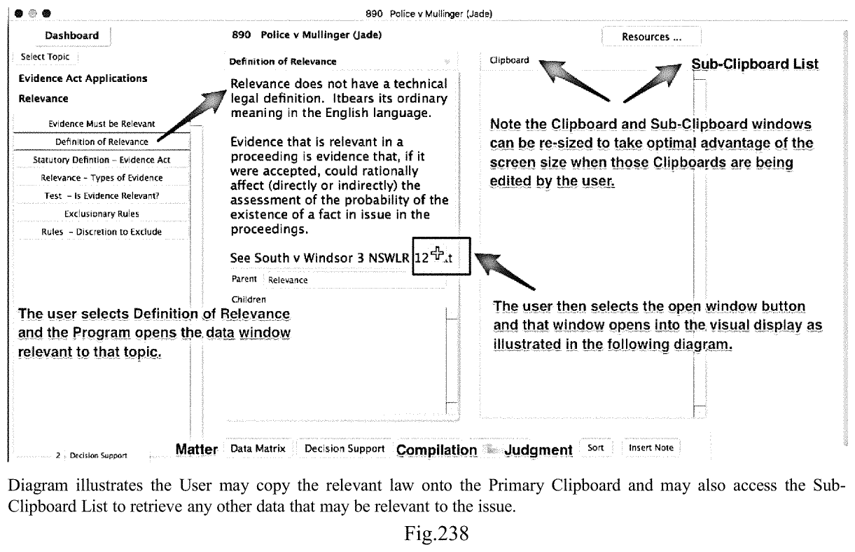

FIG. 238: Illustrates the User may copy the relevant law onto the Primary Clipboard and may also access the Sub-Clipboard List to retrieve any other data that may be relevant to the issue.

Example: a document such as a contract may be brought into the visual display to assist the Decision-Maker having immediate access to the document in question in relation to arguments of evidence. that document may also be viewed and examined by the Decision-Maker returning to Matters interface and brining the document into the Visual Display.

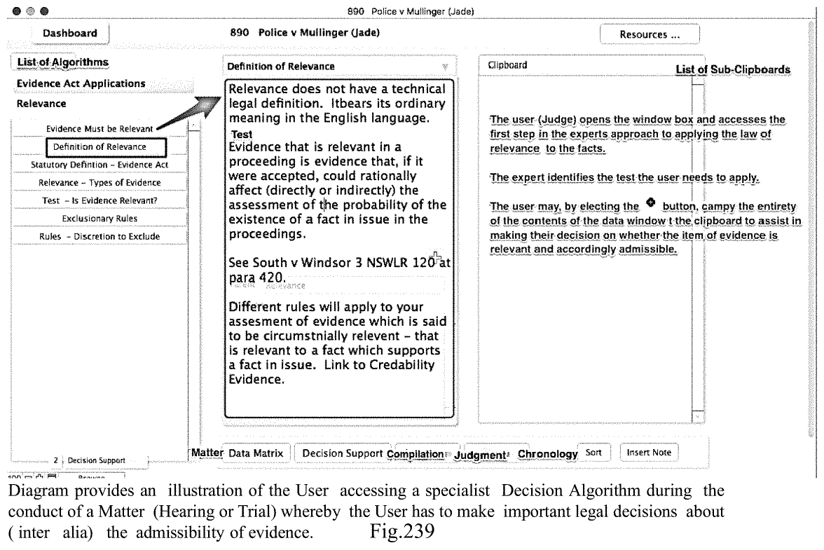

FIG. 239: Illustrates provides an illustration of the User accessing a specialist Decision Algorithm during the conduct of a Matter (Hearing or Trial) whereby the User has to make important legal decisions about (inter alia) the admissibility of evidence.

The rules of evidence are very technical and they are frequently the source of legal error in their application, particularly as a Judge does not usually have time ti adjourn the proceedings to legally research how that rule of evidence applies, and because they may not have the same level of expertise as the expert whose algorithm they can immediately access from their list of algorithms.

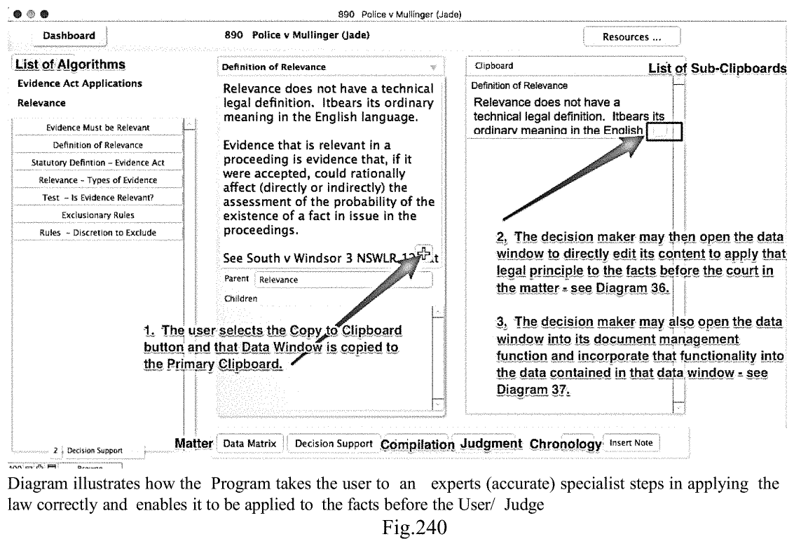

FIG. 240: Illustrates how the Program takes the user to an experts (accurate) specialist steps in applying the law correctly and enables it to be applied to the facts before the User/Judge.

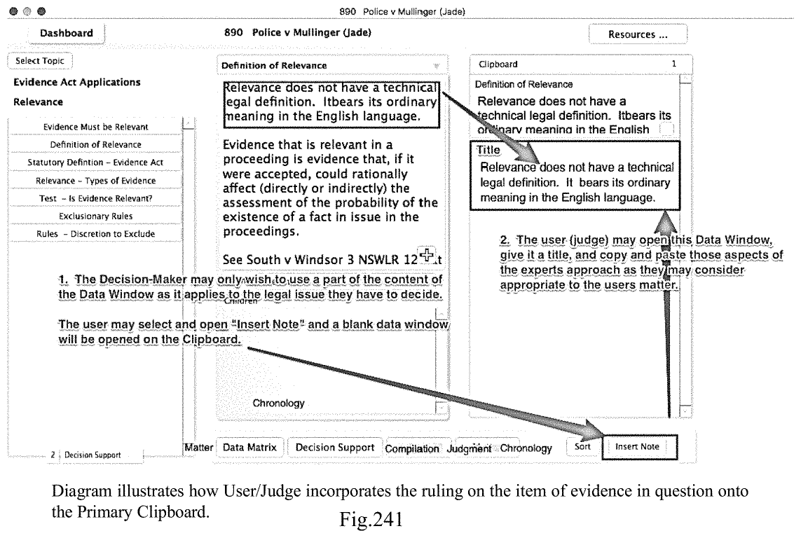

FIG. 241: Illustrates how User/Judge incorporates the ruling on the item of evidence in question onto the Primary Clipboard.

All evidential rulings may be Tagged with the Tag “Evidential Rulings” and incorporated into a Summary Sub-Clipboard.

Such a Summary is very useful to the User/Judges efficient management of a Matter (Hearing or Trial) because it is frequently the case that a later Evidential ruling may well depend on one which has been made earlier in the Matter and the User/Judge can immediately return to all of the evidential rulings as well as the reasons and legal principles that underpinned that earlier ruling.

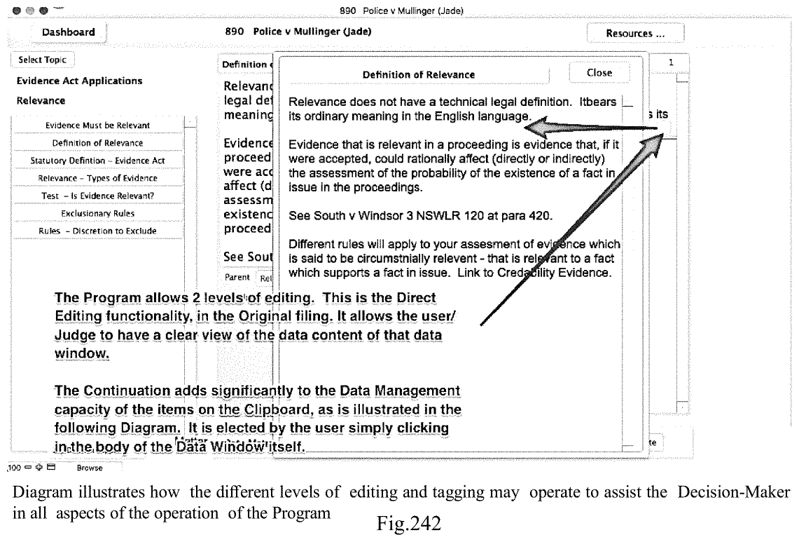

FIG. 242: Illustrates how the different levels of editing and tagging may operate to assist the Decision-Maker in all aspects of the operation of the Program.

Example: in allowing the Decision-Maker user to tag all of the different types of applications that are made during the Course of the Matter (hearing or Trial) so they may brought into their relevant Sub-Clipboards immediately and without any further manual organisation of those data items by the Judge.

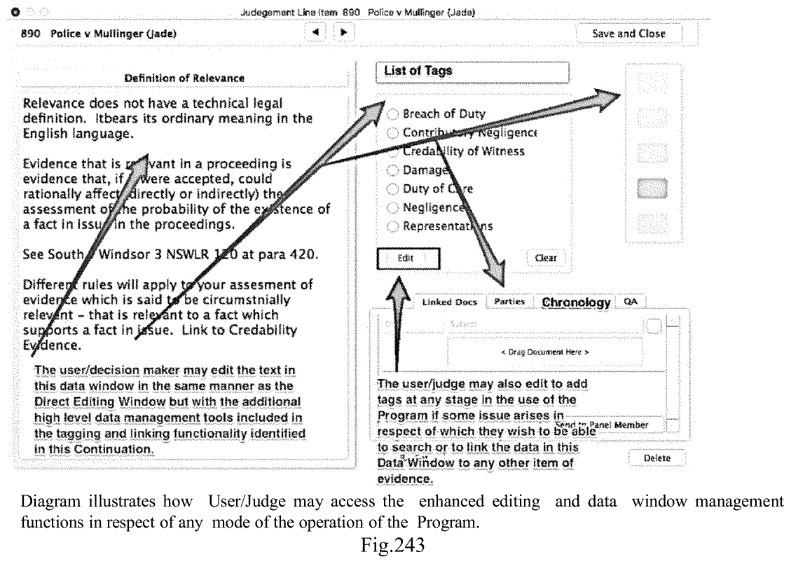

FIG. 243: Illustrates how User/Judge may access the enhanced editing and data window management functions in respect of any mode of the operation of the Program.

In this example, the User/Judge would select a tag “Evidence Rulings” rom the List of Tags, that would enable the Judge/User to immediately and at any stage in the operation of the Program (at any stage during the course of a Matter, Hearing or Trial, immediately generate a Sub-Clipboard containing all evidential rulings and the reasons for those rulings.

Any Clipboard is able to be copied into a document in a variety of formats (Word, ePub, etc).

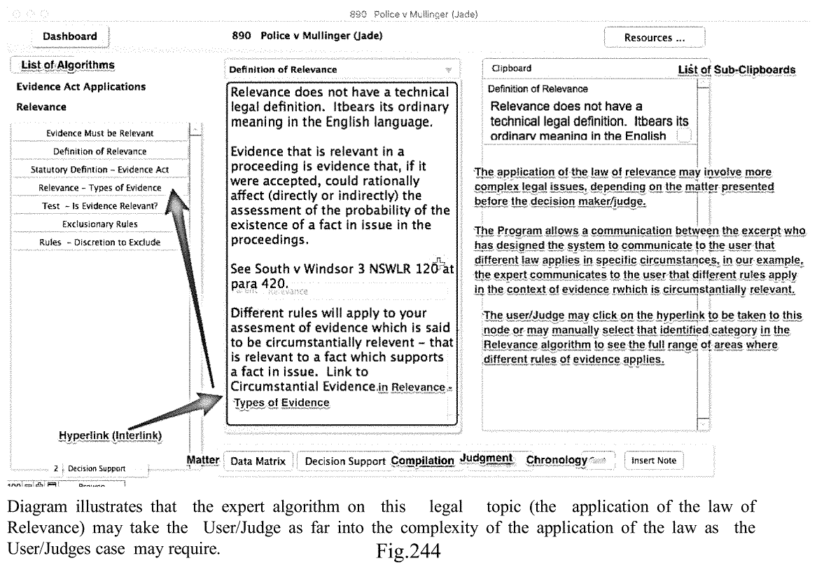

FIG. 244: Illustrates that the expert algorithm on this legal topic (the application of the law of Relevance) may take the User/Judge as far into the complexity of the application of the law as the User/Judges case may require.

Each of the principle topics that apply in the Application of the Law of Relevance may have many sub-categories reflecting the sophistication and specialist knowledge of the author of the specialist algorithm and the algorithm will take the User/Judge as far into the experts approach as may be required y the facts presented to the User/Judge. That will significantly enhance the Users identification of and correct application of the applicable legal principles.

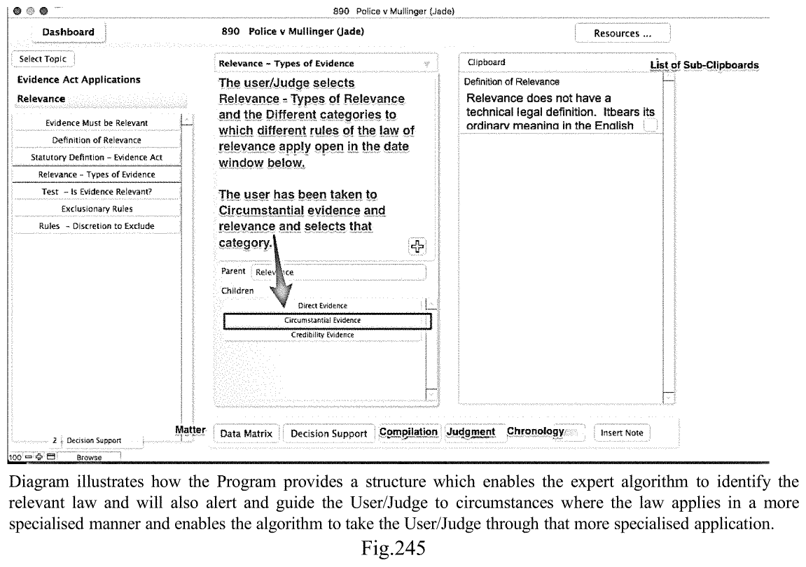

FIG. 245: Illustrates how the Program provides a structure which enables the expert algorithm to identify the relevant law and will also alert and guide the User/Judge to circumstances where the law applies in a more specialised manner and enables the algorithm to take the User/Judge through that more specialised application.

Example: the expert algorithm will draw an inexperienced users attention to the fact that the law of relevance can have different applications considering whether it is Direct Evidence, Circumstantial Evidence or Credibility Evidence. The program will them take the User through a legally correct application of the specific form to which its more complex application relates.

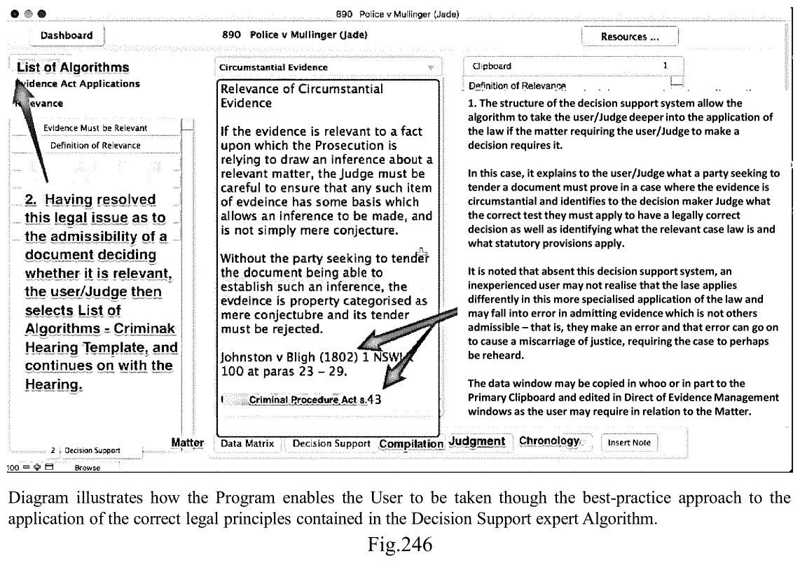

FIG. 246: Illustrates how the Program enables the User to be taken though the best-practice approach to the application of the correct legal principles contained in the Decision Support expert Algorithm.

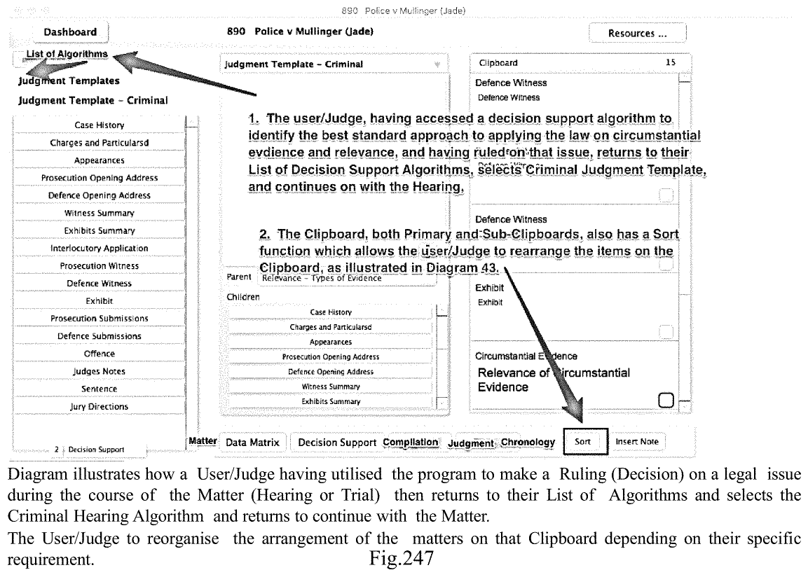

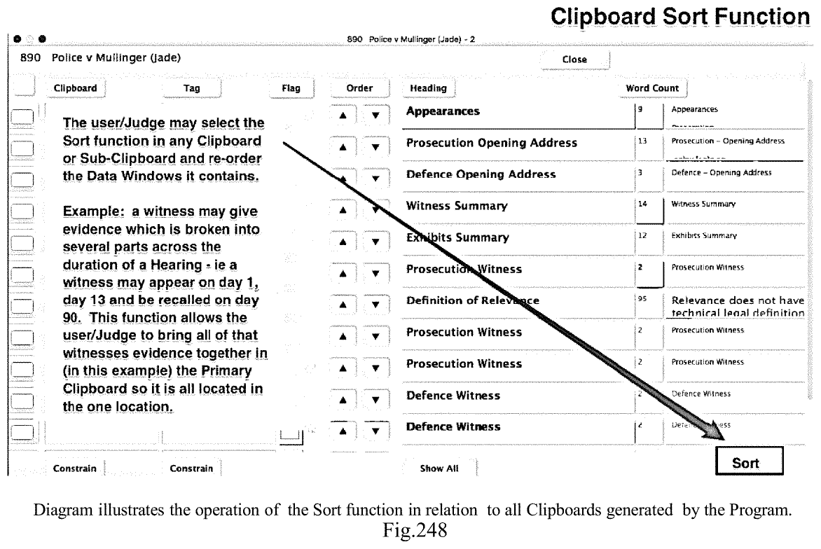

FIG. 247: Illustrates how a User/Judge having utilised the program to make a Ruling (Decision) on a legal issue during the course of the Matter (Hearing or Trial) then returns to their List of Algorithms and selects the Criminal Hearing Algorithm and returns to continue with the Matter.

The Program also has a Sort function in respect of all Clipboard, Primary and Sub-Clipboards, which enables the User/Judge to reorganise the arrangement of the matters on that Clipboard depending on their specific requirements.

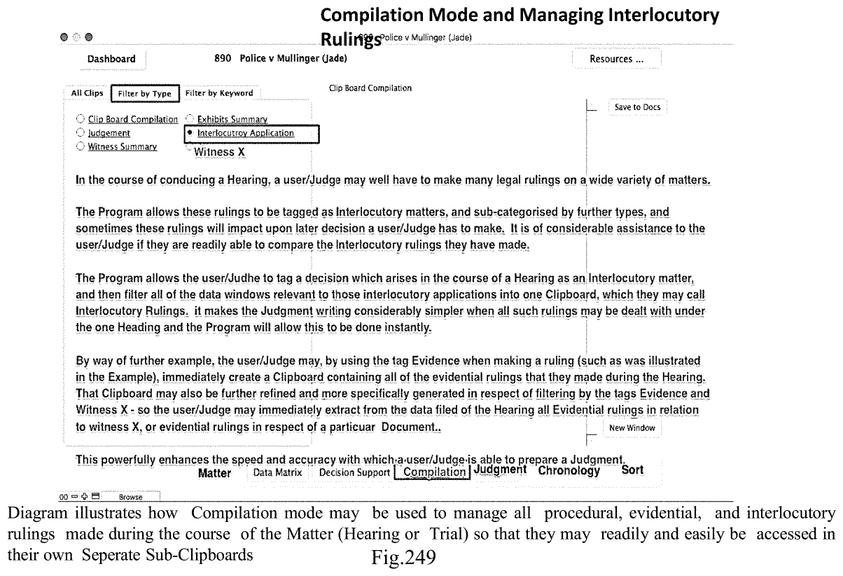

FIG. 248: Illustrates the operation of the Sort function in relation to data windows contained in the Primary Clipboard or any Sub-Clipboard enabling the User/Judge the capacity to structure the data windows/items of evidence in the most conductive way to assist in preparation of their Judgment or their personal preference.

FIG. 249: Illustrates how Compilation mode may be used to manage all procedural, evidential, and interlocutory rulings made during the course of the Matter (Hearing or Trial) so that they may readily and easily be accessed in their own Separate Sub-Clipboards.

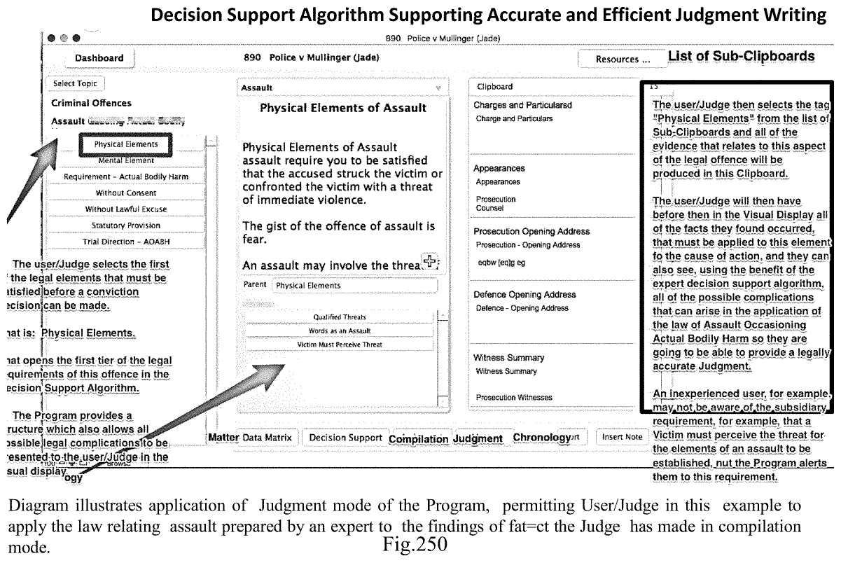

FIG. 250: Illustrates application of Judgment mode of the Program, permitting User/Judge in this example to apply the law relating assault prepared by an expert to the findings of fact the Judge has made in compilation mode.

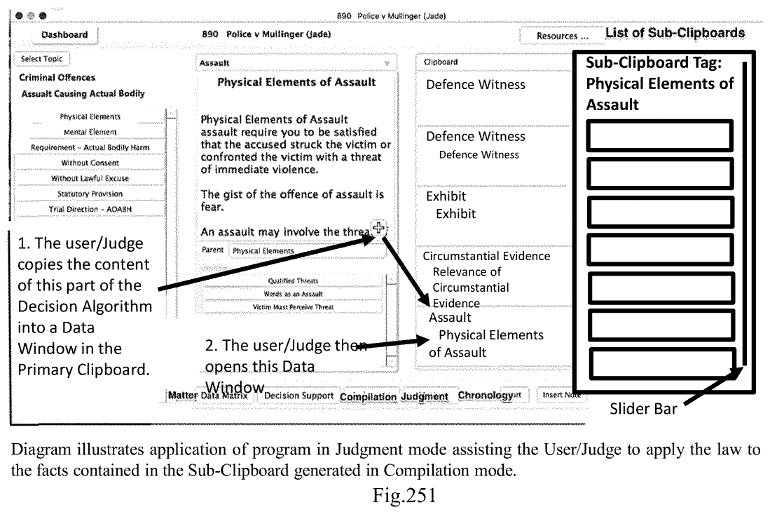

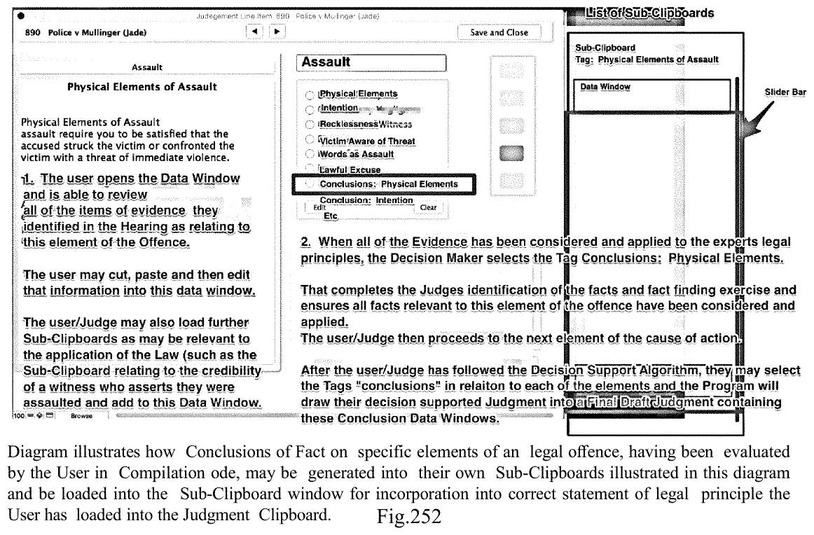

FIG. 251: Illustrates application of program in Judgment mode assisting the User/Judge to apply the law to the facts contained in the Sub-Clipboard generated in Compilation mode.

FIG. 252: Illustrates how Conclusions of Fact on specific elements of an legal offence, having been evaluated by the User in Compilation ode, may be generated into their own Sub-Clipboard as illustrated in this diagram and be loaded into the Sub-Clipboard window for incorporation into the correct statement of legal principle the User has loaded into the Judgment Clipboard.

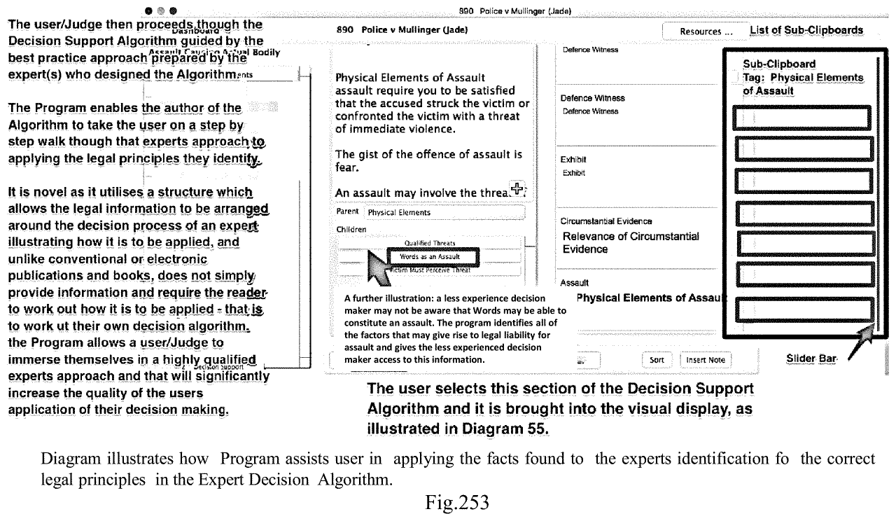

FIG. 253: Illustrates how Program assists user in applying the facts found to the experts dentification of the correct legal principles in the Expert Decision Algorithm.

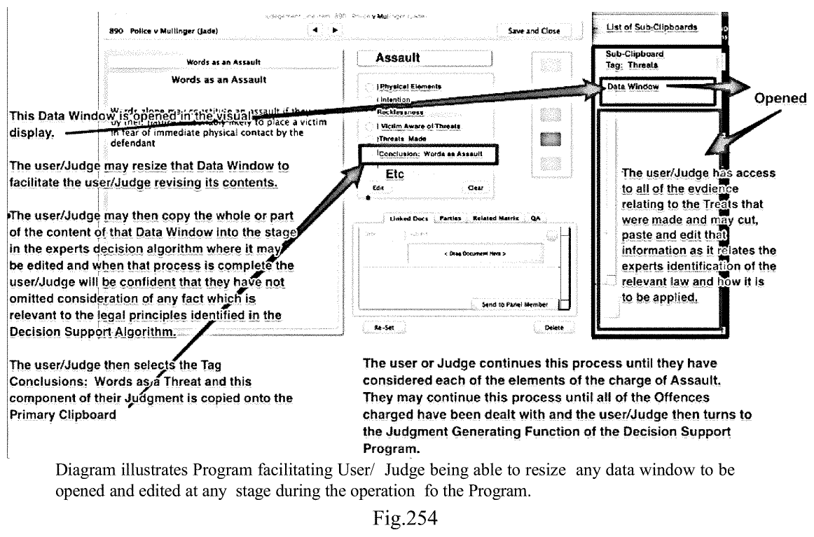

FIG. 254: Illustrates Program facilitating User/Judge being able to resize any data window to be opened and edited at any stage during the operation of the Program.

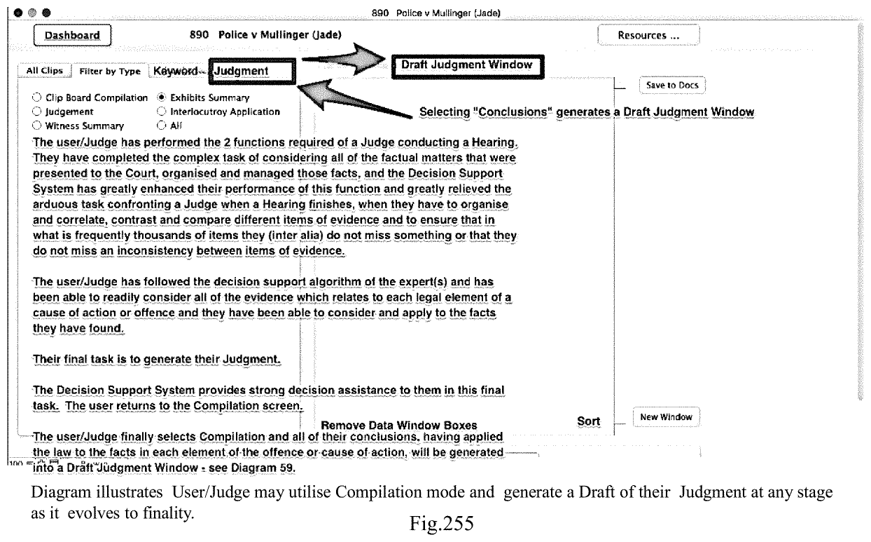

FIG. 255: Illustrates User/Judge may utilise Compilation mode and generate a Draft of their Judgment at any stage as it evolves to finality.

That Judgment may be exported to a variety of formats including Word, ePub, etc., that will contain the text of the Judgments, and only such document's that the User/Judge may elect to be included in the Draft Judgment Sub-Clipboard prior to exporting the Draft Judgment.

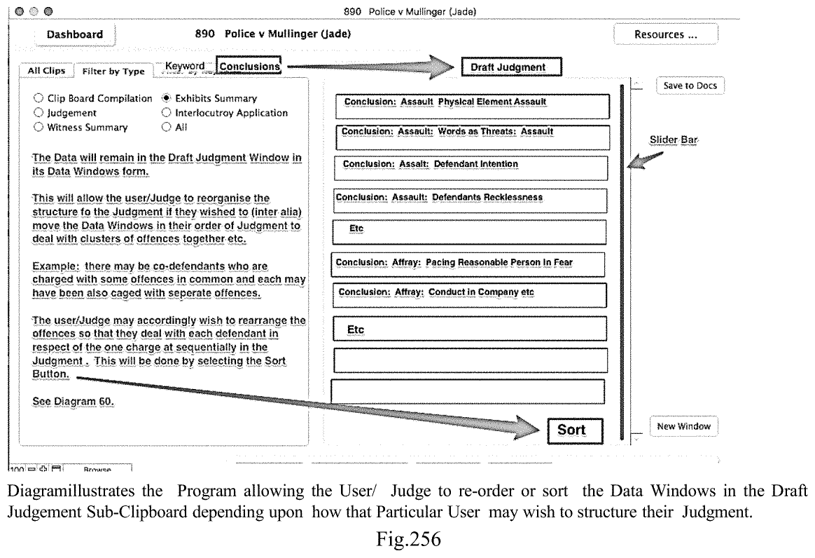

FIG. 256: Illustrates the Program allowing the User/Judge to re-order or sort the Data Windows in the Draft Judgment Sub-Clipboard depending upon how that Particular User may wish to structure their Judgment.

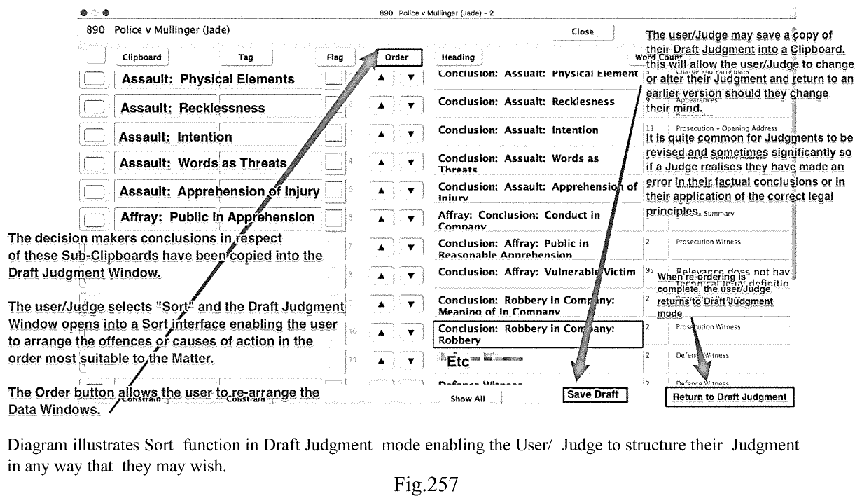

FIG. 257: Illustrates Sort function in Draft Judgment mode enabling the User/Judge to structure their Judgment in any way that they may wish.

### Detailed Description of Third Embodiment—In Use

the reader may be assisted by the following observations in respect of how the Decision Support System is asserted to operate in a practical context. The example that is used is of a Judge hearing a Matter. It will be appreciated that the expert system can be applied to other areas of endeavour.

A Matter is defined in this legal example as a trial or hearing. It may also include a lawyer preparing a matter for trial or hearing.

Other applications include a medical application being the investigation of a patients medical condition, the doctor making findings of fact in relation to a patients presentation and symptoms and then using the expert diagnostic algorithm of a specialist medical practitioner to ensure a best practice diagnoses and treatment regime.

There are two tasks confronting persons responsible for persons making decisions in any complex decision system. They are:
1. the Judges fact finding function: and, after that has been completed,
2. The Judges application of the relevant law to the facts.

It is noted that this delineation is not absolute as a Judge also has to apply the law to the facts in plural interlocutory matters—ie where, during the course of a Hearing or Trial the Judge May have to make rulings on many matters such as the law relating to whether a particular document is admissible in accordance with the law of Evidence, or whether a person may object to giving evidence on the grounds of Privilege etc. Nevertheless, the 2 key functions of a Judge are to find the facts and then apply the law to those facts so found.

1. The Use of the Decision Support System in the Judges Fact Finding Function.

Example: a legal matter comprising a hearing or trial, hereafter a “Matter”. A Matter comprises a process through which the Judge/User must hear, compare, evaluate, interrelate, and ultimately make decisions about which facts they determine happened, which they reject as happening as a party or parties assert, and they must provide cogent reasons by reference to the evidence put before the court (the entire data field of all information presented to the court), including the evidence of all witnesses, tendered documents, experts reports, multimedia forms of evidence etc as video and electronically stored evidence, as a matter progresses before them.

This is referred to as the “Fact Finding” task of a Judge and it is both a complex and difficult exercise.

The Findings of Fact made by a Judge are frequently a source of appealable error when an appeal court finds that the Judge:
failed to take into account a significant fact or facts;
failed to identify a material inconsistency between items of evidence;
failed to give reasons grounded or explained in the evidence why they came to a particular factual conclusion.

The larger the case the more exacting the process of fact finding becomes and there may be many thousands of items of evidence the User/Judge has to correlate, evaluate, assess its evidential weight, consider its implication in respect of multiple other items of evidence, and ultimately make decisions in relation thereto.

Further, a Matter will also require a Judge to make multiple decisions applying the law to specific issues which arise as a Matter progresses. A Judge will have to address a wide range of legal topics, including a vast array of evidential issues (for example: rulings on character evidence, credibility evidence, coincidence evidence, tendency evidence, identification evidence, the application of the law of legal privilege, the standards of proof, the complex law relating to documentary evidence, hearsay evidence and opinion evidence etc)

In what are called “interlocutory rulings” (rulings made as the course of a Matter progresses), the Judge has to ascertain what the correct application of the law is before they can apply it to the specific interlocutory application or legal decision on that point. Such rulings occur frequently across the course of the Matter and each may be understood as a “decision point” or a “decision node”.

It is worth noting that these rulings are generally made immediately when the issue arises during the course of a matter as there is often insufficient time for a Judge to adjourn the proceedings. These interlocutory rulings—application of the law—may be of considerable significance in the outcome of a hearing or trial. For example, if a Judge mistakenly admits an item of evidence, because they did not have a sufficient understanding of the area of law to which that ruling applied, it may taint the entire hearing or trial process, resulting in the Matter going on appeal where the Judges error of law is corrected by the Appellate Court, who may then direct the Matter be sent back to the originating Court to be reheard in its entirety with the Judge directed to apply the law correctly as it has been identified by the appellate a court.

It goes without saying that the costs of Appellate review—to the public purse in the maintenance of the Court system, including court staff and Judges, as well as to the litigants, is a very high figure.

It is further noted that the extent of legal error is significantly increasing due to the enormous increase in the extent—the sheer amount—of law in Western Societies, and also it's depth, or complexity. The former Dean of Yale Law School, Guido Calabresi, noted in The Common Law and the Age of Statutes (1985), identified that at the time of its publication there had been an 800% increase in the amount of law which practitioners and Judges have to deal with across the preceding 40 years, and this tendency is accelerating.

What a Judge must do in applying the law, both during and after a Matter and when they are preparing their judgement, is read a plural number of resources—cases, statutes, academic articles, on line legal publication services, practice and procedure documents, etc, and derive their own decision model for solving all of these various applications of the law. I have referred to their approach as their decision algorithm. I have used the term algorithm for this process in the more contemporary definition of that term, referring more broadly to any decision process that generates an outcome and not as some expressly algebraic system. A Uses decision algorithm can usefully be described for the instant purposes as constituted by the number of decision points that person is able to access when they are applying that particular legal rule. By way of example, a Judge inexperienced in a Negligence action may only have 5 decision points in their understanding or their algorithm of how to apply one of the sub-set of rules relating to whether a Negligence claim will succeed in a Matter—whether the defendant to the Matter owed the plaintiff a Duty of Care. An experienced lawyer/judge who has years of dealing with Negligence claims may have 50 decision points in respect of their understanding of and ability to apply this legal principle, whether the defendant owes the plaintiff a duty of care, in their application of this area of law. They may be aware, for instance, that special factual circumstances will give rise to different rules that apply, and the inexperienced User may be entirely unaware that such circumstances have that legal effect. The expert author of the Decision Algorithm may have specialised knowledge of how technical issues in relation to the pleadings in a Matter may operate to limit the evidence that can be used by a particular party, and they may be aware of more specialised applications of the law, for example that contributory negligence may be applicable in relation to the assessment of the plaintiff's damages. In short, the approach they take to applying the law to the facts is considerably more sophisticated and nuanced than that of the less experienced user and the application of their decision algorithm will inevitable generate a higher quality decision output that is inevitable going to present a more thorough and expert decision outcome.

Lawyers and Judges all have to work out for themselves what the own decision algorithm is going to be for every item of law they had to apply. This becomes increasingly problematic when the amount and complexity of the law is on a very steep trajectory and there are studies which assert many cases decided contain legal and or factual error but many are simply not pursued on appeal, either because the party does not identify them, or because of the very high cost of funding an appeal and re-hearing. The likelihood of error of course increases when complex legal issues have to be applied under time pressure, as will frequently occur during interlocutory legal rulings in the course of a Matter. The greater the sophistication and the number of decision points in a Lawyer or Judges decision algorithm, the more valuable it is, as it is much more likely to generate a higher quality (that is, legally accurate and consistent) judgement outcome then a less sophisticated decision system with a reduced number of decision points. The significantly enhanced value of expert decision systems is why the cost of specialist services and advice (for example leading lawyers, medical specialists, specialist engineers etc) is often considerably higher than that of generalist practitioners in all fields of endeavour. It is because the specialist has a more complex, sophisticated, nuanced and deeper knowledge of how the law is applied then less knowledgeable and experienced, or new, users.

The programme provides a system which permits the experts decision algorithm on the legal topic to be extracted (in practical effect) from where it is reposed (in their head) and mapped into the System in a way which enables the expert algorithm to guide the less experienced User through the experts decision points. The expert algorithm, further, takes the User from the more general application of legal principle through to its more complex applications to the facts before the User, and will take the User only to that point in the complexity of the application of the law on that topic as is required by the factual matters which present themselves to the User in the Matter.

The program presents a visual display that allows the User to maintain the context or location of key steps of the experts decision process, as it maintains these key steps in the visual display in the left hand window of the visual display in its operation in Judgment mode, where the Judge is assisted in applying the facts they have found in the specific matter before them to the decision points in the experts algorithm. The Decision Support System allows the User to progress through the experts application of the expert's approach to applying the law to the facts found by the User at each decision point or node in the experts algorithm as the User progresses deeper, ie progresses through, the experts algorithm. The programs maintenance of the key decision points in the left Window of the visual display assists in maintaining the Users context and the System assists the User in that it takes them only to those legal resources applicable to the law at that specific issue and thereby assists in not overwhelming the User by immersing them in legal resources data that does not apply to that specific issue to which the User is applying the facts.

The program allows the user to progress through the steps in the expert algorithm in the middle left Window.

The System also allows the User to apply algorithms in Decision Support Mode, as a Matter is proceeding before a Judge and the Judge May have to draw on expert Decision Support Algorithms in respect of (inter alia) the law of evidence. In the case of a medical diagnoses, the User's reasons for the conclusions are made transparent at every step of a diagnostic process and the decision algorithm will draw their attention to and explain specific matters they may have to take into account that apply to diagnosis of a complex issue that the less experienced user may have been entirely ignorant of, the System thereby facilitating the quality of the less experienced Users diagnostic process and accordingly resulting in a better quality diagnoses than that which would be likely to occur should a medical practitioner rely upon their own less sophisticated decision algorithm.

Returning to the lawyer/Judge example, as noted, there are two tasks confronting a Judge in a Matter.

(1) They must do what is called “Find that Facts”. They must identify what evidence in a Matter the Judge “finds”—that is, ultimately determines—to have occurred.

Witnesses invariably give different accounts of many issues in a Hearing. There can be complex and competing issues for the Judge to decide. A meeting at may have occurred where 10 people attended, multiple issues were discussed, and different accounts were given by different witnesses. There may be issues that arise in relation to complex documentary matters: when a particular was produced, when certain amendments to documents were made, and the Matter may have had tendered hundreds and sometimes thousands of documents. There may be multiple expert reports, and expert testimony given by experts at court which differs markedly.

Further, evidence on all of these issues does not harmoniously fold out in an ordered sequence in relation to each legal matter the judges must determine: evidence will emerge on a particular factual matter from different witnesses in both the plaintiff and the defendant's case, and a Judge will have to make assessments about the credibility and believability of witnesses from many plural sources in a case—for example, the date a witness asserted they created a document may be inconsistent with a facsimile copy of that document bearing an earlier date, leading the Judge to doubt the honesty or credibility of that witness. A Judge will risk falling into appealable error if they simply assert that they preferred the credibility of witness X over witness Y. They must give reasons for that conclusion, and the reasons which generate that impression may be subtle and occur at different points in the evidence emerging in the conduct of the Matter. At the end of a trial it can be extremely difficult to reconstruct the factual issues that created the Judges impression.

This illustrates that the factfinding function of a Judge is very complex. There may be thousands of items of evidence in respect of which a Judge has to immerse themselves in, be able to cross reference, correlate, compare, contrast, and organise these various items to fulfil their judicial task of finding facts.

This Decision Support System provides a means of datafication of the items of evidence, whether items of witness testimony, documents, parts of documents, video and multimedia evidence etc which can be linked to the evidence in a specific data window allowing the Judge to immediately bring into the visible display the item to which the contents of that display relates.

The Program provides an unprecedented level of control over the entire field of the data before the Judge in the Matter.

Embodiments of the invention allow the Judge to enter tags onto each item of evidence as the evidence is unfolding before them during the conduct of a Matter. It also permits the Judge to use the tagging function at any stage during the conduct of the Matter and when reviewing the evidence prior to concluding the Fact Finding aspect of the Matter. The complexity of the interrelationships between the items of evidence in a Matter is high. A judge may have to constantly review transcription evidence, relying on their memory and notes, and it is fair to describe this exercise of fact finding as frequently laborious and highly time-consuming, and research makes it very clear that there are limits to how effectively humans are able to process multi-variable decision processes and they start to lose their ability to accurately evaluate these interrelationships.

The program allows the User/Judge to utilise tags which will both be automatically incorporated into the Users tagging resources List when and expert decision algorithm is imported into the Program. For example, a User/Judge imports a high level experts Decision Support Algorithm in relation to the law of Negligence. That Decision Support Algorithm may have (inter alia) a tag for each element of the course of action in Negligence, Duty of Care; Breach of Duty: Damage; and Causation. It may contain those plural tags that the author(s) of that algorithm require the User/Judge to make Factual Findings in that respect thereof to be adequately able to apply the law, in this case, of Negligence in a matter. The Judge may also generate their own tags any topic that appears to the Judge to be useful in respect of the specific Matter proceeding before them.

The Tagging functionality allows the Judge to tag all of the evidence, which can be done readily and easily, as the evidence unfolds in the Matter. That allows the Judge, in compilation Mode of the Program, to filter and extract all of the items in evidence in the Matter into plural Sub-Clipboards, literally at the touch of a button. The Judge can do so with a high level of confidence that they will have a comprehensive compilation of all of the data items relevance to that element of the course of action when are they select the tag “Duty of Care” to be applied as a filter extracting all items so tag from the entire data field of the Program into its own Sub-Clipboard, which can be given the name the Judge may consider appropriate, likely “Duty of Care”. This will save considerable judicial time and significantly increased the accuracy of the Judges Fact Finding task.

That Program allows the Judge to create their own Tags at any point in the use of the Decision System. They made, for example, identify a particular document in respect of which they wish to track all witnesses evidence; accordingly they may create a tag, for example, Credibility: Witness X, which they may use it to identify all data items where the credibility of Witness X may be raised. That may be in relation to the evidence of another witness, the content of some particular document, etc and selecting that tag in the Compilation mode of the System will enable the Judge to create a Sub-Clipboard containing every item of evidence which touches on the credibility of Witness X, and that Sub-Clipboard, “Credibility of Witness X”, will allow the Judge to have access to every item of evidence which allowed the Judge to draw their conclusion—that is make their finding if fact—about that witnesses credibility or believability.

The system allows multiple tags to be applied to every item of evidence in any data window. The User/Judge may also incorporate multiple tags into a single tag by holding down the Shift key when the User is selecting tags from the List of Tags.

The tagging functionality extends to colour tagging each item of data in a data window and the colour tags may be defined by the User in relation to their specified criteria. For example a red colour tag may be assigned to a specific data window to draw the attention of the Judge to the specific item of data contained in that data window—ie because the Judge has concerns in relation to it, and wants (inter alia) more information in relation to it. Blue may signify the Judge has formed the view that the evidence in the data window is settled and ready to be incorporated into their Judgment, and yellow may be selected to mark all evidentiary rulings, etc. It is a visual tool that will assist the Judge when reviewing the progress of the matter on the Primary Clipboard, which represents the chronological progression of all items of evidence as the Matter progresses.

The tagging window also allows a Judge to create a Chronology or timeline of the matter as it progresses. A Chronology or timeline is a critical resource in the Judges efficient management of the facts in a matter and a critical tool when organising and evaluating complex facts, as the date and time a particular event occurred will be of significance in the Judges understanding of how a matter factually evolved. Its importance as a tool in analysing and evaluating the facts is such that it is difficult to imagine any Judge in a complex matter being able to make confident factual findings without it. The User of the system, if of the view that an item of evidence is of significance, can enter a date, time and short description of the evidence in the data window, and the System will generate a complex and comprehensive timeline as the matter progresses. This will assist the Judge in their correct understanding of how the facts evolved and will be frequently referred to during the course of the Hearing (ie a Judge being able to assert to a witness, for example, that their evidence cannot be correct because the document about which they were giving edict was not in existence at the time that witness was asserting, or draw to the Judges attention that there is competing evidence about the date the said document was created. Chronologies are very valuable tools and the more detailed the Chronology the greater it's use is to the Judge in their fact finding function.

As the Chronology is of such significance and is likely to be constantly referred to during the course of a hearing, and when the Judge is making their factual findings and also when they are applying the law to the facts (is writing their Judgment) that the Decision Support System makes it a constant feature in every visual interface of the system.

The tagging window also allows the Judge to tag or link to that data window any document tendered in the Hearing and which has been entered into the Document management operation of the system in the Matters models the program. It may be used to link a particular witnesses evidence to a specific document (or part of a document extracted in the Document section of the Matters mode). For example, it may link a particular witnesses evidence to a contract, or to the travel records of an airline, etc. At any point where the Judge is reviewing that witnesses evidence, on that particular point, as recorded in that data window, the Judge may open and view any such linked documents in a data window which will open that document adjacent to the data window containing the linked document when the link button is executed. That functionality significantly enhances the ease of the Judicial function because those documents are immediate and readily available to the Judge as related to the witnesses evidence on that point.

The Judge may also link any digital resource to that data window using the hyperlink function.

The Compilation function of the system allows the Judge a very high level of control over their ability to manage, organise, analyse, compare and concatenate the data which has emerged across the course of the Matter. A Judge may multi-tag Witness X and Contract Y, enabling the Judge to extract all of Witnesses X's evidence about the contract from the totality of the evidence given by Witness X. The Judge may create tags identifying Expert X, Y and Z evidence on a specific issue of their evidence, (ie Witness X and Tensile Strength of Bridge Supports) and create a similar tag for the other expert evidence. That will allow the Judge to use the filtering process in the Tagging function to extract from the Hearing all of those items of each experts evidence relevant to that specific issue, to greatly facilitate the Judges ability to compare and evaluate the evidence on that point from each Expert. For example the specific evidence they gave and any documents which were relying upon may be drawn into the visual display in their respective Sub-Clipboards generated. Y these tags and the data windows containing their evidence on point, and any of the documents they specifically relied upon, can be opened in the same visual display, and the User may then create a new data window contains the Users conclusions on this issue and the System has allowed them to draw their factual conclusions predicated on the full identification of the evidence they have given and the documents they have relied upon.

The System also permits any number of Sub-Clipboards to be generated a visual display, permits the Judge to resize all such clipboards, and to select and open data windows from each of the Sub-Clipboards which may also be opened in the visual display and re-sized as appropriate, enabling the Judge to review plural items of evidence related to an issue in respect of which the Judge has to make a decision. The Decision Support System will allow the User to have a powerful, immediate and compressive capacity to compare, contrast, and evaluate the evidence from multiple sources during a Matter and in this interface of the system the Judge may also open into the visual display any linked document or item of multi-media contained in the documents location of the System. By way of example, the Judge could bring into the visual display CCTV footage of an assault in a park, and also have accessible all of the evidence of the 5 witnesses who were in the vicinity and each of which had given evidence of what they say occurred during the melee.

It is respectfully brought to the Examiners attention that this invention has the potential to very markedly increase the quality, consistency and thoroughness of the Judges capacity to take into account all items of evidence related to the matter in which they have to make a factual finding, noting that human processing capacity decreases markedly as the number of potential variables increases and the Decision Support System ensures all variables are collected and made available in a form which allows them to be readily analysed. It will minimise the risk a Judge will miss a potentially important item of evidence from multi variable items of evidence bearing on the decision they have to make.

After considering all concatenations and combinations of the tagged categories, the Judge may create from them a Sub-Clipboard containing their findings, or conclusions, of fact. A new Sub-Clipboard may be created in the Compilation mode of the Decision Support System for this purpose. The Judge may tag these Sub-Clipboards as Element of action and Conclusions (or such nomenclature as the Judge may determine) for example Duty of Care: Conclusions.

The Judge may apply tags to data windows at any stage of the operation of the Decision System.

[2] Applying the Facts Found to the Correct Legal Principles

After the Judges Fact Finding exercise has been completed, the second task the Judge must perform is the application of the law to the facts. The Judge, absent the support of the Decision Support System, must ensure that they have sufficient expertise or knowledge of the many legal issues to apply the correct legal principles without making any legal errors.

To do this, the Judge enters the Judgment mode of the program. In other applications of the program it may be described as the Report mode of the program.

The Judgment mode of the program presents in the right hand side of the visual display 2 columns with windows which allow the User to progress through the expert decision algorithm, a Judgment Clipboard and a Sub-Clipboard Window which enables the User to load into this visual display any Sub-Clipboard the User has generated in the compilation mode of the Decision Support System.

In the Judgment mode of the Decision Support System, the User is able to apply the facts they have found utilising the Compilation mode to the experts Decision Support Algorithm thereby generating the ultimate decision outcome of the system, in this case, their Judgment.

By way of an In-Use Description, the Judge is presiding over a case in Negligence. The Judge has made their findings of fact, guided by the tags imported from the Decision Support Algorithm into the tagging system, in the manner previously described. The tags relate to the legal elements of that cause of action and they are: Duty of Care; Breach of Duty; Damage; and Causation.

The Judge, when ready to decide whether the Negligence claim will succeed, opens program in Judgment mode, selects their List of Algorithms, selects Negligence and the system will open on that List of algorithm display, each of the topics the Judge has to make a decision by reference to the facts before them in the Matter to come to a conclusion about whether each element is satisfied.

The User selects the first of the elements, and the system opens that part of the experts algorithm in the visual display. By way of example, the expert system will in the first of the experts decision points in the data window, inform the User of the general applicable principle, that is, in what factual circumstances a Duty of Care arises. The algorithm in that decision point data window, will indicate the test to be applied, that being (for example) “a duty of care will arise when it is reasonably foreseeable that a persons conduct will cause injury or harm to a person”, and that experts data window on this decision point in their algorithm will include any case authority, statutory or regulatory rules that impact upon this issue, all of which may be hyperlinked so the Judge could readily access the relevant law. The experts algorithm will accordingly provide the judge with all the relevant legal data necessary to solve the problem, and that data is all linked to the expert steps necessary to solve the legal problem—ie to decide whether a Duty of Care arises.

That data window will also communicate to the Judge that they also need to consider the key subtopics contained in the following data window which is brought into in the Visual display by the expert algorithm to properly apply the law in relation to Duty of Care. That list will contain a bar and the Judge will elect each such topic, in which case its Data window and related subtopics will be moved sequentially up in the windows in this column of the display. Each sub-window may contain a list of the experts defined relevant sub-criteria: for example the first may be that there are special circumstances where the facts found generate a different application of the general principle if the plaintiff and defendant are in special relationships and the system will bring those relationships into the visual display as the User progresses deeper in to the decision algorithm. Such relationships may include teacher and student, that data window will provide the legal statement of that duty of care, supported by relevant case law and the experts explanation of how the law should be approached. This may be selected by the User if this factual circumstance arises in relation to the facts they have found and the user will elect that data window and load it into the Judgment Window. The User will then access the Duty of care findings on the Clipboard, or a further clipboard headed “Plaintiff and Defendants Relationship” with the Defendant, and will load their findings of fact in respect of that issue into the Sub-Clipboard. Once there, the Judge will be able to access and review all of their findings if fact on this point, and cut and paste the relevant conclusions into the statement of legal principle in the Judgment window. The Judge may edit the content of the stat window in the judgment window as may be appropriate, removing any extraneous material. After the end of the experts decision chain on special relationships and the Duty of Care, the System will return to User to the next matter they must take into account in the experts decision algorithm. If the User does not have any evidence in their factual findings of special relationships, as guided by the expert algorithm, they simply proceed to the next identified step in the experts decision algorithm and their Judgment is being constructed in the Judgment window.

The Judge proceeds through each stage of the experts approach to applying the law in relation to the factual findings the Judge has made in the Matter and the Judgment will proceed with a very high level of decision support.

The Judgment so generated will allow each User to have the benefit of very high level and expert decision algorithms improving their identification of, and best practice application of, the law on that particular topic (ie Negligence or in a criminal context Assault Occasioning Actual Bodily Harm) and will generate a Judgment in which the User will have had the support of a high level expert in how the law is to be applied, as well as the support of the Decision Support System in comprehensively and with a very enhanced level of accuracy, identified all of the items of evidence relevant to each legal issue.

The Judge will have had presented to them their findings of fact in the visual display related to the legal issues explained by a high level expert, which also contains all of the statutory, legislative and other plural resources which apply to that legal issue.

The User may avail themselves of the experts advice in the identification and correct application of the law they have to apply and this assist them in generating a high quality decision output, their Judgment. That Judgment will contain all of the legal material necessary for the Judge to correctly apply the law and an explanation of how that is properly done, and the Judge will have access to the data related to that specific legal issue in the Sub-Clipboard window.

The Judge will proceed through their Judgment Writing process, applying a best practice experts application of the legal principles from all relevant points in the process of preparing their judgment and the Examiner is respectfully reminded that the Decision Support System may be using an algorithm that has 100 decision points, where this particular User may only have (say) 30. The Decision output quality will accordingly be significantly enhanced, and the quality of their decision outputs significantly improved, and the Judge will have all material facts available to be incorporated in to the Judgment at each related point in the Decision Algorithm. A judgment so generated will be comprehensive in its application of the law, very considerably less likely to contain errors of law or fact, will reduce the cost to the public purse associated with appellate review of factual and legal errors, and save litigants considerable time, effort and cost in their use of the legal system.

It is further noted that the use of this Decision System has a significant advantage in the Users application of the law because it will take the User through the expert algorithm only to the extent required by the facts found by the User/Judge. For example, if the facts found by the User/Judge do not fall into any of the special categories identified in the experts algorithm, the User proceeds without having their application of the law confused by legal discussion in texts and other sources and or case law here it may not be clear to the user that not was being applied in relation to the special relationships category. The algorithm simply makes clear that if there are no such identified special categories the User continues in the next identified step in the experts algorithm.

It is noted that I am oversimplifying this illustration of how the algorithm applies the law. A fully worked example would take many pages of description and this In-Use application description is intended to highlight key aspects of the System to illustrate its operation in the practical context in which it would be used.

A Decision Algorithm that is prepared by a high level expert is anticipated to be one prepared by a person who will have practiced in relation to Negligence Matters for many years, and has integrated many decision points into their approach to applying the law of negligence to the facts, is highly experienced in matters of legal procedure, practice and pleadings which arise in Negligence Matters, and who has integrated all of these various legal resources into their own decision algorithm.

Until such expertise is acquired, a User will invariably have the quality of their decision output enhanced and perhaps significantly enhanced by their access to the decision support algorithm of the expert when deciding a Negligence Matter.

It is further noted that application of the experts decision algorithm will improve (upskill) the quality of the User/Judges knowledge of that area of the law to which the expert Algorithm applies. It is akin to having (for example) an expert mechanic looking over the shoulder of an apprentice or recently qualified mechanic, the less experienced having read the textbook and practical guidelines about a difficult mechanical repair job in the vehicle, but has the advantage of the expert who can point out subtleties and perhaps unusual problems and forestall the less experienced mechanic making a mistake when they are performing a complex task in relation to repairing a vehicle. It is respectfully submitted that everyone knows how useful that can be and how it can alter the outcome of the less experienced (in this case) mechanics work on a vehicle, minimise the risk of the less experienced mechanic falling into error, and will increase significantly that junior mechanics store of knowledge about the procedure, and produce a better and safer level of service to the vehicle in question.

The User/Judge may follow the same process in respect of applying the expert algorithms to the facts found as the Judge progresses in relation to each cause of action argued by the parties in the Matter. For example, it may be that they sequentially move through decision support algorithms in relation to other causes of action such as negligence (as illustrated), breach of contract, breach of Sale of Goods Act, misrepresentation and fraud. One of the consequences noted in the explosion of the amount and complexity of the law is that it is very difficult for a Judge or lawyer to maintain a high level of expertise across a wide range of legal subjects—so the capacity the Decision Support System has to provide the. The support of a high quality best practice algorithm prepared by a recognised expert gives them significant decision support which will significantly enhance the quality and consistency of judicial decision making by less experienced Users of the Program.

It is also noted that the System allows the generation if an expert Decision Algorithm by more than 1 expert. It is possible for (say) the leading 20 oncologists in the country to work jointly on a Decision Support Algorithm in respect of diagnosis and treatment of a particular form of cancer and that Decision Algorithm would contain all the decision points of these multitude of specialists producing an extremely powerful diagnoses and treatment tool that could be available to assist medical practitioners worldwide. As noted in the original filing, for example, it could mean that a doctor or nurse medical practitioner in a very remote part of Africa could have the advantage of a Harvard infections disease experts approach to diagnoses and treatment of an ebola outbreak and the decision support algorithm of a world leading epidemiologist (s) on how to best contain such an outbreak. Further it would allow such a User to forward a copy of their Report and all the steps taken to send that report in real time via email to governmental agencies such as the Centre for Disease Control and Protection in Atlanta, Ga., who will immediately be appraised of what steps the User has taken, the reasons why, and the factual background that informed those decision choices. It is respectfully submitted that the benefits of such a decision support system in terms of rapid and effective response needs little further amplification.

It is further noted that a specific Decision Support Algorithm my receive accreditation by some professional body and such accreditation may reduce the insurance burden on professional users who confirm they used it in their decision making activities, as the insurer will have some confidence that specified matters in the experts approach will be taken into account by the User and that the Decision Support Algorithm complies with the accreditation agencies best practice guidelines.

A User may also utilise a Decision Support Algorithm at any stage in the operation of the program.

This would occur, for example, if an argument was raised on an objection to tender an item of evidence that such item was not relevant as it was asserted it could not inform the Courts decision about whether a Duty of Care is established.

The User/Judge may load the expert algorithm in Negligence into the visual display, look at the law relating to Duty of Care, and be able to reference that to respond to the evidential objection by stating (for example) the item if evidence is relevant because it related to how “reasonably foreseeable” the injury was.

It is respectfully submitted that this invention presents a novel system which will greatly enhance a Uses ability to manage the complex factual data in a Hearing/Trial. It will significantly reduce the time a Judge will have to take in determining their factual findings after a Matter, and it will enable thorough and comprehensive analysis of the facts in the matter. It is asserted that this system will operate to significantly reduce the risk of appeal correction with the attendant and very significant costs that this imposes on both of the public purse and to the litigants.

Further, in providing a system to enable a best standard identification and application of the legal principles a Judge is required to apply to the facts, the Decision System will significantly enhance the quality and consistency of judicial decision-making as it allows an expert's approach to the application of the law to be applied by the User and it is asserted that this System provides a novel structure allowing a User/Judge to assemble a high standard Judgment that has a significantly enhanced level of legal accuracy than they would generally otherwise able to generate as well as increasing the quality of the factual evaluations necessary in their fact-finding function.

Clearly, they misapplication of legal principle is also a very common source of intervention of a Matter on appeal. That risk will be significantly reduced when a Decision Maker has the benefit of an expert's approach to the application of the law provided by an expert algorithm supported by the identified ways in which the fact-finding task is also highly supported within this Decision Support System.

The Decision Support System is modular. It allows the user to select a variety of highly specialised Decision Algorithms from what will ultimately be a very wide range of sources to enable them to highly customise their Decision System with those specialist algorithms that directly support their specific decision tasks.

It is also submitted that this Decision System may offer a considerable enhancement to the availability of expert knowledge in the world. It will allow any User of the System, who may acquire specialised knowledge on any topic to generate a Decision Algorithm on that topic and make it available immediately to a potentially worldwide group of Users of the System.

For example, a person in one country may be working on a very specialised technique in respect of control of infections, and have derived a particular procedure and set of expert steps for that purpose (say) in a childrens clinical environment, and may use the System to generate a decision algorithm on point which can be immediately made available to assist other medical personnel around the world dealing with similar issues.

It is argued that it has the potential to rapidly increase the topics of expert processes that could be used to direct and guide other Users in how to apply that specific process contained in that decision algorithm themselves.

The decision support system will also allow a specific decision algorithm to be exported as a run time, enabling a particular decision algorithm to be able to be sold as a discrete item and able to be used by a user having purchased it from some external source on their laptop or device. This would enable the program to be utilised to make available to Uses more discreet expert decision algorithms such as conveyancing or particular legal processes such as dividing fences applications etc.

The program is able to run on plural devices, including desktop computers, laptop computers, tablet computers and smart phones.

The System is not limited to use in respect of Legal Matters such as trials and hearings. In law, it has wider application, for example, allowing an experts Decision Algorithm in respect of the compilation of specific types of contracts to be generated. The System would operate by importing into the System with the decision support algorithm relevant to that category of contract tags identifying the matters in respect of which the User will need to collate data as well as the first step in that expert algorithm advising the user in relation to the detailed aspects of the data necessary to collect prior to it being assembled in the Report mode, illustrated in the current diagrams as the Judgement mode. The System will thereby enable a high level of legal information and assistance to be provided in respect of a Uses preparation of appropriate clauses in the preparation of that particular type of contract, for example, a standard franchise agreement contract. In this application, the Decision Support System would Generate a considerably better quality contract and in such operation may be designed to provide access to expert knowledge to non-legal users of the program. The program could be designed to provide non-lawyers with assistance in respect of matters for which they may. Ot be able to afford or may not wish to retain lawyers and would generate for them a considerably higher quality decision output, the preparation of a contract that better reflects their commercial interests, than if they did not have access to such a level of decision support.

A further illustration of the use of the Decision Support System in a non-legal context is its application supporting standardised but complex investigation processes, particularly where such investigations may require many investigation claims assessors assessing multiple claims such as arose by way of example after the catastrophic earthquake that almost destroyed the New Zealand city of Christchurch. The algorithm would contain the parent insurance companies best standard approach to the process it required it's investigators to follow and provide them with support in ensuring they collected all the data relevant to the advancement if the claim. The plural items of data may be collected, tagged, and then applied in the manner described to the steps and decisions the expert insurance claims assessor has designed. That is particularly useful when many of the Users may be inexperienced with that company or the application of its assessment processes. It is noted in that respect that many claims assessors were employed on short notice and from counties such as Australia to fly in and to perform the massive insurance assessment work that resulted from the extensive property damage and many of these persons were, for example, persons with no prior insurance investigation experience being retired police officers etc as there were simply inadequate specialist assessors to perform the necessary work. The Decision Support System would ensure that a quality and standardised approach would be generated and applied by all Users all the Users, that all documents necessary for the claim to be assessed by Management would be generated during the process, and the Report generated would be consistent and thorough facilitation of the assessment. The System would strongly support the assessors quality of fact finding and their required organisation and evaluation of the facts, how those facts informed their assessment of the claims made against the insurance company and generate a fully reasoned report that addressed all of the specialised concerns the company required and would allow the report to be very rapidly generated and which would render totally transparent the assessors reasons for the recommendations they made.

Embodiments of the system bus described provide a user—in this instance-a Judge with a Judgment assembly System which provides such a high level of support in both aspects of a Judges function: finding the facts and then applying them to a high quality expert application of the law, as well as providing assistance to the Judge in applying complex legal decisions to matters arising during the course of the Matter, and this Decision. support System has equal application in relation to other complex decision systems as illustrated, as well as comprising a structure which will allow multiple experts to combine their research, knowledge and expertise into a decision algorithm that could powerfully exceed the capacity of a single expert.

### INDUSTRIAL APPLICABILITY

Embodiments of the present application facilitate the management of decision making processes—particularly but not only where the decision making process benefits from reference to expert decision making and where large volumes of information need to be visualized and interrelated visually. The visualisation allows the user to make complex linkages between many and varied data items.

1. A decision support system for a matter; the matter of indeterminate duration; and during the course of the matter third-party inputs are received consecutively and periodically for the duration of the matter; The decision support system allowing replication of an expert's approach to a complex problem; said system comprising:
a primary clipboard into which data is entered by the user into one or more data windows;
generated multiple sub-clipboards from the primary clipboard and wherein
all sub-clipboards contain plural data windows;
a database of public information comprising data modules for selective input into a Data Matrix relevant to the matter;
preconfigured specialist algorithms assembled by an expert knowledgeable in decision-making in relation to the matter; the specialist algorithms contained in a Decision Support mode of the decision support system;
an input system by which the third-party inputs comprising each item of data presented to the User as a matter progresses are consecutively and periodically assembled by a user at first instance on to the primary clipboard;
an input system by which the user selects portions of the public information and portions of the preconfigured specialist algorithms and assembles them at first instance onto the primary clipboard;
and wherein the selections and assembly made in response to all items of data received during the matter are entered onto the primary clipboard;
thereby to assemble a primary clipboard of public information portions, specialist algorithm portions and third-party input portions (define) in a continuous, timestamp format so as to present a chronological record of all items of data and decisions made by the user as the matter progresses with the assistance of the specialist algorithms and data modules relevant to the matter and as selected by the user as the matter progresses; and wherein
the items of the data on the primary clipboard are
tagged by the user with tags during the course of the matter or by the User when the user is preparing their decision or report;
and wherein the tag is a categorising device by which an item of data is characterised according to a criterion selected by the user from the Decision Support System;
and wherein Every item of data entered into the Primary Clipboard is assembled by the User as the matter progresses; every item of data able to be edited by way of both imported and user generated multiple tags to enable the content of the Primary Clipboard to be filtered into the Sub-Clipboards in accordance with the tags selected by the User; and
wherein the User generates a specialist display of the system in which the User may populate a Report (in the instant legal application, a Judgment) which display presents the selected decision support algorithm, as well as a sub-clipboard into which the User may select any sub clipboard containing the fact findings the User has made which are relevant to the selected decision support algorithm; and which display allows the user to assemble their Report (in the In-Use example a Judgment) by copying relevant data windows (or data from data window) from the decision support algorithm into the Report (or Judgment) Clipboard and then to populate that data window in the Report or Judgment Clipboard with data from the sub-clipboard in the adjacent display of the program containing the Users findings of fact, and which allows the User to so work sequentially though the topics from the expert decision algorithm on which the User must make an outcome decision; and thereby generating a Report (or Judgment) as the ultimate outcome of the process of applying the expert decision topics (in the in-use example the law) to the facts across the matter, being those facts identified as relevant to every decision in the decision algorithm through the assistance provided in other models of operation of the system.

2. The decision support system of claim 1 incorporating further Plural integrated tagging functionality including colour tagging data windows, linking data windows to documents and other media saved into the decision system and generation of a chronology of all items of significance in the matter.

3. The decision support system of claim 2 wherein an item of data may be tagged with more than one tag thereby categorising the item of data according to more than one criterion.

4. The decision support system of claim 3 wherein all items of data which are tagged by a tag according to a particular criterion may be assembled by the system, on command of the user, into a sub clipboard or independent data window in a compilation mode of the system's operation.

5. The decision support system of claim 4; the system extracting from the field of data collected by the user onto the Primary Clipboard during the Matter into Sub-Clipboards containing all data windows related to the Users selected tagging criteria.

6. The decision support system of claim 5 wherein the system allows a user to select one or more sub clipboards in a particular visual display and from those sources open and compare multiple data windows to view them at the same time then each independent data window displaying links to related documents which a user may also bring into that specialist display; items of data tagged according to a different criterion so that the user is enabled to select one or more sub clipboards in a particular way to view more than one independent data window from multiple Sub Clipboards at the same time; each independent data window displaying items of data tagged according to a different criterion in each independent data window.

7. The decision support system of claim 1 wherein the matter relates to a legal investigation in the form of a court case.

8. The decision support system of claim 1 wherein the matter relates to an investigation process relating to a medical investigation.

9. The decision support system of claim 1 wherein the matter relates to a scientific investigation.

10. The decision support system of claim 1 wherein the matter relates to an insurance investigation.

11. The decision support system of claim 1 wherein a user may invoke steps in the specialist algorithm in order from less complex to more complex-only to the level needed for the particular matter.

12. The decision support system of claim 1 wherein the criterion for a tag may be determined by the user or automatically imported, as part of a specialist decision algorithm which is imported into the decision system by the user.

13. The decision support system of claim 1 wherein Tags may be imported by the user.

14. The decision support system of claim 1; said system comprising the primary clipboard which allows all data relevant to the matter from plural third-party sources from the system in data matrix and decision support mode to be input into the primary clipboard through a system of data windows which permits a user a powerful system to manage, link, and compare all of the data on the primary clipboard in any configuration selected by the user utilising the tagging function in each data window.

15. The decision support system of claim 1 wherein the system allows an expert decision algorithm to contain tags which will be automatically incorporated into the tagging functionality of the system which relates to the plural factual matters the user has to decide to correctly apply the law and wherein the user will also be able to add their own plural tags as they may consider appropriate.

16. The decision support system of claim 1 wherein the system has colour functionality for each data window.

17. The system of claim 16 wherein the user may attach a colour to each data window to operate as a reminder to the user when reviewing the items in the primary clipboard in accordance with a user selected criteria by reference to the colours.

18. The decision support system of claim 1 wherein the data window in the selection of the chronology button permits the user to incorporate the event in the data window into a timeline which will be generated during the conduct of the matter.

19. The decision support system of claim 18 wherein the chronology will be available to the user in every mode of the system.

20. The decision support system of claim 1 wherein there are at least 6 visual interfaces/modes of operation comprising Matters, Data Matrix, Decision Support, Compilation, Judgment and Chronology

21. The decision support system of claim 20 wherein the specialist algorithms are assembled by an expert knowledgeable in decision-making in relation to the matter contained in Decision Support mode of the Program.

22. The decision support system of claim 1 is modular and permits the user to generate and export from the system their own data matrix modules and their own decision support modules to be available for incorporation by other users of the system into their respective decision support algorithm lists or their data matrix, of public information.
